(19) United States
(12) Patent Application Publication
(10) Pub. No.: US 2021/0355121 A1
(43) Pub. Date: Nov. 18, 2021

(54) FUSED TRICYCLIC KRAS INHIBITORS

(71) Applicant: Incyte Corporation, Wilmington, DE (US)

(72) Inventors: Wenyu ZHU, Wilmington, DE (US); Xiaozhao WANG, Mt. Laurel, NJ (US); Artem SHVARTSBART, Wilmington, DE (US); Chao Qi, Wilmington, DE (US); Rocco Policarpo, Wilmington, DE (US); Wenqing Yao, Wilmington, DE (US)

(21) Appl. No.: 17/231,547

(22) Filed: Apr. 15, 2021

Related U.S. Application Data
(60) Provisional application No. 63/011,089, filed on Apr. 16, 2020.
(60) Provisional application No. 63/146,899, filed on Feb. 8, 2021.

(51) Int. Cl. C07D 471/04; C07D 519/00; A61P 35/00; A61K 9/00

(52) U.S. Cl. CPC C07D 471/04; A61K 9/0053; A61P 35/00; C07D 519/00

(57) ABSTRACT

Disclosed are compounds of Formula I, methods of using the compounds for inhibiting KRAS activity and pharmaceutical compositions comprising such compounds. The compounds are useful in treating, preventing or ameliorating diseases or disorders associated with KRAS activity such as cancer.

### RELATED APPLICATIONS

This application is related to U.S. Provisional Application No. 63/011,089 filed on Apr. 16, 2020 and U.S. Provisional Application No. 63/146,899 filed on Feb. 8, 2021, the entire contents of which are hereby incorporated by reference in their entirety.

### FIELD OF THE INVENTION

The disclosure provides compounds as well as their compositions and methods of use. The compounds modulate KRAS activity and are useful in the treatment of various diseases including cancer.

### BACKGROUND OF THE INVENTION

Ras proteins are part of the family of small GTPases that are activated by growth factors and various extracellular stimuli. The Ras family regulates intracellular signaling pathways responsible for growth, migration, survival and differentiation of cells. Activation of RAS proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (Simanshu, D. et al. Cell 170.1 (2017):17-33).

The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (Simanshu, D. et al. Cell 170.1 (2017):17-33). KRAS mutations are prevalent amongst the top three most deadly cancer types: pancreatic (97%), colorectal (44%), and lung (30%) (Cox, A. D. et al. Nat Rev Drug Discov (2014) 13:828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12 V mutations are associated with pancreatic cancers (30%), followed by colorectal adenocarcinomas (27%) and lung adenocarcinomas (23%) (Cox, A. D. et al. Nat Rev Drug Discov (2014) 13:828-51). In contrast, KRAS G12C mutations predominate in non-small cell lung cancer (NSCLC) comprising 11-16% of lung adenocarcinomas, and 2-5% of pancreatic and colorectal adenocarcinomas (Cox, A. D. et al. Nat. Rev. Drug Discov. (2014) 13:828-51). Genomic studies across hundreds of cancer cell lines have demonstrated that cancer cells harboring KRAS mutations are highly dependent on KRAS function for cell growth and survival (McDonald, R. et al. Cell 170 (2017): 577-592). The role of mutant KRAS as an oncogenic driver is further supported by extensive in vivo experimental evidence showing mutant KRAS is required for early tumour onset and maintenance in animal models (Cox, A. D. et al. Nat Rev Drug Discov (2014) 13:828-51).

Taken together, these findings suggest that KRAS mutations play a critical role in human cancers; development of inhibitors targeting mutant KRAS may therefore be useful in the clinical treatment of diseases that are characterized by a KRAS mutation.

### SUMMARY

The present disclosure provides, inter alia, a compound of Formula I:

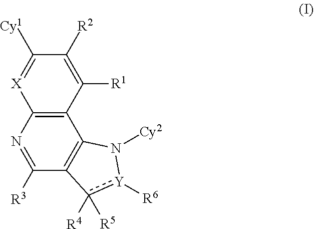

or a pharmaceutically acceptable salt thereof, wherein constituent variables are defined herein.

The present disclosure further provides a pharmaceutical composition comprising a compound of the disclosure, or a pharmaceutically acceptable salt thereof, and at least one pharmaceutically acceptable carrier or excipient.

The present disclosure further provides methods of inhibiting KRAS activity, which comprises administering to an individual a compound of the disclosure, or a pharmaceutically acceptable salt thereof. The present disclosure also provides uses of the compounds described herein in the manufacture of a medicament for use in therapy. The present disclosure also provides the compounds described herein for use in therapy.

The present disclosure further provides methods of treating a disease or disorder in a patient comprising administering to the patient a therapeutically effective amount of a compound of the disclosure, or a pharmaceutically acceptable salt thereof.

### DETAILED DESCRIPTION

### Compounds

In an aspect, provided herein is a compound of Formula I:

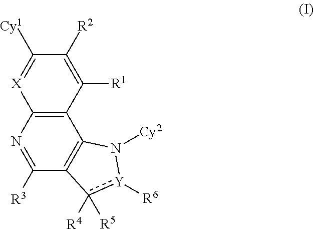

or a pharmaceutically acceptable salt thereof,

wherein:

each [symbol] independently represents a single bond or a double bond;

X is N or CR⁷;

Y is N or C;

R¹ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, and BR^{h1}Rⁱ¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R^{g};

R² is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, and BR^{h2}Rⁱ²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;

Cy¹ is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R¹⁰;

R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{f3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{j3}, NR^{c3}C(O)R^{b3}, NR^{c3}C(O)OR^{a3}, NR^{c3}C(O)NR^{c3}R^{d3}, C(═NR^{e3})R^{b3}, C(═NOR^{a3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, and BR^{h3}Rⁱ³; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³⁰;

when R⁴R⁵C [symbol] YR⁶ is a single bond and Y is C, then YR⁶ is selected from C═O and C═S; and

R⁴ is selected from H, D, C₁₋₆ alkyl, C₂₋₆ alkenyl, C₂₋₆ alkynyl, C₁₋₆ haloalkyl, C₃₋₆ cycloalkyl, 4-6 membered heterocycloalkyl, phenyl, 5-6 membered heteroaryl, halo, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{d4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, and BR^{h4}Rⁱ⁴; wherein said C₁₋₆ alkyl, C₂₋₆ alkenyl, C₂₋₆ alkynyl, C₃₋₆ cycloalkyl, 4-6 membered heterocycloalkyl, phenyl, and 5-6 membered heteroaryl, are each optionally substituted with 1, 2, 3, or 4 substituents independently selected from R^{g};

R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NOR^{a5})R^{b5}, C(═NR^{e5})NR^{c5}R^{d5}, NR^{c5}C(═NR^{e5})NR^{c5}R^{d5} NR^{c5}C(═NR^{e5})R^{b5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, and BR^{h5}Rⁱ⁵; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵⁰;

when R⁴R⁵C [symbol] YR⁶ is a double bond and Y is N, then R⁴ and R⁶ are absent;

when R⁴R⁵C⁵ [symbol] YR⁶ is a double bond and Y is C, then R⁴ is absent; and

R⁶ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NOR^{a6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, and BR^{h6}Rⁱ⁶; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶⁰;

R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NOR^{a7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, and BR^{h7}Rⁱ⁷; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷⁰;

Cy² is selected from C₃₋₁₀ cycloalkyl, 4-14 membered heterocycloalkyl, C₆₋₁₀ aryl and 5-10 membered heteroaryl; wherein the 4-14 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-14 membered heterocycloalkyl is optionally substituted by oxo to form a carbonyl group; and wherein the C₃₋₁₀ cycloalkyl, 4-14 membered heterocycloalkyl, C₆₋₁₀ aryl and 5-10 membered heteroaryl are each optionally substituted with 1, 2, 3 or 4 substituents independently selected from R²⁰;

each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a10}, SR^{a10}, C(O)R^{b10}, C(O)NR^{c10}R^{d10}C(O)OR^{a10}C(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, C(═NR^{e10})R^{b10}, C(═NOR^{a10})R^{b10}, C(═NR^{e10})NR^{c10}R^{d10}, NR^{c10}C(═NR^{e10})NR^{c10}R^{d10}, NR^{c10}S(O)R^{b10}, NR^{c10}S(O)₂R^{b10}, NR^{c10}S(O)₂NR^{c10}R^{d10}S(O)R^{b10}, S(O)NR^{c10}R^{d10}, S(O)₂R^{b10}, S(O)₂NR^{c10}R^{d10}, and BR^{h10}Rⁱ¹⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;

each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, and BR^{h11}Rⁱ¹¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²; each R¹² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)R^{b12}, NR^{c12}S(O)₂R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, and BR^{h12}Rⁱ¹²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};

each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a20}, SR^{a20}, C(O)R^{b20}, C(O)NR^{c20}R^{d20}, C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, C(═NR^{e20})R^{b20}, C(═NOR^{a20})R^{b20}, C(═NR^{e20})NR^{c20}R^{d20}, NR^{c20}C(═NR^{e20})NR^{c20}R^{d20}, NR^{c20}S(O)R^{b20}, NR^{c20}S(O)₂R^{b20}, NR^{c20}S(O)₂NR^{c20}R^{d20}, S(O)R^{b20}, S(O)NR^{c20}R^{d20}, S(O)₂R^{b20}, S(O)₂NR^{d20}R^{d20}, and BR^{h20}Rⁱ²⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;

each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)R^{b21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, and BR^{h21}Rⁱ²¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R^{g};

each R²² is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)R^{b22}, NR^{c22}S(O)₂R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, and BR^{h22}Rⁱ²²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²³;

each R²³ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a23}, SR^{a23}, C(O)R^{b23}, C(O)NR^{c23}R^{d23}, C(O)OR^{a23}, OC(O)R^{b23}, OC(O)NR^{c23}R^{d23}, NR^{c23}R^{d23}, NR^{c23}C(O)R^{b23}, NR^{c23}C(O)OR^{a23}, NR^{c23}C(O)NR^{c23}R^{d23}, NR^{c23}S(O)R^{b23}, NR^{c23}S(O)₂R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, and BR^{h23}Rⁱ²³; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²⁴;

each R²⁴ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a24}, SR^{a24}, C(O)R^{b24}, C(O)NR^{c24}R^{d24}, C(O)OR^{a24}, OC(O)R^{b24}, OC(O)NR^{c24}R^{d24}, NR^{c24}R^{d24}, NR^{c24}C(O)R^{b24}, NR^{c24}C(O)OR^{a24}, NR^{c24}C(O)NR^{c24}R^{d24}, NR^{c24}S(O)R^{b24}, NR^{c24}S(O)₂R^{b24}, NR^{c24}S(O)₂NR^{c24}R^{d24}, S(O)R^{b24}, S(O)NR^{c24}R^{d24}, S(O)₂R^{b24}, S(O)₂NR^{c24}R^{d24}, and BR^{h24}Rⁱ²⁴; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};

each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a30}, SR^{a30}, C(O)R^{b30}, C(O)NR^{c30}R^{d30}, C(O)OR^{a30}, OC(O)R^{b30}, OC(O)NR^{c30}R^{d30}, NR^{c30}R^{d30}, NR^{c30}C(O)R^{b30}, NR^{c30}C(O)OR^{a30}, NR^{c30}C(O)NR^{c30}R^{d30}, NR^{c30}S(O)R^{b30}, NR^{c30}S(O)₂R^{b30}, NR^{c30}S(O)₂NR^{c30}R^{d30}, S(O)R^{b30}, S(O)NR^{c30}R^{d30}, S(O)₂R^{b30}, S(O)₂NR^{c30}R^{d30}, and BR^{h30}Rⁱ³⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³¹;

each R³¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a31}, SR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR^{c31}C(O)R^{b31}, NR^{c31}C(O)OR^{a31}, NR^{c31}C(O)NR^{c31}R^{d31}, NR^{c31}S(O)R^{b31}, NR^{c31}S(O)₂R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, and BR^{h31}Rⁱ³¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³²;

each R³² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a32}, SR^{a32}, C(O)R^{b32}, C(O)NR^{c32}R^{d32}, C(O)OR^{a32}, OC(O)R^{b32}, OC(O)NR^{c32}R^{d32}, NR^{c32}R^{d32}, NR^{c32}C(O)R^{b32}, NR^{c32}C(O)OR^{a32}, NR^{c32}C(O)NR^{c32}R^{d32}, NR^{c32}S(O)R^{b32}, NR^{c32}S(O)₂R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32}, and BR^{h32}Rⁱ³²; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};

each R⁵⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a50}, SR^{a50}, C(O)R^{b50}, C(O)NR^{c50}R^{d50}, C(O)OR^{a50}, OC(O)R^{b50}, OC(O)NR^{c50}R^{d50}, NR^{c50}R^{d50}, NR^{c50}C(O)R^{b50}, NR^{c50}C(O)OR^{a50}, NR^{c50}C(O)NR^{c50}R^{d50}, NR^{c50}S(O)R^{b50}, NR^{c50}S(O)₂R^{b50}, NR^{c50}S(O)₂NR^{c50}R^{d50}, S(O)R^{b50}, S(O)NR^{c50}R^{d50}, S(O)₂R^{b50}, S(O)₂NR^{c50}R^{d50}, and BR^{h50}Rⁱ⁵⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵¹;

each R⁵¹ is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c51}C(O)NR^{c51}R^{d51}, NR^{c51}S(O)R^{b51}, NR^{c51}S(O)₂R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, and BR^{h51}Rⁱ⁵¹; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R⁵²;

each R⁵² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a52}, SR^{a52}, C(O)R^{b52}, C(O)NR^{c52}R^{d52}, C(O)OR^{a52}, OC(O)R^{b52}, OC(O)NR^{c52}R^{d52}, NR^{c52}R^{d52}, NR^{c52}C(O)R^{b52}, NR^{c52}C(O)OR^{a52}, NR^{c52}C(O)NR^{c52}R^{d52}, NR^{c52}S(O)R^{b52}, NR^{c52}S(O)₂R^{b52}, NR^{c52}S(O)₂NR^{c52}R^{d52}, S(O)R^{b52}, S(O)NR^{c52}R^{d52}, S(O)₂R^{b52}, S(O)₂NR^{c52}R^{d52}, and BR^{h52}Rⁱ⁵²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};

each R⁶⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a60}, SR^{a60}, C(O)R^{b60}, C(O)NR^{c60}R^{d60}C(O)OR^{a60}, OC(O)R^{b60}, OC(O)NR^{c60}R^{d60}, NR^{c60}R^{d60}, NR^{c60}C(O)R^{b60}, NR^{c60}C(O)OR^{a60}, NR^{c60}C(O)NR^{c60}R^{d60}, NR^{c60}S(O)R^{b60}, NR^{c60}S(O)₂R^{b60}, NR^{c60}S(O)₂NR^{c60}R^{d60}, S(O)R^{b60}, S(O)NR^{c60}R^{d60}, S(O)₂R^{b60}, S(O)₂NR^{c60}R^{d60}, and BR^{h60}Rⁱ⁶⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R^{g};

each R⁷⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a70}, SR^{a70}, C(O)R^{b70}, C(O)NR^{c70}R^{d70}, C(O)OR^{a70}, OC(O)R^{b70}, OC(O)NR^{c70}R^{d70}, NR^{c70}R^{d70}, NR^{c70}C(O)R^{b70}, NR^{c70}C(O)OR^{a70}, NR^{c70}C(O)NR^{c70}R^{d70}, NR^{c70}S(O)R^{b70}, NR^{c70}S(O)₂R^{b70}, NR^{c70}S(O)₂NR^{c70}R^{d70}, S(O)R^{b70}, S(O)NR^{c70}R^{d70}, S(O)₂R^{b70}, S(O)₂NR^{c70}R^{d70}, and BR^{h70}Rⁱ⁷⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷¹;

each R⁷¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)R^{b71}, NR^{c71}S(O)₂R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, and BR^{h71}Rⁱ⁷¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷²;

each R⁷² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a72}, SR^{a72}, C(O)R^{b72}, C(O)NR^{c72}R^{d72}, C(O)OR^{a72}, OC(O)R^{b72}, OC(O)NR^{c72}R^{d72}, NR^{c72}R^{d72}, NR^{c72}C(O)R^{b72}, NR^{c72}C(O)OR^{a72}, NR^{c72}C(O)NR^{c72}R^{d72}, NR^{c72}S(O)R^{b72}, NR^{c72}S(O)₂R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72}, and BR^{h72}Rⁱ⁷²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};

each R^{a1}, R^{b1}, R^{c1}, and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};

or any R^{c1} and R^{d1} attached to the same N atom, together with the N atom to which they are attached, form a 4-, 5-, 6- or 7-membered heterocycloalkyl group optionally substituted with 1, 2 or 3 substituents independently selected from R^{g};

each R^{h1} and Rⁱ¹ is independently selected from OH, C₁₋₆ alkoxy, and C₁₋₆ haloalkoxy; or any R^{h1} and Rⁱ¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a2}, R^{b2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²²;

or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R²²;

each R^{e2} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h2} and Rⁱ² is independently selected from OH, C₁₋₆ alkoxy, and C₁₋₆ haloalkoxy; or any R^{h2} and Rⁱ² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a3}, R^{b3}, R^{c3} and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{e3} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{f3} and R^{j3} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, CB-10 aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{h3} and Rⁱ³ is independently selected from OH, C₁₋₆ alkoxy, and C₁₋₆ haloalkoxy; or any R^{h3} and Rⁱ³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a4}, R^{b4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};

or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R^{g};

each R^{h4} and Rⁱ⁴ is independently selected from OH, C₁₋₆ alkoxy, and C₁₋₆ haloalkoxy; or any R^{h4} and Rⁱ⁴ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a5}, R^{b5}, R^{c5} and R^{d5} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁵⁰;

or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R⁵⁰;

each R^{e5} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h5} and Rⁱ⁵ is independently selected from OH, C₁₋₆ alkoxy, and C₁₋₆ haloalkoxy; or any R^{h5} and Rⁱ⁵ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a6}, R^{b6}, R^{c6} and R^{d6} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶⁰;

or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R⁶⁰;

each R^{e6} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h6} and Rⁱ⁶ is independently selected from OH, C₁₋₆ alkoxy, and C₁₋₆ haloalkoxy; or any R^{h6} and Rⁱ⁶ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a7}, R^{b7}, R^{c7} and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁷⁰;

or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R⁷⁰;

each R^{e7} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h7} and Rⁱ⁷ is independently selected from OH, C₁₋₆ alkoxy, and C₁₋₆ haloalkoxy; or any R^{h7} and Rⁱ⁷ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a10}, R^{b10}, R^{c10} and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹; or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R¹¹;

each R^{e10} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h10} and Rⁱ¹⁰ is independently selected from OH, C₁₋₆ alkoxy, and C₁₋₆ haloalkoxy; or any R^{hi0} and Rⁱ¹⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a11}, R^{b11}, R^{c11} and R^{d11}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;

or any R^{c11} and R^{d11} attached to the same N atom, together with the N atom to which they are attached, form a 4-, 5-, 6- or 7-membered heterocycloalkyl group optionally substituted with 1, 2 or 3 substituents independently selected from R¹²;

each R^{h11} and Rⁱ¹¹ is independently selected from OH, C₁₋₆ alkoxy, and C₁₋₆ haloalkoxy; or any R^{h11} and Rⁱ¹¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a12}, R^{b12}, R^{c12} and R^{d12}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};

each R^{h12} and Rⁱ¹² is independently selected from OH, C₁₋₆ alkoxy, and C₁₋₆ haloalkoxy; or any R^{h12} and Rⁱ¹² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²¹;

or any R^{o20} and R^{d20} attached to the same N atom, together with the N atom to which they are attached, form a 4-, 5-, 6- or 7-membered heterocycloalkyl group optionally substituted with 1, 2, 3, or 4 substituents independently selected from R²¹;

each R^{e20} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h20} and Rⁱ²⁰ is independently selected from OH, C₁₋₆ alkoxy, and C₁₋₆ haloalkoxy; or any R^{h20} and Rⁱ²⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a21}, R^{b21}, R^{c21} and R^{d21}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R^{g};

or any R^{c21} and R^{d21} attached to the same N atom, together with the N atom to which they are attached, form a 4-, 5-, 6- or 7-membered heterocycloalkyl group optionally substituted with 1, 2 or 3 substituents independently selected from R^{g};

each R^{h21} and Rⁱ²¹ is independently selected from OH, C₁₋₆ alkoxy, and C₁₋₆ haloalkoxy; or any R^{h21} and Rⁱ²¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a22}, R^{b22}, R^{c22} and R^{d22} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²³;

or any R^{c22} and R^{d22} attached to the same N atom, together with the N atom to which they are attached, form a 4-, 5-, 6- or 7-membered heterocycloalkyl group optionally substituted with 1, 2, 3, or 4 substituents independently selected from R²³;

each R^{h22} and Rⁱ²² is independently selected from OH, C₁₋₆ alkoxy, and C₁₋₆ haloalkoxy; or any R^{h22} and Rⁱ²² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a23}, R^{b23}, R^{c23} and R^{d23}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²⁴;

or any R^{c23} and R^{d23} attached to the same N atom, together with the N atom to which they are attached, form a 4-, 5-, 6- or 7-membered heterocycloalkyl group optionally substituted with 1, 2 or 3 substituents independently selected from R²⁴;

each R^{h23} and Rⁱ²³ is independently selected from OH, C₁₋₆ alkoxy, and C₁₋₆ haloalkoxy; or any R^{h23} and Rⁱ²³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a24}, R^{b24}, R^{c24} and R^{d24}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; each R^{h24} and Rⁱ²⁴ is independently selected from OH, C₁₋₆ alkoxy, and C₁₋₆ haloalkoxy; or any R^{h24} and Rⁱ²⁴ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a30}, R^{b30}, R^{c30} and R^{d30} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³¹;

or any R^{c30} and R^{d30} attached to the same N atom, together with the N atom to which they are attached, form a 4-, 5-, 6- or 7-membered heterocycloalkyl group optionally substituted with 1, 2, 3, or 4 substituents independently selected from R³¹;

each R^{h30} and Rⁱ³⁰ is independently selected from OH, C₁₋₆ alkoxy, and C₁₋₆ haloalkoxy; or any R^{h30} and Rⁱ³⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a31}, R^{b31}, R^{c31} and R^{d31}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R³²;

or any R^{c31} and R^{d31} attached to the same N atom, together with the N atom to which they are attached, form a 4-, 5-, 6- or 7-membered heterocycloalkyl group optionally substituted with 1, 2 or 3 substituents independently selected from R³²;

each R^{h31} and Rⁱ³¹ is independently selected from OH, C₁₋₆ alkoxy, and C₁₋₆ haloalkoxy; or any R^{h31} and Rⁱ³¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a32}, R^{b32}, R^{c32} and R^{d32}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};

each R^{h32} and Rⁱ³² is independently selected from OH, C₁₋₆ alkoxy, and C₁₋₆ haloalkoxy; or any R^{h32} and Rⁱ³² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a50}, R^{b50}, R^{c50} and R^{d5}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆_10 aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R⁵¹;

or any R^{c50} and R^{d50} attached to the same N atom, together with the N atom to which they are attached, form a 4-, 5-, 6- or 7-membered heterocycloalkyl group optionally substituted with 1, 2 or 3 substituents independently selected from R⁵¹;

each R^{h50} and Rⁱ⁵⁰ is independently selected from OH, C₁₋₆ alkoxy, and C₁₋₆ haloalkoxy; or any R^{h50} and Rⁱ⁵⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a51}, R^{b51}, R^{c51} and R^{d51}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R⁵²;

or any R^{c51} and R^{d51} attached to the same N atom, together with the N atom to which they are attached, form a 4-, 5-, 6- or 7-membered heterocycloalkyl group optionally substituted with 1, 2, 3, or 4 substituents independently selected from R⁵²;

each R^{h51} and Rⁱ⁵¹ is independently selected from OH, C₁₋₆ alkoxy, and C₁₋₆ haloalkoxy; or any R^{h51} and Rⁱ⁵¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a52}, R^{b52}, R^{c52} and R^{d52}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};

each R^{h52} and Rⁱ⁵² is independently selected from OH, C₁₋₆ alkoxy, and C₁₋₆ haloalkoxy; or any R^{h52} and Rⁱ⁵² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a60}, R^{b60}, R^{c60} and R^{d60} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};

or any R^{c60} and R^{d60} attached to the same N atom, together with the N atom to which they are attached, form a 4-, 5-, 6- or 7-membered heterocycloalkyl group optionally substituted with 1, 2, 3, or 4 substituents independently selected from R^{g};

each R^{h60} and Rⁱ⁶⁰ is independently selected from OH, C₁₋₆ alkoxy, and C₁₋₆ haloalkoxy; or any R^{h60} and Rⁱ⁶⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a70}, R^{b70}, R^{c70} and R^{d70} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁷¹;

or any R^{c70} and R^{d70} attached to the same N atom, together with the N atom to which they are attached, form a 4-, 5-, 6- or 7-membered heterocycloalkyl group optionally substituted with 1, 2, 3, or 4 substituents independently selected from R⁷¹; each R^{h70} and Rⁱ⁷⁰ is independently selected from OH, C₁₋₆ alkoxy, and C₁₋₆ haloalkoxy; or any R^{h70} and Rⁱ⁷⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a71}, R^{b71}, R^{c71} and R^{d71}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R⁷²;

or any R^{c71} and R^{d71} attached to the same N atom, together with the N atom to which they are attached, form a 4-, 5-, 6- or 7-membered heterocycloalkyl group optionally substituted with 1, 2 or 3 substituents independently selected from R⁷²;

each R^{h71} and Rⁱ⁷¹ is independently selected from OH, C₁₋₆ alkoxy, and C₁₋₆ haloalkoxy; or any R^{h71} and Rⁱ⁷¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a72}, R^{b72}, R^{c72} and R^{d72}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};

each R^{h72} and Rⁱ⁷² is independently selected from OH, C₁₋₆ alkoxy, and C₁₋₆ haloalkoxy; or any R^{h72} and Rⁱ⁷² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; and

each R⁹ is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;

provided that, when R⁴R⁵C [symbol] YR⁶ is a double bond and Y is N, then Cy¹ is other than 3,5-dimethylisoxazol-4-yl.

In another aspect, provided herein is a compound of Formula I:

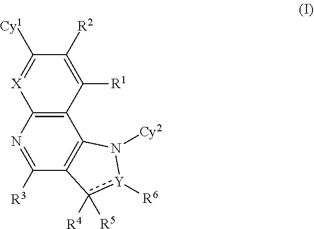

or a pharmaceutically acceptable salt thereof,

wherein:

each [symbol] independently represents a single bond or a double bond;

X is N or CR⁷;

Y is N or C;

R¹ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, and BR^{h1}Rⁱ¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R^{g};

R² is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, and BR^{h2}Rⁱ²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;

Cy¹ is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R¹⁰;

R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR², SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{j3}, NR^{c3}C(O)R^{b3}, NR^{c3}C(O)OR^{a3}, NR^{c3}C(O)NR^{c3}R^{d3}, C(═NR^{e3})R^{b3}, C(═NOR^{a3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, and BR^{h3}Rⁱ³; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³⁰;

when R⁴R⁵C [symbol] YR⁶ is a single bond and Y is C, then YR⁶ is selected from C═O and C═S; and

R⁴ is selected from H, D, C₁₋₆ alkyl, C₂₋₆ alkenyl, C₂₋₆ alkynyl, C₁₋₆ haloalkyl, C₃₋₆ cycloalkyl, 4-6 membered heterocycloalkyl, phenyl, 5-6 membered heteroaryl, halo, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, and BR^{h4}Rⁱ⁴; wherein said C₁₋₆ alkyl, C₂₋₆ alkenyl, C₂₋₆ alkynyl, C₃₋₆ cycloalkyl, 4-6 membered heterocycloalkyl, phenyl, and 5-6 membered heteroaryl, are each optionally substituted with 1, 2, 3, or 4 substituents independently selected from R^{g};

R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NOR^{a5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)₂R^{b5}, NR^{c5}S(O)₂NR^{c5}R^{d5}S(O)R^{b5}, S(O)NR^{c5}R^{d5}, S(O)₂R^{b5}, S(O)₂NR^{c5}R^{d5}, and BR^{h5}Rⁱ⁵; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵⁰;

when R⁴R⁵C [symbol] R⁶ is a double bond and Y is N, then R⁴ and R⁶ are absent;

when R⁴R⁵C [symbol] YR⁶ is a double bond and Y is C, then R⁴ is absent; and

R⁶ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NOR^{a6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, and BR^{h6}Rⁱ⁶; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶⁰;

R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NOR^{a7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, and BR^{h7}Rⁱ⁷; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷⁰;

Cy² is selected from C₃₋₁₀ cycloalkyl, 4-14 membered heterocycloalkyl, C₆₋₁₀ aryl and 5-10 membered heteroaryl; wherein the 4-14 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-14 membered heterocycloalkyl is optionally substituted by oxo to form a carbonyl group; and wherein the C₃₋₁₀ cycloalkyl, 4-14 membered heterocycloalkyl, C₆₋₁₀ aryl and 5-10 membered heteroaryl are each optionally substituted with 1, 2, 3 or 4 substituents independently selected from R²⁰;

each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, C(═NR^{e10})R^{b10}, C(═NOR^{a10})R^{b10}, C(═NR^{e10})NR^{c10}R^{d10}, NR^{c10}C(═NR^{e10})NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, S(O)₂NR^{c10}R^{d10}, and BR^{h10}Rⁱ¹⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;

each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, and BR^{h11}Rⁱ¹¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²;

each R¹² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)R^{b12}, NR^{c12}S(O)₂R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, and BR^{h12}Rⁱ¹²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};

each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a20}, SR^{a20}, C(O)R^{b20}, C(O)NR^{c20}R^{d20}C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, C(═NR^{e20})R^{b20}, C(═NOR^{a20})R^{b20}, C(═NR^{e20})NR^{c20}R^{d20}, NR^{c20}C(═NR^{e20})NR^{c20}R^{d20}, NR^{c20}S(O)R^{b20}, NR^{c20}S(O)₂R^{b20}, NR^{c20}S(O)₂NR^{c20}R^{d20}, S(O)R^{b20}, S(O)NR^{c20}R^{d20}, S(O)₂R^{b20}, S(O)₂NR^{c20}R^{d20}, and BR^{h20}Rⁱ²⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;

each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)R^{b21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, and BR^{h21}Rⁱ²¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R^{g};

each R²² is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)R^{b22}, NR^{c22}S(O)₂R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, and BR^{h22}Rⁱ²²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²³;

each R²³ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a23}, SR^{a23}, C(O)R^{b23}, C(O)NR^{c23}R^{d23}, C(O)OR^{a23}, OC(O)R^{b23}, OC(O)NR^{c23}R^{d23}, NR^{c23}R^{d23}, NR^{c23}C(O)R^{b23}, NR^{c23}C(O)OR^{a23}, NR^{c23}C(O)NR^{c23}R^{d23}, NR^{c23}S(O)R^{b23}, NR^{c23}S(O)₂R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, and BR^{h23}Rⁱ²³; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²⁴;

each R²⁴ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a24}, SR^{a24}, C(O)R^{b24}, C(O)NR^{c24}R^{d24}, C(O)OR^{a24}, OC(O)R^{b24}, OC(O)NR^{c24}R^{d24}, NR^{c24}R^{d24}, NR^{c24}C(O)R^{b24}, NR^{c24}C(O)OR^{a24}, NR^{c24}C(O)NR^{c24}R^{d24}, NR^{c24}S(O)R^{b24}, NR^{c24}S(O)₂R^{b24}, NR^{c24}S(O)₂NR^{c24}R^{d24}, S(O)R^{b24}, S(O)NR^{c24}R^{d24}, S(O)₂R^{b24}, S(O)₂NR^{c24}R^{d24}, and BR^{h24}Rⁱ²⁴; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g}; each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a30}, SR^{a30}, C(O)R^{b30}, C(O)NR^{c30}R^{d30}, C(O)OR^{a30}, OC(O)R^{b30}, OC(O)NR^{c30}R^{d30}, NR^{c30}R^{d30}, NR^{c30}C(O)R^{b30}, NR^{c30}C(O)OR^{a30}, NR^{c30}C(O)NR^{c20}R^{d30}, NR^{c30}S(O)R^{b30}, NR^{c30}S(O)₂R^{b30}, NR^{c30}S(O)₂NR^{c30}R^{d30}, S(O)R^{b30}, S(O)NR^{c30}R^{d30}, S(O)₂R^{b30}, S(O)₂NR^{c30}R^{d30}, and BR^{h30}Rⁱ³⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³¹;

each R³¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a31}, SR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR^{c31}C(O)R^{b31}, NR^{c21}C(O)OR^{a31}, NR^{c31}C(O)NR^{c31}R^{d31}, NR^{c31}S(O)R^{b31}, NR^{c31}S(O)₂R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, and BR^{h31}Rⁱ³¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³²;

each R³² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a32}, SR^{a32}, C(O)R^{b32}, C(O)NR^{c32}R^{d32}, C(O)OR^{a32}, OC(O)R^{b32}, OC(O)NR^{c32}R^{d32}, NR^{c32}R^{d32}, NR^{c32}C(O)R^{b32}, NR^{c2}C(O)OR^{a32}, NR^{c32}C(O)NR^{c32}R^{d32}, NR^{c2}S(O)R^{b32}, NR^{c32}S(O)₂R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32}, and BR^{h32}Rⁱ³²; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g}; each R⁵⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a50}, SR^{a50}, C(O)R^{b50}, C(O)NR^{c50}R^{d50}, C(O)OR^{a50}, OC(O)R^{b50}, OC(O)NR^{c50}R^{d50}, NR^{c50}R^{d50}, NR^{c50}C(O)R^{b50}, NR^{c50}C(O)OR^{a50}, NR^{c50}C(O)NR^{c50}R^{d50}, NR^{c50}S(O)R^{b50}, NR^{c50}S(O)₂R^{b50}, NR^{c50}S(O)₂NR^{c50}R^{d50}, S(O)R^{b50}, S(O)NR^{c50}R^{d50}, S(O)₂R^{b50}, S(O)₂NR^{c50}R^{d50}, and BR^{h50}Rⁱ⁵⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵¹;

each R⁵¹ is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c51}C(O)NR^{c51}R^{d51}, NR^{c51}S(O)R^{b51}, NR^{c51}S(O)₂R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, and BR^{h51}Rⁱ⁵¹; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R⁵²;

each R⁵² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a52}, SR^{a52}, C(O)R^{b52}, C(O)NR^{c52}R^{d52}, C(O)OR^{a52}, OC(O)R^{b52}, OC(O)NR^{c52}R^{d52}, NR^{c52}R^{d52}, NR^{c52}C(O)R^{b52}, NR^{c52}C(O)OR^{a52}, NR^{c52}C(O)NR^{c52}R^{d52}, NR^{c52}S(O)R^{b52}, NR^{c52}S(O)₂R^{b52}, NR^{c52}S(O)₂NR^{c52}R^{d52}, S(O)R^{b52}, S(O)NR^{c52}R^{d52}, S(O)₂R^{b52}, S(O)₂NR^{c52}R^{d52}, and BR^{h52}Rⁱ⁵²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};

each R⁶⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a60}, SR^{a60}, C(O)R^{b60}, C(O)NR^{c60}R^{d60}, C(O)OR^{a60}, OC(O)R^{b60}, OC(O)NR^{c60}R^{d60}, NR^{c60}R^{d60}, NR^{c60}C(O)R^{b60}, NR^{c60}C(O)OR^{a60}, NR^{c60}C(O)NR^{c60}R^{d60}, NR^{c60}S(O)R^{b60}, NR^{c60}S(O)₂R^{b60}, NR^{c60}S(O)₂NR^{c60}R^{d60}, S(O)R^{b60}, S(O)NR^{d60}R^{d60}, S(O)₂R^{b60}S(O)₂NR^{c60}R^{d60}, and BR^{h60}Rⁱ⁶⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶¹;

each R⁶¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)R^{b61}, NR^{c61}S(O)₂R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, and BR^{h61}Rⁱ⁶¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶²;

each R⁶² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62}, NR^{c62}C(O)NR^{c62}R^{d62}, NR^{c62}S(O)R^{b62}, NR^{c62}S(O)₂R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, and BR^{h62}Rⁱ⁶²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};

each R⁷⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a70}, SR^{a70}, C(O)R^{b70}, C(O)NR^{c70}R^{d70}, C(O)OR^{a70}, OC(O)R^{b70}, OC(O)NR^{c70}R^{d70}, NR^{c70}R^{d70}, NR^{c70}C(O)R^{b70}, NR^{c70}C(O)OR^{a70}, NR^{c70}C(O)NR^{c70}R^{d70}, NR^{c70}S(O)R^{b70}, NR^{c70}S(O)₂R^{b70}, NR^{c70}S(O)₂NR^{c70}R^{d70}, S(O)R^{b70}, S(O)NR^{c70}R^{d70}, S(O)₂R^{b70}, S(O)₂NR^{d70}R^{d70}, and BR^{h70}Rⁱ⁷⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷¹;

each R⁷¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)R^{b71}, NR^{c71}S(O)₂R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, and BR^{h71}Rⁱ⁷¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷²;

each R⁷² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a72}, SR^{a72}, C(O)R^{b72}, C(O)NR^{c72}R^{d72}, C(O)OR^{a72}, OC(O)R^{b72}, OC(O)NR^{c72}R^{d72}, NR^{c72}R^{d72}, NR^{c72}C(O)R^{b72}, NR^{c72}C(O)OR^{a72}, NR^{c72}C(O)NR^{c72}R^{d72}, NR^{c72}S(O)R^{b72}, NR^{c72}S(O)₂R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72}, and BR^{h72}Rⁱ⁷²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};

each R^{a1}, R^{b1}, R^{c1}, and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};

or any R^{c1} and R^{d1} attached to the same N atom, together with the N atom to which they are attached, form a 4-, 5-, 6- or 7-membered heterocycloalkyl group optionally substituted with 1, 2 or 3 substituents independently selected from R^{g};

each R^{h1} and Rⁱ¹ is independently selected from OH, C₁₋₆ alkoxy, and C₁₋₆ haloalkoxy; or any R^{h1} and Rⁱ¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a2}, R^{b2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²²;

or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R²²;

each R^{e2} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h2} and Rⁱ² is independently selected from OH, C₁₋₆ alkoxy, and C₁₋₆ haloalkoxy; or any R^{h2} and Rⁱ² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a3}, R^{b3}, R^{c3} and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{e3} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{f3} and R^{j3} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₃ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{h3} and Rⁱ³ is independently selected from OH, C₁₋₆ alkoxy, and C₁₋₆ haloalkoxy; or any R^{h3} and Rⁱ³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a4}, R^{b4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};

or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R^{g};

each R^{h4} and Rⁱ⁴ is independently selected from OH, C₁₋₆ alkoxy, and C₁₋₆ haloalkoxy; or any R^{h4} and Rⁱ⁴ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; each R^{a5}, R^{b5}, R^{c5} and R^{d5} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁵⁰;

or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R⁵⁰;

each R^{e5} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h5} and Rⁱ⁵ is independently selected from OH, C₁₋₆ alkoxy, and C₁₋₆ haloalkoxy; or any R^{h5} and Rⁱ⁵ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; each R^{a6}, R^{b6}, R^{c6} and R^{d6} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶⁰;

or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R⁶⁰;

each R^{e6} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h6} and Rⁱ⁶ is independently selected from OH, C₁₋₆ alkoxy, and C₁₋₆ haloalkoxy; or any R^{h6} and Rⁱ⁶ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a7}, R^{b7}, R^{c7} and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁷⁰;

or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R⁷⁰;

each R^{e7} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h7} and Rⁱ⁷ is independently selected from OH, C₁₋₆ alkoxy, and C₁₋₆ haloalkoxy; or any R^{h7} and Rⁱ⁷ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a10}, R^{b10}, R^{c10} and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;

or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R¹¹;

each R^{e10} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h10} and Rⁱ¹⁰ is independently selected from OH, C₁₋₆ alkoxy, and C₁₋₆ haloalkoxy; or any R^{h10} and Rⁱ¹⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a11}, R^{b11}, R^{c11} and R^{d11}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;

or any R^{c11} and R^{d11} attached to the same N atom, together with the N atom to which they are attached, form a 4-, 5-, 6- or 7-membered heterocycloalkyl group optionally substituted with 1, 2 or 3 substituents independently selected from R¹²;

each R^{h11} and Rⁱ¹¹ is independently selected from OH, C₁₋₆ alkoxy, and C₁₋₆ haloalkoxy; or any R^{h11} and Rⁱ¹¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a12}, R^{b12}, R^{c12} and R^{d12}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; each R^{h12} and Rⁱ¹² is independently selected from OH, C₁₋₆ alkoxy, and C₁₋₆ haloalkoxy; or any R^{h12} and Rⁱ¹² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²¹;

or any R^{c20} and R^{d20} attached to the same N atom, together with the N atom to which they are attached, form a 4-, 5-, 6- or 7-membered heterocycloalkyl group optionally substituted with 1, 2, 3, or 4 substituents independently selected from R²¹;

each R^{e20} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h20} and Rⁱ²⁰ is independently selected from OH, C₁₋₆ alkoxy, and C₁₋₆ haloalkoxy; or any R^{h20} and Rⁱ²⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a21}, R^{b21}, R^{c21} and R^{d21}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R^{g};

or any R^{c21} and R^{d21} attached to the same N atom, together with the N atom to which they are attached, form a 4-, 5-, 6- or 7-membered heterocycloalkyl group optionally substituted with 1, 2 or 3 substituents independently selected from R^{g};

each R^{h21} and Rⁱ²¹ is independently selected from OH, C₁₋₆ alkoxy, and C₁₋₆ haloalkoxy; or any R^{h21} and Rⁱ²¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a22}, R^{b22}, R^{c22} and R^{d22} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²³;

or any R^{c22} and R^{d22} attached to the same N atom, together with the N atom to which they are attached, form a 4-, 5-, 6- or 7-membered heterocycloalkyl group optionally substituted with 1, 2, 3, or 4 substituents independently selected from R²³;

each R^{h22} and Rⁱ²² is independently selected from OH, C₁₋₆ alkoxy, and C₁₋₆ haloalkoxy; or any R^{h22} and Rⁱ²² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a23}, R^{b23}, R^{c23} and R^{d23}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²⁴;

or any R^{c23} and R^{d23} attached to the same N atom, together with the N atom to which they are attached, form a 4-, 5-, 6- or 7-membered heterocycloalkyl group optionally substituted with 1, 2 or 3 substituents independently selected from R²⁴;

each R^{h23} and Rⁱ²³ is independently selected from OH, C₁₋₆ alkoxy, and C₁₋₆ haloalkoxy; or any R^{h23} and Rⁱ²³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a24}, R^{b24}, R^{c24} and R^{d24}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};

each R^{h24} and Rⁱ²⁴ is independently selected from OH, C₁₋₆ alkoxy, and C₁₋₆ haloalkoxy; or any R^{h24} and Rⁱ²⁴ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a30}, R^{b30}, R^{c30} and R^{d30} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³¹;

or any R^{c30} and R^{d30} attached to the same N atom, together with the N atom to which they are attached, form a 4-, 5-, 6- or 7-membered heterocycloalkyl group optionally substituted with 1, 2, 3, or 4 substituents independently selected from R³¹;

each R^{h30} and Rⁱ³⁰ is independently selected from OH, C₁₋₆ alkoxy, and C₁₋₆ haloalkoxy; or any R^{h30} and Rⁱ³⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a31}, R^{b31}, R^{c31} and R^{d31}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R³²;

or any R^{c31} and R^{d31} attached to the same N atom, together with the N atom to which they are attached, form a 4-, 5-, 6- or 7-membered heterocycloalkyl group optionally substituted with 1, 2 or 3 substituents independently selected from R³²;

each R^{h31} and Rⁱ³¹ is independently selected from OH, C₁₋₆ alkoxy, and C₁₋₆ haloalkoxy; or any R^{h31} and Rⁱ³¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a32}, R^{b32}, R^{c32} and R^{d32}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; each R^{h32} and Rⁱ³² is independently selected from OH, C₁₋₆ alkoxy, and C₁₋₆ haloalkoxy; or any R^{h32} and Rⁱ³² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a50}, R^{b50}, R^{c50} and R^{d50}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R⁵¹;

or any R^{c50} and R^{d50} attached to the same N atom, together with the N atom to which they are attached, form a 4-, 5-, 6- or 7-membered heterocycloalkyl group optionally substituted with 1, 2 or 3 substituents independently selected from R⁵¹;

each R^{h50} and Rⁱ⁵⁰ is independently selected from OH, C₁₋₆ alkoxy, and C₁₋₆ haloalkoxy; or any R^{h50} and Rⁱ⁵⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a51}, R^{b51}, R^{c51} and R^{d51}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R⁵²;

or any R^{c51} and R^{d51} attached to the same N atom, together with the N atom to which they are attached, form a 4-, 5-, 6- or 7-membered heterocycloalkyl group optionally substituted with 1, 2, 3, or 4 substituents independently selected from R⁵²;

each R^{h51} and Rⁱ⁵¹ is independently selected from OH, C₁₋₆ alkoxy, and C₁₋₆ haloalkoxy; or any R^{h51} and Rⁱ⁵¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a52}, R^{b52}, R^{c52} and R^{d52}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};

each R^{h52} and Rⁱ⁵² is independently selected from OH, C₁₋₆ alkoxy, and C₁₋₆ haloalkoxy; or any R^{h52} and Rⁱ⁵² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a60}, R^{b60}, R^{c60} and R^{d60} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶¹;

or any R^{c60} and R^{d60} attached to the same N atom, together with the N atom to which they are attached, form a 4-, 5-, 6- or 7-membered heterocycloalkyl group optionally substituted with 1, 2, 3, or 4 substituents independently selected from R⁶¹;

each R^{h60} and Rⁱ⁶⁰ is independently selected from OH, C₁₋₆ alkoxy, and C₁₋₆ haloalkoxy; or any R^{h60} and Rⁱ⁶⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a61}, R^{b61}, R^{c61} and R^{d61}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R⁶²;

or any R^{c61} and R^{d61} attached to the same N atom, together with the N atom to which they are attached, form a 4-, 5-, 6- or 7-membered heterocycloalkyl group optionally substituted with 1, 2 or 3 substituents independently selected from R⁶²;

each R^{h61} and Rⁱ⁶¹ is independently selected from OH, C₁₋₆ alkoxy, and C₁₋₆ haloalkoxy; or any R^{h61} and Rⁱ⁶¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a62}, R^{b62}, R^{c62} and R^{d62}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};

or any R^{c62} and R^{d62} attached to the same N atom, together with the N atom to which they are attached, form a 4-, 5-, 6- or 7-membered heterocycloalkyl group optionally substituted with 1, 2, 3, or 4 substituents independently selected from R^{g}; each R^{h62} and Rⁱ⁶² is independently selected from OH, C₁₋₆ alkoxy, and C₁₋₆ haloalkoxy; or any R^{h62} and Rⁱ⁶² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a70}, R^{b70}, R^{c70} and R^{d70} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁷¹;

or any R^{c70} and R^{d70} attached to the same N atom, together with the N atom to which they are attached, form a 4-, 5-, 6- or 7-membered heterocycloalkyl group optionally substituted with 1, 2, 3, or 4 substituents independently selected from R⁷¹;

each R^{h70} and Rⁱ⁷⁰ is independently selected from OH, C₁₋₆ alkoxy, and C₁₋₆ haloalkoxy; or any R^{h70} and Rⁱ⁷⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a71}, R^{b71}, R^{c71} and R^{d71}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R⁷²;

or any R^{c71} and R^{d71} attached to the same N atom, together with the N atom to which they are attached, form a 4-, 5-, 6- or 7-membered heterocycloalkyl group optionally substituted with 1, 2 or 3 substituents independently selected from R⁷²;

each R^{h71} and Rⁱ⁷¹ is independently selected from OH, C₁₋₆ alkoxy, and C₁₋₆ haloalkoxy; or any R^{h71} and Rⁱ⁷¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a72}, R^{b72}, R^{c72} and R^{d72}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; each R^{h72} and Rⁱ⁷² is independently selected from OH, C₁₋₆ alkoxy, and C₁₋₆ haloalkoxy; or any R^{h72} and Rⁱ⁷² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; and

R^{g} is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;

provided that, when R⁴R⁵C [symbol] YR⁶ is a double bond and Y is N, then Cy¹ is other than 3,5-dimethylisoxazol-4-yl.

In an embodiment of Formula I, or a pharmaceutically acceptable salt thereof,

each [symbol] independently represents a single bond or a double bond;

X is N or CR⁷;

Y is N or C;

R¹ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, CB-10 aryl, 5-10 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, and BR^{h1}Rⁱ¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R^{g};

R² is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, and BR^{h2}Rⁱ²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;

Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3 or 4 substituents independently selected from R¹⁰;

R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{f3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{j3}, NR^{c3}C(O)R^{b3}, NR^{c3}C(O)OR^{a3}, NR^{c3}C(O)NR^{c3}R^{d3}, C(═NR^{e3})R^{b3}, C(═NOR^{a3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, and BR^{h3}Rⁱ³; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³⁰;

when R⁴R⁵C [symbol] YR⁶ is a single bond and Y is C, then YR⁶ is selected from C═O and C═S; and

R⁴ is selected from H, D, C₁₋₆ alkyl, C₂₋₆ alkenyl, C₂₋₆ alkynyl, C₁₋₆ haloalkyl, C₃₋₆ cycloalkyl, 4-6 membered heterocycloalkyl, phenyl, 5-6 membered heteroaryl, halo, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, and BR^{h4}Rⁱ⁴;

R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a5}, SR^{a5}, C(O)R^{b5}, C(O)NR^{c5}R^{d5}C(O)OR^{a5}′ OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NOR^{a5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, and BR^{h5}Rⁱ⁵; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independent selected from R⁵⁰;

when R⁴R⁵C [symbol] YR⁶ is a double bond and Y is N, then R⁴ and R⁶ are absent;

when R⁴R⁵C [symbol] YR⁶ is a double bond and Y is C, then R⁴ is absent; and

R⁶ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NOR^{a6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, and BR^{h6}Rⁱ⁶; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶⁰;

R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NOR^{a7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, and BR^{h7}Rⁱ⁷; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷⁰;

Cy² is selected from C₃₋₁₀ cycloalkyl, 4-14 membered heterocycloalkyl, C₆₋₁₀ aryl and 5-10 membered heteroaryl; wherein the 4-14 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-14 membered heterocycloalkyl is optionally substituted by oxo to form a carbonyl group; and wherein the C₃₋₁₀ cycloalkyl, 4-14 membered heterocycloalkyl, C₆₋₁₀ aryl and 5-10 membered heteroaryl are each optionally substituted with 1, 2, 3 or 4 substituents independently selected from R²⁰;

each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, C(═NR^{e10})R^{b10}, C(═NOR^{a10})R^{b10}, C(═NR^{e10})NR^{c10}R^{d10}, NR^{c10}C(═NR^{e10})NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, S(O)₂NR^{c10}R^{d10}, and BR^{h10}Rⁱ¹⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;

each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, and BR^{h11}Rⁱ¹¹;

each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a20}, SR^{a20}, C(O)R^{b20}, C(O)NR^{c20}R^{d20}, C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, C(═NR^{e20})R^{b20}, C(═NOR^{a20})R^{b20}, C(═NR^{e20})NR^{c20}R^{d20}, NR^{c20}C(═NR^{e20})NR^{c20}R^{d20}, NR^{c20}S(O)R^{b20}, NR^{c20}S(O)₂R^{b20}, NR^{c20}S(O)₂NR^{c20}R^{d20}, S(O)R^{b20}, S(O)NR^{c20}R^{d20}, S(O)₂R^{b20}, S(O)₂NR^{c20}R^{d20}, and BR^{h20}Rⁱ²⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;

each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)R^{b21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, and BR^{h21}Rⁱ²¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R^{g};

each R²² is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)R^{b22}, NR^{c22}S(O)₂R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, and BR^{h22}Rⁱ²²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²³;

each R²³ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a23}, SR^{a23}, C(O)R^{b23}, C(O)NR^{c23}R^{d23}, C(O)OR^{a23}, OC(O)R^{b23}, OC(O)NR^{c23}R^{d23}, NR^{c23}R^{d23}, NR^{c23}C(O)R^{b23}, NR^{c23}C(O)OR^{a23}, NR^{c23}C(O)NR^{c23}R^{d23}, NR^{c23}S(O)R^{b23}, NR^{c23}S(O)₂R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, and BR^{h23}Rⁱ²³;

each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a30}, SR^{a30}, C(O)R^{b30}, C(O)NR^{c30}R^{d30}, C(O)OR^{a30}, OC(O)R^{b30}, OC(O)NR^{c30}R^{d30}, NR^{c30}R^{d30}, NR^{c30}C(O)R^{b30}, NR^{c30}C(O)OR^{a30}, NR^{c30}C(O)NR^{c30}R^{d30}, NR^{c30}S(O)R^{b30}, NR^{c30}S(O)₂R^{b30}, NR^{c30}S(O)₂NR^{c30}R^{d30}, S(O)R^{b30}, S(O)NR^{c3}R^{d30}, S(O)₂R^{b30}, S(O)₂NR^{c30}R^{d30}, and BR^{h30}Rⁱ³⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³¹;

each R³¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a31}, SR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR^{c31}C(O)R^{b31}, NR^{c21}C(O)OR^{a31}, NR^{c31}C(O)NR^{c31}R^{d31}, NR^{c31}S(O)R^{b31}, NR^{c31}S(O)₂R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, and BR^{h31}Rⁱ³¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³²;

each R³² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a32}, SR^{a32}, C(O)R^{b32}, C(O)NR^{c32}R^{d32}, C(O)OR^{a32}, OC(O)R^{b32}, OC(O)NR^{c32}R^{d32}, NR^{c32}R^{d32}, NR^{c32}C(O)R^{b32}, NR^{c32}C(O)OR^{a32}, NR^{c32}C(O)NR^{c32}R^{d32}, NR^{c32}S(O)R^{b32}, NR^{c32}S(O)₂R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32}, and BR^{h32}Rⁱ³²;

each R⁵⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a50}, SR^{a50}, C(O)R^{b50}, C(O)NR^{c50}R^{d50}, C(O)OR^{a50}, OC(O)R^{b50}, OC(O)NR^{c50}R^{d50}, NR^{c50}R^{d50}, NR^{c50}C(O)R^{b50}, NR^{c50}C(O)OR^{a50}, NR^{c50}C(O)NR^{c50}R^{d50}, NR^{c50}S(O)R^{b50}, NR^{c50}S(O)₂R^{b50}, NR^{c50}S(O)₂NR^{c50}R^{d50}, S(O)R^{b50}, S(O)NR^{c50}R^{d50}, S(O)₂R^{b50}, S(O)₂NR^{c50}R^{d50}, and BR^{h50}Rⁱ⁵⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵¹;

each R⁵¹ is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c51}C(O)NR^{c51}R^{d51}, NR^{c51}S(O)R^{b51}, NR^{c51}S(O)₂R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, and BR^{h51}Rⁱ⁵¹;

each R⁶⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a60}, SR^{a60}, C(O)R^{b60}, C(O)NR^{c60}R^{d60}, C(O)OR^{a60}, OC(O)R^{b60}, OC(O)NR^{c60}R^{d60}, NR^{c60}R^{d60}, NR^{c60}C(O)R^{b60}, NR^{c60}C(O)OR^{a60}, NR^{c60}C(O)NR^{c60}R^{d60}, NR^{c60}S(O)R^{b60}, NR^{c60}S(O)₂R^{b60}, NR^{c60}S(O)₂NR^{c60}R^{d60}, S(O)R^{b60}, S(O)NR^{c60}R^{d60}, S(O)₂R^{b60}, S(O)₂NR^{c60}R^{d60}, and BR^{h60}Rⁱ⁶⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶¹;

each R⁶¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)R^{b61}, NR^{c61}S(O)₂R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, and BR^{h61}Rⁱ⁶¹;

each R⁷⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a7}, SR^{a70}, C(O)R^{b70}, C(O)NR^{c70}R^{d70}, C(O)OR^{a70}, OC(O)R^{b70}, OC(O)NR^{c70}R^{d70}, NR^{d70}R^{d70}, NR^{c70}C(O)R^{b70}, NR^{c70}C(O)OR^{a70}, NR^{c70}C(O)NR^{c70}R^{d70}, NR^{c70}S(O)R^{b70}, NR^{c70}S(O)₂R^{b70}, NR^{c70}S(O)₂NR^{c70}R^{d70}, S(O)R^{b70}, S(O)NR^{c70}R^{d70}, S(O)₂R^{b70}, S(O)₂NR^{c70}R^{d70}, and BR^{h70}Rⁱ⁷⁰; each R^{a1}, R^{b1}, R^{c1}, and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};

or any R^{c1} and R^{d1} attached to the same N atom, together with the N atom to which they are attached, form a 4-, 5-, 6- or 7-membered heterocycloalkyl group optionally substituted with 1, 2 or 3 substituents independently selected from R^{g};

each R^{h1} and Rⁱ¹ is independently selected from OH, C₁₋₆ alkoxy, and C₁₋₆ haloalkoxy; or any R^{h1} and Rⁱ¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a2}, R^{b2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²²;

or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R²²;

each R^{e2} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h2} and Rⁱ² is independently selected from OH, C₁₋₆ alkoxy, and C₁₋₆ haloalkoxy; or any R^{h2} and Rⁱ² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a3}, R^{b3}, R^{c3} and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R³⁰; each R^{e3} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{f3} and R^{j3} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{h3} and Rⁱ³ is independently selected from OH, C₁₋₆ alkoxy, and C₁₋₆ haloalkoxy; or any R^{h3} and Rⁱ³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a4}, R^{b4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀cycloalkyl, 4-10 membered heterocycloalkyl, C₆₋₁₀ aryl and 5-10 membered heteroaryl, are each optionally substituted with 1, 2, 3, or 4 substituents independently selected from R^{g};

or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R^{g};

each R^{h4} and Rⁱ⁴ is independently selected from OH, C₁₋₆ alkoxy, and C₁₋₆ haloalkoxy; or any R^{h4} and Rⁱ⁴ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a5}, R^{b5}, R^{c5} and R^{d5} is independently selected from H, C₁₋₃ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀cycloalkyl, 4-10 membered heterocycloalkyl, C₆₋₁₀ aryl and 5-10 membered heteroaryl, are each optionally substituted with 1, 2, 3, or 4 substituents independently selected from R⁵⁰;

or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R⁵⁰;

each R^{e5} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h5} and Rⁱ⁵ is independently selected from OH, C₁₋₆ alkoxy, and C₁₋₆ haloalkoxy; or any R^{h5} and Rⁱ⁵ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a6}, R^{b6}, R^{c6} and R^{d6} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀cycloalkyl, 4-10 membered heterocycloalkyl, C₆₋₁₀ aryl and 5-10 membered heteroaryl, are each optionally substituted with 1, 2, 3, or 4 substituents independently selected from R⁶⁰;

or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R⁶⁰;

each R^{e6} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h6} and Rⁱ⁶ is independently selected from OH, C₁₋₆ alkoxy, and C₁₋₆ haloalkoxy; or any R^{h6} and Rⁱ⁶ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a7}, R^{b7}, R^{c7} and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀cycloalkyl, 4-10 membered heterocycloalkyl, C₆₋₁₀ aryl and 5-10 membered heteroaryl, are each optionally substituted with 1, 2, 3, or 4 substituents independently selected from R⁷⁰;

or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R⁷⁰;

each R^{e7} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h7} and Rⁱ⁷ is independently selected from OH, C₁₋₆ alkoxy, and C₁₋₆ haloalkoxy; or any R^{h7} and Rⁱ⁷ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a10}, R^{b10}, R^{c10} and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;

or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R¹¹;

each R^{e10} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h10} and Rⁱ¹⁰ is independently selected from OH, C₁₋₆ alkoxy, and C₁₋₆ haloalkoxy; or any R^{hi0} and Rⁱ¹⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a11}, R^{b11}, R^{c11} and R^{d11}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;

or any R^{c11} and R^{d11} attached to the same N atom, together with the N atom to which they are attached, form a 4-, 5-, 6- or 7-membered heterocycloalkyl group;

each R^{h11} and Rⁱ¹¹ is independently selected from OH, C₁₋₆ alkoxy, and C₁₋₆ haloalkoxy; or any R^{h11} and Rⁱ¹¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²¹;

or any R^{c20} and R^{d20} attached to the same N atom, together with the N atom to which they are attached, form a 4-, 5-, 6- or 7-membered heterocycloalkyl group optionally substituted with 1, 2, 3, or 4 substituents independently selected from R²¹;

each R^{e20} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;

each R^{h20} and Rⁱ²⁰ is independently selected from OH, C₁₋₆ alkoxy, and C₁₋₆ haloalkoxy; or any R^{h20} and Rⁱ²⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a21}, R^{b21}, R^{c21} and R^{d21}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R^{g};

or any R^{c21} and R^{d21} attached to the same N atom, together with the N atom to which they are attached, form a 4-, 5-, 6- or 7-membered heterocycloalkyl group optionally substituted with 1, 2 or 3 substituents independently selected from R^{g};

each R^{h21} and Rⁱ²¹ is independently selected from OH, C₁₋₆ alkoxy, and C₁₋₆ haloalkoxy; or any R^{h21} and Rⁱ²¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a22}, R^{b22}, R^{c22} and R^{d22} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²³;

or any R^{c22} and R^{d22} attached to the same N atom, together with the N atom to which they are attached, form a 4-, 5-, 6- or 7-membered heterocycloalkyl group optionally substituted with 1, 2, 3, or 4 substituents independently selected from R²³;

each R^{h22} and Rⁱ²² is independently selected from OH, C₁₋₆ alkoxy, and C₁₋₆ haloalkoxy; or any R^{h22} and Rⁱ²² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a23}, R^{b23}, R^{c23} and R^{d23}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;

or any R^{c23} and R^{d23} attached to the same N atom, together with the N atom to which they are attached, form a 4-, 5-, 6- or 7-membered heterocycloalkyl group;

each R^{h23} and Rⁱ²³ is independently selected from OH, C₁₋₆ alkoxy, and C₁₋₆ haloalkoxy; or any R^{h23} and Rⁱ²³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a30}, R^{b30}, R^{c30} and R^{d30} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³¹;

or any R^{c30} and R^{d30} attached to the same N atom, together with the N atom to which they are attached, form a 4-, 5-, 6- or 7-membered heterocycloalkyl group optionally substituted with 1, 2, 3, or 4 substituents independently selected from R³¹;

each R^{h30} and Rⁱ³⁰ is independently selected from OH, C₁₋₆ alkoxy, and C₁₋₆ haloalkoxy; or any R^{h30} and Rⁱ³⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a31}, R^{b31}, R^{c31} and R^{d31}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R³²;

or any R^{c31} and R^{d31} attached to the same N atom, together with the N atom to which they are attached, form a 4-, 5-, 6- or 7-membered heterocycloalkyl group optionally substituted with 1, 2 or 3 substituents independently selected from R³²;

each R^{h31} and Rⁱ³¹ is independently selected from OH, C₁₋₆ alkoxy, and C₁₋₆ haloalkoxy; or any R^{h31} and Rⁱ³¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a32}, R^{b32}, R^{c32} and R^{d32}, is independently selected from H, C₁₋₆ alkyl, C₂₋₆ alkenyl, C₂₋₆ alkynyl and C₁₋₆ haloalkyl;

each R^{h32} and Rⁱ³² is independently selected from OH, C₁₋₆ alkoxy, and C₁₋₆ haloalkoxy; or any R^{h32} and Rⁱ³² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a50}, R^{b50}, R^{c50} and R^{d50}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R⁵¹;

or any R^{c50} and R^{d50} attached to the same N atom, together with the N atom to which they are attached, form a 4-, 5-, 6- or 7-membered heterocycloalkyl group optionally substituted with 1, 2 or 3 substituents independently selected from R⁵¹;

each R^{h50} and Rⁱ⁵⁰ is independently selected from OH, C₁₋₆ alkoxy, and C₁₋₆ haloalkoxy; or any R^{h50} and Rⁱ⁵⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a51}, R^{b51}, R^{c51} and R^{d51}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;

or any R^{c51} and R^{d51} attached to the same N atom, together with the N atom to which they are attached, form a 4-, 5-, 6- or 7-membered heterocycloalkyl group;

each R^{h51} and Rⁱ⁵¹ is independently selected from OH, C₁₋₆ alkoxy, and C₁₋₆ haloalkoxy; or any R^{h51} and Rⁱ⁵¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a60}, R^{b60}, R^{c60} and R^{d60} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶¹;

or any R^{c60} and R^{d60} attached to the same N atom, together with the N atom to which they are attached, form a 4-, 5-, 6- or 7-membered heterocycloalkyl group optionally substituted with 1, 2, 3, or 4 substituents independently selected from R⁶¹;

each R^{h60} and Rⁱ⁶ is independently selected from OH, C₁₋₆ alkoxy, and C₁₋₆ haloalkoxy; or any R^{h60} and Rⁱ⁶ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a61}, R^{b61}, R^{c61} and R^{d61}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;

or any R^{c61} and R^{d61} attached to the same N atom, together with the N atom to which they are attached, form a 4-, 5-, 6- or 7-membered heterocycloalkyl group;

each R^{h61} and Rⁱ⁶¹ is independently selected from OH, C₁₋₆ alkoxy, and C₁₋₆ haloalkoxy; or any R^{h61} and Rⁱ⁶¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;

each R^{a70}, R^{b70}, R^{c70} and R^{d70} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;

or any R^{c70} and R^{d70} attached to the same N atom, together with the N atom to which they are attached, form a 4-, 5-, 6- or 7-membered heterocycloalkyl group;

each R^{h70} and Rⁱ⁷⁰ is independently selected from OH, C₁₋₆ alkoxy, and C₁₋₆ haloalkoxy; or any R^{h70} and Rⁱ⁷⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; and

each R⁹ is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆alkyl)aminocarbonylamino.

In another embodiment, the compound of Formula I is a compound of Formula Ia:

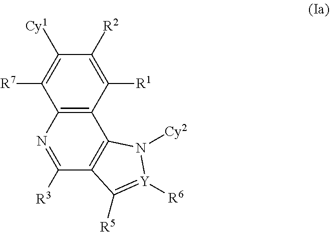

or a pharmaceutically acceptable salt thereof, wherein:

Y is N or C;

R¹ is selected from H, D, C₁₋₆ alkyl, C₁₋₆ haloalkyl, halo, and CN;

R² is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;

Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3 or 4 substituents independently selected from R¹⁰;

R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{f3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{j3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³⁰;

R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a5}, SR^{a5}, C(O)R^{b5}, C(O)NR^{c5}R^{d5}C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}S(O)₂R^{b5}, and S(O)₂NR^{c5}R^{d5}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵⁰;

when R⁴R⁵C [symbol] YR⁶ is a double bond and Y is N, then R⁴ and R⁶ are absent;

when R⁴R⁵C [symbol] YR⁶ is a double bond and Y is C, then R⁴ is absent; and

R⁶ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂R^{b6}, NR^{c6}S(O)₂NR^{c6}R^{d6}, S(O)₂R^{b6}, and S(O)₂NR^{c6}R^{d6}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶⁰;

R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7};

Cy² is selected from C₃₋₁₀ cycloalkyl, 4-14 membered heterocycloalkyl, C₆₋₁₀ aryl and 5-10 membered heteroaryl; wherein the 4-14 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-14 membered heterocycloalkyl is optionally substituted by oxo to form a carbonyl group; and wherein the C₃₋₁₀ cycloalkyl, 4-14 membered heterocycloalkyl, C₆₋₁₀ aryl and 5-10 membered heteroaryl are each optionally substituted with 1, 2, 3 or 4 substituents independently selected from R²⁰;

each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};

each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a20}, SR^{a20}, C(O)R^{b20}, C(O)NR^{c20}R^{d20}C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, NR^{c20}S(O)₂R^{b20}, NR^{c20}S(O)₂NR^{c20}R^{d20}, S(O)₂R^{b20}, and S(O)₂NR^{c20}R^{d20}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;

each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};

each R²² is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a22}, SR^{a22}, C(O)R^{b22}, C(O)NR^{c22}R^{d22}, C(O)OR^{a22}, C(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)₂R^{b22}, NR^{c22}S(O)₂NR^{c22}R^{d22}, S(O)₂R^{b22}, and S(O)₂NR^{c22}R^{d22};

each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a30}, SR^{a30}, C(O)R^{b30}, C(O)NR^{c30}R^{d30}, C(O)OR^{a30}, OC(O)R^{b30}, OC(O)NR^{c30}R^{d30}, NR^{c30}R^{d30}, NR^{c30}C(O)R^{b30}, NR^{c30}C(O)OR^{a30}, NR^{c30}C(O)NR^{c30}R^{d30}, NR^{c30}S(O)₂R^{b30}, NR^{c30}S(O)₂NR^{c30}R^{d30}, S(O)₂R^{b30}, and S(O)₂NR^{c30}R^{d30}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³¹;

each R³¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a31}, SR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR^{c31}C(O)R^{b31}, NR^{c31}C(O)OR^{a31}, NR^{c31}C(O)NR^{c31}R^{d31}, NR^{c31}S(O)₂R^{b31}, NR^{c31}S(O)₂NR^{c31}R^{d31}, S(O)₂R^{b31}, and S(O)₂NR^{c31}R^{d31};

each R⁵⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a50}, SR^{a50}, C(O)R^{b50}, C(O)NR^{c50}R^{d50}C(O)OR^{a50}, OC(O)R^{b50}, OC(O)NR^{c50}R^{d50}, NR^{c50}R^{d50}, NR^{c50}C(O)R^{b50}, NR^{c50}C(O)OR^{a50}, NR^{c50}C(O)NR^{c50}R^{d50}, NR^{c50}S(O)₂R^{b50}, NR^{c50}S(O)₂NR^{c50}R^{d50}, S(O)₂R^{b50}, and S(O)₂NR^{c50}R^{d50};

each R⁶⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a6}, SR^{a60}, C(O)R^{b60}, C(O)NR^{c60}R^{d60}, C(O)OR^{a60}, OC(O)R^{b60}, OC(O)NR^{c60}R^{d60}, NR^{c60}R^{d60}, NR^{c60}C(O)R^{b60}, NR^{c60}C(O)OR^{a60}, NR^{c60}C(O)NR^{c60}R^{d60}, NR^{c60}S(O)₂R^{b60}, NR^{c60}S(O)₂NR^{c60}R^{d60}, S(O)₂R^{b60}, and S(O)₂NR^{c60}R^{d60};

each R^{a2}, R^{b2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²²;

or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R²²;

each R^{a3}, R^{b3}, R^{c3} and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{f3} and R^{j3} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{a5}, R^{b5}, R^{c5} and R^{d5} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁵⁰;

or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R⁵⁰;

each R^{a6}, R^{b6}, R^{c6} and R^{d6} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶⁰;

or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R⁶⁰;

each R^{a7}, R^{b7}, R^{c7} and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;

or any R^{c7} and R^{d7} attached to the same N atom, together with the N atom to which they are attached, form a 4-, 5-, 6- or 7-membered heterocycloalkyl group;

each R^{a10}, R^{b10}, R^{c10} and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;

or any R^{c10} and R^{d10} attached to the same N atom, together with the N atom to which they are attached, form a 4-, 5-, 6- or 7-membered heterocycloalkyl group;

each R^{a20}, R^{b20}, R^{c20} and R^{d20} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²¹;

or any R^{c20} and R^{d20} attached to the same N atom, together with the N atom to which they are attached, form a 4-, 5-, 6- or 7-membered heterocycloalkyl group optionally substituted with 1, 2, 3, or 4 substituents independently selected from R²¹;

each R^{a21}, R^{b21}, R^{c21} and R^{d21}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;

or any R^{c21} and R^{d21} attached to the same N atom, together with the N atom to which they are attached, form a 4-, 5-, 6- or 7-membered heterocycloalkyl group;

each R^{a22}, R^{b22}, R^{c22} and R^{d22} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;

or any R^{c22} and R^{d22} attached to the same N atom, together with the N atom to which they are attached, form a 4-, 5-, 6- or 7-membered heterocycloalkyl group;

each R^{a30}, R^{b30}, R^{c30} and R^{d30} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³¹;

or any R^{c30} and R^{d30} attached to the same N atom, together with the N atom to which they are attached, form a 4-, 5-, 6- or 7-membered heterocycloalkyl group optionally substituted with 1, 2, 3, or 4 substituents independently selected from R³¹;

each R^{a31}, R^{b31}, R^{c31} and R^{d31}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;

or any R^{c31} and R^{d31} attached to the same N atom, together with the N atom to which they are attached, form a 4-, 5-, 6- or 7-membered heterocycloalkyl group;

each R^{a50}, R^{b50}, R^{c50} and R^{d50}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;

or any R^{c50} and R^{d50}, attached to the same N atom, together with the N atom to which they are attached, form a 4-, 5-, 6- or 7-membered heterocycloalkyl group; and each R^{a60}, R^{b60}, R^{c60} and R^{d60} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;

or any R^{c60} and R^{d60} attached to the same N atom, together with the N atom to which they are attached, form a 4-, 5-, 6- or 7-membered heterocycloalkyl group.

In an embodiment of Formula Ia, or a pharmaceutically acceptable salt thereof,

Y is N or C;

R¹ is selected from H, D, and C₁₋₆ alkyl;

R² is selected from H, C₁₋₆ alkyl, C₁₋₆ haloalkyl, halo, D, CN, OR^{a2}, and NR^{c2}R^{d2} wherein said C₁₋₆ alkyl, is optionally substituted with 1 or 2 substituents independently selected from R²²;

Cy¹ is selected from C₆₋₁₀ aryl and 6-10 membered heteroaryl; wherein the 6-10 membered heteroaryl has at least one ring-forming carbon atom and 1, 2, 3, or 4 ring-forming heteroatoms independently selected from N, O, and S; wherein a ring-forming carbon atom of 6-10 membered heteroaryl is optionally substituted by oxo to form a carbonyl group; and wherein the C₆₋₁₀ aryl and 6-10 membered heteroaryl are each optionally substituted with 1, 2, or 3 substituents independently selected from R¹⁰;

R³ is selected from H, C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{f3}, and NR^{c3}R^{j3}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R³⁰;

R⁵ is selected from H, C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a5}, C(O)NR^{c5}R^{d5}, and NR^{c5}R^{d5}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R⁵⁰;

when R⁴R⁵C [symbol] R⁶ is a double bond and Y is N, then R⁴ and R⁶ are absent;

when R⁴R⁵C [symbol] YR⁶ is a double bond and Y is C, then R⁴ is absent; and

R⁶ is selected from H, C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a6}, and NR^{c6}R^{d6}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R⁶⁰;

R⁷ is selected from H, C₁₋₆ alkyl, C₁₋₆ haloalkyl, halo, D, and CN;

Cy² is selected from 4-10 membered heterocycloalkyl; wherein the 4-10 membered heterocycloalkyl has at least one ring-forming carbon atom and 1, 2, 3, or 4 ring-forming heteroatoms independently selected from N, O, and S; wherein a ring-forming carbon atom of the 4-10 membered heterocycloalkyl is optionally substituted by oxo to form a carbonyl group; and wherein the 4-10 membered heterocycloalkyl, is optionally substituted with 1, 2 or 3 substituents independently selected from R²⁰;

each R¹⁰ is independently selected from C₁₋₆ alkyl, C₁₋₆ haloalkyl, halo, D, CN, OR^{a10}, and NR^{c10}R^{d10};

each R²⁰ is independently selected from C₁₋₆ alkyl, C₂₋₆ alkenyl, C₂₋₆ alkynyl, C₁₋₆ haloalkyl, halo, D, CN, OR^{a20}, C(O)R^{b20}, C(O)NR^{c20}R^{d20}, and NR^{c20}R^{d20}; wherein said C₁₋₆ alkyl, C₂₋₆ alkenyl, and C₂₋₆ alkynyl, are each optionally substituted with 1, 2, or 3 substituents independently selected from R²¹;

each R²¹ is independently selected from C₁₋₆ alkyl, C₁₋₆ haloalkyl, halo, D, CN, OR^{a21} and NR^{c21}R^{d21};

each R²² is independently selected from C₁₋₆ alkyl, C₁₋₆ haloalkyl, halo, D, CN, OR^{a22} and NR^{c22}R^{d22};

each R³⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a30}, C(O)NR^{c30}R^{d30}, and NR^{c30}R^{d30}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R³¹;

each R³¹ is independently selected from C₁₋₆ alkyl, C₁₋₆ haloalkyl, halo, D, CN, OR^{a31}, and NR^{c31}R^{d31}.

each R⁵⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a5}, and NR^{c50}R^{d50};

each R⁶⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a60}, C(O)NR^{c60}R^{d60}, C(O)OR^{a60}, and NR^{c60}R^{d60};

each R^{a2}, R^{c2} and R^{d2} is independently selected from H, C₁₋₆ alkyl, and C₁₋₆ haloalkyl; wherein said C₁₋₆ alkyl is optionally substituted with 1 or 2 substituents independently selected from R²²;

each R^{c3} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³⁰;

each R^{f3} and R^{j3} is independently selected from C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³⁰;

or any R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;

each R^{a5}, R^{c5} and R^{d5} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R⁵⁰;

or any R^{c5} and R^{d5} attached to the same N atom, together with the N atom to which they are attached, form a 4-, 5-, or 6-membered heterocycloalkyl group optionally substituted with 1, 2, or 3 substituents independently selected from R⁵⁰;

each R^{a6}, R^{c6} and R^{d6} is independently selected from H, C₁₋₃ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R⁶⁰;

or any R^{c6} and R^{d6} attached to the same N atom, together with the N atom to which they are attached, form a 4-, 5-, or 6-membered heterocycloalkyl group optionally substituted with 1, 2, or 3 substituents independently selected from R⁶⁰;

each R^{a10}, R^{c10} and R^{d10} is independently selected from H, C₁₋₆ alkyl, and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1, 2, or 3 substituents independently selected from R²¹; each R^{a21}, R^{c21} and R^{d21}, is independently selected from H, C₁₋₆ alkyl, and C₁₋₆ haloalkyl;

each R^{a22}, R^{c22} and R^{d22} is independently selected from H, C₁₋₆ alkyl, and C₁₋₆ haloalkyl;

each R^{a30}, R^{c30} and R^{d30} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³¹;

or any R^{c30} and R^{d30} attached to the same N atom, together with the N atom to which they are attached, form a 4-, 5-, or 6-membered heterocycloalkyl group optionally substituted with 1, 2, or 3 substituents independently selected from R³¹;

each R^{a31}, R^{c31} and R^{d31}, is independently selected from H, C₁₋₆ alkyl, and C₁₋₆ haloalkyl;

each R^{a50}, R^{c50} and R^{d50}, is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl;

or any R^{c50} and R^{d50} attached to the same N atom, together with the N atom to which they are attached, form a 4-, 5-, or 6-membered heterocycloalkyl group; and each R^{a60}, R^{c60} and R^{d60} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl;

or any R^{c60} and R^{d60} attached to the same N atom, together with the N atom to which they are attached, form a 4-, 5-, or 6-membered heterocycloalkyl group.

In another embodiment of Formula Ia, or a pharmaceutically acceptable salt thereof,

Y is N or C;

R¹ is H;

R² is selected from H, C₁₋₆ alkyl, C₁₋₆ haloalkyl, halo, D, and CN;

Cy¹ is selected from C₆₋₁₀ aryl and 6-10 membered heteroaryl; wherein the 6-10 membered heteroaryl has at least one ring-forming carbon atom and 1 or 2 ring-forming heteroatoms independently selected from N and O; and wherein the C₆₋₁₀ aryl and 6-10 membered heteroaryl are each optionally substituted with 1, 2, or 3 substituents independently selected from R¹⁰;

R³ is selected from H, C₁₋₆ alkyl, C₁₋₆ haloalkyl, C₃₋₁₀ cycloalkyl, 4-10 membered heterocycloalkyl, halo, D, CN, OR^{f3}, and NR^{c3}R^{j3}; wherein said C₁₋₆ alkyl, C₃₋₁₀ cycloalkyl, and 4-10 membered heterocycloalkyl, are each optionally substituted with 1, 2, or 3 substituents independently selected from R³⁰;

R⁵ is selected from H, C₁₋₆ alkyl, C₁₋₆ haloalkyl, halo, D, and CN;

when R⁴R⁵C [symbol] YR⁶ is a double bond and Y is N, then R⁶ is absent;

R⁶ is selected from H, C₁₋₆ alkyl, C₁₋₆ haloalkyl, halo, D, and CN;

R⁷ is selected from H, C₁₋₆ alkyl, C₁₋₆ haloalkyl, halo, D, and CN;

Cy² is selected from 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted with 1, 2 or 3 substituents independently selected from R²⁰;

each R¹⁰ is independently selected from C₁₋₆ alkyl, C₁₋₆ haloalkyl, halo, D, CN, OR^{a10}, and NR^{c10}R^{d10};

each R²⁰ is independently selected from C₁₋₆ alkyl, C₁₋₆ haloalkyl, halo, D, CN, OR^{a20}, C(O)R^{b20}, C(O)NR^{c20}R^{d20}, and NR^{c20}R^{d20}; wherein said C₁₋₆ alkyl, is optionally substituted with 1 or 2 substituents independently selected from R²¹;

each R²¹ is independently selected from C₁₋₆ alkyl, C₁₋₆ haloalkyl, halo, D, CN, OR^{a21}, and NR^{c21}, R^{d21};

each R³⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a30}, C(O)NR^{c30}R^{d30}, and NR^{c30}R^{d30}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R³¹;

each R³¹ is independently selected from C₁₋₆ alkyl, C₁₋₆ haloalkyl, halo, D, CN, OR^{a31}, and NR^{c31}R^{d31};

each R^{c3} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³⁰;

each R^{f3} and R^{j3} is independently selected from C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³⁰;

or any R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;

each R^{a10}, R^{c10} and R^{d10} is independently selected from H, C₁₋₆ alkyl, and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1, 2, or 3 substituents independently selected from R²¹; each R^{a21}, R^{c21} and R^{d21}, is independently selected from H, C₁₋₆ alkyl, and C₁₋₆ haloalkyl;

each R^{a30}, R^{c30} and R^{d30} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³¹;

or any R^{c0} and R^{d30} attached to the same N atom, together with the N atom to which they are attached, form a 4-, 5-, or 6-membered heterocycloalkyl group optionally substituted with 1, 2, or 3 substituents independently selected from R³¹; and

each R^{a31}, R^{c31} and R^{d31}, is independently selected from H, C₁₋₆ alkyl, and C₁₋₆ haloalkyl.

In an embodiment,

[symbol] represents a single bond or a double bond;

X is N or CR⁷;

Y is N or C;

R¹ is selected from H, D, C₁₋₆ alkyl, C₂₋₆ alkenyl, C₂₋₆ alkynyl, C₁₋₆ haloalkyl, halo, CN, OR^{a1}, SR^{a1}, C(O)R^{b1}, C(O)NR^{c1}R^{d1}C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl, are each optionally substituted with 1, 2, 3, or 4 substituents independently selected from R^{g};

R² is selected from H, C₁₋₆ alkyl, C₂₋₆ alkenyl, C₂₋₆ alkynyl, C₁₋₆ haloalkyl, halo, D, CN, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, S(O)₂R^{b2}, and S(O)₂NR^{c2}R^{d2}; wherein said C₁₋₆ alkyl, C₂₋₆ alkenyl, and C₂₋₆ alkynyl, are each optionally substituted with 1, 2, 3, or 4 substituents independently selected from R²²;

Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3 or 4 substituents independently selected from R¹⁰;

R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{f3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{j3}, NR^{c3}C(O)R^{b3}, NR^{c3}C(O)OR^{a3}, NR^{c3}C(O)NR^{c3}R^{d3}, NR^{c3}S(O)₂R^{b3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³⁰;

when R⁴R⁵C [symbol] YR⁶ is a single bond and Y is C, then YR⁶ is selected from C═O and C═S; and

R⁴ is selected from H, D, C₁₋₆ alkyl, and C₁₋₆ haloalkyl; wherein said C₁₋₆ alkyl is optionally substituted with 1 or 2 substituents independently selected from R^{g};

R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, S(O)₂R^{b5}, and S(O)₂NR^{c5}R^{d5}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁵⁰;

when R⁴R⁵C [symbol] YR⁶ is a double bond and Y is N, then R⁴ and R⁶ are absent;

when R⁴R⁵C [symbol] YR⁶ is a double bond and Y is C, then R⁴ is absent; and

R⁶ is selected from H, D, C₁₋₆ alkyl, C₂₋₆ alkenyl, C₂₋₆ alkynyl, C₁₋₆ haloalkyl, C₃₋₆ cycloalkyl, 4-6 membered heterocycloalkyl, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂R^{b6}, S(O)₂R^{b6}, and S(O)₂NR^{c6}R^{d6}; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R⁶⁰;

R⁷ is selected from H, C₁₋₆ alkyl, C₂₋₆ alkenyl, C₂₋₆ alkynyl, C₁₋₆ haloalkyl, C₃₋₆ cycloalkyl, and 4-6 membered heterocycloalkyl, halo, D, CN, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}NR^{c7}S(O)₂R^{b7}, S(O)₂R^{b7}, and S(O)₂NR^{c7}R^{d7}; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R⁷⁰;

Cy² is 4-14 membered heterocycloalkyl; wherein the 4-14 membered heterocycloalkyl has at least one ring-forming carbon atom and 1, 2, 3, or 4 ring-forming heteroatoms independently selected from N, O, and S; wherein the N and S are optionally oxidized; wherein a ring-forming carbon atom of 4-14 membered heterocycloalkyl is optionally substituted by oxo to form a carbonyl group; and wherein the 4-14 membered heterocycloalkyl, is optionally substituted with 1, 2, 3 or 4 substituents independently selected from R²⁰;

each R¹⁰ is independently selected from C₁₋₆ alkyl, C₂₋₆ alkenyl, C₂₋₆ alkynyl, C₁₋₆ haloalkyl, C₃₋₆ cycloalkyl, 4-6 membered heterocycloalkyl, halo, D, CN, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, S(O)₂R^{b10} and S(O)₂NR^{c10}R^{d10}; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R¹¹;

each R¹¹ is independently selected from C₁₋₆ alkyl, C₁₋₆ haloalkyl, halo, D, CN, OR^{a11}, SR^{a11}, C C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, S(O)₂R^{b11} and S(O)₂NR^{c11}R^{d11};

each R²⁰ is independently selected from C₁₋₆ alkyl, C₂₋₆ alkenyl, C₂₋₆ alkynyl, C₁₋₆ haloalkyl, C₃₋₆ cycloalkyl, 4-6 membered heterocycloalkyl, halo, D, CN, OR^{a20}, SR^{a20}, C(O)R^{b20}, C(O)NR^{c20}R^{d20}, C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, NR^{c20}S(O)₂R^{b20}, S(O)₂R^{b20}, and S(O)₂NR^{c20}R^{d20}; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R²¹;

each R²¹ is independently selected from C₁₋₆ alkyl, C₂₋₆ alkenyl, C₂₋₆ alkynyl, C₁₋₆ haloalkyl, halo, D, CN, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, S(O)₂R^{b21}, and S(O)₂NR^{c21}R^{d21};

each R²² is independently selected from C₁₋₆ alkyl, C₂₋₆ alkenyl, C₂₋₆ alkynyl, C₁₋₆ haloalkyl, halo, D, CN, OR^{a22}, SR^{a22}, C(O)R^{b22}, C(O)NR^{c22}R^{d22}, C(O)OR^{a22}, OC(O)R^{b22}OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)₂R^{b22}, S(O)₂R^{b22}, and S(O)₂NR^{c22}R^{d22};

each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a30}, SR^{a30}, C(O)R^{b30}, C(O)NR^{c30}R^{d30}, C(O)OR^{a30}, OC(O)R^{b30}, OC(O)NR^{c30}R^{d30}, NR^{c30}R^{d30}, NR^{c30}C(O)R^{b30}, NR^{c30}C(O)OR^{a30}, NR^{c30}C(O)NROR^{d30} NR^{c30}S(O)₂R^{b30}, S(O)₂R^{b30}, and S(O)₂NR^{c30}R^{d30}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R³¹;

each R³¹ is independently selected from C₁₋₆ alkyl, C₂₋₆ alkenyl, C₂₋₆ alkynyl, C₁₋₆ haloalkyl, C₃₋₆ cycloalkyl, 4-6 membered heterocycloalkyl, halo, D, CN, OR^{a31}, SR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR^{c31}C(O)R^{b31}, NR^{c31}C(O)OR^{a31}, NR^{c31}C(O)NR^{c31}R^{d31}, NR^{c31}S(O)₂R^{b31}, S(O)₂R^{b31}, and S(O)₂NR^{c31}R^{d31}; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R³²;

each R³² is independently selected from C₁₋₆ alkyl, C₂₋₆ alkenyl, C₂₋₆ alkynyl, C₁₋₆ haloalkyl, halo, D, CN, OR^{a32}, SR^{a32}, C(O)R^{b32}, C(O)NR^{c32}R^{d32}, C(O)OR^{a32}, OC(O)R^{b32}OC(O)NR^{c32}R^{d32}, NR^{c32}R^{d32}, NR^{c32}C(O)R^{b32}, NR^{c32}C(O)OR^{a32}, NR^{c32}C(O)NR^{c32}R^{d32}, NR^{c32}S(O)₂R^{b32}, S(O)₂R^{b32}, and S(O)₂NR^{c32}R^{d32};

each R⁵⁰ is independently selected from C₁₋₆ alkyl, C₂₋₆ alkenyl, C₂₋₆ alkynyl, C₁₋₆ haloalkyl, halo, D, CN, OR^{a50}, SR^{a50}, C(O)R^{b50}, C(O)NR^{c50}R^{d50}, C(O)OR^{a50}, OC(O)R^{b50}, OC(O)NR^{c50}R^{d50}, NR^{c50}R^{d50}, NR^{c50}C(O)R^{b50}, NR^{c50}C(O)OR^{a50}, NR^{c50}C(O)NR^{c50}R^{d50}, NR^{c50}S(O)₂R^{b50}, S(O)₂R^{b50}, and S(O)₂NR^{c50}R^{d50};

each R⁶⁰ is independently selected from C₁₋₆ alkyl, C₂₋₆ alkenyl, C₂₋₆ alkynyl, C₁₋₆ haloalkyl, halo, D, CN, OR^{a60}, SR^{a60}, C(O)R^{b60}, C(O)NR^{c60}R^{d60}, C(O)OR^{a60}, OC(O)R^{b60}, OC(O)NR^{c60}R^{d60}, NR^{c60}R^{d60}, NR^{c60}C(O)R^{b60}, NR^{c60}C(O)OR^{a60}, NR^{c60}C(O)NR^{c60}R^{d60}, NR^{c60}S(O)₂R^{b60}, S(O)₂R^{b60}, and S(O)₂NR^{c60}R^{d60};

each R⁷⁰ is independently selected from C₁₋₆ alkyl, C₂₋₆ alkenyl, C₂₋₆ alkynyl, C₁₋₆ haloalkyl, halo, D, CN, OR^{a70}, SR^{a70}, C(O)R^{b70}, C(O)NR^{c70}R^{d70}, C(O)OR^{a70}, OC(O)R^{b70}, OC(O)NR^{c70}R^{d70}, NR^{c70}R^{d70}, NR^{c70}C(O)R^{b70}, NR^{c70}C(O)OR^{a70}, NR^{c70}C(O)NR^{c70}R^{d70}, NR^{c70}S(O)₂R^{b70}, S(O)₂R^{b70}, and S(O)₂NR^{d70}R^{d70};

each R^{a1}, R^{b1}, R^{c1}, and R^{d1} is independently selected from H, C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1, 2, 3, or 4 substituents independently selected from R^{g};

each R^{a2}, R^{b2}, R^{c2} and R^{d2} is independently selected from H, C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1, 2, 3, or 4 substituents independently selected from R²²;

each R^{a3}, R^{b3}, R^{c3} and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{f3} and R^{j3} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;

or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰;

each R^{a5}, R^{b5}, R^{c5} and R^{d5} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁵⁰;

or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R⁵⁰;

each R^{a6}, R^{b6}, R^{c6}, and R^{d6} is independently selected from H, C₁₋₆ alkyl, C₂₋₆ alkenyl, C₂₋₆ alkynyl, C₁₋₆ haloalkyl, C₃₋₆ cycloalkyl, and 4-6 membered heterocycloalkyl; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R⁶⁰;

or any R^{c6} and R^{d6} attached to the same N atom, together with the N atom to which they are attached, form a 4-, 5-, or 6-membered heterocycloalkyl group optionally substituted with 1, 2 or 3 substituents independently selected from R⁶⁰;
each R^{a7}, R^{b7}, R^{c7} and R^{d7} is independently selected from H, C₁₋₆ alkyl, C₂₋₆ alkenyl, C₂₋₆ alkynyl, C₁₋₆ haloalkyl, C₃₋₆ cycloalkyl, and 4-6 membered heterocycloalkyl; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R⁷⁰;

or any R^{c7} and R^{d7} attached to the same N atom, together with the N atom to which they are attached, form a 4-, 5-, or 6-membered heterocycloalkyl group optionally substituted with 1, 2, 3, or 4 substituents independently selected from R⁷⁰;

each R^{a10}, R^{b10}, R^{c10} and R^{d10} is independently selected from H, C₁₋₆ alkyl, C₂₋₆ alkenyl, C₂₋₆ alkynyl, C₁₋₆ haloalkyl, C₃₋₆ cycloalkyl, and 4-6 membered heterocycloalkyl; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R¹¹;

or any R^{c10} and R^{d10} attached to the same N atom, together with the N atom to which they are attached, form a 4-, 5-, or 6-membered heterocycloalkyl group optionally substituted with 1, 2, 3, or 4 substituents independently selected from R¹¹;

each R^{a11}, R^{b11}, R^{c11} and R^{d11}, is independently selected from H, C₁₋₆ alkyl, C₂₋₆ alkenyl, C₂₋₆ alkynyl, and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, C₁₋₆ haloalkyl, C₃₋₆ cycloalkyl, and 4-6 membered heterocycloalkyl; wherein said C₁₋₆ alkyl, C₂₋₆ alkenyl, C₂₋₆ alkynyl, C₃₋₆ cycloalkyl, and 4-6 membered heterocycloalkyl, are each optionally substituted with 1, 2, 3, or 4 substituents independently selected from R²¹;

or any R^{c20} and R^{d20} attached to the same N atom, together with the N atom to which they are attached, form a 4-, 5-, or 6-membered heterocycloalkyl group optionally substituted with 1, 2, 3, or 4 substituents independently selected from R²¹;

each R^{a21}, R^{b21}, R^{c21} and R^{d21}, is independently selected from H, C₁₋₆ alkyl, C₂₋₆ alkenyl, C₂₋₆ alkynyl, and C₁₋₆ haloalkyl;

each R^{a22}, R^{b22}, R^{c22} and R^{d22} is independently selected from H, C₁₋₆ alkyl, C₂₋₆ alkenyl, C₂₋₆ alkynyl, and C₁₋₆ haloalkyl;

each R^{a30}, R^{b30}, R^{c30} and R^{d30} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³¹; or any R^{c30} and R^{d30} attached to the same N atom, together with the N atom to which they are attached, form a 4-, 5-, 6- or 7-membered heterocycloalkyl group optionally substituted with 1, 2, 3, or 4 substituents independently selected from R³¹;

each R^{a31}, R^{b31}, R^{c31} and R^{d31}, is independently selected from H, C₁₋₆ alkyl, C₂₋₆ alkenyl, C₂₋₆ alkynyl, C₁₋₆ haloalkyl, C₃₋₆ cycloalkyl, and 4-6 membered heterocycloalkyl; wherein said C₁₋₆ alkyl C₂₋₆ alkenyl, C₂₋₆ alkynyl, C₃₋₆ cycloalkyl, and 4-6 membered heterocycloalkyl are each optionally substituted with 1, 2, 3, or 4 substituents independently selected from R³²;

or any R^{c31} and R^{d31} attached to the same N atom, together with the N atom to which they are attached, form a 4-, 5-, or 6-membered heterocycloalkyl group optionally substituted with 1, 2 or 3 substituents independently selected from R³²;

each R^{a32}, R^{b32}, R^{c32} and R^{d32}, is independently selected from H, C₁₋₆ alkyl, C₂₋₆ alkenyl, C₂₋₆ alkynyl and C₁₋₆ haloalkyl;

each R^{a50}, R^{b50}, R^{c50} and R^{d50}, is independently selected from H, C₁₋₆ alkyl, C₂₋₆ alkenyl, C₂₋₆ alkynyl, and C₁₋₆ haloalkyl;

each R^{a60}, R^{b60}, R^{c60} and R^{d60} is independently selected from H, C₁₋₆ alkyl, C₂₋₆ alkenyl, C₂₋₆ alkynyl, and C₁₋₆ haloalkyl;
each R^{a70}, R^{b70}, R^{c70} and R^{d70} is independently selected from H, C₁₋₆ alkyl, C₂₋₆ alkenyl, C₂₋₆ alkynyl, and C₁₋₆ haloalkyl; and

each R^{g} is independently selected from D, OH, CN, halo, C₁₋₆ alkyl, C₂₋₆ alkenyl, C₂₋₆ alkynyl, C₁₋₆ haloalkyl, C₃₋₆ cycloalkyl, C₁₋₆ alkoxy, C₁₋₆ haloalkoxy, C₁₋₃ alkoxy-C₁₋₃ alkyl, C₁₋₃ HO—C₁₋₃ alkyl, cyano-C₁₋₃ alkyl, H₂N—C₁₋₃ alkyl, amino, C₁₋₆ alkylamino, di(C₁₋₆ alkyl)amino, C₁₋₆ alkylsulfonyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, C₁₋₆ alkylaminocarbonyloxy, di(C₁₋₆ alkyl)aminocarbonyloxy, C₁₋₆ alkylaminocarbonylamino, and di(C₁₋₆ alkyl)aminocarbonylamino.

In an embodiment of Formula I, or a pharmaceutically acceptable salt thereof,

[symbol] represents a single bond or a double bond;

X is N or CR⁷;

Y is N or C;

R¹ is selected from H, D, C₁₋₆ alkyl, C₁₋₆ haloalkyl, halo, and CN;

R² is selected from H, C₁₋₆ alkyl, C₂₋₆ alkenyl, C₂₋₆ alkynyl, C₁₋₆ haloalkyl, halo, D, CN, OR^{a2}, and NR^{c2}R^{d2}; wherein said C₁₋₆ alkyl, C₂₋₆ alkenyl, and C₂₋₆ alkynyl, are each optionally substituted with 1, 2, 3, or 4 substituents independently selected from R²²;

Cy¹ is selected from C₆₋₁₀ aryl and 6-10 membered heteroaryl; wherein the 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl is optionally substituted by oxo to form a carbonyl group; and wherein the C₆₋₁₀ aryl and 6-10 membered heteroaryl are each optionally substituted with 1, 2, 3 or 4 substituents independently selected from R¹⁰;

R³ is selected from H, C₁₋₆ alkyl, C₁₋₆ haloalkyl, 4-10 membered heterocycloalkyl, C₆₋₁₀ aryl, 5-10 membered heteroaryl, halo, D, CN, OR^{f3}, and NR^{c3}R^{j3}; wherein said C₁₋₆ alkyl, 4-10 membered heterocycloalkyl, C₆₋₁₀ aryl, and 5-10 membered heteroaryl, are each optionally substituted with 1, 2, 3, or 4 substituents independently selected from R³⁰;

R⁵ is selected from H, C₁₋₆ alkyl, C₁₋₆ haloalkyl, C₆₋₁₀ aryl, 5-10 membered heteroaryl, and D; wherein said C₁₋₆ alkyl, C₆₋₁₀ aryl, and 5-10 membered heteroaryl, are each optionally substituted with 1, 2, 3, or 4 substituents independently selected from R⁵⁰;

when R⁴R⁵C [symbol] YR⁶ is a double bond and Y is N, then R⁴ and R⁶ are absent;

when R⁴R⁵C [symbol] YR⁶ is a double bond and Y is C, then R⁴ is absent; and

R⁶ is selected from H, D, C₁₋₆ alkyl, C₁₋₆ haloalkyl, C₃₋₆ cycloalkyl, and 4-6 membered heterocycloalkyl; wherein said C₁₋₆ alkyl, C₃₋₆ cycloalkyl, and 4-6 membered heterocycloalkyl, are each optionally substituted with 1, 2, 3, or 4 substituents independently selected from R⁶⁰;

R⁷ is selected from H, C₁₋₆ alkyl, C₁₋₆ haloalkyl, halo, D, and CN;

Cy² is 4-14 membered heterocycloalkyl; wherein the 4-14 membered heterocycloalkyl has at least one ring-forming carbon atom and 1, 2, 3, or 4 ring-forming heteroatoms independently selected from N, O, and S; and wherein the 4-14 membered heterocycloalkyl, is optionally substituted with 1, 2, 3 or 4 substituents independently selected from R²⁰;

each R¹⁰ is independently selected from C₁₋₆ alkyl, C₁₋₆ haloalkyl, halo, D, CN, OR^{a10}, and NR^{c10}R^{d10};

each R²⁰ is independently selected from C₁₋₆ alkyl, C₁₋₆ haloalkyl, halo, D, CN, OR^{a20}, C(O)R^{b20}, C(O)NR^{c20}R^{d20}, C(O)OR^{a20} and NR^{c20}R^{d20}; wherein said C₁₋₆ alkyl, is optionally substituted with 1, 2, 3, or 4 substituents independently selected from R²¹;

each R²¹ is independently selected from C₁₋₆ alkyl, C₁₋₆ haloalkyl, halo, D, CN, OR^{a21} and NR^{c21}R^{d21};

each R²² is independently selected from C₁₋₆ alkyl, C₁₋₆ haloalkyl, halo, D, CN, OR^{a22} and NR^{c22}R^{d22};

each R³⁰ is independently selected from C₁₋₆ alkyl, C₁₋₆ haloalkyl, 4-10 membered heterocycloalkyl, 5-10 membered heteroaryl, halo, D, CN, OR^{a30}, and NR^{c30}R^{d30}; wherein said C₁₋₆ alkyl, 4-10 membered heterocycloalkyl, and 5-10 membered heteroaryl, are each optionally substituted with 1, 2, 3, or 4 substituents independently selected from R³¹;

each R³¹ is independently selected from C₁₋₆ alkyl, C₁₋₆ haloalkyl, halo, D, CN, OR^{a31}, and NR^{c31}R^{d31};

each R⁵⁰ is independently selected from C₁₋₆ alkyl, C₁₋₆ haloalkyl, halo, D, CN, OR^{a50}, and NR^{c50}R^{d50};

each R⁶⁰ is independently selected from C₁₋₆ alkyl, C₁₋₆ haloalkyl, halo, D, CN, OR^{a60}, C(O)R^{b60}, C(O)NR^{c6}R^{d60}, C(O)OR^{a60}, and NR^{c60}R^{d60};

each R^{a2}, R^{c2} and R^{d2} is independently selected from H, C₁₋₆ alkyl, and C₁₋₆ haloalkyl;

each R^{c3} is independently selected from H, C₁₋₆ alkyl, C₁₋₆ haloalkyl, 4-10 membered heterocycloalkyl, and 5-10 membered heteroaryl; wherein said C₁₋₆ alkyl 4-10 membered heterocycloalkyl, and 5-10 membered heteroaryl, are each optionally substituted with 1, 2, 3, or 4 substituents independently selected from R³⁰;

each R^{f3} and R^{j3} is independently selected from C₁₋₆ alkyl, C₁₋₆ haloalkyl, 4-10 membered heterocycloalkyl, and 5-10 membered heteroaryl; wherein said C₁₋₆ alkyl, 4-10 membered heterocycloalkyl, and 5-10 membered heteroaryl, are each optionally substituted with 1, 2, 3, or 4 substituents independently selected from R³⁰;

or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰; each R^{a10}, R^{b10}, R^{c10} and R^{d10} is independently selected from H, C₁₋₆ alkyl, and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1, 2, 3, or 4 substituents independently selected from R²¹;

each R^{a21}, R^{b21}, R^{c21} and R^{d21}, is independently selected from H, C₁₋₆ alkyl, and C₁₋₆ haloalkyl;

each R^{a30}, R^{b30}, R^{c30} and R^{d30}, is independently selected from H, C₁₋₆ alkyl, and C₁₋₆ haloalkyl;

each R^{a31}, R^{b31}, R^{c31} and R^{d31}, is independently selected from H, C₁₋₆ alkyl, and C₁₋₆ haloalkyl;

each R^{a50}, R^{b50}, R^{c50} and R^{d50}, is independently selected from H, C₁₋₆ alkyl, and C₁₋₆ haloalkyl; and

each R^{a60}, R^{b60}, R^{c60} and R^{d60} is independently selected from H, C₁₋₆ alkyl, and C₁₋₆ haloalkyl.

In another embodiment of Formula I, or a pharmaceutically acceptable salt thereof,

[symbol] represents a single bond or a double bond;

X is CR⁷;

Y is N or C;

R¹ is H;

R² is selected from H, C₁₋₆ alkyl, C₁₋₆ haloalkyl, halo, D, and CN; wherein said C₁₋₆ alkyl, is optionally substituted with 1 or 2 substituents independently selected from R²²;

Cy¹ is selected from C₆₋₁₀ aryl and 6-10 membered heteroaryl; wherein the 6-10 membered heteroaryl each has at least one ring-forming carbon atom and 1 or 2 ring-forming heteroatoms independently selected from N, and O; and wherein the C₆₋₁₀ aryl and 6-10 membered heteroaryl are each optionally substituted with 1, 2, or 3 substituents independently selected from R¹⁰;

R³ is selected from H, C₁₋₆ alkyl, C₁₋₆ haloalkyl, 4-6 membered heterocycloalkyl, OR^{f3}, and NR^{c3}R^{j3}; wherein said C₁₋₆ alkyl and 4-6 membered heterocycloalkyl, are each optionally substituted with 1 or 2 substituents independently selected from R³⁰;

R⁵ is selected from H, C₁₋₆ alkyl, C₁₋₆ haloalkyl, phenyl, 5-6 membered heteroaryl, and D; wherein said C₁₋₆ alkyl, phenyl, and 5-6 membered heteroaryl, are each optionally substituted with 1 or 2 substituents independently selected from R⁵⁰;

when R⁴R⁵C [symbol] YR⁶ is a double bond and Y is N, then R⁴ and R⁶ are absent;

when R⁴R⁵C [symbol] YR⁶ is a double bond and Y is C, then R⁴ is absent; and

R⁶ is selected from H, D, C₁₋₆ alkyl, and C₁₋₆ haloalkyl; wherein said C₁₋₆ alkyl is optionally substituted with 1 or 2 substituents independently selected from R6;

R⁷ is selected from halo;

Cy² is 4-8 membered heterocycloalkyl; wherein the 4-8 membered heterocycloalkyl has at least one ring-forming carbon atom and 1 or 2 ring-forming heteroatoms independently selected from N and O; and wherein the 4-8 membered heterocycloalkyl, is optionally substituted with 1 or 2 substituents independently selected from R²⁰;

each R¹⁰ is independently selected from C₁₋₆ alkyl, C₁₋₆ haloalkyl, halo, D, CN, OR^{a10}, and NR^{c10}R^{d10};

each R²⁰ is independently selected from C₁₋₆ alkyl, C₁₋₆ haloalkyl, halo, D, CN, and C(O)R^{b20}; wherein said C₁₋₆ alkyl, is optionally substituted with 1 or 2 substituents independently selected from R²¹;

each R²¹ is independently selected from C₁₋₆ alkyl, C₁₋₆ haloalkyl, halo, D, CN, OR^{a21} and NR^{c21}R^{d21};

each R²² is independently selected from C₁₋₆ alkyl, C₁₋₆ haloalkyl, halo, D, CN, OR^{a22} and NR^{c22}R^{d22};

each R³⁰ is independently selected from C₁₋₆ alkyl, C₁₋₆ haloalkyl, 4-6 membered heterocycloalkyl, 5-6 membered heteroaryl, halo, D, CN, OR^{a30}, and NR^{c30}R^{d30}; wherein said C₁₋₆ alkyl, 4-6 membered heterocycloalkyl, and 5-6 membered heteroaryl, are each optionally substituted with 1 or 2 substituents independently selected from R³¹;

each R³¹ is independently selected from C₁₋₆ alkyl, C₁₋₆ haloalkyl, halo, D, and CN;

each R⁵⁰ is independently selected from C₁₋₆ alkyl, C₁₋₆ haloalkyl, halo, D, and CN;

each R⁶⁰ is independently selected from C₁₋₆ alkyl, C₁₋₆ haloalkyl, halo, D, CN, OR^{a60}, C(O)R^{b60}, C(O)NR^{c60}R^{d60}, C(O)OR^{a60}, and NR^{c60}R^{d60};

each R^{c3} is independently selected from H, C₁₋₆ alkyl, and C₁₋₆ haloalkyl; wherein said C₁₋₆ alkyl is optionally substituted with 1 or 2 substituents independently selected from R³⁰;

each R^{f3} and R^{j3} is independently selected from C₁₋₆ alkyl, and C₁₋₆ haloalkyl; wherein said C₁₋₆ alkyl, is optionally substituted with 1 or 2 substituents independently selected from R³⁰;

each R^{a10}, R^{b10}, R^{c10} and R^{d10} is independently selected from H, C₁₋₆ alkyl, and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1 or 2 substituents independently selected from R²¹;

each R^{a21}, R^{b21}, R^{c21} and R^{d21}, is independently selected from H, C₁₋₆ alkyl, and C₁₋₆ haloalkyl;

each R^{a30}, R^{b30}, R^{c30} and R^{d30}, is independently selected from H, C₁₋₆ alkyl, and C₁₋₆ haloalkyl; and

each R^{a60}, R^{b60}, R^{c60} and R^{d60} is independently selected from H, C₁₋₆ alkyl, and C₁₋₆ haloalkyl.

In another embodiment, the compound of Formula I is a compound of Formula II:

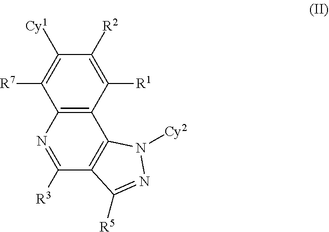

or a pharmaceutically acceptable salt thereof.

In yet another embodiment,

R¹ is selected from H, D, C₁₋₆ alkyl, C₂₋₆ alkenyl, C₂₋₆ alkynyl, C₁₋₆ haloalkyl, halo, CN, OR^{a1}, and NR^{c1}R^{d1};

R² is selected from H, C₁₋₆ alkyl, C₂₋₆ alkenyl, C₂₋₆ alkynyl, C₁₋₆ haloalkyl, halo, D, CN, OR^{a2}, and NR^{c2}R^{d2};

Cy¹ is selected from C₆₋₁₀ aryl and 6-10 membered heteroaryl; wherein the 6-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 6-10 membered heteroaryl is optionally substituted by oxo to form a carbonyl group; and wherein the C₆₋₁₀ aryl and 6-10 membered heteroaryl are each optionally substituted with 1 or 2 substituents independently selected from R¹⁰;

R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{f3}, C(O)R^{b3}, C(O)NR^{c3}R^{d3}, C(O)OR^{a3}, OC(O)R^{b3}, OC(O)NR^{c3}R^{d3}, NR^{c3}R^{j3}, NR^{c3}C(O)R^{b3}, NR^{c3}C(O)OR^{a3}, and S(O)₂R^{b3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1 or 2 substituents independently selected from R³⁰;

R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, and S(O)₂R^{b5}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1 or 2 substituents independently selected from R⁵⁰;

R⁷ is selected from H, C₁₋₆ alkyl, C₂₋₆ alkenyl, C₂₋₆ alkynyl, C₁₋₆ haloalkyl, halo, D, CN, OR^{a7}, and NR^{c7}R^{d7};

Cy² is 4-10 membered heterocycloalkyl; wherein the 4-10 membered heterocycloalkyl has at least one ring-forming carbon atom and 1, 2, 3, or 4 ring-forming heteroatoms independently selected from N, O, and S; wherein the N and S are optionally oxidized; wherein a ring-forming carbon atom of 4-10 membered heterocycloalkyl is optionally substituted by oxo to form a carbonyl group; and wherein the 4-10 membered heterocycloalkyl, is optionally substituted with 1 or 2 substituents independently selected from R²⁰;

each R¹⁰ is independently selected from C₁₋₆ alkyl, C₂₋₆ alkenyl, C₂₋₆ alkynyl, C₁₋₆ haloalkyl, halo, D, CN, OR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, and S(O)₂Rb¹⁰;

each R²⁰ is independently selected from C₁₋₆ alkyl, C₂₋₆ alkenyl, C₂₋₆ alkynyl, C₁₋₆ haloalkyl, halo, D, CN, OR^{a20}, C(O)R^{b20}, C(O)NR^{c20}R^{d20}, C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, and S(O)₂R^{b20};

each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a30}, C(O)R^{b30}, C(O)NR^{c30}R^{d30}C(O)OR^{a30}, OC(O)R^{b30}, OC(O)NR^{c30}R^{d30}, NR^{c30}R^{d30}, NR^{c30}C(O)R^{b30}, NR^{c30}C(O)OR^{a30}, and S(O)₂R^{b30}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1 or 2 substituents independently selected from R³¹;

each R³¹ is independently selected from C₁₋₆ alkyl, C₂₋₆ alkenyl, C₂₋₆ alkynyl, C₁₋₆ haloalkyl, halo, D, CN, OR^{a31}, and NR^{c31}R^{d31};

each R⁵⁰ is independently selected from C₁₋₆ alkyl, C₂₋₆ alkenyl, C₂₋₆ alkynyl, C₁₋₆ haloalkyl, halo, D, CN, OR^{a50}, and NR^{c50}R^{d50};

each R^{a1}, R^{c1}, and R^{d1} is independently selected from H, C₁₋₆ alkyl, C₂₋₆ alkenyl, C₂₋₆ alkynyl, and C₁₋₆ haloalkyl;

each R^{a2}, R^{c2} and R^{d2} is independently selected from H, C₁₋₆ alkyl, C₂₋₆ alkenyl, C₂₋₆ alkynyl, and C₁₋₆ haloalkyl;

each R^{a3}, R^{b3}, R^{c3} and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1 or 2 substituents independently selected from R³⁰;

or any R^{c3} and R^{d3} attached to the same N atom, together with the N atom to which they are attached, form a 4-, 5-, or 6-membered heterocycloalkyl group optionally substituted with 1 or 2 substituents independently selected from R³⁰;

each R^{a5}, R^{b5}, R^{c5} and R^{d5} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1 or 2 substituents independently selected from R⁵⁰;

or any R^{c5} and R^{d5} attached to the same N atom, together with the N atom to which they are attached, form a 4-, 5-, or 6-membered heterocycloalkyl group optionally substituted with 1 or 2 substituents independently selected from R⁵⁰;

each R^{a7}, R^{c7} and R^{d7} is independently selected from H, C₁₋₆ alkyl, C₂₋₆ alkenyl, C₂₋₆ alkynyl, and C₁₋₆ haloalkyl;

each R^{a10}, R^{b10}, R^{c10} and R^{d10} is independently selected from H, C₁₋₆ alkyl, C₂₋₆ alkenyl, C₂₋₆ alkynyl, and C₁₋₆ haloalkyl;

each R^{a20}, R^{b20}, R^{c20} and R^{d20} is independently selected from H, C₁₋₆ alkyl, C₂₋₆ alkenyl, C₂₋₆ alkynyl, and C₁₋₆ haloalkyl;

each R^{a30}, R^{b30}, R^{c30} and R^{d30} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1 or 2 substituents independently selected from R³¹;

or any R^{c30} and R^{d30} attached to the same N atom, together with the N atom to which they are attached, form a 4-, 5-, or 6-membered heterocycloalkyl group optionally substituted with 1 or 2 substituents independently selected from R³¹;

each R^{a31}, R^{c31} and R^{d31}, is independently selected from H, C₁₋₆ alkyl, C₂₋₆ alkenyl, C₂₋₆ alkynyl, and C₁₋₆ haloalkyl; and

each R^{a50}, R^{c50} and R^{d50}, is independently selected from H, C₁₋₆ alkyl, C₂₋₆ alkenyl, C₂₋₆ alkynyl, and C₁₋₆ haloalkyl.

In still another embodiment,

R¹ is selected from H, D, and C₁₋₃ alkyl;

R² is selected from H, C₁₋₃ alkyl, C₁₋₃ haloalkyl, halo, D, and CN;

Cy¹ is C₆₋₁₀ aryl; and wherein the C₆₋₁₀ aryl is optionally substituted with 1 or 2 substituents independently selected from R¹⁰;

R³ is selected from H, C₁₋₃ alkyl, 4-6 membered heterocycloalkyl, and D; wherein said C₁₋₃ alkyl and 4-6 membered heterocycloalkyl, are each optionally substituted with 1 or 2 substituents independently selected from R³⁰;

R⁵ is selected from H, C₁₋₃ alkyl, and D;

R⁷ is selected from H, C₁₋₃ alkyl, C₁₋₃ haloalkyl, halo, D, and CN;

Cy² is 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted with 1 or 2 substituents independently selected from R²⁰;

each R¹⁰ is independently selected from C₁₋₃ alkyl, C₁₋₃ haloalkyl, halo, D, CN, and OR^{a10};

each R²⁰ is independently selected from C₁₋₃ alkyl, D, and C(O)R^{b20};

each R³⁰ is independently selected from C₁₋₃ alkyl, C₁₋₃ haloalkyl, halo, D, CN, OR^{a30}, and NR^{c30}R^{d30};

each R^{a10} is independently selected from H and C₁₋₃ alkyl;

each R^{b20} is independently selected from H, C₁₋₃ alkyl, and C₂₋₃ alkenyl; and each R^{a30}, R^{c30} and R^{d30} is independently selected from H, C₁₋₃ alkyl, and C₁₋₃ haloalkyl.

In an embodiment,

X is CR⁷;

R¹ is selected from H;

R² is selected from H, C₁₋₃ haloalkyl, and halo;

Cy¹ is C₁₀ aryl; and wherein the C₁₀ aryl is optionally substituted with 1 or 2 substituents independently selected from R¹⁰;

R³ is selected from H and 4-6 membered heterocycloalkyl; wherein said 4-6 membered heterocycloalkyl, is optionally substituted with 1 or 2 substituents independently selected from R³⁰;

R⁵ is H;

R⁴R⁵C [symbol] R⁶ is a double bond, Y is N, and R⁴ and R⁶ are absent;

R⁷ is selected from H or halo;

Cy² is 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted with 1 or 2 substituents independently selected from R²⁰;

each R¹⁰ is independently selected from OR^{a10};

each R²⁰ is independently selected from C(O)R^{b20};

each R³⁰ is independently selected from NR^{c30}R^{d30};

each R^{a10} is independently selected from H and C₁₋₃ alkyl;

each R^{b20} is C₁₋₃ alkyl or C₂₋₄ alkenyl; and

each R^{c30} and R^{d30} is independently selected from C₁₋₃ alkyl.

In another embodiment of Formula I, or a pharmaceutically acceptable salt thereof,

[symbol] represents a single bond a single bond or a double bond;

X is CH or C-halo;

Y is N or C;

R¹ is H;

R² is selected from H, C₁₋₆ alkyl, C₁₋₆ haloalkyl, halo, and CN; wherein said C₁₋₆ alkyl is optionally substituted with 1 or 2 substituents independently selected from D, CN, OH, O(C₁₋₆ alkyl), NH₂, NH(C₁₋₆ alkyl), and N(C₁₋₆ alkyl)₂;

Cy¹ is selected from C₆₋₁₀ aryl and 6-10 membered heteroaryl; wherein the 6-10 membered heteroaryl each has at least one ring-forming carbon atom and 1 or 2 ring-forming heteroatoms independently selected from N, and O; and wherein the C₆₋₁₀ aryl and 6-10 membered heteroaryl are each optionally substituted with 1, 2, or 3 substituents independently selected from OH, halo, C₁₋₆ alkyl, and C₁₋₆ haloalkyl;

R³ is selected from H, C₁₋₆ alkyl, and 4-6 membered heterocycloalkyl, and O(C₁₋₆ alkyl); wherein said C₁₋₆ alkyl and 4-6 membered heterocycloalkyl are each optionally substituted with 1 or 2 substituents independently selected from R³⁰;

R⁵ is selected from H, phenyl, and 5-6 membered heteroaryl; wherein said phenyl and 5-6 membered heteroaryl are each optionally substituted with 1 or 2 substituents independently selected from C₁₋₆ alkyl;

when R⁴R⁵C [symbol] YR⁶ is a double bond and Y is N, then R⁴ and R⁶ are absent;

when R⁴R⁵C [symbol] YR⁶ is a double bond and Y is C, then R⁴ is absent; and

R⁶ is selected from H, C₁₋₆ alkyl, and 5-10 membered heteroaryl; wherein said C₁₋₆ alkyl and 5-10 membered heteroaryl are each optionally substituted with 1 or 2 substituents independently selected from C₁₋₆ alkyl, C(O)N(C₁₋₆ alkyl)₂, and C(O)OC₁₋₆ alkyl;

Cy² is 4-8 membered heterocycloalkyl; wherein the 4-8 membered heterocycloalkyl has at least one ring-forming carbon atom and 1 or 2 ring-forming heteroatoms independently selected from N and O; and wherein the 4-8 membered heterocycloalkyl is optionally substituted with 1 or 2 substituents independently selected from C₁₋₆ alkyl-CN and C(O)R^{b20};
each R³⁰ is independently selected from C₁₋₆ alkyl, 4-6 membered heterocycloalkyl, halo, and N(C₁₋₆ alkyl)₂; wherein said 4-6 membered heterocycloalkyl is optionally substituted with 1 or 2 substituents independently selected from C₁₋₆ alkyl; and

each R^{b20} is independently selected from C₂₋₆ alkenyl and C₂₋₆ alkynyl; wherein said C₂₋₆ alkenyl and C₂₋₆ alkynyl are each optionally substituted with 1 or 2 substituents independently selected from C₁₋₆ alkyl, C₁₋₆ alkylO(C₁₋₆ alkyl), C₁₋₆ haloalkyl, halo, and C₁₋₆ alkyl-N(C₁₋₆ alkyl)₂.

In another embodiment, the compound of Formula I is a compound of Formula III:

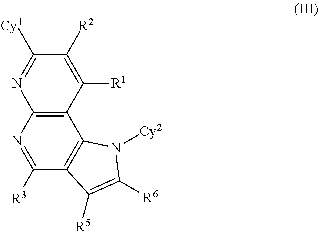

or a pharmaceutically acceptable salt thereof.

In yet another embodiment, wherein the compound of Formula I is a compound of Formula IV:

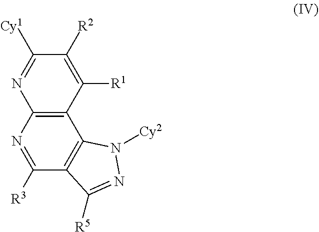

or a pharmaceutically acceptable salt thereof.

In still another embodiment, the compound of Formula I is a compound of Formula V:

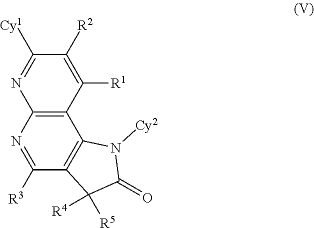

or a pharmaceutically acceptable salt thereof.

In an embodiment, the compound of Formula I is a compound of Formula VI:

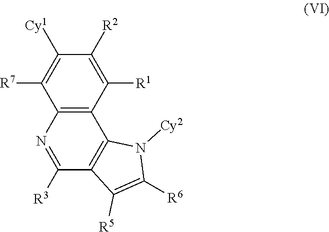

or a pharmaceutically acceptable salt thereof.

In another embodiment, the compound of Formula I is a compound of Formula VII:

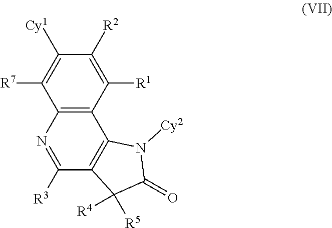

or a pharmaceutically acceptable salt thereof.

In yet another embodiment, X is CR⁷. In still another embodiment, X is N.

In an embodiment, R⁴R⁵C [symbol] YR⁶ is a double bond, Y is N, and R⁴ and R⁶ are absent. In another embodiment, R⁴R⁵C [symbol] YR⁶ is a single bond and YR⁶ is C═O. In an embodiment, R⁴R⁵C [symbol] YR⁶ is a double bond, Y is C, and R⁴ is absent.

In yet another embodiment, R¹ is selected from H, D, C₁₋₆ alkyl, C₁₋₆ haloalkyl, halo, OR^{a1}, and NR^{c1}R^{d1}. In yet another embodiment, R¹ is selected from H, D, C₁₋₆ alkyl, C₁₋₆ haloalkyl, halo, and CN. In still another embodiment, R¹ is selected from H, D, and C₁₋₃ alkyl.

In still another embodiment, R¹ is H.

In an embodiment, R² is selected from H, C₁₋₆ alkyl, C₂₋₆ alkenyl, C₂₋₆ alkynyl, C₁₋₆ haloalkyl, halo, D, CN, OR^{a2}, and NR^{c2}R^{d2}; wherein said C₁₋₆ alkyl, C₂₋₆ alkenyl, and C₂₋₆ alkynyl, are each optionally substituted with 1, 2, 3, or 4 substituents independently selected from R²². In another embodiment, R² is selected from H, C₁₋₆ alkyl, C₁₋₆ haloalkyl, halo, D, and CN; wherein said C₁₋₆ alkyl, is optionally substituted with 1 or 2 substituents independently selected from R²². In another embodiment, R² is selected from C₁₋₆ alkyl and halo; wherein said C₁₋₆ alkyl, is optionally substituted with 1 or 2 substituents independently selected from R²².

In an embodiment, R² is selected from H, D, C₁₋₆ alkyl, C₁₋₆ haloalkyl, halo, OR^{a1}, and NR^{c1}R^{d1}. In another embodiment, R² is selected from H, D, C₁₋₆ alkyl, and halo. In still another embodiment, R² is selected from H, D, C₁₋₆ alkyl, C₁₋₆ haloalkyl, halo, and CN. In an embodiment, R² is selected from H, D, C₁₋₂ alkyl, C₁₋₂ haloalkyl, halo, and CN. In yet another embodiment, R² is halo. In another embodiment, R² is chloro.

In an embodiment, each R²² is independently selected from C₁₋₆ alkyl, C₁₋₆ haloalkyl, halo, D, CN, OR^{a22}, and NR^{c22}R^{d22}. In an embodiment, each R²² is independently selected from C₁₋₆ alkyl, C₁₋₆ haloalkyl, halo, and CN. In an embodiment, R²² is CN.

In still another embodiment, Cy¹ is selected from C₆₋₁₀ aryl and 6-10 membered heteroaryl; wherein the 6-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 6-10 membered heteroaryl is optionally substituted by oxo to form a carbonyl group; and wherein the C₆₋₁₀ aryl and 6-10 membered heteroaryl are each optionally substituted with 1 or 2 substituents independently selected from R¹⁰.

In an embodiment, Cy¹ is C₆₋₁₀ aryl optionally substituted with 1 or 2 substituents independently selected from R¹⁰. In another embodiment, Cy¹ is C₆₋₁₀ aryl optionally substituted with 1 or 2 substituents independently selected from R¹⁰. In yet another embodiment, Cy¹ is C₆₋₁₀ aryl optionally substituted once with R¹⁰. In yet another embodiment, Cy¹ is naphthalenyl optionally substituted once with R¹⁰. In yet another embodiment, Cy¹ is 3-hydroxy-naphthalen-1-yl.

In an embodiment, Cy¹ is selected from C₆₋₁₀ aryl and 6-10 membered heteroaryl; wherein the 6-10 membered heteroaryl each has at least one ring-forming carbon atom and 1 or 2 ring-forming heteroatoms independently selected from N, and O; and wherein the C₆₋₁₀ aryl and 6-10 membered heteroaryl are each optionally substituted with 1, 2, or 3 substituents independently selected from R¹⁰. In yet another embodiment, Cy¹ is selected from naphthalenyl and 1H-indazolyl optionally substituted with 1 or 2 substituents independently selected from R¹⁰.

In yet another embodiment, Cy¹ is 5-10 membered heteroaryl provided that Cy¹ is other than 3,5-dimethylisoxazol-4-yl. In another embodiment, Cy¹ is other than 3,5-dimethylisoxazol-4-yl.

In yet another embodiment, each R¹⁰ is independently selected from C₁₋₆ alkyl, C₂₋₆ alkenyl, C₂₋₆ alkynyl, C₁₋₆ haloalkyl, halo, D, CN, OR^{a10}, and NR^{c10}R^{d10}; wherein said C₁₋₆ alkyl, C₂₋₆ alkenyl, and C₂₋₆ alkynyl, are each optionally substituted with 1 or 2 substituents independently selected from R¹¹.

In still another embodiment, each R¹⁰ is independently selected from C₁₋₆ alkyl, C₁₋₆ haloalkyl, halo, D, CN, OR^{a10}, and NR^{c10}R^{d10}. In another embodiment, each R¹⁰ is independently selected from C₁₋₆ alkyl, C₁₋₆ haloalkyl, halo, and OR^{a10}. In an embodiment, each R¹⁰ is independently selected from C₁₋₆ alkyl, halo, and OR^{a10}. In another embodiment, each R¹⁰ is independently selected from methyl, chloro, fluoro, trifluoromethyl, and hydroxyl. In another embodiment, each R¹⁰ is independently selected from methyl, fluoro, and hydroxyl.

In an embodiment, each R¹⁰ is independently selected from C₁₋₃ alkyl, C₁₋₃ haloalkyl, halo, D, CN, and OR^{a10}. In an embodiment, each R¹⁰ is independently selected from halo and OR^{a1}°. In an embodiment, each R¹⁰ is independently selected from halo and OH. In an embodiment, R¹⁰ is OH.

In still another embodiment, each R¹¹ is independently selected from C₁₋₆ alkyl, C₁₋₆ haloalkyl, halo, D, CN, OR^{a1}, and NR^{c11}R^{d11}.

In still another embodiment, R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, OR^{f3}, C(O)R^{b3}, C(O)NR^{c3}R^{d3}, C(O)OR^{a3}, OC(O)R^{b3}, OC(O)NR^{c3}R^{d3}, and NR^{c3}R^{j3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1 or 2 substituents independently selected from R³⁰.

In an embodiment, R³ is selected from H, C₃₋₁₀ cycloalkyl, 4-10 membered heterocycloalkyl, C₆₋₁₀ aryl, 5-10 membered heteroaryl, halo, and OR⁶; wherein said C₃₋₁₀ cycloalkyl, 4-10 membered heterocycloalkyl, C₆₋₁₀ aryl, and 5-10 membered heteroaryl, are each optionally substituted with 1 or 2 substituents independently selected from R³⁰. In an embodiment, R³ is selected from H, 4-10 membered heterocycloalkyl, C₆₋₁₀ aryl, and OR^{f3}; wherein said 4-10 membered heterocycloalkyl, and C₆₋₁₀ aryl, are each optionally substituted with 1 or 2 substituents independently selected from R³⁰.

In another embodiment, R³ is H or 4-7 membered heterocycloalkyl; wherein said 4-7 membered heterocycloalkyl is optionally substituted with 1 or 2 substituents independently selected from R³⁰. In yet another embodiment, R³ is 4-7 membered heterocycloalkyl; wherein said 4-7 membered heterocycloalkyl is optionally substituted with 1 or 2 substituents independently selected from R³⁰. In still another embodiment, R³ is 4 membered heterocycloalkyl; optionally substituted with 1 or 2 substituents independently selected from R³⁰.

In another embodiment, R³ is selected from H, 4-6 membered heterocycloalkyl, and OR^{f3}; wherein said 4-6 membered heterocycloalkyl is optionally substituted with 1 or 2 substituents independently selected from R³⁰. In an embodiment, R³ is 4 membered heterocycloalkyl; optionally substituted once with R³⁰. In another embodiment, R³ is selected from H, and 3-(dimethylamino)azetidin-1-yl. In another embodiment, R³ is selected from H, 3-(dimethylamino)azetidin-1-yl, and —(S)-1-methylpyrrolidin-2-yl)methoxy. In another embodiment, R³ is 3-(dimethylamino)azetidin-1-yl. In still another embodiment, R³ is H.

In an embodiment, each R³⁰ is independently selected from C₁₋₆ alkyl, C₁₋₆ haloalkyl, 4-10 membered heterocycloalkyl, 5-10 membered heteroaryl, halo, D, CN, OR^{a30}, and NR^{c30}R^{d30}; wherein said C₁₋₆ alkyl, 4-10 membered heterocycloalkyl, and 5-10 membered heteroaryl, are each optionally substituted with 1, 2, 3, or 4 substituents independently selected from R³¹. In an embodiment, each R³⁰ is independently selected from C₁₋₆ alkyl, C₁₋₆ haloalkyl, 4-6 membered heterocycloalkyl, 5-6 membered heteroaryl, halo, D, CN, OR^{a30}, and NR^{c30}R^{d30}; wherein said C₁₋₆ alkyl, 4-6 membered heterocycloalkyl, and 5-6 membered heteroaryl, are each optionally substituted with 1 or 2 substituents independently selected from R³¹.

In another embodiment, R³⁰ is NR^{c30}R^{d30}. In yet another embodiment, R³⁰ is NR^{c30}R^{d30}; and R^{c30} and R^{d30}, are each independently C₁₋₃ alkyl.

In another embodiment, each R³⁰ is independently selected from 4-10 membered heterocycloalkyl, 5-10 membered heteroaryl, halo, OR^{a30}, and NR^{c30}R^{d30}; wherein said 4-10 membered heterocycloalkyl, and 5-10 membered heteroaryl, are each optionally substituted with 1 or 2 substituents independently selected from R³¹.

In still another embodiment, each R³¹ is independently selected from C₁₋₆ alkyl, halo, D, CN, OR^{a31}, and NR^{c31}R^{d31}. In an embodiment, each R³¹ is independently selected from C₁₋₆ alkyl, C₁₋₆ haloalkyl, halo, D, and CN. In an embodiment, each R³¹ is independently C₁₋₆ alkyl. In another embodiment, each R³¹ is independently methyl.

In another embodiment, each R^{a3}, R^{b3}, R^{c3} and R^{d3} is independently selected from H, C₁₋₆ alkyl, and C₁₋₆ haloalkyl; wherein said C₁₋₆ alkyl, is optionally substituted with 1 or 2 substituents independently selected from R³⁰. In another embodiment, each R^{f3} and R^{j3} is independently selected from C₁₋₆ alkyl, and C₁₋₆ haloalkyl; wherein said C₁₋₆ alkyl, is optionally substituted with 1 or 2 substituents independently selected from R³⁰.

In yet another embodiment, each R^{a3}, is independently C₁₋₆ alkyl; wherein said C₁₋₆ alkyl, is optionally substituted with 1 substituent independently selected from R³⁰. In still another embodiment, each R^{a3}, is independently methyl; wherein said methyl, is substituted with 1 substituent independently selected from R³⁰.

In another embodiment, each R³ is independently C₁₋₆ alkyl; wherein said C₁₋₆ alkyl is optionally substituted with 1 substituent independently selected from R³⁰. In still another embodiment, each R³ is independently methyl; wherein said methyl is substituted with 1 substituent independently selected from R³⁰.

In an embodiment, R⁴ is selected from H, D, C₁₋₆ alkyl, C₂₋₆ alkenyl, C₂₋₆ alkynyl, C₁₋₆ haloalkyl, C₃₋₆ cycloalkyl, 4-6 membered heterocycloalkyl, phenyl, 5-6 membered heteroaryl, halo, CN, OR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)₂R^{b4}, S(O)₂R^{b4}, and S(O)₂NR^{c4}R^{d4}.

In another embodiment, R⁴ is selected from H, D, C₁₋₆ alkyl, C₁₋₆ haloalkyl, C₃₋₆ cycloalkyl, 4-6 membered heterocycloalkyl, phenyl, 5-6 membered heteroaryl, halo, CN, OR^{a4}, C(O)R^{b4}, C(O)NR^{c4}R^{d4}, C(O)OR^{a}a, and OC(O)R^{b4}. In still another embodiment, R⁴ is selected from H, D, C₁₋₆ alkyl, C₁₋₆ haloalkyl, halo, and CN. In still another embodiment, R⁴ is H.

In an embodiment, R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, halo, D, CN, OR^{a5}, SR^{a5}, C(O)R^{b5}, C(O)NR^{c5}R^{d5}, C(O)OR^{a5}, OC(O)R^{b5}, NR^{c5}R^{d5}, and NR^{c5}C(O)R^{b5}. In another embodiment, R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C-10 aryl, 5-10 membered heteroaryl, D, CN, and halo. In yet another embodiment, R⁵ is selected from H, C₁₋₆ alkyl, C₁₋₆ haloalkyl, D, CN, and halo. In still another embodiment, R⁵ is H or C₁₋₃ alkyl. In another embodiment, R⁵ is H.

In an embodiment, R⁵ is selected from H, C₁₋₆ alkyl, C₁₋₆ haloalkyl, CB-10 aryl, 5-10 membered heteroaryl, and D; wherein said C₁₋₆ alkyl, C₆₋₁₀ aryl, and 5-10 membered heteroaryl, are each optionally substituted with 1, 2, 3, or 4 substituents independently selected from R⁵⁰. In another embodiment, R⁵ is selected from H, C₁₋₆ alkyl, C₁₋₆ haloalkyl, phenyl, 5-6 membered heteroaryl, and D; wherein said C₁₋₆ alkyl, phenyl, and 5-6 membered heteroaryl are each optionally substituted with 1 or 2 substituents independently selected from R⁵⁰.

In an embodiment, R⁵ is selected from H, C₁₋₆ alkyl, C₁₋₆ haloalkyl, C₃₋₁₀ cycloalkyl, 4-10 membered heterocycloalkyl, CB-10 aryl, 5-10 membered heteroaryl, halo, D, CN, OR^{a5}, C(O)NR^{c5}R^{d5}, and NR^{c5}R^{d5}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R⁵⁰. In another embodiment, R⁵ is selected from H, C₁₋₆ alkyl, C₁₋₆ haloalkyl, 4-10 membered heterocycloalkyl, C₆₋₁₀ aryl, 5-10 membered heteroaryl, halo, CN, OR^{a5}, and C(O)NR^{c5}R^{d5}; wherein said C₁₋₆ alkyl, 4-10 membered heterocycloalkyl, C₆₋₁₀ aryl, and 5-10 membered heteroaryl, are each optionally substituted with 1, 2, or 3 substituents independently selected from R⁵⁰.

In another embodiment, each R⁵⁰ is independently selected from C₁₋₆ alkyl, C₁₋₆ haloalkyl, halo, D, CN, OR^{a50}, and NR^{c50}R^{d50}. In an embodiment, each R⁵⁰ is independently selected from C₁₋₆ alkyl, C₁₋₆ haloalkyl, halo, D, and CN. In an embodiment, each R⁵⁰ is C₁₋₆ alkyl.

In an embodiment, each R⁵⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a50}, and NR^{c50}R^{d50}. In another embodiment, each R⁵⁰ is independently selected 10 from C₁₋₆ alkyl, C₁₋₆ haloalkyl, C₆₋₁₀ aryl, 5-10 membered heteroaryl, halo, CN, OR^{a50}, and NR^{c50}R^{d50}.

In an embodiment, each R⁵¹ is independently selected from C₁₋₆ alkyl, C₁₋₆ haloalkyl, halo, D, CN, OR^{a51}, and NR^{c51}R^{d51}. In another embodiment, each R⁵¹ is independently selected from C₁₋₆ alkyl, C₁₋₆ haloalkyl, halo, D, and CN.

In an embodiment, R⁶ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, D, CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6} In another embodiment, R⁶ is selected from H, D, C₁₋₆ alkyl, C₁₋₆ haloalkyl, OR^{a6}, and NR^{c6}R^{d6} In another embodiment, R⁶ is selected from H, D, C₁₋₆ alkyl, and C₁₋₆ haloalkyl.

In an embodiment, R⁶ is selected from H, D, C₁₋₆ alkyl, C₁₋₆ haloalkyl, C₃₋₆ cycloalkyl, and 4-6 membered heterocycloalkyl; wherein said C₁₋₆ alkyl, C₃₋₆ cycloalkyl, and 4-6 membered heterocycloalkyl are each optionally substituted with 1, 2, 3, or 4 substituents independently selected from R⁶⁰. In another embodiment, R⁶ is selected from H, D, C₁₋₆ alkyl, and C₁₋₆ haloalkyl; wherein said C₁₋₆ alkyl is optionally substituted with 1 or 2 substituents independently selected from R⁶⁰.

In yet another embodiment, R⁶ is selected from H, C₁₋₆ alkyl, C₁₋₆ haloalkyl, C₃₋₁₀ cycloalkyl, 4-10 membered heterocycloalkyl, CB-10 aryl, 5-10 membered heteroaryl, halo, D, CN, OR^{a6}, and NR^{c6}R^{d6}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R⁶⁰.

In an embodiment, each R⁶⁰ is independently selected from C₁₋₆ alkyl, C₁₋₆ haloalkyl, halo, D, CN, OR^{a60}, C(O)R^{b60}, C(O)NR^{c60}R^{d60}, C(O)OR^{a60}, and NR^{c60}R^{d60}. In another embodiment, each R⁶⁰ is independently selected from C₁₋₆ alkyl, C₁₋₆ haloalkyl, CN, C(O)NR^{c60}R^{d60}, and C(O)OR^{a60}.

In an embodiment, each R⁶⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a0}, C(O)NR^{c60}R^{d60}, C(O)OR^{a60}, and NR^{c60}R^{d60}. In another embodiment, each R⁶⁰ is independently selected from C₁₋₆ alkyl, C₁₋₆ haloalkyl, C₆₋₁₀ aryl, halo, D, CN, OR^{a60}, C(O)NR^{c60}R^{d60}, C(O)OR^{a60}, and NR^{c60}R^{d60}.

In yet another embodiment, R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, D, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}. In still another embodiment, R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, and halo. In an embodiment, R⁷ is selected from H, D, C₁₋₃ alkyl, C₁₋₃ haloalkyl, CN, and halo. In still another embodiment, R⁷ is halo. In still another embodiment, R⁷ is fluoro.

In an embodiment, Cy² is 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted with 1 or 2 substituents independently selected from R²⁰;

In another embodiment, Cy² is selected from C₃₋₆ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₆ cycloalkyl, 4-10 membered heterocycloalkyl, C₆₋₁₀ aryl and 5-10 membered heteroaryl are each optionally substituted with 1 or 2 substituents independently selected from R²⁰.

In another embodiment Cy² is 4-10 membered heterocycloalkyl wherein the 4-10 membered heterocycloalkyl has at least one ring-forming carbon atom and 1 or 2 ring-forming heteroatoms independently selected from N and O; and wherein the 4-10 membered heterocycloalkyl, is optionally substituted 1 or 2 substituents independently selected from R²⁰.

In yet another embodiment, Cy² is 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted once with R²⁰;

In yet another embodiment, Cy² is selected from 4-(piperidin-1-yl)prop-2-en-1-one, 3-(piperidin-1-yl)prop-2-en-1-one, 3-azetidin-1-yl)prop-2-en-1-one, and 3-pyrrolidin-1-yl)prop-2-en-1-one. In still another embodiment, Cy² is 4-(piperidin-1-yl)prop-2-en-1-one. In an embodiment, Cy² is 3-(piperidin-1-yl)prop-2-en-1-one. In another embodiment, Cy² is 3-(azetidin-1-yl)prop-2-en-1-one. In yet another embodiment, Cy² is 3-(pyrrolidin-1-yl)prop-2-en-1-one.

In an embodiment, Cy² is 4-6 membered heterocycloalkyl optionally substituted with one or two R²⁰. In yet another embodiment, R²⁰ is C(O)R^{b20}.

In an embodiment, Cy² is selected from

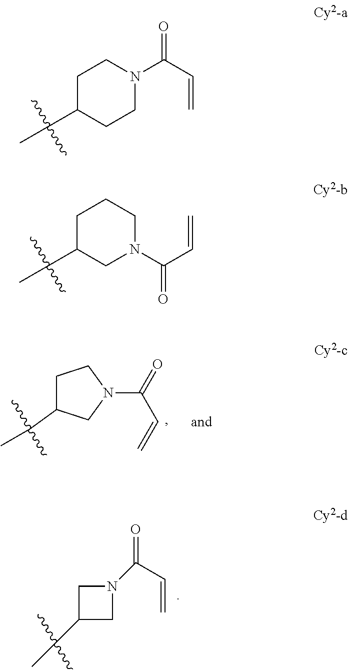

In another embodiment Cy² is Cy²-a. In yet another embodiment Cy² is Cy²-b. In still another embodiment Cy² is Cy²-c. In an embodiment Cy² is Cy²-d.

In yet another embodiment, Cy² is selected from

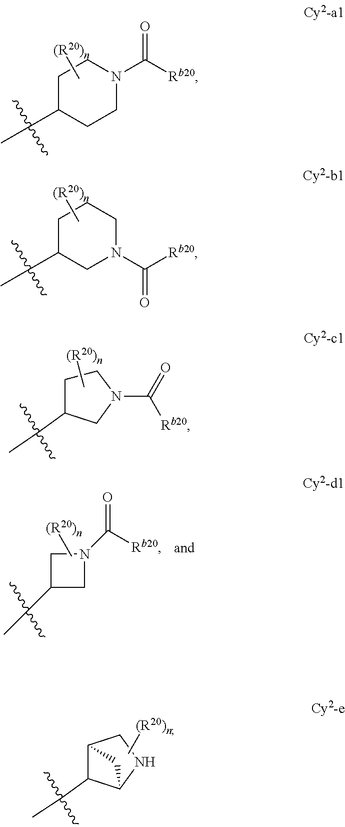

wherein n is 0, 1 or 2.

In an embodiment, Cy² is Cy²-a1. In another embodiment, Cy² is Cy²-b1. In yet another embodiment, Cy² is Cy²-c1. In still another embodiment, Cy² is Cy²-d1. In an embodiment, Cy² is Cy²-e.

In an embodiment, n is 0. In another embodiment, n is 1. In yet another embodiment, n is 2.

In an embodiment, each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a20}, C(O)R^{b20}, C(O)NR^{c20}R^{d20}, C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, NR^{c20}S(O)₂R^{b20}, NR^{c20}S(O)₂NR^{c20}R^{d20}, S(O)₂R^{b20}, and S(O)₂NR^{c20}R^{d20}.

In yet another embodiment, each R²⁰ is independently selected from C(O)R^{b20}, C(O)NR^{c20}R^{d20}, and C(O)OR^{a20}. In still another embodiment, each R²⁰ is C(O)R^{b20}.

In an embodiment, each R²⁰ is independently selected from C₁₋₆ alkyl, C₁₋₆ haloalkyl, halo, D, CN, OR^{a20}, C(O)R^{b20}, C(O)NR^{c20}R^{d20}, C(O)OR^{a20} and NR^{c20}R^{d20}; wherein said C₁₋₆ alkyl is optionally substituted with 1, 2, 3, or 4 substituents independently selected from R²¹.

In an embodiment, each R²⁰ is independently selected from C₁₋₆ alkyl, C₁₋₆ haloalkyl, halo, D, CN, and C(O)R^{b20}; wherein said C₁₋₆ alkyl, is optionally substituted with 1 or 2 substituents independently selected from R²¹. In an embodiment, each R²⁰ is independently selected from C₁₋₆ alkyl, CN, and C(O)R^{b20}; wherein said C₁₋₆ alkyl, is optionally substituted with 1 or 2 substituents independently selected from R²¹.

In another embodiment, each R²¹ is independently selected from C₁₋₆ alkyl, C₁₋₆ haloalkyl, halo, D, CN, OR^{a21}, and NR^{c21}R^{d21}. In another embodiment, each R²¹ is independently selected from C₁₋₆ alkyl, C₁₋₆ haloalkyl, halo, D, CN, and OR^{a21}. In another embodiment, R²¹ is CN.

In another embodiment, each R^{g} is independently selected from D, OH, CN, halo, C₁₋₆ alkyl, C₂₋₆ alkenyl, C₂₋₆ alkynyl, C₁₋₆ haloalkyl, C₃₋₆ cycloalkyl, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, C₁₋₆ alkylthio, C₁₋₆ alkylsulfonyl, carbamyl, C₁₋₆ alkylcarbamyl, and di(C₁₋₆ alkyl)carbamyl.

In an embodiment of Formula Ia, or a pharmaceutically acceptable salt thereof,

Y is N or C;

R¹ is H;

R² is selected from H, C₁₋₆ alkyl, C₁₋₆ haloalkyl, and halo, wherein alkyl is optionally substituted once with CN;

Cy¹ is selected from C₆₋₁₀ aryl and 6-10 membered heteroaryl; wherein the 6-10 membered heteroaryl has at least one ring-forming carbon atom and 1 or 2 ring-forming heteroatoms independently selected from N and O; and wherein the C₆₋₁₀ aryl and 6-10 membered heteroaryl are each optionally substituted with 1, 2, or 3 substituents independently selected from OH, halo, C₁₋₆ alkyl, C₁₋₆ haloalkyl, and CN;

R³ is selected from H, C₃₋₁₀ cycloalkyl, 4-10 membered heterocycloalkyl, halo, and OC₁₋₆ alkyl; wherein said OC₁₋₆ alkyl, C₃₋₁₀ cycloalkyl, and 4-10 membered heterocycloalkyl, are each optionally substituted with 1, 2, or 3 substituents independently selected from N(C₁₋₆ alkyl)₂, C₁₋₆ alkyl, and 4-6 membered heterocycloalkyl optionally substituted with C₁₋₆ alkyl;

R⁵ is selected from H, C₁₋₆ alkyl, C₆₋₁₀ aryl, 5-6 membered heteroaryl, C₁₋₆ haloalkyl, halo, C(O)NH(C₁₋₆ alkyl), and 4-6 membered heterocycloalkyl, wherein heteroaryl, heterocycloalkyl, and alkyl are optionally substituted with 1 or 2 substituents selected from C₁₋₆ alkyl, OH, C₆₋₁₀ aryl, and N(C₁₋₆ alkyl)₂;
when Y is N, then R⁶ is absent;

R⁶ is selected from H, C₁₋₆ alkyl, 5-6 membered heteroaryl, and C₁₋₆ haloalkyl, wherein alkyl and heteroayl are optionally substituted with 1 or 2 substituents selected from C₁₋₆ alkyl, C(O)OC₁₋₆ alkyl, C(O)N(C₁₋₆ alkyl)₂, C₆₋₁₀ aryl, and C(O)(4-6 membered heterocycloalkyl);

R⁷ is selected from H and halo; and

Cy² is selected from 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted with 1, 2 or 3 substituents independently selected from C(O)C₂₋₆ alkenyl, C(O)C₂₋₆ alkynyl, C₁₋₆ alkyl, wherein alkenyl and alkyl are optionally substituted one or two times with a substituent selected from CN, N(C₁₋₆ alkyl)₂, OC₁₋₆ alkyl, and halo.

In an embodiment, the compound of Formula I is
1-(4-(8-chloro-6-fluoro-7-(3-hydroxynaphthalen-1-yl)-1H-pyrazolo[4,3-c]-quinolin-1-yl)-piperidin-1-yl)prop-2-en-1-one; or
1-(4-(8-chloro-4-(3-(dimethylamino)azetidin-1-yl)-6-fluoro-7-(3-hydroxynaphthalen-1-yl)-1H-pyrazolo[4,3-c]quinolin-1-yl)piperidin-1-yl)prop-2-en-1-one;

or a pharmaceutically acceptable salt thereof.

In another embodiment, the compound of Formula I is selected from
2-((2S,4S)-1-acryloyl-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-methoxybut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4,4-difluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4,4-difluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-1-(but-2-ynoyl)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-methoxybut-2-enoyl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-fluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4,4-difluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-(2-fluoroacryloyl)piperidin-2-yl)acetonitrile;
2-((2S,4S)-1-acryloyl-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)-1-((E)-4-(dimethylamino)but-2-enoyl)piperidin-2-yl)acetonitrile;
2-((2S,4S)-1-(but-2-ynoyl)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)-1-((E)-4-methoxybut-2-enoyl)piperidin-2-yl)acetonitrile;
2-((2S,4S)-1-acryloyl-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile;
2-((2S,4S)-1-acryloyl-4-(8-chloro-4-(3-(dimethylamino)azetidin-1-yl)-6-fluoro-7-(5-fluoroquinolin-8-yl)-1H-pyrazolo[4,3-c]quinolin-1-yl)piperidin-2-yl)acetonitrile;
2-((2S,4S)-1-acryloyl-4-(8-chloro-4-(3-(dimethylamino)azetidin-1-yl)-6-fluoro-7-(isoquinolin-4-yl)-1H-pyrazolo[4,3-c]quinolin-1-yl)piperidin-2-yl)acetonitrile;
2-((2S,4S)-1-acryloyl-4-(8-chloro-7-(2-chloro-3-methylphenyl)-4-(3-(dimethylamino)azetidin-1-yl)-6-fluoro-1H-pyrazolo[4,3-c]quinolin-1-yl)piperidin-2-yl)acetonitrile;
2-((2S,4S)-4-(8-chloro-7-(2-chloro-3-methylphenyl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-1-acryloyl-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile;
2-((2S,4S)-1-(but-2-ynoyl)-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile;
2-((2S,4S)-1-acryloyl-4-(8-chloro-4-(3-(dimethylamino)azetidin-1-yl)-6-fluoro-7-(3-methyl-2-(trifluoromethyl) phenyl)-1H-pyrazolo[4,3-c]quinolin-1-yl)piperidin-2-yl)acetonitrile;
2-((2S,4S)-1-acryloyl-4-(8-chloro-6-fluoro-7-(3-methyl-2-(trifluoromethyl)phenyl)-4-(((S)-1-methylpyrrolidin-2-yl)methoxy)-1H-pyrazolo[4,3-c]quinolin-1-yl)piperidin-2-yl)acetonitrile;
methyl 3-(1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinolin-2-yl)propanoate;
3-(1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinolin-2-yl)-N,N-dimethylpropanamide;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-2-propyl-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-2-(1-methyl-1H-pyrazol-4-yl)-4-(((S)-1-methylpyrrolidin-2-yl)methoxy)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-phenyl-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-(pyridin-3-yl)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-3-(2-methyloxazol-5-yl)-4-(((S)-1-methylpyrrolidin-2-yl)methoxy)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-(2-methylthiazol-5-yl)-1H-pyrrolo[3,2-c]quinolin-8-yl)propanenitrile; and
3-(1-(2-azabicyclo[2.1.1]hexan-5-yl)-6-fluoro-7-(3-hydroxynaphthalen-1-yl)-3-(1-methyl-1H-pyrazol-4-yl)-4-(((S)-1-methylpyrrolidin-2-yl)methoxy)-1H-pyrrolo[3,2-c]quinolin-8-yl)propanenitrile;

or a pharmaceutically acceptable salt thereof.

In yet another embodiment, the compound of Formula I is selected from the group consisting of
3-(1-(2-azabicyclo[2.1.1]hexan-5-yl)-6-fluoro-7-(3-hydroxynaphthalen-1-yl)-2-(1-methyl-1H-pyrazol-3-yl)-4-(((S)-1-methylpyrrolidin-2-yl)methoxy)-1H-pyrrolo[3,2-c]quinolin-8-yl)propanenitrile;
3-(2-benzyl-1-(2-azabicyclo[2.1.1]hexan-5-yl)-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-(1H-pyrazol-4-yl)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-(6-oxo-1,6-dihydropyridin-3-yl)-1H-pyrrolo[3,2-c]quinolin-8-yl)propanenitrile;
3-(1-(2-azabicyclo[2.1.1]hexan-5-yl)-3-chloro-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile;
1-(2-azabicyclo[2.1.1]hexan-5-yl)-8-(2-cyanoethyl)-6-fluoro-N-(2-hydroxyethyl)-7-(3-hydroxynaphthalen-1-yl)-4-(((S)-1-methylpyrrolidin-2-yl)methoxy)-1H-pyrrolo[3,2-c]quinoline-3-carboxamide;
N-Benzyl-1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinoline-3-carboxamide;
3-(1-(2-Azabicyclo[2.1.1]hexan-5-yl)-6-fluoro-3-(hydroxymethyl)-7-(3-hydroxynaphthalen-1-yl)-4-(((S)-1-methylpyrrolidin-2-yl)methoxy)-1H-pyrrolo[3,2-c]quinolin-8-yl)propanenitrile;
2-((2S,4S)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)-1-((E)-4-methoxybut-2-enoyl)piperidin-2-yl)acetonitrile;
3-(1-((1R,4R,5S)-2-azabicyclo[2.1.1]hexan-5-yl)-8-(2-cyanoethyl)-4-ethoxy-6-fluoro-7-(3-hydroxynaphthalen-1-yl)-1H-pyrrolo[3,2-c]quinolin-2-yl)-N,N-dimethylpropanamide;
methyl 3-(1-((1R,4R,5S)-2-azabicyclo[2.1.1]hexan-5-yl)-8-(2-cyanoethyl)-3-(3-(dimethylamino)azetidin-1-yl)-6-fluoro-7-(3-hydroxynaphthalen-1-yl)-4-methoxy-1H-pyrrolo[3,2-c]quinolin-2-yl)propanoate;
3-(2-(3-(azetidin-1-yl)-3-oxopropyl)-1-(2-azabicyclo[2.1.1]hexan-5-yl)-6-fluoro-7-(7-fluoronaphthalen-1-yl)-4-(((S)-1-methylpyrrolidin-2-yl)methoxy)-1H-pyrrolo[3,2-c]quinolin-8-yl)propanenitrile;
8-(1-((2S,4S)-2-(cyanomethyl)-1-((E)-4-fluorobut-2-enoyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile;
8-(1-((2S,4S)-2-(cyanomethyl)-1-(2-fluoroacryloyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile;
8-(1-((2S,4S)-1-(but-2-ynoyl)-2-(cyanomethyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile;
8-(1-((2S,4S)-2-(cyanomethyl)-1-((E)-4-methoxybut-2-enoyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile;
8-(1-((2S,4S)-2-(cyanomethyl)-1-((E)-4-fluorobut-2-enoyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile;
8-(1-((2S,4S)-2-(cyanomethyl)-1-(2-fluoroacryloyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile;
8-(1-((2S,4S)-2-(cyanomethyl)-1-((E)-4-methoxybut-2-enoyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile;
8-(1-((2S,4S)-2-(cyanomethyl)-1-((E)-4-fluorobut-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
8-(1-((2S,4S)-2-(cyanomethyl)-1-(2-fluoroacryloyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
8-(1-((2S,4S)-1-(but-2-ynoyl)-2-(cyanomethyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
8-(1-((2S,4S)-2-(cyanomethyl)-1-((E)-4-methoxybut-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
8-(1-((2S,4S)-2-(cyanomethyl)-1-((E)-4-(dimethylamino)but-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
2-((2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-fluorobut-2-enoyl)piperidin-2-yl)acetonitrile;
2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-methoxybut-2-enoyl)piperidin-2-yl)acetonitrile;
2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-(dimethylamino)but-2-enoyl)piperidin-2-yl)acetonitrile;
2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-fluorobut-2-enoyl)piperidin-2-yl)acetonitrile;
2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-(2-fluoroacryloyl)piperidin-2-yl)acetonitrile;
2-((2S,4S)-1-(but-2-ynoyl)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidin-2-yl)acetonitrile;
2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-methoxybut-2-enoyl)piperidin-2-yl)acetonitrile;
2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile;
2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)piperidin-2-yl)acetonitrile;
2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-methoxybut-2-enoyl)piperidin-2-yl)acetonitrile;
2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile;
2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-((E)-4-methoxybut-2-enoyl)piperidin-2-yl)acetonitrile;
2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile;
2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-((E)-4-methoxybut-2-enoyl)piperidin-2-yl)acetonitrile;
2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-fluorobut-2-enoyl)piperidin-2-yl)acetonitrile;
2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)piperidin-2-yl)acetonitrile;
2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-methoxybut-2-enoyl)piperidin-2-yl)acetonitrile;
2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-(dimethylamino)but-2-enoyl)piperidin-2-yl)acetonitrile; and
2-((2S,4S)-1-(but-2-ynoyl)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)piperidin-2-yl)acetonitrile;

or a pharmaceutically acceptable salt thereof.

In another aspect, provided herein is a pharmaceutical composition comprising the compound of Formula I, or a pharmaceutically acceptable salt thereof, and a pharmaceutically acceptable carrier.

In an aspect, provided herein is a method of inhibiting a KRAS protein harboring a G12C mutation, said method comprising contacting a compound of the instant disclosure with KRAS.

In another aspect, provided herein is a method of inhibiting a KRAS protein harboring a G12D mutation, said method comprising contacting a compound of the instant disclosure with KRAS.

In yet another aspect, provided herein is a method of inhibiting a KRAS protein harboring a G12V mutation, said method comprising contacting a compound of the instant disclosure with KRAS.

In an embodiment, compounds of the Formulae herein are compounds of the Formulae or pharmaceutically acceptable salts thereof.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula I can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkoxy,” employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. The term “Cₙ₋ₘ dialkoxy” refers to a linking group of formula —O—(Cₙ₋ₘ alkyl)-O—, the alkyl group of which has n to m carbons. Example dialkyoxy groups include —OCH₂CH₂O— and OCH₂CH₂CH₂O—. In some embodiments, the two O atoms of a Cₙ₋ₘ dialkoxy group may be attached to the same B atom to form a 5- or 6-membered heterocycloalkyl group.

The term “alkylthio,” employed alone or in combination with other terms, refers to a group of formula —S-alkyl, wherein the alkyl group is as defined above.

The term “amino,” employed alone or in combination with other terms, refers to a group of formula —NH₂, wherein the hydrogen atoms may be substituted with a substituent described herein. For example, “alkylamino” can refer to —NH(alkyl) and —N(alkyl)₂.

The term “carbonyl,” employed alone or in combination with other terms, refers to a —C(═O)— group, which also may be written as C(O).

The term “cyano” or “nitrile” refers to a group of formula —C≡N, which also may be written as —CN.

The term “carbamyl,” as used herein, refers to a —NHC(O)O— or —OC(O)NH— group, wherein the carbon atom is doubly bound to one oxygen atom, and singly bound to a nitrogen and second oxygen atom.

The terms “halo” or “halogen,” used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “haloalkoxy,” employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” or “oxy” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “sulfonyl” refers to a —SO₂— group wherein a sulfur atom is doubly bound to two oxygen atoms.

The term “sulfinyl” refers to a —SO— group wherein a sulfur atom is doubly bound to one oxygen atom.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized 71 (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, isoxazolyl, thiazolyl, imidazolyl, furanyl, thio-phenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, isoindolyl, benzothiophenyl, benzofuranyl, benzisoxazolyl, imidazo[1,2-b]thiazolyl, purinyl, and the like. In some embodiments, the heteroaryl group is pyridone (e.g., 2-pyridone).

A five-membered heteroaryl ring is a heteroaryl group having five ring atoms wherein one or more (e.g., 1, 2 or 3) ring atoms are independently selected from N, O and S.

Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S.

Exemplary six-membered ring heteroaryls are pyridyl, pyrazinyl, pyrimidinyl, triazinyl, isoindolyl, and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “COₙ-m cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 2,5-diazabicyclo[2.2.1]heptanyl; pyrrolidinyl; hexahydropyrrolo[3,4-b]pyrrol-1(2H)-yl; 1,6-dihydropyridinyl; morpholinyl; azetidinyl; piperazinyl; and 4,7-diazaspiro[2.5]octan-7-yl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge.

Example prototropic tautomers include ketone—enol pairs, amide—imidic acid pairs, lactam-lactim pairs, enamine—imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et. al J. Med. Chem. 2011, 54, 201-210; R. Xu et. al. J. Label Compd. Radiopharm. 2015, 58, 308-312).

The term “compound” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected.

Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds provided herein can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high-performance liquid chromatography (HPLC) or thin layer chromatography (TLC).

The Schemes below provide general guidance in connection with preparing the compounds of the present disclosure. One skilled in the art would understand that the preparations shown in the Schemes can be modified or optimized using general knowledge of organic chemistry to prepare various compounds provided herein.

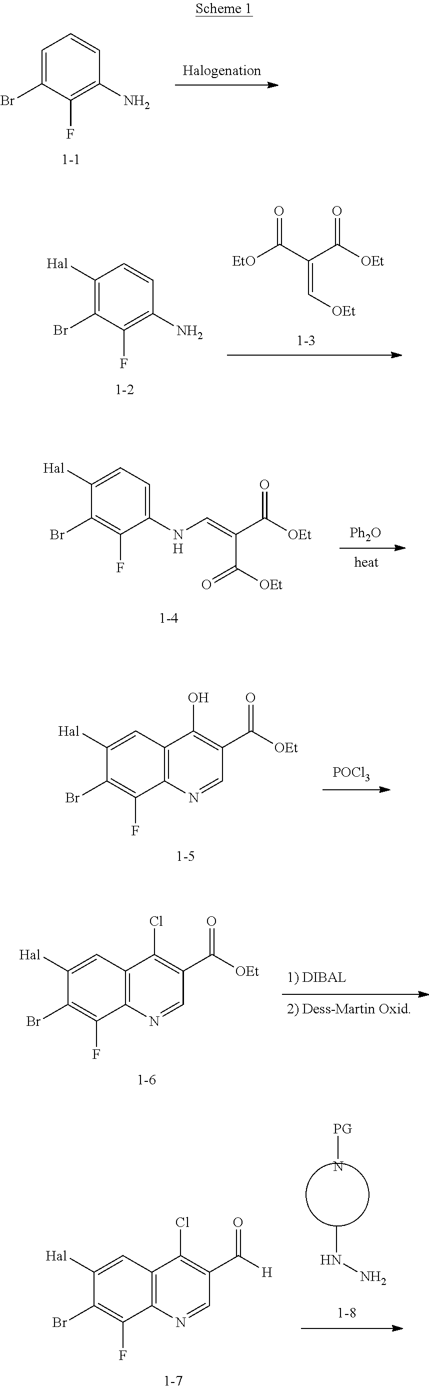

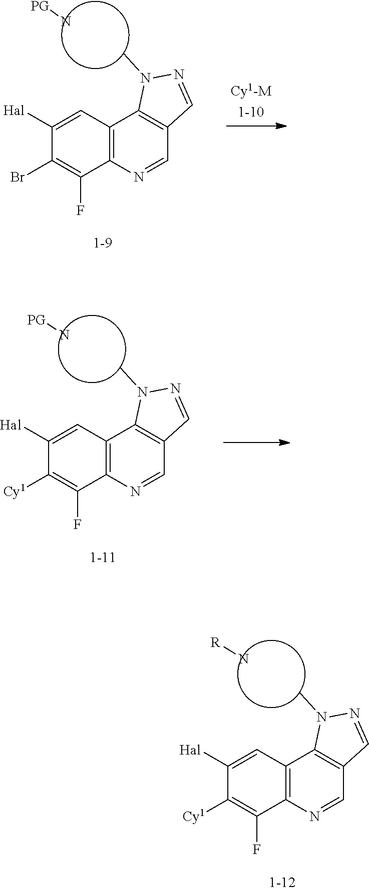

Compounds of formula 1-12 can be prepared via the synthetic route outlined in Scheme 1. Halogenation of commercially available starting material 1-1 with an appropriate reagent, such as N-Chlorosuccinimide (NCS), affords intermediate 1-2 (Hal is a halide, such as F, Cl, Br, or I). Intermediate 1-4 can then be prepared by condensation of intermediate 1-2 with diethyl 2-(ethoxymethylene)malonate (1-3), followed by cyclized by heating in an appropriate high-boiling solvent (e.g., Ph₂O) to yield quinolone 1-5. Treatment of intermediate 1-5 with POCl₃ yields intermediate 1-6. Reduction of ethyl ester with reducing reagent (such as DIBAL) followed by oxidation of alcohol with appropriate reagent, such as Dess-Martin Periodinane affords intermediate 1-7. Cyclization reaction of with hydrazine 1-8 (PG is an appropriate protecting group, such as Boc) gives tricyclic adduct 1-9. Compound 1-11 can then be prepared by coupling of 1-9 with an adduct of formula 1-10, in which M is a boronic acid, boronic ester or an appropriately substituted metal [e.g., M is B(OR)₂, Sn(Alkyl)₃, or Zn-Hal], under standard Suzuki Cross-Coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palldium catalyst). Removal of the protecting group in 1-11 and subsequent functionalization of the resulting adduct (such as coupling with acid chloride, e.g. acryloyl chloride) affords the desired product 1-12.

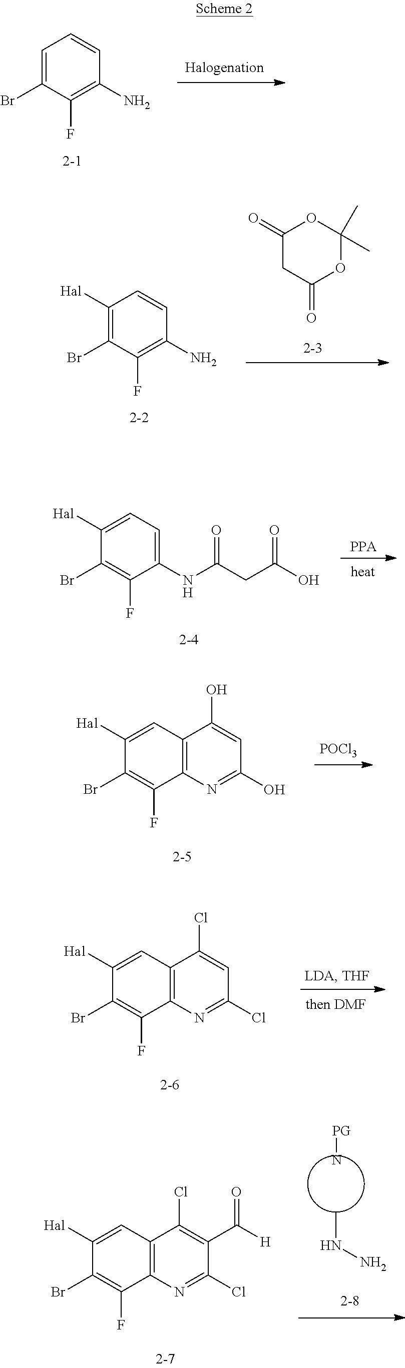

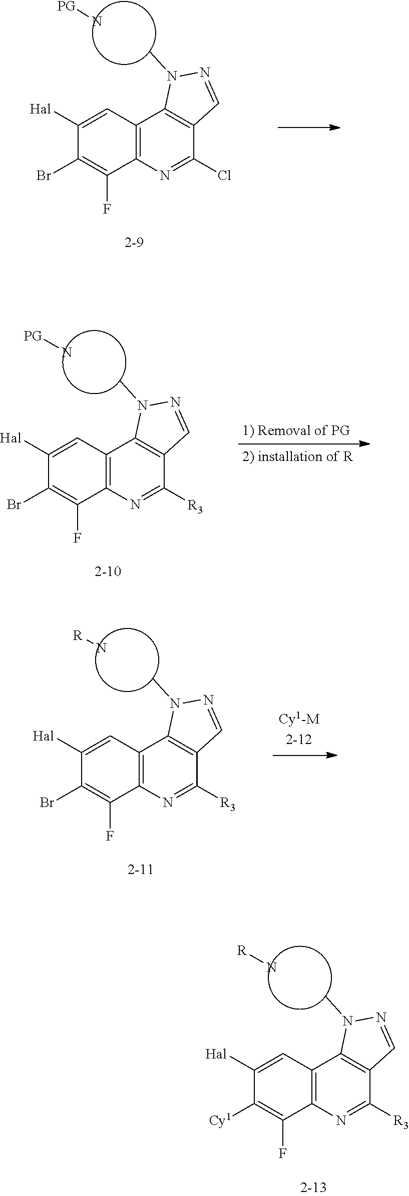

Compounds of formula 2-13 can be prepared via the synthetic route outlined in Scheme 2. Halogenation of commercially available starting material 2-1 with an appropriate reagent, such as N-Chlorosuccinimide (NCS), affords intermediate 2-2 (Hal is a halide, such as F, Cl, Br, or I). Compound 2-4 can be prepared by treating 2-2 with reagents such as 2,2-dimethyl-1,3-dioxane-4,6-dione (2-3). Intermediate 2-4 can undergo a cyclization reaction (in Polyphosphoric acid in thermal condition) to deliver the compound 2-5, which can be treated with an appropriate reagent (e.g. POCl₃) to afford compound 2-6. Intermediate 2-6 can be treated with appropriate reagent (such as LDA in THF, then DMF) to generate compound 2-7. Condensation of intermediate 2-7 with hydrazine 2-8 (PG is an appropriate protecting group, such as Boc) can be carried out to generate compound 2-9. The R³ group in 2-10 can then be installed via a suitable transformation, such as a S_{N}Ar reaction or a coupling reaction. Intermediate 2-10 can first undergo a deprotection of protecting group PG, followed by functionalization of the resulting amine (such as coupling with acid chloride, e.g. acryloyl chloride) then afford compound 2-11. The desired product 2-13 can be prepared by a cross coupling reaction between 2-11 and an adduct of formula 2-12, in which M is a boronic acid, boronic ester or an appropriately substituted metal [e.g., M is B(OR)₂, Sn(Alkyl)₃, or Zn-Hal], under standard Suzuki Cross-Coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palldium catalyst). The order of the above described chemical reactions can be rearranged as appropriate to suite the preparation of different analogues.

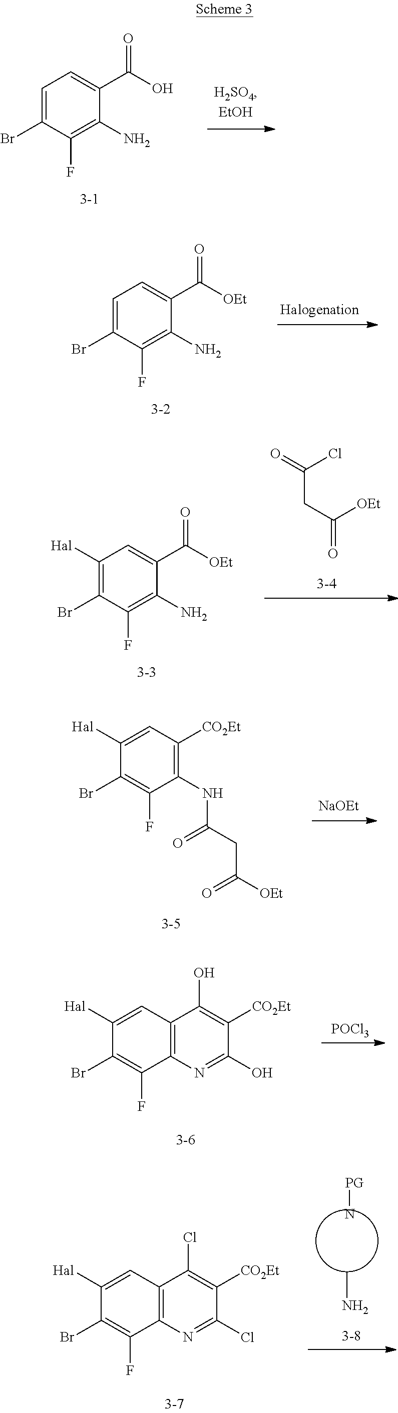

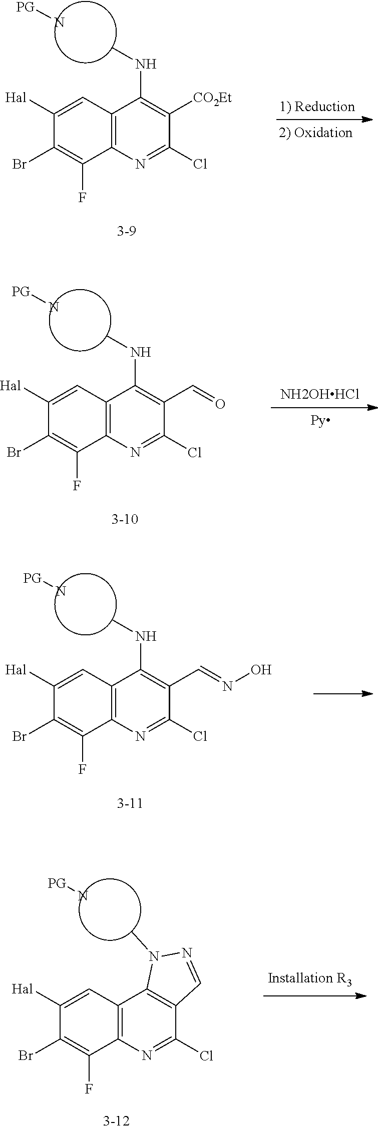

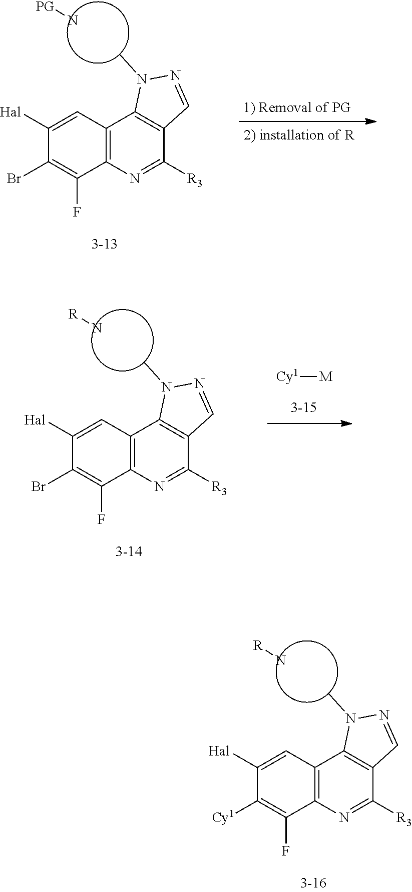

Compounds of formula 3-16 can be prepared via the synthetic route outlined in Scheme 3. Esterification of commercially available starting material 3-1 with H₂SO₄ in ethanol. Halogenation of compound 3-2 with an appropriate reagent, such as N-chlorosuccinimide (NCS), affords intermediate 3-3 (Hal is a halide, such as F, Cl, Br, or I). Compound 3-5 can be prepared by treating 3-3 with reagents such as ethyl malonyl chloride (3-4). Intermediate 3-5 can undergo a cyclization reaction (such as sodium ethoxide in ethanol) to deliver the compound 3-6, which can be treated with an appropriate reagent (e.g. POCl₃) to afford compound 3-7. Condensation of intermediate 3-7 with amine 3-8 (PG is an appropriate protecting group, such as Boc) can be carried out to generate compound 3-9. Reduction of ester with reducing reagent (such as DIBAL), followed by oxidation of intermediate with oxidation reagent (such as Dess-Martin periodinane) to yield aldehyde 3-10. Treatment of intermediate 3-10 with hydroxylamine hydrochloride and pyridine get compound 3-11. Intermediate 3-11 can undergo a cyclization reaction (such as methanesulfonyl chloride, aminopyridine in DCM) to deliver the compound 3-12. The R³ group in 3-13 can then be installed via a suitable transformation, such as a S_{N}Ar reaction or a coupling reaction. Intermediate 3-13 can first undergo a deprotection of protecting group PG, followed by functionalization of the resulting amine (such as coupling with acid chloride, e.g. acryloyl chloride) then afford compound 3-14. The desired product 3-16 can be prepared by a cross coupling reaction between 3-14 and an adduct of formula 3-15, in which M is a boronic acid, boronic ester or an appropriately substituted metal [e.g., M is B(OR)₂, Sn(Alkyl)₃, or Zn-Hal], under standard Suzuki Cross-Coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palldium catalyst). The order of the above described chemical reactions can be rearranged as appropriate to suite the preparation of different analogues.

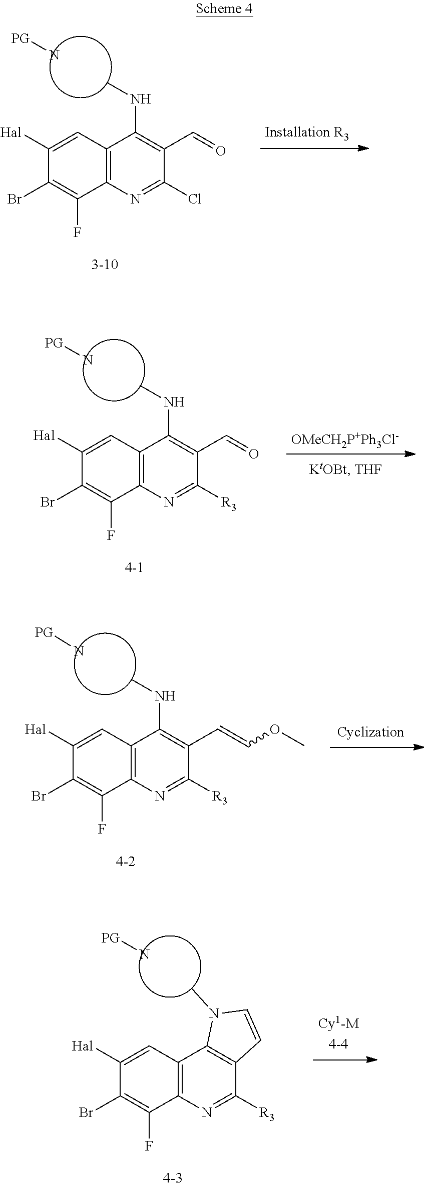

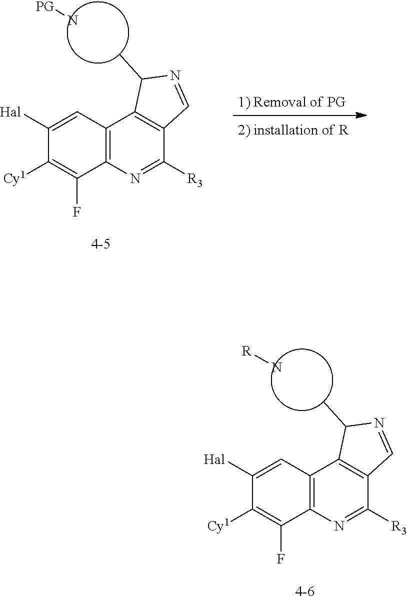

Compounds of formula 4-6 can be prepared via the synthetic route outlined in Scheme 4. Intermediate 3-10 is converted to compound 4-1 via a suitable transformation, such as a S_{N}Ar reaction or a coupling reaction. Wittig reaction of aldehyde 4-1 with (methoxymethyl)triphenylphosphonium chloride and potassium tert-butoxide in THF get compound 4-2. Intermediate 4-2 can undergo a cyclization reaction (such as TFA in DCM) to deliver the compound 4-3. Intermediate 4-5 can be prepared by a cross coupling reaction between 4-3 and an adduct of formula 4-4, in which M is a boronic acid, boronic ester or an appropriately substituted metal [e.g., M is B(OR)₂, Sn(Alkyl)₃, or Zn-Hal], under standard Suzuki Cross-Coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palldium catalyst). Compound 4-5 can first undergo a deprotection of protecting group PG, followed by functionalization of the resulting amine (such as coupling with acid chloride, e.g. acryloyl chloride) then afford compound 4-6. The order of the above described chemical reactions can be rearranged as appropriate to suite the preparation of different analogues.

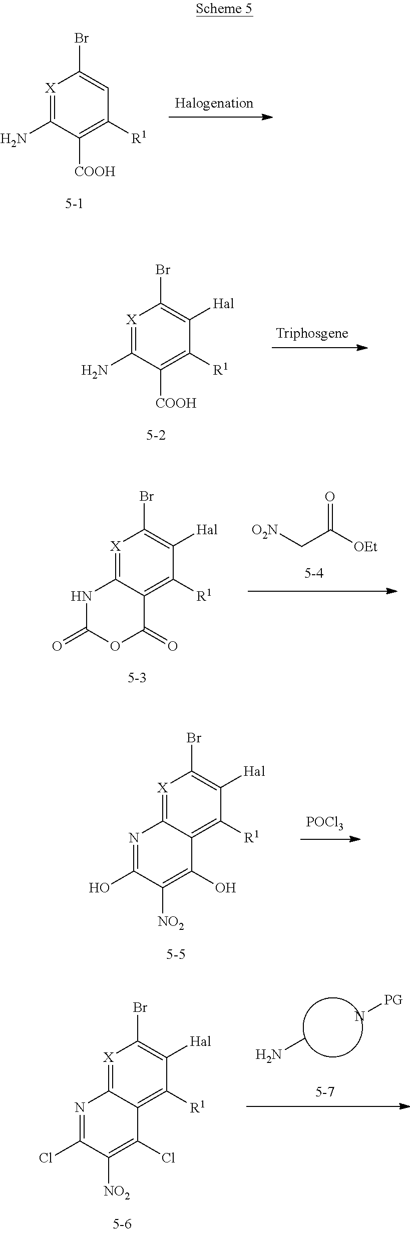

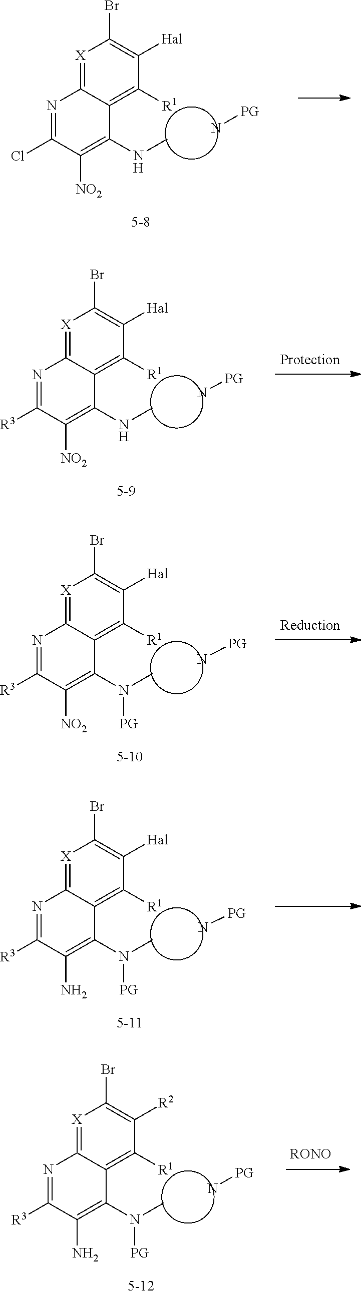

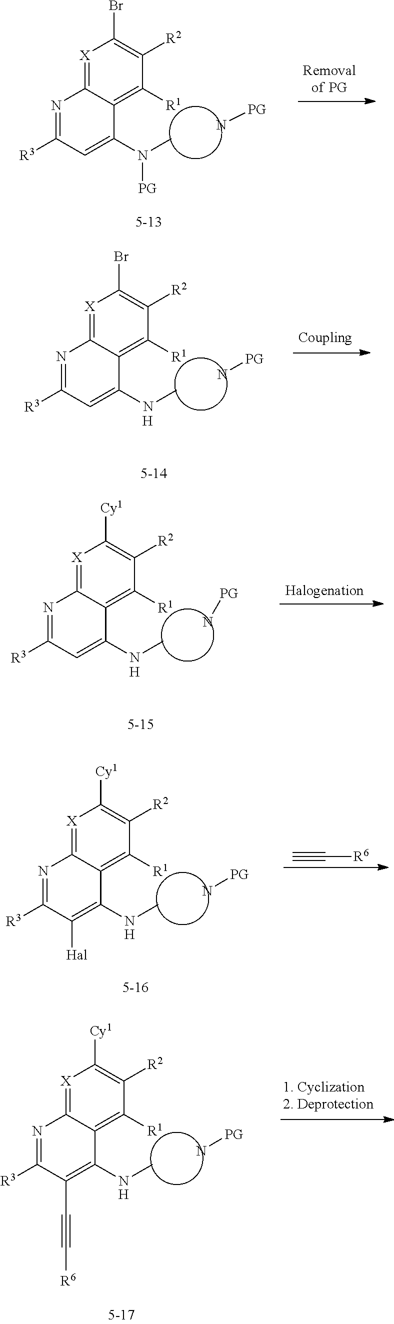

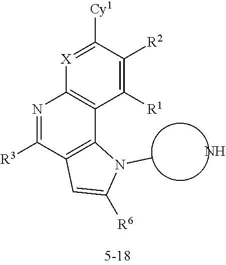

Compounds of formula 5-18 can be prepared via the synthetic route outlined in Scheme 5. Halogenation of starting material 5-1 with an appropriate reagent, such as N-chloro-succinimide (NCS), affords intermediate 5-2 (Hal is a halide, such as F, Cl, Br, or I). Compound 5-3 can be prepared by treating 5-2 with reagents such as triphosgene. Intermediate 5-3 can then react with ester 5-4 to deliver the nitro compound 5-5, which can be treated with an appropriate reagent (e.g. POCl₃) to afford compound 5-6. A S_{N}Ar reaction of intermediate 5-6 with amine 5-7 (PG is an appropriate protecting group, such as Boc) can be carried out to generate compound 5-8. The R³ group in 5-9 can then be installed via a suitable transformation, such as a S_{N}Ar reaction or a coupling reaction. Protection of the amino group affords intermediate 5-10, which can be reduced in the presence reducing agents (e.g. Fe in acetic acid) to provide 5-11. The halogen of 5-11 (Hal) can optionally be converted to R² via transition metal mediated coupling or other suitable method to obtain 5-12. Diazotization and reduction of the amino group in 5-12 affords intermediate 5-13, which after protecting group (PG) removal provides 5-14. Coupling of the bromo in 5-14 gives 5-15, which can be halogenated to provide intermediate 5-16. Sonagashira coupling affords 5-17, which after cyclization and deprotection provides compounds of the formula 5-18.

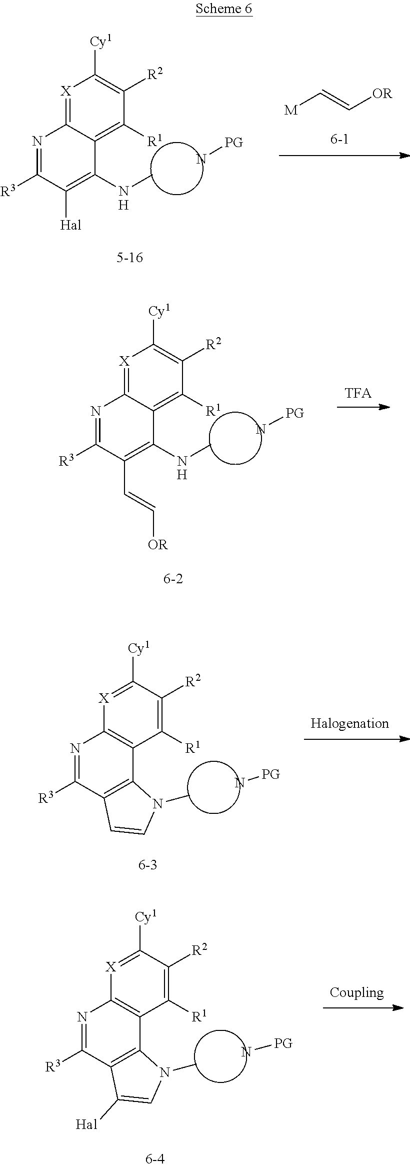

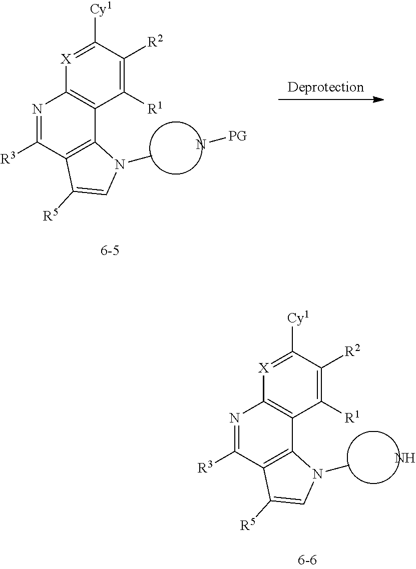

Compounds of the formula 6-6 can be prepared via the synthetic route outlined in Scheme 6. Coupling of 5-16 with an M (B, Sn, Si, Zn) substituted vinyl ether 6-1 affords intermediates 6-2, which upon treatment under acidic conditions (e.g., TFA) leads to 6-3. Halogenation of 6-3 provides 6-4, which can be converted to derivatives 6-5 via coupling or other suitable transformation. Deprotection of 6-5 then affords compounds of the formula 6-6.

### KRAS Protein

The Ras family is comprised of three members: KRAS, NRAS and HRAS. RAS mutant cancers account for about 25% of human cancers. KRAS is the most frequently mutated isoform in human cancers: 85% of all RAS mutations are in KRAS, 12% in NRAS, and 3% in HRAS (Simanshu, D. et al. Cell 170.1 (2017):17-33). KRAS mutations are prevalent amongst the top three most deadly cancer types: pancreatic (97%), colorectal (44%), and lung (30%) (Cox, A. D. et al. Nat Rev Drug Discov (2014) 13:828-51). The majority of RAS mutations occur at amino acid residues/codons 12, 13, and 61; Codon 12 mutations are most frequent in KRAS. The frequency of specific mutations varied between RAS genes and G12D mutations are most predominant in KRAS whereas Q61R and G12R mutations are most frequent in NRAS and HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) (Cox, A. D. et al. Nat Rev Drug Discov (2014) 13:828-51). In contrast, KRAS G12C mutations predominate in non-small cell lung cancer (NSCLC) comprising 11-16% of lung adenocarcinomas (nearly half of mutant KRAS is G12C), as well as 2-5% of pancreatic and colorectal adenocarcinomas, respectively (Cox, A. D. et al. Nat. Rev. Drug Discov. (2014) 13:828-51). Using shRNA knockdown thousands of genes across hundreds of cancer cell lines, genomic studies have demonstrated that cancer cells exhibiting KRAS mutations are highly dependent on KRAS function for cell growth (McDonald, R. et al. Cell 170 (2017): 577-592). Taken together, these findings suggested that KRAS mutations play a critical role in human cancers, therefore development of the inhibitors targeting mutant KRAS may be useful in the clinical treatment of diseases that have characterized by a KRAS mutation.

### Methods of Use

The cancer types in which KRAS harboring G12C, G12V, and G12D mutations are implicated include, but are not limited to: carcinomas (e.g., pancreatic, colorectal, lung, bladder, gastric, esophageal, breast, head and neck, cervical skin, thyroid); hematopoietic malignancies (e.g., myeloproliferative neoplasms (MPN), myelodysplastic syndrome (MDS), chronic and juvenile myelomonocytic leukemia (CMML and JMML), acute myeloid leukemia (AML), acute lymphocytic leukemia (ALL) and multiple myeloma (MM)); and other neoplasms (e.g., glioblastoma and sarcomas). In addition, KRAS mutations were found in acquired resistance to anti-EGFR therapy (Knickelbein, K. et al. Genes & Cancer, (2015): 4-12). KRAS mutations were found in immunological and inflammatory disorders (Fernandez-Medarde, A. et al. Genes & Cancer, (2011): 344-358) such as Ras-associated lymphoproliferative disorder (RALD) orjuvenile myelomonocytic leukemia (JMML) caused by somatic mutations of KRAS or NRAS.

Compounds of the present disclosure can inhibit the activity of the KRAS protein. For example, compounds of the present disclosure can be used to inhibit activity of KRAS in a cell or in an individual or patient in need of inhibition of the enzyme by administering an inhibiting amount of one or more compounds of the present disclosure to the cell, individual, or patient.

As KRAS inhibitors, the compounds of the present disclosure are useful in the treatment of various diseases associated with abnormal expression or activity of KRAS. Compounds which inhibit KRAS will be useful in providing a means of preventing the growth or inducing apoptosis in tumors, or by inhibiting angiogenesis. It is therefore anticipated that compounds of the present disclosure will prove useful in treating or preventing proliferative disorders such as cancers. In particular, tumors with activating mutants of receptor tyrosine kinases or upregulation of receptor tyrosine kinases may be particularly sensitive to the inhibitors.

In an aspect, provided herein is a method of inhibiting KRAS activity, said method comprising contacting a compound of the instant disclosure with KRAS. In an embodiment, the contacting comprises administering the compound to a patient.

In another aspect, provided herein a is method of treating a disease or disorder associated with inhibition of KRAS interaction, said method comprising administering to a patient in need thereof a therapeutically effective amount of a compound of any of the formulae disclosed herein, or pharmaceutically acceptable salt thereof.

In an embodiment, the disease or disorder is an immunological or inflammatory disorder.

In another embodiment, the immunological or inflammatory disorder is Ras-associated lymphoproliferative disorder and juvenile myelomonocytic leukemia caused by somatic mutations of KRAS.

In an aspect, provided herein is a method of treating a disease or disorder associated with inhibiting a KRAS protein harboring a G12C mutation, said method comprising administering to a patient in need thereof a therapeutically effective amount of a compound of any of the formulae disclosed herein, or pharmaceutically acceptable salt thereof.

In yet another aspect, provided herein is a method for treating a cancer in a patient, said method comprising administering to the patient a therapeutically effective amount of any one of the compounds disclosed herein, or pharmaceutically acceptable salt thereof.

In an embodiment, the cancer is selected from carcinomas, hematological cancers, sarcomas, and glioblastoma.

In another embodiment, the hematological cancer is selected from myeloproliferative neoplasms, myelodysplastic syndrome, chronic and juvenile myelomonocytic leukemia, acute myeloid leukemia, acute lymphocytic leukemia, and multiple myeloma.

In yet another embodiment, the carcinoma is selected from pancreatic, colorectal, lung, bladder, gastric, esophageal, breast, head and neck, cervical, skin, and thyroid.

In still another aspect, provided herein is a method of treating a disease or disorder associated with inhibiting a KRAS protein harboring a G12C mutation, said method comprising administering to a patient in need thereof a therapeutically effective amount of the compound of any of the formulae disclosed herein, or a pharmaceutically acceptable salt thereof.

In another aspect, provided herein is a method of treating cancer in a patient in need thereof comprising administering to the patient a therapeutically effective amount of the compounds disclosed herein wherein the cancer is characterized by an interaction with a KRAS protein harboring a G12C mutation.

In another aspect, provided herein is a method for treating a disease or disorder associated with inhibition of KRAS interaction or a mutant thereof in a patient in need thereof comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC).

In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In certain embodiments, the disclosure provides a method for treating a KRAS-mediated disorder in a patient in need thereof, comprising the step of administering to said patient a compound according to the invention, or a pharmaceutically acceptable composition thereof.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome, myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compounds of the present disclosure can also be useful in the inhibition of tumor metastases.

In addition to oncogenic neoplasms, the compounds of the invention are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a patient suffering from a skeletal and chondrocyte disorder.

In some embodiments, compounds described herein can be used to treat Alzheimer's disease, HIV, or tuberculosis.

As used herein, the term “8p11 myeloproliferative syndrome” is meant to refer to myeloid/lymphoid neoplasms associated with eosinophilia and abnormalities of FGFR1.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” KRAS with a compound described herein includes the administration of a compound described herein to an individual or patient, such as a human, having KRAS, as well as, for example, introducing a compound described herein into a sample containing a cellular or purified preparation containing KRAS.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” as used herein, comprises the prevention of at least one symptom associated with or caused by the state, disease or disorder being prevented.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

### Combination Therapies

### I. Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of CDK2-associated diseases, disorders or conditions. Other agents such as therapeutic antibodies can be used in combination with the compounds of the present disclosure for treatment of CDK2-associated diseases, disorders or conditions. The one or more additional pharmaceutical agents can be administered to a patient simultaneously or sequentially.

In some embodiments, the CDK2 inhibitor is administered or used in combination with a BCL2 inhibitor or a CDK4/6 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, flt-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2; e.g., ruxolitinib or baricitinib; or JAK1; e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a PI3K6 inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor.

In some embodiments, the compound or salt described herein is administered with a JAK1 inhibitor, which is selective over JAK2.

In addition, for treating cancer and other proliferative diseases, compounds described herein can be used in combination with targeted therapies such as, e.g., c-MET inhibitors (e.g., capmatinib), an anti-CD19 antibody (e.g., tafasitamab), an ALK2 inhibitor (e.g., INCB00928); or combinations thereof.

Example antibodies for use in combination therapy include, but are not limited to, trastuzumab (e.g., anti-HER2), ranibizumab (e.g., anti-VEGF-A), bevacizumab (AVASTIN™ e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, Ierozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Smi1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-1 agonists, oncolytic virotherapy and immunomodulating small molecules, including thalidomide or JAK1/2 inhibitor, PI3K6 inhibitor and the like. The compounds can be administered in combination with one or more anti-cancer drugs, such as a chemotherapeutic agent.

Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, B1853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a CDK2 inhibitor of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, Anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

### II. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (1B1308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. No. 7,488,802, 7,943,743, 8,008,449, 8,168,757, 8,217, 149, or 10,308,644; U.S. Publ. Nos. 2017/0145025, 2017/0174671, 2017/0174679, 2017/0320875, 2017/0342060, 2017/0362253, 2018/0016260, 2018/0057486, 2018/0177784, 2018/0177870, 2018/0179179, 2018/0179201, 2018/0179202, 2018/0273519, 2019/0040082, 2019/0062345, 2019/0071439, 2019/0127467, 2019/0144439, 2019/0202824, 2019/0225601, 2019/0300524, or 2019/0345170; or PCT Pub. Nos. WO 03042402, WO 2008156712, WO 2010089411, WO 2010036959, WO 2011066342, WO 2011159877, WO 2011082400, or WO 2011161699, which are each incorporated herein by reference in their entirety. In some embodiments, the inhibitor of PD-L1 is INCB086550.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012 (INCMGA0012; retifanlimab). In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559.

In some embodiments, the anti-PD-L1 antibody is MEDI4736. In some embodiments, the anti-PD-L1 antibody is FAZ053. In some embodiments, the anti-PD-L1 antibody is KN035.

In some embodiments, the anti-PD-L1 antibody is CS1001. In some embodiments, the anti-PD-L1 antibody is SHR-1316. In some embodiments, the anti-PD-L1 antibody is CBT-502.

In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MED11873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MED10562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound in a single or continuous dosage form, or they can be administered simultaneously or sequentially as separate dosage forms.

### Formulation, Dosage Forms and Administration

When employed as pharmaceuticals, the compounds of the present disclosure can be administered in the form of pharmaceutical compositions. Thus, the present disclosure provides a composition comprising a compound of Formula I, II, or any of the formulas as described herein, a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the invention. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### Labeled Compounds and Assay Methods

Another aspect of the present invention relates to labeled compounds of the disclosure (radio-labeled, fluorescent-labeled, etc.) that would be useful not only in imaging techniques but also in assays, both in vitro and in vivo, for localizing and quantitating KRAS protein in tissue samples, including human, and for identifying KRAS ligands by inhibition binding of a labeled compound. Substitution of one or more of the atoms of the compounds of the present disclosure can also be useful in generating differentiated ADME (Adsorption, Distribution, Metabolism and Excretion). Accordingly, the present invention includes KRAS binding assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula I, II, or any formulae provided herein can be optionally substituted with deuterium atoms, such as —CD₃ being substituted for —CH₃). In some embodiments, alkyl groups in Formula I, II, or any formulae provided herein can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro adenosine receptor labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the invention can be used in a screening assay to identify and/or evaluate compounds. For example, a newly synthesized or identified compound (i.e., test compound) which is labeled can be evaluated for its ability to bind a KRAS protein by monitoring its concentration variation when contacting with the KRAS, through tracking of the labeling. For example, a test compound (labeled) can be evaluated for its ability to reduce binding of another compound which is known to bind to a KRAS protein (i.e., standard compound). Accordingly, the ability of a test compound to compete with the standard compound for binding to the KRAS protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, e.g., in the treatment or prevention of diseases or disorders associated with the activity of KRAS, such as cancer or infections, which include one or more containers containing a pharmaceutical composition comprising a therapeutically effective amount of a compound of Formula I, II, or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

The compounds of the Examples have been found to inhibit the activity of KRAS according to at least one assay described herein.

### EXAMPLES

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004). The compounds separated were typically subjected to analytical liquid chromatography mass spectrometry (LCMS) for purity check.

The compounds separated were typically subjected to analytical liquid chromatography mass spectrometry (LCMS) for purity check under the following conditions: Instrument; Agilent 1100 series, LC/MSD, Column: Waters Sunfire™ C₁₈ 5 μm particle size, 2.1×5.0 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm particle size, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature [see “Preparative LCMS Purification: Improved Compound Specific Method Optimization”, K. Blom, B. Glass, R. Sparks, A. Combs, J. Comb. Chem., 6, 874-883 (2004)]. Typically, the flow rate used with the 30×100 mm column was 60 mL/minute.

pH=10 purifications: Waters XBridge C₁₈ 5 μm particle size, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature [See “Preparative LCMS Purification: Improved Compound Specific Method Optimization”, K. Blom, B. Glass, R. Sparks, A. Combs, J. Comb. Chem., 6, 874-883 (2004)]. Typically, the flow rate used with 30×100 mm column was 60 mL/minute.”

The following abbreviations may be used herein: AcOH (acetic acid); Ac₂O (acetic anhydride); aq. (aqueous); atm. (atmosphere(s)); Boc (t-butoxycarbonyl); br (broad); Cbz (carboxybenzyl); calc. (calculated); d (doublet); dd (doublet of doublets); DBU (1,8-diazabicyclo[5.4.0]undec-7-ene); DCM (dichloromethane); DIAD (N, N′-diisopropyl azidodicarboxylate); DIEA (N,N-diisopropylethylamine); DIBAL-H (diisobutylaluminium hydride); DMF (N, N-dimethylformamide); EtOH (ethanol); EtOAc (ethyl acetate); FCC (flash column chromatography); g (gram(s)); h (hour(s)); HATU (N, N, N′, N′-tetramethyl-O-(7-azabenzotriazol-1-yl)uronium hexafluorophosphate); HCl (hydrochloric acid); HPLC (high performance liquid chromatography); Hz (hertz); J (coupling constant); LCMS (liquid chromatography—mass spectrometry); LDA (lithium diisopropylamide); m (multiplet); M (molar); mCPBA (3-chloroperoxybenzoic acid); MS (Mass spectrometry); Me (methyl); MeCN (acetonitrile); MeOH (methanol); mg (milligram(s)); min. (minutes(s)); mL (milliliter(s)); mmol (millimole(s)); N (normal); NCS (N-chlorosuccinimide); NEt₃ (triethylamine); nM (nanomolar); NMP (N-methylpyrrolidinone); NMR (nuclear magnetic resonance spectroscopy); OTf (trifluoromethanesulfonate); Ph (phenyl); pM (picomolar); PPT (precipitate); RP-HPLC (reverse phase high performance liquid chromatography); r.t. (room temperature), s (singlet); t (triplet or tertiary); TBS (tert-butyldimethylsilyl); tert (tertiary); tt (triplet of triplets); TFA (trifluoroacetic acid); THF (tetrahydrofuran); μg (microgram(s)); μL (microliter(s)); μM (micromolar); wt % (weight percent). Brine is saturated aqueous sodium chloride. In vacuo is under vacuum.

The compounds of the present disclosure can be isolated in free-base or pharmaceutical salt form. In the examples provided herein, the compounds are isolated as the corresponding TFA salt.

### Example 1. 1-(4-(8-chloro-6-fluoro-7-(2-fluoro-6-hydroxyphenyl)-1H-pyrazolo[4,3-c]quinolin-1-yl)piperidin-1-yl)prop-2-en-1-one

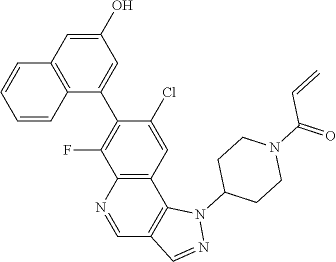 [SMILES: C=CC(=O)N1CCC(n2ncc3cnc4c(F)c(-c5cc(O)cc6ccccc56)c(Cl)cc4c32)CC1]

### Step 1: 3-bromo-4-chloro-2-fluoroaniline

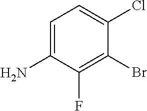

To a solution of 3-bromo-2-fluoroaniline (46.8 g, 246 mmol) in DMF (246 ml) was added NCS (34.5 g, 259 mmol) portionwise, and the resultant mixture stirred at room temperature overnight. The mixture was poured onto ice-water (400 mL) and extracted with ethyl acetate. The organic layer was washed with water (2×), brine, dried over Na₂SO₄, filtered and concentrated. The crude was purified with silica gel column (0-30% ethyl acetate in hexanes) to give the desired product as brown oil which solidified on standing (38 g, 69%). LC-MS calculated for C₆H₅BrClFN (M+H)⁺: m/z=223.9, 225.9; found 223.9, 225.9.

### Step 2: ethyl 7-bromo-6-chloro-8-fluoro-4-hydroxyquinoline-3-carboxylate

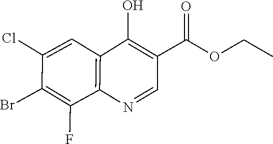 [SMILES: CCOC(=O)c1cnc2c(F)c(Br)c(Cl)cc2c1O]

A mixture of 3-bromo-4-chloro-2-fluoroaniline (6.03 g, 26.9 mmol), diethyl 2-(ethoxymethylene)malonate (6.39 g, 29.6 mmol) and EtOH (54 ml) was stirred at 80° C. for 16 h. The mixture was allowed to cool to room temperature. The reaction mixture was concentrated and the residue was diluted with heptane, and stirred for 20 min at room temperature, by which time a solid had precipitated from solution. The solid was collected by filtration, washed with heptane and dried under vacuum to give a solid. To a round bottom flask charged with diethyl 2-(((3-bromo-4-chloro-2-fluorophenyl)amino)methylene)malonate (9.8 g, 24.83 mmol) was added phenyl ether (43 mL). The resulting solution was stirred at 230° C. for 10 h. The reaction was cooled to 40° C. with stirring. The resulting solid was collected by filtration, washed with diethyl ether (3×50 mL) and dried under vacuum to afford crude ethyl 7-bromo-6-chloro-8-fluoro-4-hydroxyquinoline-3-carboxylate (6.46 g, 69%) as beige solid, which was used without purification. LC-MS calculated for C₁₂H₉BrClFNO₃ (M+H)⁺: m/z=347.9, 349.9; found 347.9, 349.9.

### Step 3: ethyl 7-bromo-4,6-dichloro-8-fluoroquinoline-3-carboxylate

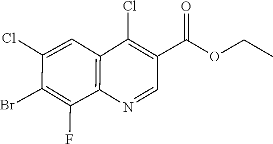 [SMILES: CCOC(=O)c1cnc2c(F)c(Br)c(Cl)cc2c1Cl]

To a round bottom flask charged with ethyl 7-bromo-6-chloro-8-fluoro-4-hydroxyquinoline-3-carboxylate (6.46 g, 18.53 mmol) was added POCl₃ (34.5 ml, 371 mmol). The resulting mixture was heated at 110° C. for 4 h. The mixture was diluted with toluene and evaporated under vacuum. The residue was dissolved in DCM and poured into ice water and neutralized with sat. NaHCO₃. The organic layer was separated and dried over Na₂SO₄, filtered and concentrated to give the desired product (5.8 g, 85%). LC-MS calculated for C₁₂H₈BrCl₂FNO₂ (M+H)⁺: m/z=365.9, 367.9; found 365.9, 367.9.

### Step 4. (7-bromo-4,6-dichloro-8-fluoroquinolin-3-yl)methanol

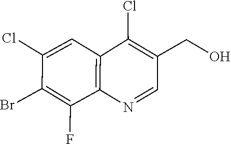 [SMILES: OCc1cnc2c(F)c(Br)c(Cl)cc2c1Cl]

1.0 M DIBAL-H in DCM (7.77 ml, 7.77 mmol) was added to ethyl 7-bromo-4,6-dichloro-8-fluoroquinoline-3-carboxylate (0.95 g, 2.59 mmol) in CH2Cl₂ (14.88 ml) at room temperature. The mixture was stirred at 0° C. overnight. 1.0 M NaOH solution was added to reaction mixture, the resulting precipitate was filtered. The aqueous layer was extracted with DCM. The combined organic layers were washed with brine, dried and evaporated. The residue was purified with flash chromatography (eluting with a gradient of 0-30% ethyl acetate in hexanes) to give the desired product (0.60 g, 71%). LC-MS calculated for C₁₀H₆BrCl₂FNO (M+H)⁺: m/z=323.9, 325.9; found 323.9, 325.9.

### Step 5: 7-bromo-4,6-dichloro-8-fluoroquinoline-3-carbaldehyde

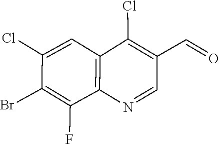 [SMILES: O=Cc1cnc2c(F)c(Br)c(Cl)cc2c1Cl]

To a solution of (7-bromo-4,6-dichloro-8-fluoroquinolin-3-yl)methanol (340 mg, 1.046 mmol) in DCM (6 ml) was added dess-martinperiodinane (533 mg, 1.256 mmol). The resulting mixture was stirred at room temperature for 1 h. The reaction was diluted with DCM and saturated NaHCO₃ solution and stirred for 10 mins. The organic layer was separated and dried over Na₂SO₄, filtered and concentrated. The crude was purified with flash chromatography (eluting with a gradient of 0-30% ethyl acetate in hexanes) to give the desired product (0.20 g, 59.2%). LC-MS calculated for C₁₀H₄BrCl₂FNO (M+H)⁺: m/z=321.9, 323.9; found 321.7, 323.7.

### Step 6. 7-bromo-8-chloro-6-fluoro-1-(piperidin-4-yl)-1H-pyrazolo[4,3-c]quinoline

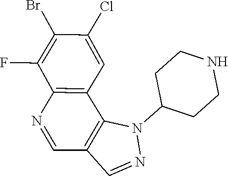 [SMILES: Fc1c(Br)c(Cl)cc2c1ncc1cnn(C3CCNCC3)c12]

To a microwave vial was added 7-bromo-4,6-dichloro-8-fluoroquinoline-3-carbaldehyde (51 mg, 0.158 mmol), tert-butyl 4-hydrazinylpiperidine-1-carboxylate (40.8 mg, 0.190 mmol) and 1,1,1,3,3,3-hexafluoro-2-propanol (1.0 ml). The vial was heated at 90° C. for 20 min and 150° C. 40 min. The reaction mixture was diluted with methanol and purified with prep-LCMS (pH 2) to give the desired product (36 mg, 59%). LC-MS calculated for C₁₅H₁₄BrClFN₄ (M+H)⁺: m/z=383.0, 385.0; found 383.0, 385.0.

### Step 7. 1-(4-(7-bromo-8-chloro-6-fluoro-1H-pyrazolo[4,3-c]quinolin-1-yl)piperidin-1-yl)prop-2-en-1-one

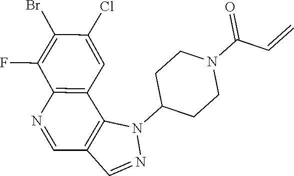 [SMILES: C=CC(=O)N1CCC(n2ncc3cnc4c(F)c(Br)c(Cl)cc4c32)CC1]

To a solution of 7-bromo-8-chloro-6-fluoro-1-(piperidin-4-yl)-1H-pyrazolo[4,3-c]quinoline (36 mg, 0.094 mmol) in DCM (1.0 ml) was added DIEA (32.8 μl, 0.188 mmol), followed by 1.0 M acryloyl chloride (113 μl, 0.113 mmol). After stirring at 0° C. for 1 h, the solvent was removed and the residue was diluted with methanol and purified with prep-LCMS (pH 2 acetonitrile/water+TFA) to give the desired product (25 mg, 61%). LC-MS calculated for C₁₈H₁₆BrClFN₄O (M+H)⁺: m/z=437.0, 439.0; found 437.1, 439.1.

### Step 8. 1-(4-(8-chloro-6-fluoro-7-(3-hydroxynaphthalen-1-yl)-1H-pyrazolo[4,3-c]quinolin-1-yl)piperidin-1-yl)prop-2-en-1-one

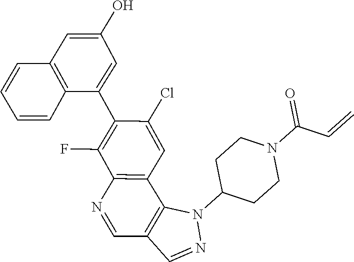 [SMILES: C=CC(=O)N1CCC(n2ncc3cnc4c(F)c(-c5cc(O)cc6ccccc56)c(Cl)cc4c32)CC1]

A mixture of 1-(4-(7-bromo-8-chloro-6-fluoro-1H-pyrazolo[4,3-c]quinolin-1-yl)piperidin-1-yl)prop-2-en-1-one (10 mg, 0.023 mmol), 4-(4,4,5,5-tetramethyl-1,3,2-dioxaborolan-2-yl)naphthalen-2-ol (12.34 mg, 0.046 mmol), tetrakis (2.64 mg, 2.285 μmol) and sodium carbonate (6.05 mg, 0.057 mmol) in 1,4-dioxane (1.0 mL)/water (0.200 mL) was stirred at 90° C. for 2 h. The residue was dissolved in methanol and 1 N HCl and purified with prep-LCMS (pH 2, acetonitrile/water+TFA) to give the desired product as white solid (3.2 mg, 30%). LC-MS calculated for C₂₈H₂₃ClFN₄O₂ (M+H)⁺: m/z=501.1; found 501.1.

### Example 2. 1-(4-(8-chloro-4-(3-(dimethylamino)azetidin-1-yl)-6-fluoro-7-(3-hydroxynaphthalen-1-yl)-1H-pyrazolo[4,3-c]quinolin-1-yl)piperidin-1-yl)prop-2-en-1-one

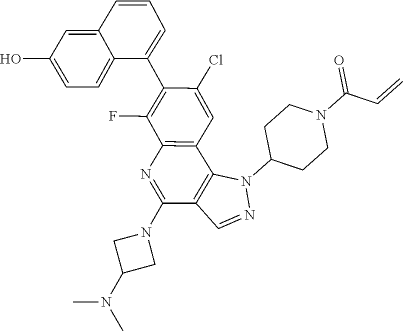 [SMILES: C=CC(=O)N1CCC(n2ncc3c(N4CC(N(C)C)C4)nc4c(F)c(-c5cccc6cc(O)ccc56)c(Cl)cc4c32)CC1]

### Step 1: 3-bromo-4-chloro-2-fluoroaniline

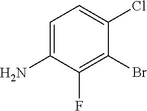

To a solution of 3-bromo-2-fluoroaniline (46.8 g, 246 mmol) in DMF (246 ml) was added NCS (34.5 g, 259 mmol) portionwise, and the resultant mixture stirred at room temperature overnight. The mixture was poured onto ice-water (400 mL) and extracted with ethyl acetate. The organic layer was washed with water (2×), brine, dried over Na₂SO₄, filtered and concentrated. The crude was purified with silica gel column (0-30% ethyl acetate in hexanes) to give the desired product as brown oil which solidified on standing (38 g, 69%). LC-MS calculated for C₆HsBrClFN (M+H)⁺: m/z=223.9, 225.9; found 223.9, 225.9.

### Step 2: 7-bromo-6-chloro-8-fluoroquinoline-2,4-diol

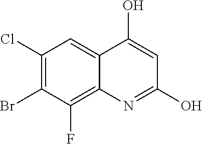 [SMILES: Oc1cc(O)c2cc(Cl)c(Br)c(F)c2n1]

A mixture containing 3-bromo-4-chloro-2-fluoroaniline (1.25 g, 5.57 mmol) and 2,2-dimethyl-1,3-dioxane-4,6-dione (0.803 g, 5.57 mmol) was stirred at 80° C. for 2 h, 2,2-dimethyl-1,3-dioxane-4,6-dione (0.803 g, 5.57 mmol) and 1,4-dioxane (4 ml) was added. After stirring at 80° C. another 2 hours, the mixture was cooled to 23° C. and then ethyl acetate (100 mL) was added. The mixture was extracted with 1.0 M aqueous sodium hydroxide solution (100 mL). The basic aqueous layer was washed with ethyl acetate (50 mL). The washed layer was brought to pH 2 with 6 M aqueous hydrochloric acid solution. The acidic aqueous solution was extracted with ethyl acetate (3×60 mL). The organic layers were combined and the combined solution was dried with magnesium sulfate. The dried solution was filtered an the filtrate was concentrated to afford the title compound as a white solid.

A mixture containing 3-((3-bromo-4-chloro-2-fluorophenyl)amino)-3-oxopropanoic acid (1.61 g, 5.19 mmol) and polyphosphoric acid (30 g) was heated to 100° C. After 2 hours, the mixture was cooled to 23° C. and then poured into ice water (200 mL) resulting in the formation of a solid. The mixture was stirred overnight and then filtered. The filter cake was collected to provide the title compound (1.16 g, 71%) as beige solid which was used without purification. LC-MS calculated for C₉H₅BrClFNO₂ (M+H)⁺: m/z=291.9, 293.9; found 291.8, 293.8.

### Step 3: 7-bromo-2,4,6-trichloro-8-fluoroquinoline

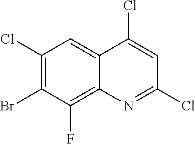 [SMILES: Fc1c(Br)c(Cl)cc2c(Cl)cc(Cl)nc12]

POCl₃ (9.94 ml, 107 mmol) was added to 7-bromo-6-chloro-8-fluoroquinoline-2,4-diol (5.2 g, 17.78 mmol) in toluene (60 ml) at room temperature. The mixture was heated at 110° C. with stirring for 2.5 h. The solvents were removed by evaporation. Toluene (15 mL) was added and the solvents evaporated. The residue was taken up in DCM (100 mL) and poured into ice-cold sat NaHCO3 (150 mL). The mixture was extracted with DCM (2×). The combined organic layers were washed with brine, dried and evaporated. The crude was purified with flash chromatography (eluting with a gradient 0-35% DCM in hexanes) to give the title compound as white solid (2.4 g, 41.0%). LC-MS calculated for C₉H₃BrCl₃FN (M+H)⁺: m/z=327.8, 329.8, 331.8; found 327.8, 329.7, 331.8.

### Step 4. 7-bromo-2,4,6-trichloro-8-fluoroquinoline-3-carbaldehyde

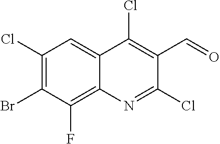 [SMILES: O=Cc1c(Cl)nc2c(F)c(Br)c(Cl)cc2c1Cl]

A stirred solution of 7-bromo-2,4,6-trichloro-8-fluoroquinoline (1.45 g, 4.40 mmol) in THF (44 mL) was cooled to −78° C., to which was added dropwise 2.00 M LDA (2.42 ml, 4.84 mmol) under nitrogen atmosphere, stirred for 30 min, and then was added DMF (1.704 ml, 22.01 mmol). The reaction mixture was stirred at −78° C. for 3 hrs, allowed to warm to room temperature, quenched with saturated NH₄Cl solution, diluted with water, and extracted with ethyl acetate. The combined organic extract was washed with water, brine, and dried (Na₂SO₄), and the solvent was evaporated to furnish the residue which was chromatographed (10% ethyl acetate/hexanes) to afford the title compound as yellow solid (0.7 g, 45%). LC-MS calculated for C₁₀H₃BrCl₃FNO (M+H)⁺: m/z=355.8, 357.8, 359.8; found 355.9, 357.9, 359.9.

### Step 5: 1-(7-bromo-8-chloro-6-fluoro-1-(piperidin-4-yl)-1H-pyrazolo[4,3-c]quinolin-4-yl)-N,N-dimethylazetidin-3-amine

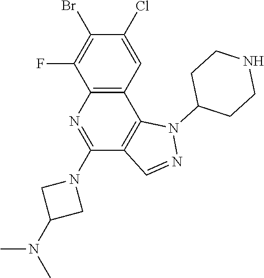 [SMILES: CN(C)C1CN(c2nc3c(F)c(Br)c(Cl)cc3c3c2cnn3C2CCNCC2)C1]

To a microwave vial was added 7-bromo-2,4,6-trichloro-8-fluoroquinoline-3-carbaldehyde (81 mg, 0.227 mmol) and tert-butyl 4-hydrazinylpiperidine-1-carboxylate hydrochloride (57.1 mg, 0.227 mmol), 2-propanol (1 ml). The vial was heated at 90° C. for 20 min and 140° C. 40 min. To the reaction vial was added N,N-dimethylazetidin-3-amine dihydrochloride (58.8 mg, 0.340 mmol) and DIEA (39.6 μl, 0.227 mmol). The vial was heated at 150° C. for 1h in microwave processor. After cooling to room temperature, TFA (0.5 mL) was added and stirred for 1 h. LCMS showed total conversion of SM. The reaction mixture was diluted with methanol and purified with prep-LCMS (pH 2 acetonitrile/water+TFA) to give the compound C (36 mg, 38.0%). LC-MS calculated for C₂₀H₂₄BrClFN₆ (M+H)⁺: m/z=481.1, 483.1; found 481.1, 483.1.

### Step 6. 1-(4-(7-bromo-8-chloro-4-(3-(dimethylamino)azetidin-1-yl)-6-fluoro-1H-pyrazolo[4,3-c]quinolin-1-yl)piperidin-1-yl)prop-2-en-1-one

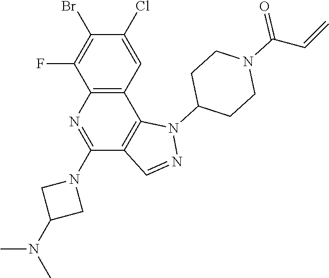 [SMILES: C=CC(=O)N1CCC(n2ncc3c(N4CC(N(C)C)C4)nc4c(F)c(Br)c(Cl)cc4c32)CC1]

To a solution of 1-(7-bromo-8-chloro-6-fluoro-1-(piperidin-4-yl)-1H-pyrazolo[4,3-c]quinolin-4-yl)-N,N-dimethylazetidin-3-amine (46 mg, 0.095 mmol) in DCM (1.0 ml). DIEA (33.4 μl, 0.191 mmol) was added to reaction vial, followed by 1.0 M acryloyl chloride (115 μl, 0.115 mmol). After stirring at 0° C. for 1 h, the solvent was removed and the residue was diluted with methanol and purified with prep-LCMS to give the desired product (15 mg, 29%). LC-MS calculated for C₂₃H₂₆BrClFN₆O (M+H)⁺: m/z=535.1, 537.1; found 535.1, 537.1.

### Step 7. 1-(4-(8-chloro-4-(3-(dimethylamino)azetidin-1-yl)-6-fluoro-7-(3-hydroxynaphthalen-1-yl)-1H-pyrazolo[4,3-c]quinolin-1-yl)piperidin-1-yl)prop-2-en-1-one

A mixture of 1-(4-(7-bromo-8-chloro-4-(3-(dimethylamino)azetidin-1-yl)-6-fluoro-1H-pyrazolo[4,3-c]quinolin-1-yl)piperidin-1-yl)prop-2-en-1-one (15 mg, 0.028 mmol), 4-(4,4,5,5-tetramethyl-1,3,2-dioxaborolan-2-yl)naphthalen-2-ol (15.12 mg, 0.056 mmol), tetrakis (3.23 mg, 2.80 μmol) and sodium carbonate (7.42 mg, 0.070 mmol) in 1,4-dioxane (1.0 mL)/water (0.200 mL) was stirred at 90° C. for 2 h. The residue was dissolved in methanol and 1 N HCl and purified with prep-LCMS (pH 2 acetonitrile/water+TFA) to give the title compound as white solid (5.0 mg, 30%). LC-MS calculated for C₃₃H₃₃ClFN₆O₂ (M+H)⁺: m/z=599.1; found 599.3.

### Example 3a and Example 3b. 2-((2S,4S)-1-acryloyl-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile

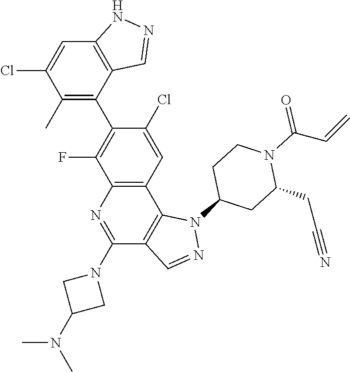 [SMILES: C=CC(=O)N1CC[C@H](n2ncc3c(N4CC(N(C)C)C4)nc4c(F)c(-c5c(C)c(Cl)cc6[nH]ncc56)c(Cl)cc4c32)C[C@H]1CC#N]

### Step 1: methyl 2-amino-4-bromo-5-chloro-3-fluorobenzoate

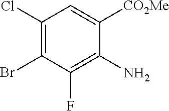 [SMILES: COC(=O)c1cc(Cl)c(Br)c(F)c1N]

Sulfuric acid (7.76 ml, 146 mmol) was added slowly to a solution of 2-amino-4-bromo-5-chloro-3-fluorobenzoic acid (19.5 g, 72.8 mmol) in MeOH (146 ml) at r.t. The resulting mixture was heated to 80° C. overnight. The mixture was then cooled to r.t. and slowly poured into sat'd NaHCO₃. The mixture was stirred at r.t. for 30 min then extracted with EtOAc. The organic layer was dried over MgSO₄, filtered, concentrated, and used in the next step without further purification. LC-MS calculated for C₃H₇BrClFNO₂ (M+H)⁺: m/z=281.9, 283.9; found 281.9, 283.9.

### Step 2: ethyl 7-bromo-6-chloro-8-fluoro-4-hydroxy-2-oxo-1,2-dihydroquinoline-3-carboxylate

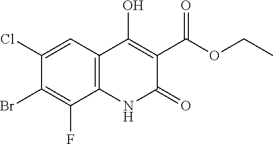 [SMILES: CCOC(=O)c1c(O)c2cc(Cl)c(Br)c(F)c2[nH]c1=O]

Ethyl 3-chloro-3-oxopropanoate (9.60 ml, 75.0 mmol) was added dropwise to a solution of methyl 2-amino-4-bromo-5-chloro-3-fluorobenzoate (19.25 g, 68.1 mmol) and TEA (14.25 ml, 102 mmol) in DCM (150 mL) at rt. After stirring for 1 h, additional ethyl 3-chloro-3-oxopropanoate (1.745 ml, 13.63 mmol) added. After stirring for another 1 h, the reaction was quenched with water then extracted with ethyl acetate. The organic layer was dried, filtered, then concentrated. The concentrated residue was redissolved in EtOH (150 ml) and sodium ethoxide in ethanol (53.4 ml, 143 mmol) was added. stirred at r.t. for 1 h. The reaction mixture was poured into water (1 L) and acidify to pH ˜3, The resulting precipitate was collected via filtration to give the desired product (18.39 g, 74.0%). LC-MS calculated for C₁₂H₉BrClFNO₄ (M+H)⁺: m/z=363.9, 365.9; found 363.9, 365.9.

### Step 3: ethyl 7-bromo-2,4,6-trichloro-8-fluoroquinoline-3-carboxylate

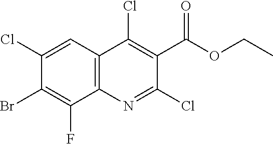 [SMILES: CCOC(=O)c1c(Cl)nc2c(F)c(Br)c(Cl)cc2c1Cl]

Ethyl 7-bromo-6-chloro-8-fluoro-2,4-dihydroxyquinoline-3-carboxylate (2.0 g, 5.49 mmol) was dissolved in POCl₃ (10.2 ml, 110 mmol), and DIEA (1.92 ml, 10.97 mmol) was added. The resulting mixture was stirred at 100° C. for 2h. After cooling to r.t., the reaction was quenched by slowly pouring into rapidly stirred ice water (˜250 mL), stirred for 30 min then collected solids via filtration to yield the desired product as a brown solid (1.66 g, 75%). LC-MS calculated for C₁₂H₇BrCl₃FNO₂ (M+H)⁺: m/z=399.9, 401.9, 403.9; found 399.9, 401.9, 403.9.

### Step 4. tert-butyl (R)-6-cyano-5-hydroxy-3-oxohexanoate

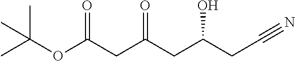 [SMILES: CC(C)(C)OC(=O)CC(=O)C[C@H](O)CC#N]

To a solution of 2.0 M LDA (100 ml, 200 mmol) in anhydrous THF (223 ml) was cooled to −78° C. for 1 h, and then tert-butyl acetate (26.9 ml, 200 mmol) was added dropwise with stirring over 20 min. After an additional 40 minutes maintained at −78° C., a solution of ethyl (R)-4-cyano-3-hydroxybutanoate (10.5 g, 66.8 mmol) was added dropwise. The mixture was allowed to stir at −40° C. for 4 h, and then an appropriate amount of HCl (2 M) was added to the mixture, keeping pH ˜6. During this quench, the temperature of the mixture was maintained at −10° C. Upon completion, the temperature of the mixture was cooled to 0° C.

The mixture was extracted with ethyl acetate (3×100 mL). The combined organic layer was washed with NaHCO₃ (100 mL) and brine (100 mL), dried over anhydrous Na₂SO₄, and evaporated to provide the material as yellow oil (15.0 g, 99%).

### Step 5. tert-butyl (2S,4R)-2-(2-(tert-butoxy)-2-oxoethyl)-4-hydroxypiperidine-1-carboxylate

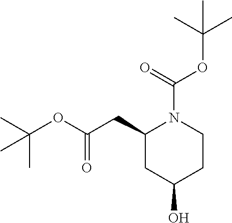 [SMILES: CC(C)(C)OC(=O)C[C@@H]1C[C@H](O)CCN1C(=O)OC(C)(C)C]

A solution of tert-butyl (R)-6-cyano-5-hydroxy-3-oxohexanoate (15.0 g, 66.0 mmol) in acetic acid (110 ml) was treated with platinum (IV) oxide hydrate (0.868 g, 3.30 mmol). The Parr bottle was evacuated and backfilled with H₂ three times and stirred under a H₂ atmosphere (45 psi, recharged 4 times) at 22° C. for 3h. The mixture was filtered through Celite and the filter cake was washed with EtOH. The filtrate was concentrated to yield product with a ˜9:1 cis:trans diastereomer ratio. The residue was dissolved in methanol (100 mL) then Boc-anhydride (15.3 ml, 66.0 mmol), sodium carbonate (13.99 g, 132 mmol) was added. The reaction mixture was stirred at room temperature overnight. The mixture was filtered and concentrated. The residue was purified with silica gel column to give the desired product (11.7 g, 56%). LCMS (product+Na⁺) calculated for C₁₆H₂₉NNaO₅ (M+Na)⁺: m/z=338.2; found: 338.2.

### Step 6. tert-butyl (2S,4S)-4-azido-2-(2-(tert-butoxy)-2-oxoethyl)piperidine-1-carboxylate

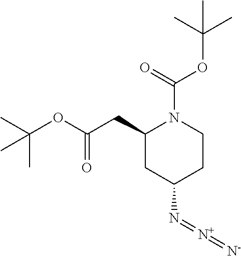 [SMILES: CC(C)(C)OC(=O)C[C@@H]1C[C@@H](N=[N+]=[N-])CCN1C(=O)OC(C)(C)C]

To a solution of tert-butyl (2S,4R)-2-(2-(tert-butoxy)-2-oxoethyl)-4-hydroxypiperidine-1-carboxylate (2.10 g, 6.66 mmol) in DCM (33 ml) at 0° C. was added Ms—Cl (0.67 mL, 8.66 mmol), After stirring for 1 h, The reaction was diluted with water and organic layer was separated and dried over Na₂SO₄, filtered and concentrated. The resulting residue was dissolved in DMF and sodium azide (1.3 g, 20 mmol) was added and the reaction mixture was heated at 70° C. for 5 h. After cooling to rt, the reaction was diluted with EtOAc and water. The organic layer was separated and dried over Na₂SO₄, filtered and concentrated. The residue was purified with silica gel column to give the desired product (1.90 g, 84%). LCMS calculated for (Product-Boc) C₁₁H₂₁N₄O₂ (M+H)⁺: m/z=241.2; found: 241.2.

### Step 7. tert-butyl (2S,4S)-4-azido-2-(2-hydroxyethyl)piperidine-1-carboxylate

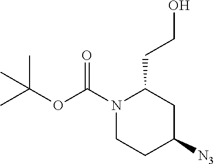 [SMILES: CC(C)(C)OC(=O)N1CC[C@H](N=[N+]=[N-])C[C@H]1CCO]

To a solution of tert-butyl (2S,4S)-4-azido-2-(2-(tert-butoxy)-2-oxoethyl)piperidine-1-carboxylate (21.4 g, 62.9 mmol) in DCM (400 ml) at −78° C. was added 1.0 M DIBAL-H in DCM (113 ml, 113 mmol). The resulting mixture was stirred at −78° C. for 2h. The reaction was quenched with methanol (38.1 ml, 943 mmol) at −78° C. Aqueous Rochelle salt solution (prepared from 126 g (6 wt) of Rochelle salt and 300 mL of water) was added to the solution at ≤10° C. The biphasic mixture was stirred vigorously for ≥1 h at 15-25° C. and separated to give organic layer. The biphasic mixture was separated. The organic layer was washed with aqueous NaCl (×2) at 15-25° C., The organic layer was dried over Na₂SO₄, filtered and concentrated. and used as is. The residue was dissolved in the methanol (300 mL) and sodium borohydride (1.43 g, 37.7 mmol) was added at 0° C. The reaction mixture was stirred at 0° C. for 1 h. The reaction was quenched with water, methanol was evaporated under reduced pressure. The reaction mixture was extracted with ethyl acetate (2×), the organic layer was washed with brine, dried over Na₂SO₄, filtered and concentrated. The crude was purified with flash chromatography (eluting with a gradient 0-50% ethyl acetate in hexanes) to give the desired product as colorless oil (14.8 g, 87%). LCMS calculated for (Product-Boc) C₇H₁₅N₄O (M+H)⁺: m/z=171.1; found: 171.1.

### Step 8. tert-butyl (2S,4S)-4-azido-2-(2-((tert-butyldimethylsilyl)oxy)ethyl)piperidine-1-carboxylate

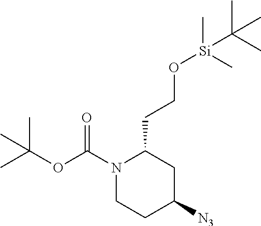 [SMILES: CC(C)(C)OC(=O)N1CC[C@H](N=[N+]=[N-])C[C@H]1CCO[Si](C)(C)C(C)(C)C]

To a solution of tert-butyl (2S,4S)-4-azido-2-(2-hydroxyethyl)piperidine-1-carboxylate (4.0 g, 14.80 mmol) in DMF (74.0 ml) was added imidazole (1.51 g, 22.2 mmol) and TBS-CI (2.90 g, 19.24 mmol). The resulting mixture was stirred at 60° C. for 1 h 15 min. The reaction mixture was diluted with EtOAc and water. The organic layer was washed with water (2×), brine, dried over Na₂SO₄, filtered and concentrated. The residue was purified with flash chromatography (0-20% ethyl acetate in hexanes) to give the desired product as colorless oil. (5.30 g, 93%). LCMS calculated for (Product-Boc) C₁₃H₂₉N₄OSi (M+H)⁺: m/z=285.2; found: 285.2.

### Step 9. tert-butyl (2S,4S)-4-amino-2-(2-((tert-butyldimethylsilyl)oxy)ethyl)piperidine-1-carboxylate

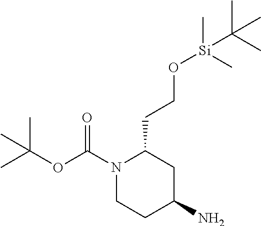 [SMILES: CC(C)(C)OC(=O)N1CC[C@H](N)C[C@H]1CCO[Si](C)(C)C(C)(C)C]

To a solution of tert-butyl (2S,4S)-4-azido-2-(2-((tert-butyldimethylsilyl)oxy)ethyl)-piperidine-1-carboxylate (5.30 g, 13.78 mmol) in methanol (70 ml) was added 10% palladium on carbon (1.47 g, 1.38 mmol). The reaction mixture was evacuated under vacuum and refilled with H₂, stirred at rt for 2 h. The reaction mixture was filtered through a pad of Celite and washed with methanol. The filtrate was concentrated to give the desired product (4.5 g, 91%). LCMS calculated for (Product-Boc) C₁₃H₃₁N₂OSi (M+H)⁺: m/z=259.2; found: 259.2.

### Step 10. ethyl 7-bromo-4-(((2S,4S)-1-(tert-butoxycarbonyl)-2-(2-((tert-butyldimethylsilyl)oxy)ethyl)piperidin-4-yl)amino)-2,6-dichloro-8-fluoroquinoline-3-carboxylate

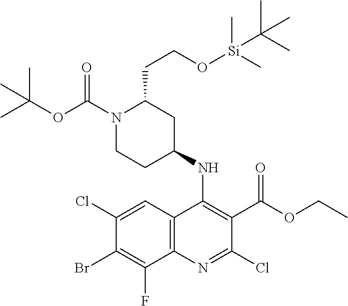 [SMILES: CCOC(=O)c1c(Cl)nc2c(F)c(Br)c(Cl)cc2c1N[C@H]1CCN(C(=O)OC(C)(C)C)[C@H](CCO[Si](C)(C)C(C)(C)C)C1]

To a solution of ethyl 7-bromo-2,4,6-trichloro-8-fluoroquinoline-3-carboxylate (8.7 g, 21.7 mmol) in DMF (80 ml) was added tert-butyl (2S,4S)-4-amino-2-(2-((tert-butyldimethylsilyl)oxy)ethyl)piperidine-1-carboxylate (9.33 g, 26.0 mmol) and DIEA (7.6 ml, 43.3 mmol). The resulting mixture was stirred at 65° C. for 5 h. After cooling to room temperature, ethyl acetate and water were added. The organic layer was washed with water (2×) and brine, dried over Na₂SO₄, filtered and concentrated. The residue was purified with flash chromatography (eluting with 0%-25% ethyl acetate in hexanes) to give the desired product as foam (14.6 g, 93%). LC-MS calculated for C₃₀H₄₄BrCl₂FN₃O₅Si (M+H)⁺: m/z=722.2, 724.2; found 722.2, 724.2.

### Step 11. tert-butyl (2S,4S)-4-((7-bromo-2,6-dichloro-8-fluoro-3-(hydroxymethyl)quinolin-4-yl)amino)-2-(2-((tert-butyldimethylsilyl)oxy)ethyl)piperidine-1-carboxylate

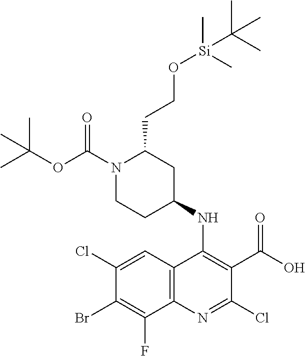 [SMILES: CC(C)(C)OC(=O)N1CC[C@H](Nc2c(C(=O)O)c(Cl)nc3c(F)c(Br)c(Cl)cc23)C[C@H]1CCO[Si](C)(C)C(C)(C)C]

To a solution of ethyl 7-bromo-4-(((2S,4S)-1-(tert-butoxycarbonyl)-2-(2-((tert-butyldimethylsilyl)oxy)ethyl)piperidin-4-yl)amino)-2,6-dichloro-8-fluoroquinoline-3-carboxylate (14.6 g, 20.18 mmol) in toluene (200 ml) at −78° C. was added 1.0 M DIBAL-H in DCM (60.5 ml, 60.5 mmol). The resulting mixture was stirred at −78° C. for 40 min and warm to 0° C. for 1 h and 20 min, quenched with methanol (6.8 ml, 167 mmol). Aqueous Rochelle salt solution (prepared from 88 g (6 wt) of Rochelle salt and 200 mL of water) was added to the solution at ≤10° C. The biphasic mixture was stirred vigorously for 21 h at 15-25° C. and separated to give organic layer. The biphasic mixture was separated. The organic layer was washed with brine, dried over Na₂SO₄, filtered and concentrated. The crude was used as is. LC-MS calculated for C₂₈H₄₂BrCl₂FN₃O₄Si (M+H)⁺: m/z=680.1, 682.1; found 680.1, 682.1.

### Step 12. tert-butyl (2S,4S)-4-((7-bromo-2,6-dichloro-8-fluoro-3-formylquinolin-4-yl)amino)-2-(2-((tert-butyldimethylsilyl)oxy)ethyl)piperidine-1-carboxylate

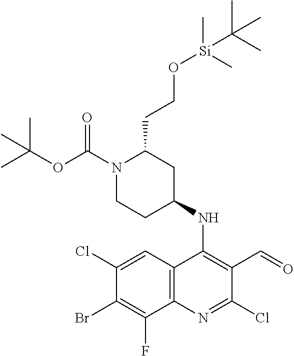 [SMILES: CC(C)(C)OC(=O)N1CC[C@H](Nc2c(C=O)c(Cl)nc3c(F)c(Br)c(Cl)cc23)C[C@H]1CCO[Si](C)(C)C(C)(C)C]

To a solution of tert-butyl (2S,4S)-4-((7-bromo-2,6-dichloro-8-fluoro-3-(hydroxymethyl)quinolin-4-yl)amino)-2-(2-((tert-butyldimethylsilyl)oxy)ethyl)piperidine-1-carboxylate (13.0 g, 19.07 mmol) in DCM (150 ml) and acetonitrile (50 ml) was added IBX (16.02 g, 57.2 mmol) and acetic acid (3.28 ml, 57.2 mmol). The resulting reaction mixture was stirred at 35° C. for 16 h. The reaction mixture was filtered and the filtrate was concentrated. The resulting residue was triturated with EtOAc, the resulting precipitate was collected via filtration, dried under vacuum to give the desired product as light yellow solid (9.4 g, 73% over 2 steps). LC-MS calculated for C₂₈H₄₀BrCl₂FN₃O₄Si (M+H)⁺: m/z=678.1, 680.1; found 678.1, 680.1.

### Step 13. tert-butyl (2S,4S)-4-((7-bromo-2,6-dichloro-8-fluoro-3-((E)-(hydroxyimino)methyl)quinolin-4-yl)amino)-2-(2-((tert-butyldimethylsilyl)oxy)ethyl)piperidine-1-carboxylate

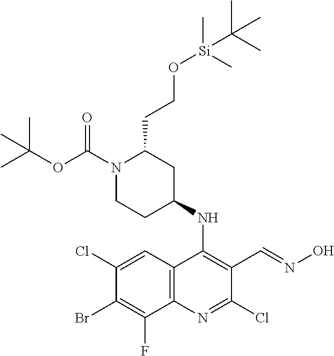 [SMILES: CC(C)(C)OC(=O)N1CC[C@H](Nc2c(/C=N/O)c(Cl)nc3c(F)c(Br)c(Cl)cc23)C[C@H]1CCO[Si](C)(C)C(C)(C)C]

To a mixture of tert-butyl (2S,4S)-4-((7-bromo-2,6-dichloro-8-fluoro-3-formylquinolin-4-yl)amino)-2-(2-((tert-butyldimethylsilyl)oxy)ethyl)piperidine-1-carboxylate (7.67 g, 11.29 mmol), DCM (56 ml) and EtOH (56 ml) was added hydroxylamine hydrochloride (2.35 g, 33.9 mmol) and pyridine (2.8 ml, 34.4 mmol). The reaction mixture was stirred at 40° C. for 16 hours. Another portion of pyridine (2.8 ml, 34.4 mmol) and hydroxylamine hydrochloride (2.35 g, 33.9 mmol) and stirred for 4 h. The solvent was evaporated in vacuo. The residue with DCM and water. The aqueous layer was extracted with DCM. The combined organic layers were washed with aqueous CuSO₄, brine, dried over MgSO₄, filtered and concentrated in vacuo. The residue was purified with column chromatography on silica gel to give the desired product (4.5 g, 57%). LC-MS calculated for C₂₈H₄₁BrCl₂FN₄O₄Si (M+H)⁺: m/z=693.1, 695.1; found 693.1, 695.1.

### Step 14. tert-butyl (2S,4S)-4-(7-bromo-4,8-dichloro-6-fluoro-1H-pyrazolo[4,3-c]quinolin-1-yl)-2-(2-((tert-butyldimethylsilyl)oxy)ethyl)piperidine-1-carboxylate

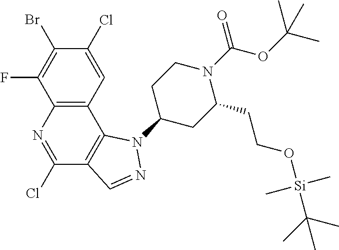 [SMILES: CC(C)(C)OC(=O)N1CC[C@H](n2ncc3c(Cl)nc4c(F)c(Br)c(Cl)cc4c32)C[C@H]1CCO[Si](C)(C)C(C)(C)C]

To a solution of (tert-butyl (2S,4S)-4-((7-bromo-2,6-dichloro-8-fluoro-3-((E)-(hydroxyimino)methyl)quinolin-4-yl)amino)-2-(2-((tert-butyldimethylsilyl)oxy)ethyl)piperidine-1-carboxylate (4.53 g, 6.52 mmol) in CH₂Cl₂ (75 mL) was added 2-aminopyridine (0.798 g, 8.48 mmol)) and Ms—Cl (0.610 ml, 7.83 mmol) at 0° C. The resulting mixtue was stirred at 0° C. for 2 hours. The reaction mixture was allowed to warm to room temperature overnight. The reaction was diluted with water. The organic layer was washed with brine, dried over MgSO₄, filtered and concentrated. The crude product was purified by column chromatography on silica gel (eluting with a gradient of 0-40% ethyl acetate in hexanes) to give the desired product (1.80 g, 41%). LC-MS calculated for C₂₈H₃₉BrCl₂FN₄O₃Si (M+H)⁺: m/z=675.1, 677.1; found 675.1, 677.1.

### Step 15. tert-butyl (2S,4S)-4-(7-bromo-8-chloro-6-fluoro-4-(methylthio)-1H-pyrazolo[4,3-c]quinolin-1-yl)-2-(2-((tert-butyldimethylsilyl)oxy)ethyl)piperidine-1-carboxylate

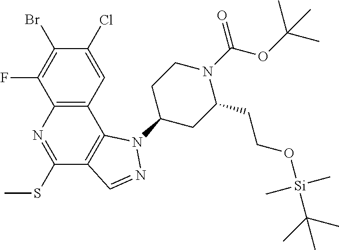 [SMILES: CSc1nc2c(F)c(Br)c(Cl)cc2c2c1cnn2[C@H]1CCN(C(=O)OC(C)(C)C)[C@H](CCO[Si](C)(C)C(C)(C)C)C1]

Sodium thiomethoxide (0.56 g, 8.00 mmol) was added to a mixture of tert-butyl (2S,4S)-4-(7-bromo-4,8-dichloro-6-fluoro-1H-pyrazolo[4,3-c]quinolin-1-yl)-2-(2-((tert-butyldimethylsilyl)-oxy)ethyl)piperidine-1-carboxylate (1.80 g, 2.67 mmol) in MeOH (26 ml)/DCM (26 ml) and then stirred at rt for 1 h. The mixture was diluted with sat'd NH₄Cl and extracted with EtOAc. The combined organic layers were dried over MgSO₄, filtered, concentrated. The crude product was purified by column chromatography on silica gel to give the desired product (1.75 g, 95%). LC-MS calculated for C₂₉H₄₂BrClFN₄O₃SSi (M+H)⁺: m/z=687.2, 689.2; found 687.2, 689.2.

### Step 16. tert-butyl (2S,4S)-4-(7-bromo-8-chloro-6-fluoro-4-(methylthio)-1H-pyrazolo[4,3-c]quinolin-1-yl)-2-(2-hydroxyethyl)piperidine-1-carboxylate

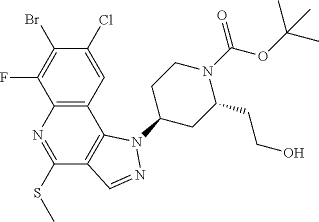 [SMILES: CSc1nc2c(F)c(Br)c(Cl)cc2c2c1cnn2[C@H]1CCN(C(=O)OC(C)(C)C)[C@H](CCO)C1]

To a solution of tert-butyl (2S,4S)-4-(7-bromo-8-chloro-6-fluoro-4-(methylthio)-1H-pyrazolo[4,3-c]quinolin-1-yl)-2-(2-((tert-butyldimethylsilyl)oxy)ethyl)piperidine-1-carboxylate (1.96 g, 2.84 mmol) in THF (28 ml) was added 1.0 M TBAF in THF (4.27 ml, 4.27 mmol). The resulting mixture was stirred at 60° C. for 1 h. After cooling to rt, the reaction mixture was diluted with water and ethyl acetate. The organic layer was separated and washed with brine, dried over Na₂SO₄, filtered and concentrated. The crude was used as is. LC-MS calculated for C₂₃H₂₈BrClFN₄O₃S (M+H)⁺: m/z=573.1, 575.1; found 573.1, 575.1.

### Step 17. tert-butyl (2S,4S)-4-(7-bromo-8-chloro-6-fluoro-4-(methylthio)-1H-pyrazolo[4,3-c]quinolin-1-yl)-2-(cyanomethyl)piperidine-1-carboxylate

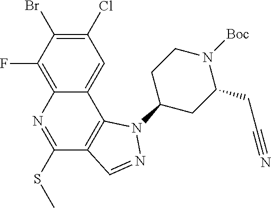 [SMILES: CSc1nc2c(F)c(Br)c(Cl)cc2c2c1cnn2[C@H]1CCN(C(=O)OC(C)(C)C)[C@H](CC#N)C1]

To a solution of tert-butyl (2S,4S)-4-(7-bromo-8-chloro-6-fluoro-4-(methylthio)-1H-pyrazolo[4,3-c]quinolin-1-yl)-2-(2-hydroxyethyl)piperidine-1-carboxylate (0.50 g, 0.871 mmol) in DCM (8 ml) was added dess-martinperiodinane (0.406 g, 0.958 mmol). The resulting mixture was stirred for 1 h, To the reaction flask was added saturated NaHCO₃ and stirred for 10 min. The organic layer was separated and dried over Na₂SO₄, filtered and concentrated. The crude was dissolved in THF (10 mL), ammonium hydroxide (1.96 ml, 14.11 mmol) was added to reaction flask, followed by iodine (0.243 g, 0.958 mmol). The resulting mixture was stirred at rt for 3 h, The reaction solution was diluted with ethyl acetate and sat'd NaS₂O₃ solution. The organic layer was separated and washed with brine, dried over Na₂SO₄, filtered and concentrated. The residue was purified with flash chromatography to give the desired product (0.40 g, 80%). LC-MS calculated for C₂₃H₂₅BrClFN₅O₂S (M+H)⁺: m/z=568.1, 570.1; found 568.1, 570.1.

### Step 18. tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate

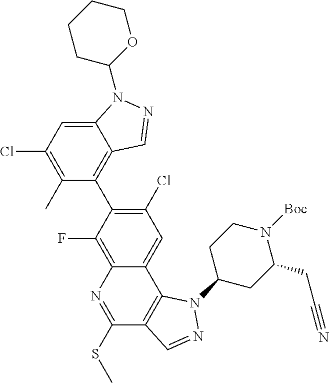 [SMILES: CSc1nc2c(F)c(-c3c(C)c(Cl)cc4c3cnn4C3CCCCO3)c(Cl)cc2c2c1cnn2[C@H]1CCN(C(=O)OC(C)(C)C)[C@H](CC#N)C1]

The vial charged with tert-butyl (2S,4S)-4-(7-bromo-8-chloro-6-fluoro-4-(methylthio)-1H-pyrazolo[4,3-c]quinolin-1-yl)-2-(cyanomethyl)piperidine-1-carboxylate (401 mg, 0.705 mmol), 6-chloro-5-methyl-1-(tetrahydro-2H-pyran-2-yl)-4-(4,4,5,5-tetramethyl-1,3,2-dioxaborolan-2-yl)-1H-indazole (319 mg, 0.846 mmol), tetrakis(triphenylphosphine)palladium(0) (122 mg, 0.106 mmol), sodium carbonate (299 mg, 2.82 mmol) and 5:1 dioxane/water (6 ml) were heated at 105° C. overnight. The mixture was diluted with brine and EtOAc, the organic layer was separated, dried over MgSO₄, filtered and concentrated. The crude product was purified by column chromatography to give the desired product (0.39 g, 75%). LC-MS calculated for C₃₆H₃₉Cl₂FN₇O₃S (M+H)⁺: m/z=738.2; found 738.2.

### Step 19. tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate

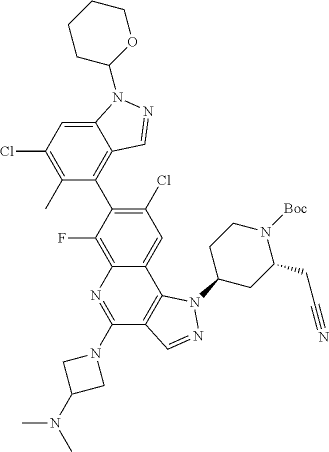 [SMILES: Cc1c(Cl)cc2c(cnn2C2CCCCO2)c1-c1c(Cl)cc2c(nc(N3CC(N(C)C)C3)c3cnn([C@H]4CCN(C(=O)OC(C)(C)C)[C@H](CC#N)C4)c32)c1F]

To a solution of tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate (0.73 g, 0.988 mmol) in DCM (10 ml) at 0° C. was added m-CPBA (0.196 g, 1.136 mmol). The reaction mixture was stirred at this temperature for 20 min. The reaction was quenched by adding sat'd Na₂S₂O₃, diluted with ethyl acetate and washed with saturated NaHCO₃, brine, filtered, dried and concentrated. The crude was dissolve in acetonitrile (8 ml) and triethylamine (0.561 ml, 4.03 mmol) and N,N-dimethylazetidin-3-amine dihydrochloride (0.261 g, 1.511 mmol) was added to reaction vial and the resulting mixture was stirred at 70° C. for 2 h. The crude was concentrated and the residue was purified by silica gel column (eluting with a gradient of 0-20% DCM in MeOH) to give the desired product (0.61 g, 77%). LC-MS calculated for C₄₀H₄₇Cl₂FN₉O₃ (M+H)⁺: m/z=790.3; found 790.3.

### Step 20. 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile

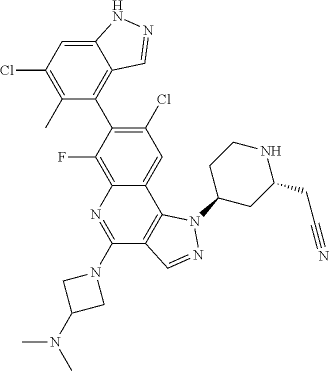 [SMILES: Cc1c(Cl)cc2[nH]ncc2c1-c1c(Cl)cc2c(nc(N3CC(N(C)C)C3)c3cnn([C@H]4CCN[C@H](CC#N)C4)c32)c1F]

To a solution of tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate (0.61 g, 0.771 mmol) in DCM (5 ml) was added TFA (4.8 ml, 61.7 mmol). After stirring for 0.5 h, the solvent was removed in vacuo, the residue was purified with prep-LCMS (XBridge C18 column, eluting with a gradient of acetonitrile/water containing 0.1% TFA, at flow rate of 60 mL/min) to give the desired product as two peaks (0.40 g, 85%).

Diastereomer 1. Peak 1. LC-MS calculated for C₃₀H₃₁Cl₂FN₉ (M+H)⁺: m/z=606.2; found 606.2.

Diastereomer 2. Peak 2. LC-MS calculated for C₃₀H₃₁Cl₂FN₉ (M+H)⁺: m/z=606.2; found 606.2.

### Step 21. 2-((2S,4S)-1-acryloyl-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile

To a solution of 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile bis(2,2,2-trifluoroacetate) (131 mg, 0.157 mmol)) in DCM (1.570 ml) was added 1.0 M acryloyl chloride in DCM (165 μl, 0.165 mmol) and DIEA (110 μl, 0.628 mmol). The resluting mixture was stirred at 0° C. for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile bis(2,2,2-trifluoroacetate) (peak1 from last step).

Example 3a. Diastereomer 1. Peak 1. LCMS calculated for C₃₃H₃₃Cl₂FN₉O (M+H)⁺ m/z=660.2; found 660.2. ¹H NMR (600 MHz, DMSO-d₆) δ 13.30 (s, 1H), 10.39 (s, 1H), 8.37 (s, 2H), 7.84 (s, 1H), 7.53 (s, 1H), 6.94 (m, 1H), 6.20 (m, 1H), 5.79 (m, 1H), 5.67 (m, 1H), 5.27 (m, 0.5H), 4.93 (s, 0.5H), 4.68 (m, 5H), 4.32 (m, 1H), 4.26-3.70 (m, 2H), 3.46 (m, 1H), 3.26-3.20 (m, 1H), 2.88 (s, 6H), 2.29 (s, 1H), 2.25 (m, 2H), 2.19 (s, 3H).

Example 3b. Diastereomer 2. Peak 2. LCMS calculated for C₃₃H₃₃Cl₂FN₉O (M+H)⁺ m/z=660.2; found 660.2.

### Example 4a and Example 4b. 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile

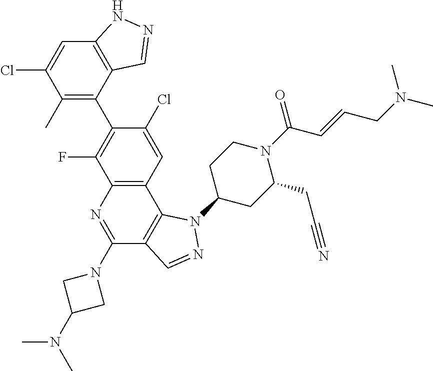 [SMILES: Cc1c(Cl)cc2[nH]ncc2c1-c1c(Cl)cc2c(nc(N3CC(N(C)C)C3)c3cnn([C@H]4CCN(C(=O)/C=C/CN(C)C)[C@H](CC#N)C4)c32)c1F]

### Step 1. 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile

To a solution of (E)-4-(dimethylamino)but-2-enoic acid hydrochloride (2.084 mg, 0.013 mmol) and 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile bis(2,2,2-trifluoroacetate) (peak2 from last step) (7 mg, 8.39 μmol) in DMF (1.0 ml) was added HATU (5.10 mg, 0.013 mmol) and DIEA (5.86 μl, 0.034 mmol). The resulting mixture was stirred at rt for 2 h. The reaction mixture was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile bis(2,2,2-trifluoroacetate) (peak1 from last step).

Example 4a. Diastereomer 1. Peak 1. LCMS calculated for C₃₆H₄₀Cl₂FN₁₀O (M+H)⁺ m/z=717.3; found 717.3.

Example 4b. Diastereomer 2. Peak 2. LCMS calculated for C₃₆H₄₀Cl₂FN₁₀O (M+H)⁺ m/z=717.3; found 717.3.

### Example 5a and Example 5b. 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-methoxybut-2-enoyl)piperidin-2-yl)acetonitrile

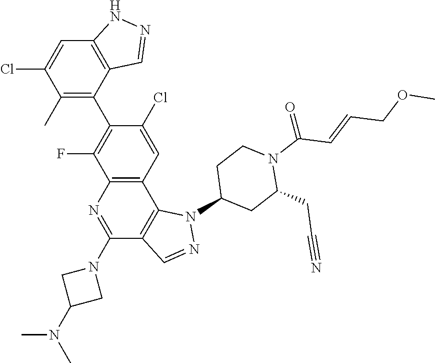 [SMILES: COC/C=C/C(=O)N1CC[C@H](n2ncc3c(N4CC(N(C)C)C4)nc4c(F)c(-c5c(C)c(Cl)cc6[nH]ncc56)c(Cl)cc4c32)C[C@H]1CC#N]

This compound was prepared according to the procedure described in Example 4a and Example 4b, step 1, replacing (E)-4-(dimethylamino)but-2-enoic acid hydrochloride with (E)-4-methoxybut-2-enoic acid.

Example 5a. Diastereomer 1. Peak 1. LCMS calculated for C₃₅H₃₇Cl₂FN₉O₂ (M+H)⁺ m/z=704.2; found 704.2. ¹H-NMR (500 MHz in DMSO-d₆) δ 8.38 (s, 2H), 7.85 (s, 1H), 7.54 (s, 1H), 6.75 (s, 2H), 5.68 (s, 0.5H), 5.27 (s, 0.5H), 4.68-4.52 (m, 4H), 4.33 (s, 1H), 4.11 (s, 2H), 3.76-3.56 (m, 3H), 3.50-3.37 (m, 1H), 3.23 (s, 3H), 3.22-3.12 (m, 1H), 2.88 (s, 6H), 2.27-2.10 (m, 4H), 2.19 (s, 3H).

Example 5b. Diastereomer 2. Peak 2. LCMS calculated for C₃₅H₃₇Cl₂FN₉O₂ (M+H)⁺ m/z=704.2; found 704.2.

### Example 6a and Example 6b. 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile

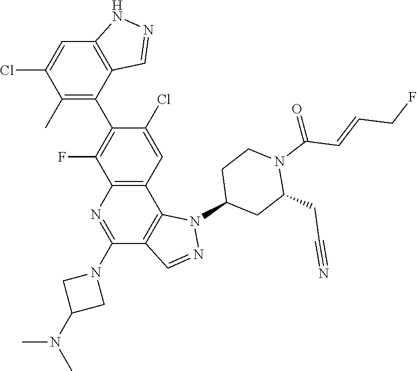 [SMILES: Cc1c(Cl)cc2[nH]ncc2c1-c1c(Cl)cc2c(nc(N3CC(N(C)C)C3)c3cnn([C@H]4CCN(C(=O)/C=C/CF)[C@H](CC#N)C4)c32)c1F]

This compound was prepared according to the procedure described in Example 4a and Example 4b, step 1, replacing (E)-4-(dimethylamino)but-2-enoic acid hydrochloride with (E)-4-fluorobut-2-enoic acid.

Example 6a. Diastereomer 1. Peak 1. LCMS calculated for C₃₄H₃₄Cl₂F₂NsO (M+H)⁺ m/z=692.2; found 692.2.

Example 6b. Diastereomer 2. Peak 2. LCMS calculated for C₃₄H₃₄Cl₂F₂NsO (M+H)⁺ m/z=692.2; found 692.2.

### Example 7a and Example 7b. 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4,4-difluorobut-2-enoyl)piperidin-2-yl)acetonitrile

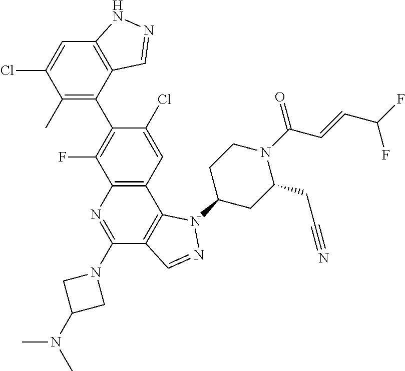 [SMILES: Cc1c(Cl)cc2[nH]ncc2c1-c1c(Cl)cc2c(nc(N3CC(N(C)C)C3)c3cnn([C@H]4CCN(C(=O)/C=C/C(F)F)[C@H](CC#N)C4)c32)c1F]

This compound was prepared according to the procedure described in Example 4a and Example 4b, step 1, replacing (E)-4-(dimethylamino)but-2-enoic acid hydrochloride with (E)-4,4-difluorobut-2-enoic acid.

Example 7a. Diastereomer 1. Peak 1. LCMS calculated for C₃₄H₃₃Cl₂F₃N₉O (M+H)⁺ m/z=710.2; found 710.2.

Example 7b. Diastereomer 2. Peak 2. LCMS calculated for C₃₄H₃₃Cl₂F₃N₉O (M+H)⁺ m/z=710.2; found 710.2.

### Example 8a and Example 8b. 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-(2-fluoroacryloyl)piperidin-2-yl)acetonitrile

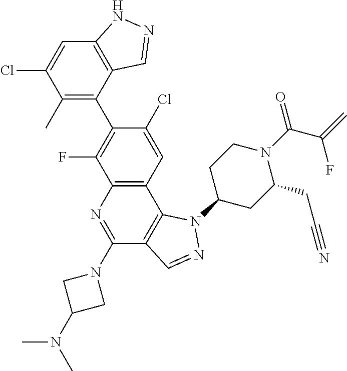 [SMILES: C=C(F)C(=O)N1CC[C@H](n2ncc3c(N4CC(N(C)C)C4)nc4c(F)c(-c5c(C)c(Cl)cc6[nH]ncc56)c(Cl)cc4c32)C[C@H]1CC#N]

This compound was prepared according to the procedure described in Example 4a and Example 4b, step 1, replacing (E)-4-(dimethylamino)but-2-enoic acid hydrochloride with 2-fluoroacrylic acid.

Example 8a. Diastereomer 1. Peak 1. LCMS calculated for C₃₃H₃₂Cl₂F₂N₉O (M+H)⁺ m/z=678.2; found 678.2. ¹H NMR (500 MHz, DMSO-d₆) δ 10.33 (s, 1H), 8.36 (m, 2H), 7.82 (s, 1H), 7.51 (s, 1H), 5.71 (m, 1H), 5.35 (d, J=3.7 Hz, 1H), 5.30 (m, 1H), 5.13 (m, 1H), 4.68 (d, J=10.4 Hz, 2H), 4.59 (m, 2H), 4.34 (s, 1H), 4.20-3.54 (m, 3H), 3.27 (m, 1H), 2.89 (s, 6H), 2.37-2.30 (m, 4H), 2.21 (s, 3H).

Example 8b. Diastereomer 2. Peak 2. LCMS calculated for C₃₃H₃₂Cl₂F₂NsO (M+H)⁺ m/z=678.2; found 678.2.

### Example 9a and Example 9b. 2-((2S,4S)-1-(but-2-ynoyl)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile

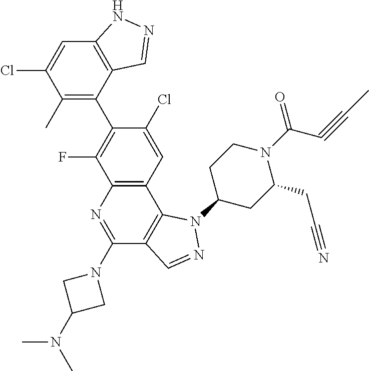 [SMILES: CC#CC(=O)N1CC[C@H](n2ncc3c(N4CC(N(C)C)C4)nc4c(F)c(-c5c(C)c(Cl)cc6[nH]ncc56)c(Cl)cc4c32)C[C@H]1CC#N]

This compound was prepared according to the procedure described in Example 4a and Example 4b, step 1, replacing (E)-4-(dimethylamino)but-2-enoic acid hydrochloride with but-2-ynoic acid.

Example 9a. Diastereomer 1. Peak 1. LCMS calculated for C₃₄H₃₃Cl₂FN₉O (M+H)⁺ m/z=672.2; found 672.2. ¹H-NMR (500 MHz in DMSO-d₆) δ 10.47 (s, 1H), 8.38, (s, 1H), 8.36 (d, J=13.0 Hz, 1H), 7.84 (s, 1H), 7.53 (d, J=5.4 Hz, 1H), 5.68 (m, 1H), 5.13 (m, 1H), 4.67-4.33 (m, 6H), 3.74-3.22 (m, 4H), 2.88 (s, 6H), 2.32-2.06 (m, 10H).

Example 9b. Diastereomer 2. Peak 2. LCMS calculated for C₃₄H₃₃Cl₂FN₉O (M+H)⁺ m/z=672.2; found 672.2.

### Example 10a and Example 10b. 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile

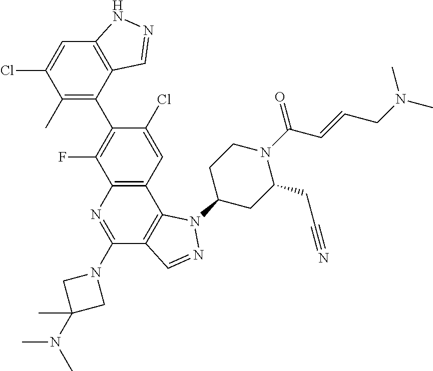 [SMILES: Cc1c(Cl)cc2[nH]ncc2c1-c1c(Cl)cc2c(nc(N3CC(C)(N(C)C)C3)c3cnn([C@H]4CCN(C(=O)/C=C/CN(C)C)[C@H](CC#N)C4)c32)c1F]

### Step 1. tert-butyl (2S,4S)-4-(8-chloro-7-(6-chloro-5-methyl-1-(tetrahydro-2H-pyran-2-yl)-1H-indazol-4-yl)-4-(3-(dimethylamino)-3-methylazetidin-1-yl)-6-fluoro-1H-pyrazolo[4,3-c]quinolin-1-yl)-2-(cyanomethyl)piperidine-1-carboxylate

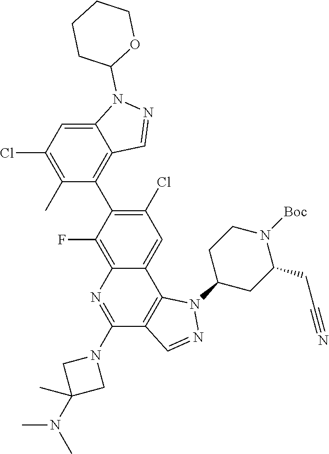 [SMILES: Cc1c(Cl)cc2c(cnn2C2CCCCO2)c1-c1c(Cl)cc2c(nc(N3CC(C)(N(C)C)C3)c3cnn([C@H]4CCN(C(=O)OC(C)(C)C)[C@H](CC#N)C4)c32)c1F]

This compound was prepared according to the procedure described in Example 3a and Example 3b, in Step 19 replacing N,N-dimethylazetidin-3-amine dihydrochloride with N,N,3-trimethylazetidin-3-amine hydrochloride. LCMS calculated for C₄₁H₄₉Cl₂FN₉O₃ (M+H)⁺ m/z=804.3; found 804.3.

### Step 2. 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile

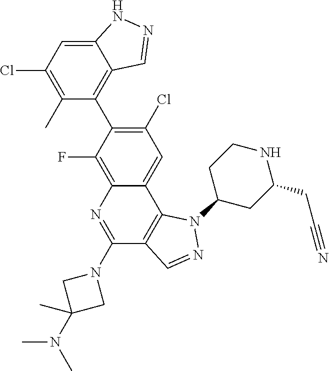 [SMILES: Cc1c(Cl)cc2[nH]ncc2c1-c1c(Cl)cc2c(nc(N3CC(C)(N(C)C)C3)c3cnn([C@H]4CCN[C@H](CC#N)C4)c32)c1F]

This compound was prepared according to the procedure described in Example 3a and Example 3b, in Step 20 replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-4-(8-chloro-7-(6-chloro-5-methyl-1-(tetrahydro-2H-pyran-2-yl)-1H-indazol-4-yl)-4-(3-(dimethylamino)-3-methylazetidin-1-yl)-6-fluoro-1H-pyrazolo[4,3-c]quinolin-1-yl)-2-(cyanomethyl)piperidine-1-carboxylate. LCMS calculated for C₃₁H₃₃Cl₂FN₉ (M+H)⁺ m/z=620.2; found 620.0.

### Step 3. 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile

To a solution of (E)-4-(dimethylamino)but-2-enoic acid hydrochloride (2.084 mg, 0.013 mmol) and 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile bis(2,2,2-trifluoroacetate) (7 mg, 8.25 μmol) (peak 2 from last step) in DMF (1.0 ml) was added HATU (5.1 mg, 0.013 mmol) and DIEA (5.9 μl, 0.034 mmol). The resulting mixture was stirred at rt for 2 h. The reaction mixture was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile bis(2,2,2-trifluoroacetate) (peak1 from last step).

Example 10a. Diastereomer 1. Peak 1. LCMS calculated for C₃₇H₄₂Cl₂FN₁₀O (M+H)⁺ m/z=731.3; found 731.3.

Example 10b. Diastereomer 2. Peak 2. LCMS calculated for C₃₇H₄₂Cl₂FN₁₀O (M+H)⁺ m/z=731.3; found 731.3.

### Example 11a and Example 11b. 2-((2S,4S)-1-(but-2-ynoyl)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile

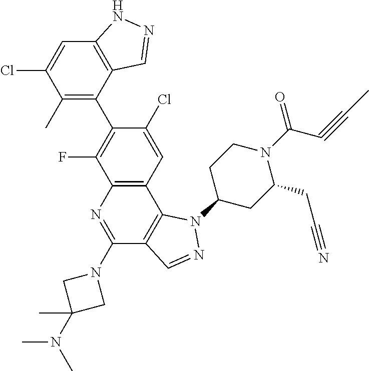 [SMILES: CC#CC(=O)N1CC[C@H](n2ncc3c(N4CC(C)(N(C)C)C4)nc4c(F)c(-c5c(C)c(Cl)cc6[nH]ncc56)c(Cl)cc4c32)C[C@H]1CC#N]

This compound was prepared according to the procedure described in Example 10a and Example 10b, step 3, replacing (E)-4-(dimethylamino)but-2-enoic acid hydrochloride with but-2-ynoic acid.

Example 11a. Diastereomer 1. Peak 1. LCMS calculated for C₃₅H₃₅Cl₂FN₉O (M+H)⁺ m/z=686.2; found 686.2.

Example 11b. Diastereomer 2. Peak 2. LCMS calculated for C₃₅H₃₅Cl₂FN₉O (M+H)⁺ m/z=686.2; found 686.2.

### Example 12a and Example 12b. 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-methoxybut-2-enoyl)piperidin-2-yl)acetonitrile

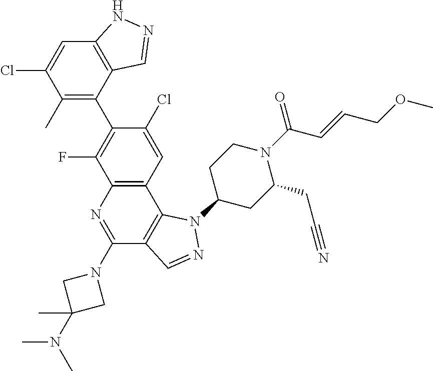 [SMILES: COC/C=C/C(=O)N1CC[C@H](n2ncc3c(N4CC(C)(N(C)C)C4)nc4c(F)c(-c5c(C)c(Cl)cc6[nH]ncc56)c(Cl)cc4c32)C[C@H]1CC#N]

This compound was prepared according to the procedure described in Example 10a and Example 10b, step 3, replacing (E)-4-(dimethylamino)but-2-enoic acid hydrochloride with (E)-4-methoxybut-2-enoic acid.

Example 12a. Diastereomer 1. Peak 1. LCMS calculated for C₃₆H₃₉Cl₂FN₉O₂ (M+H)⁺ m/z=718.2; found 718.2. ¹H NMR (600 MHz, DMSO-d₆) δ 8.36 (m, 2H), 7.84 (s, 1H), 7.53 (s, 1H), 6.81-6.69 (m, 2H), 5.68 (s, 1H), 5.27 (s, 0.5H), 4.89 (s, 0.5H), 4.68-4.20 (m, 5H), 4.10 (d, J=2.7 Hz, 2H), 3.71-3.44 (m, 1H), 3.33 (s, 3H), 3.29-3.18 (m, 2H), 2.82 (s, 6H), 2.27 (m, 3H), 2.19 (s, 3H), 2.18-2.13 (m, 1H), 1.68 (s, 3H).

Example 12b. Diastereomer 2. Peak 2. LCMS calculated for C₃₆H₃₉Cl₂FN₉O₂ (M+H)⁺ m/z=718.2; found 718.2.

### Example 13a and Example 13b. 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-fluorobut-2-enoyl)piperidin-2-yl)acetonitrile

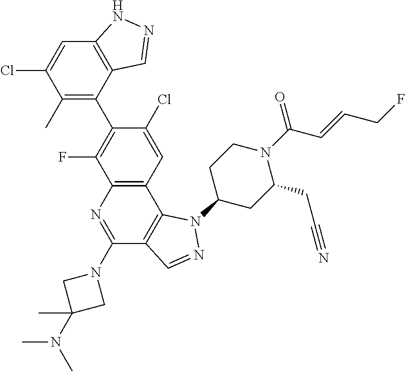 [SMILES: Cc1c(Cl)cc2[nH]ncc2c1-c1c(Cl)cc2c(nc(N3CC(C)(N(C)C)C3)c3cnn([C@H]4CCN(C(=O)/C=C/CF)[C@H](CC#N)C4)c32)c1F]

This compound was prepared according to the procedure described in Example 10a and Example 10b, step 3, replacing (E)-4-(dimethylamino)but-2-enoic acid hydrochloride with (E)-4-fluorobut-2-enoic acid.

Example 13a. Diastereomer 1. Peak 1. LCMS calculated for C₃₅H₃₆Cl₂F₂N₉O (M+H)⁺ m/z=706.2; found 706.2.

Example 13b. Diastereomer 2. Peak 2. LCMS calculated for C₃₅H₃₆Cl₂F₂N₉O (M+H)⁺ m/z=706.2; found 706.2.

### Example 14a and Example 14b. 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4,4-difluorobut-2-enoyl)piperidin-2-yl)acetonitrile

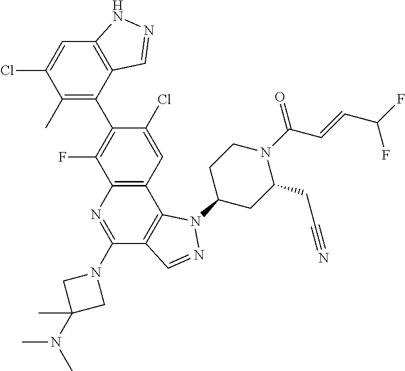 [SMILES: Cc1c(Cl)cc2[nH]ncc2c1-c1c(Cl)cc2c(nc(N3CC(C)(N(C)C)C3)c3cnn([C@H]4CCN(C(=O)/C=C/C(F)F)[C@H](CC#N)C4)c32)c1F]

This compound was prepared according to the procedure described in Example 10a and Example 10b, step 3, replacing (E)-4-(dimethylamino)but-2-enoic acid hydrochloride with (E)-4,4-difluorobut-2-enoic acid.

Example 14a. Diastereomer 1. Peak 1. LCMS calculated for C₃₅H₃₅Cl₂F₃N₉O (M+H)⁺ m/z=724.2; found 724.2.

Example 14b. Diastereomer 2. Peak 2. LCMS calculated for C₃₅H₃₅Cl₂F₃N₉O (M+H)⁺ m/z=724.2; found 724.2.

### Example 15a and Example 15b. 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-(2-fluoroacryloyl)piperidin-2-yl)acetonitrile

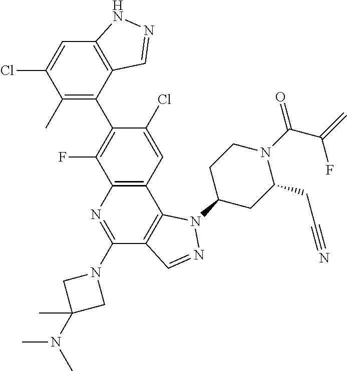 [SMILES: C=C(F)C(=O)N1CC[C@H](n2ncc3c(N4CC(C)(N(C)C)C4)nc4c(F)c(-c5c(C)c(Cl)cc6[nH]ncc56)c(Cl)cc4c32)C[C@H]1CC#N]

This compound was prepare according to the procedure described in Example 10a and Example 10b, step 3, replacing (E)-4-(dimethylamino)but-2-enoic acid hydrochloride with 2-fluoroacrylic acid.

Example 15a. Diastereomer 1. Peak 1. LCMS calculated for C₃₄H₃₄Cl₂F₂N₉O (M+H)⁺ m/z=692.2; found 692.2.

Example 15b. Diastereomer 2. Peak 2. LCMS calculated for C₃₄H₃₄Cl₂F₂N₉O (M+H)⁺ m/z=692.2; found 692.2.

### Example 16a and Example 16b. 2-((2S,4S)-1-acryloyl-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile

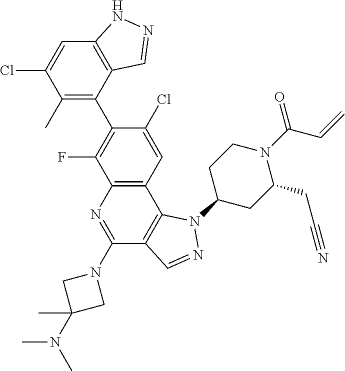 [SMILES: C=CC(=O)N1CC[C@H](n2ncc3c(N4CC(C)(N(C)C)C4)nc4c(F)c(-c5c(C)c(Cl)cc6[nH]ncc56)c(Cl)cc4c32)C[C@H]1CC#N]

This compound was prepared according to the procedure described in Example 2, step 6, replacing 1-(7-bromo-8-chloro-6-fluoro-1-(piperidin-4-yl)-1H-pyrazolo[4,3-c]quinolin-4-yl)-N,N-dimethylazetidin-3-amine with 2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile.

Example 16a. Diastereomer 1. Peak 1. LCMS calculated for C₃₄H₃₅Cl₂FN₉O (M+H)⁺ m/z=674.2; found 674.2.

Example 16b. Diastereomer 2. Peak 2. LCMS calculated for C₃₄H₃₅Cl₂FN₉O (M+H)⁺ m/z=674.2; found 674.2.

### Example 17a and Example 17b. 2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)-1-((E)-4-(dimethylamino but-2-enoyl)piperidin-2-yl)acetonitrile

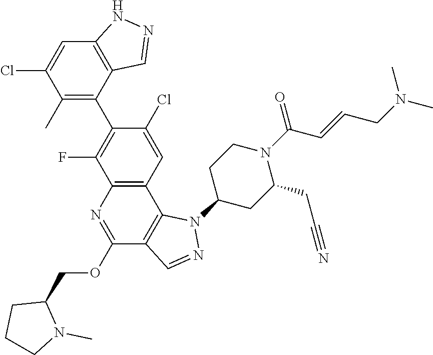 [SMILES: Cc1c(Cl)cc2[nH]ncc2c1-c1c(Cl)cc2c(nc(OC[C@@H]3CCCN3C)c3cnn([C@H]4CCN(C(=O)/C=C/CN(C)C)[C@H](CC#N)C4)c32)c1F]

### Step 1. tert-butyl (2S,4S)-4-(8-chloro-7-(6-chloro-5-methyl-1-(tetrahydro-2H-pyran-2-yl)-1H-indazol-4-yl)-6-fluoro-4-(((S)-1-methylpyrrolidin-2-yl)methoxy)-1H-pyrazolo[4,3-c]quinolin-1-yl)-2-(cyanomethyl)piperidine-1-carboxylate

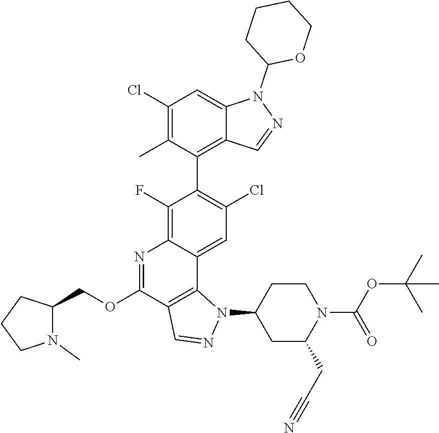 [SMILES: Cc1c(Cl)cc2c(cnn2C2CCCCO2)c1-c1c(Cl)cc2c(nc(OC[C@@H]3CCCN3C)c3cnn([C@H]4CCN(C(=O)OC(C)(C)C)[C@H](CC#N)C4)c32)c1F]

To a solution of tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate (189 mg, 0.256 mmol) in DCM (2.5 ml) was added m-CPBA (50.8 mg, 0.294 mmol) at 0° C. and then the reaction was stirred at this temperature for 20 min. The reaction was quenched by adding sat'd Na₂S₂O₃, diluted with ethyl acetate and washed with sat'd NaHCO₃, brine, filtered, dried and concentrated. The crude was dissolved in THF (2 mL), (S)-(1-methylpyrrolidin-2-yl)methanol (58.6 mg, 0.509 mmol) was added to reaction vial, followed by sodium tert-butoxide (98 mg, 1.018 mmol), and then the reaction was stirred at rt for 1 h. The solvent was removed in vacuo. The crude was used in next step without further purification. LCMS calculated for C₄₁H₄₈Cl₂FN₈O₄ (M+H)⁺ m/z=805.3; found 805.3.

### Step 2. 2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile

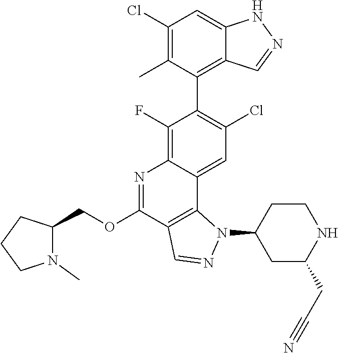 [SMILES: Cc1c(Cl)cc2[nH]ncc2c1-c1c(Cl)cc2c(nc(OC[C@@H]3CCCN3C)c3cnn([C@H]4CCN[C@H](CC#N)C4)c32)c1F]

This compound was prepared according to the procedure described in Example 3a and Example 3b, replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-4-(8-chloro-7-(6-chloro-5-methyl-1-(tetrahydro-2H-pyran-2-yl)-1H-indazol-4-yl)-6-fluoro-4-(((S)-1-methylpyrrolidin-2-yl)methoxy)-1H-pyrazolo[4,3-c]quinolin-1-yl)-2-(cyanomethyl)piperidine-1-carboxylate in Step 20. LCMS calculated for C₃₁H₃₂Cl₂FN₈O (M+H)⁺ m/z=621.2; found 621.0.

### Step 3. 2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)-1-((E)-4-(dimethylamino)but-2-enoyl)piperidin-2-yl)acetonitrile

To a solution of (E)-4-(dimethylamino)but-2-enoic acid hydrochloride (2.1 mg, 0.013 mmol) and 2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile bis(2,2,2-trifluoroacetate) (6.5 mg, 7.65 μmol) (peak 2 from last step) in DMF (1.0 ml) was added HATU (5.1 mg, 0.013 mmol) and DIEA (5.9 μl, 0.034 mmol). The resulting mixture was stirred at rt for 2 h. The reaction mixture was diluted with methanol and 1 N HCl (0.1 mL) and purified using prep-LCMS (XBridge C18 column, eluting with a gradient of acetonitrile/water containing 0.1% TFA, at flow rate of 60 mL/min) then purified again using prep-LCMS (XBridge C18 column, eluting with a gradient of acetonitrile/water containing 0.15% NH₄OH, at flow rate of 60 mL/min) to afford the desired diastereomer 1.

Diastereomer 2 was synthesized in similar way using 2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile bis(2,2,2-trifluoroacetate) (peak1 from last step).

Example 17a. Diastereomer 1. Peak 1. LCMS calculated for C₃₇H₄₁Cl₂FN₉O₂ (M+H)⁺ m/z=732.3; found 732.2.

Example 17b. Diastereomer 2. Peak 2. LCMS calculated for C₃₇H₄₁Cl₂FN₉O₂ (M+H)⁺ m/z=732.3; found 732.2.

### Example 18a and Example 18b. 2-((2S,4S)-1-(but-2-ynoyl)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile

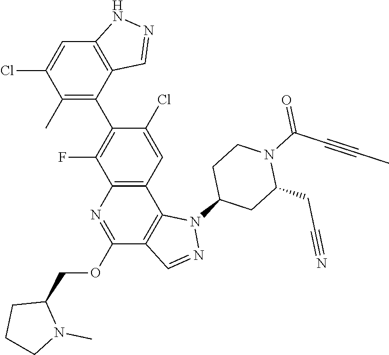 [SMILES: CC#CC(=O)N1CC[C@H](n2ncc3c(OC[C@@H]4CCCN4C)nc4c(F)c(-c5c(C)c(Cl)cc6[nH]ncc56)c(Cl)cc4c32)C[C@H]1CC#N]

This compound was prepared according to the procedure described in Example 17a and Example 17b, step 3, replacing (E)-4-(dimethylamino)but-2-enoic acid hydrochloride with but-2-ynoic acid.

Example 18a. Diastereomer 1. Peak 1. LCMS calculated for C₃₅H₃₄Cl₂FN₈O (M+H)⁺ m/z=687.2; found 687.2.

Example 18b. Diastereomer 2. Peak 2. LCMS calculated for C₃₅H₃₄Cl₂FN₈O (M+H)⁺ m/z=687.2; found 687.2.

### Example 19a and Example 19b. 2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)-1-((E)-4-methoxybut-2-enoyl)piperidin-2-yl)acetonitrile

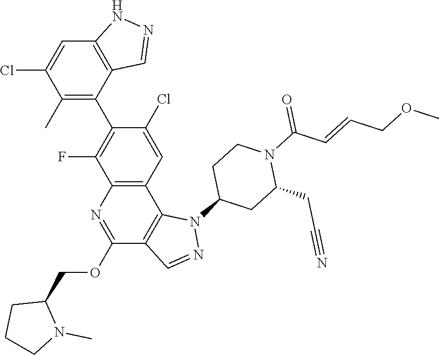 [SMILES: COC/C=C/C(=O)N1CC[C@H](n2ncc3c(OC[C@@H]4CCCN4C)nc4c(F)c(-c5c(C)c(Cl)cc6[nH]ncc56)c(Cl)cc4c32)C[C@H]1CC#N]

This compound was prepared according to the procedure described in Example 17a and Example 17b, step 3, replacing (E)-4-(dimethylamino)but-2-enoic acid hydrochloride with (E)-4-methoxybut-2-enoic acid.

Example 19a. Diastereomer 1. Peak 1. LCMS calculated for C₃₆H₃₈Cl₂FN₈O₃ (M+H)⁺ m/z=719.2; found 719.2.

Example 19b. Diastereomer 2. Peak 2. LCMS calculated for C₃₆H₃₈Cl₂FN₈O₃ (M+H)⁺ m/z=719.2; found 719.2.

### Example 20a and Example 20b. 2-((2S,4S)-1-acryloyl-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile

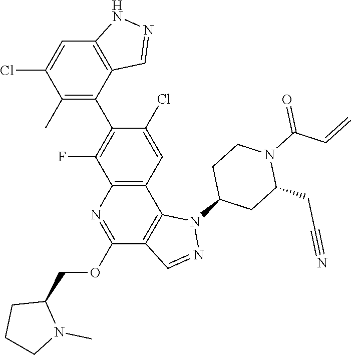 [SMILES: C=CC(=O)N1CC[C@H](n2ncc3c(OC[C@@H]4CCCN4C)nc4c(F)c(-c5c(C)c(Cl)cc6[nH]ncc56)c(Cl)cc4c32)C[C@H]1CC#N]

This compound was prepared according to the procedure described in Example 2, step 6, replacing 1-(7-bromo-8-chloro-6-fluoro-1-(piperidin-4-yl)-1H-pyrazolo[4,3-c]quinolin-4-yl)-N,N-dimethylazetidin-3-amine with 2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile.

Example 20a. Diastereomer 1. Peak 1. LCMS calculated for C₃₄H₃₄Cl₂FN₈O₂ (M+H)⁺ m/z=675.2; found 675.2.

Example 20b. Diastereomer 2. Peak 2. LCMS calculated for C₃₄H₃₄Cl₂FN₈O₂ (M+H)⁺ m/z=675.2; found 675.2.

### Example 21a and Example 21b. 2-((2S,4S)-1-acryloyl-4-(8-chloro-4-(3-(dimethylamino)azetidin-1-yl)-6-fluoro-7-(5-fluoroquinolin-8-yl)-1H-pyrazolo[4,3-c]quinolin-1-yl)piperidin-2-yl)acetonitrile

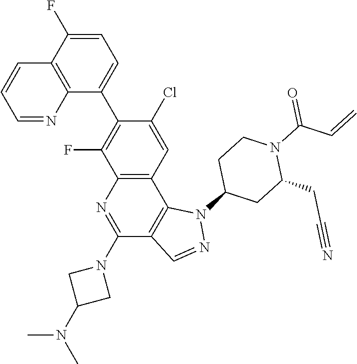 [SMILES: C=CC(=O)N1CC[C@H](n2ncc3c(N4CC(N(C)C)C4)nc4c(F)c(-c5ccc(F)c6cccnc56)c(Cl)cc4c32)C[C@H]1CC#N]

### Step 1. tert-butyl (2S,4S)-4-(7-bromo-8-chloro-4-(3-(dimethylamino)azetidin-1-yl)-6-fluoro-1H-pyrazolo[4,3-c]quinolin-1-yl)-2-(2-hydroxyethyl)piperidine-1-carboxylate

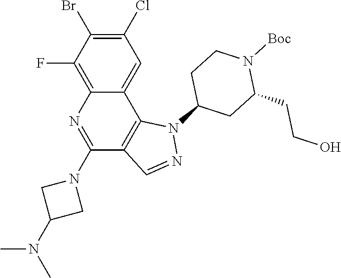 [SMILES: CN(C)C1CN(c2nc3c(F)c(Br)c(Cl)cc3c3c2cnn3[C@H]2CCN(C(=O)OC(C)(C)C)[C@H](CCO)C2)C1]

This compound was prepared according to the procedure described in Example 3a and Example 3b, in Step 18 replacing of tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate with tert-butyl (2S,4S)-4-(7-bromo-8-chloro-6-fluoro-4-(methylthio)-1H-pyrazolo[4,3-c]quinolin-1-yl)-2-(2-hydroxyethyl)piperidine-1-carboxylate. LCMS calculated for C₂₇H₃₆BrClFN₆O₃ (M+H)⁺ m/z=625.2, 627.2; found 625.2, 627.2.

### Step 2. tert-butyl (2S,4S)-4-(8-chloro-4-(3-(dimethylamino)azetidin-1-yl)-6-fluoro-7-(5-fluoroquinolin-8-yl)-1H-pyrazolo[4,3-c]quinolin-1-yl)-2-(2-hydroxyethyl)piperidine-1-carboxylate

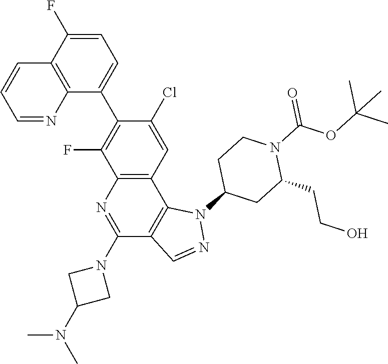 [SMILES: CN(C)C1CN(c2nc3c(F)c(-c4ccc(F)c5cccnc45)c(Cl)cc3c3c2cnn3[C@H]2CCN(C(=O)OC(C)(C)C)[C@H](CCO)C2)C1]

A mixture of tert-butyl (2S,4S)-4-(7-bromo-8-chloro-4-(3-(dimethylamino)azetidin-1-yl)-6-fluoro-1H-pyrazolo[4,3-c]quinolin-1-yl)-2-(2-hydroxyethyl)piperidine-1-carboxylate (251 mg, 0.401 mmol), (5-fluoroquinolin-8-yl)boronic acid (115 mg, 0.601 mmol), tetrakis (46.3 mg, 0.040 mmol) and sodium carbonate (106 mg, 1.002 mmol) in 1,4-dioxane (1.0 mL)/Water (0.200 mL) was stirred at 90° C. for 2 h. The reaction mixture was diluted with ethyl acetate and water. The organic layer was separated and washed with brine, dried over Na₂SO₄, filtered and concentrated. The crude was purified with flash chromatography to give the desired product (278 mg, 100%). LCMS calculated for C₃₆H₄₁ClF₂N₇O₃ (M+H)⁺ m/z=692.3; found 692.3.

### Step 3. tert-butyl (2S,4S)-4-(8-chloro-4-(3-(dimethylamino)azetidin-1-yl)-6-fluoro-7-(5-fluoroquinolin-8-yl)-1H-pyrazolo[4,3-c]quinolin-1-yl)-2-(cyanomethyl)piperidine-1-carboxylate

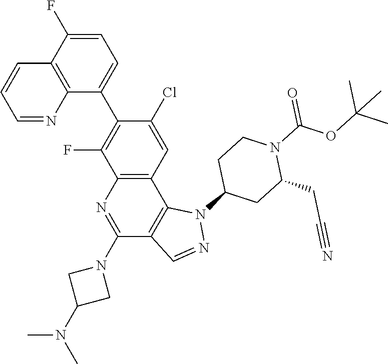 [SMILES: CN(C)C1CN(c2nc3c(F)c(-c4ccc(F)c5cccnc45)c(Cl)cc3c3c2cnn3[C@H]2CCN(C(=O)OC(C)(C)C)[C@H](CC#N)C2)C1]

This compound was prepared according to the procedure described in Example 3a and Example 3b, in Step 17 replacing tert-butyl (2S,4S)-4-(7-bromo-8-chloro-6-fluoro-4-(methylthio)-1H-pyrazolo[4,3-c]quinolin-1-yl)-2-(2-hydroxyethyl)piperidine-1-carboxylate with tert-butyl (2S,4S)-4-(8-chloro-4-(3-(dimethylamino)azetidin-1-yl)-6-fluoro-7-(5-fluoroquinolin-8-yl)-1H-pyrazolo[4,3-c]quinolin-1-yl)-2-(2-hydroxyethyl)piperidine-1-carboxylate. LCMS calculated for C₃₆H₃₈ClF₂N₈O₂ (M+H)⁺ m/z=687.3; found 687.3.

### Step 4. 2-((2S,4S)-4-(8-chloro-4-(3-(dimethylamino)azetidin-1-yl)-6-fluoro-7-(5-fluoroquinolin-8-yl)-1H-pyrazolo[4,3-c]quinolin-1-yl)piperidin-2-yl)acetonitrile

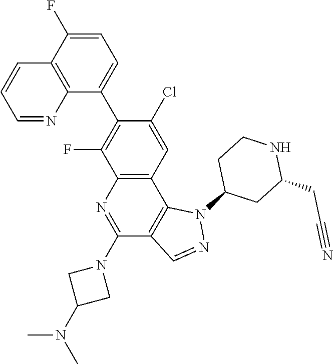 [SMILES: CN(C)C1CN(c2nc3c(F)c(-c4ccc(F)c5cccnc45)c(Cl)cc3c3c2cnn3[C@H]2CCN[C@H](CC#N)C2)C1]

To a solution of tert-butyl (2S,4S)-4-(8-chloro-4-(3-(dimethylamino)azetidin-1-yl)-6-fluoro-7-(5-fluoroquinolin-8-yl)-1H-pyrazolo[4,3-c]quinolin-1-yl)-2-(cyanomethyl)piperidine-1-carboxylate (210 mg, 0.306 mmol) in DCM (1.0 ml) was added TFA (706 μl, 9.17 mmol). After stirring for 1 h, the solvent was removed in vacuo. The crude was used in the next step without further purification. LCMS calculated for C₃₁H₃₀ClF₂N₈(M+H)⁺ m/z=587.2; found 587.2.

### Step 5. 2-((2S,4S)-1-acryloyl-4-(8-chloro-4-(3-(dimethylamino)azetidin-1-yl)-6-fluoro-7-(5-fluoroquinolin-8-yl)-1H-pyrazolo[4,3-c]quinolin-1-yl)piperidin-2-yl)acetonitrile

To a solution of 2-((2S,4S)-4-(8-chloro-4-(3-(dimethylamino)azetidin-1-yl)-6-fluoro-7-(5-fluoroquinolin-8-yl)-1H-pyrazolo[4,3-c]quinolin-1-yl)piperidin-2-yl)acetonitrile bis(2,2,2-trifluoroacetate) (11 mg, 0.013 mmol) in DCM (1.0 ml). DIEA (9.4 μl, 0.054 mmol) was added to reaction vial, followed by 0.25 M acryloyl chloride (54.0 μl, 0.013 mmol). After stirring at 0° C. for 1 h, the solvent was removed and the residue was diluted with methanol and purified using prep-LCMS (XBridge C18 column, eluting with a gradient of acetonitrile/water containing 0.1% TFA, at flow rate of 60 mL/min) to afford the desired diastereomer 1 and Diastereomer 2.

Example 21a. Diastereomer 1. Peak 1. LCMS calculated for C₃₄H₃₂ClF₂N80 (M+H)⁺ m/z=641.2; found 641.2.

Example 21b. Diastereomer 2. Peak 2. LCMS calculated for C₃₄H₃₂ClF₂N80 (M+H)⁺ m/z=641.2; found 641.2.

### Example 22. 2-((2S,4S)-1-acryloyl-4-(8-chloro-4-(3-(dimethylamino)azetidin-1-yl)-6-fluoro-7-(isoquinolin-4-yl)-1H-pyrazolo[4,3-c]quinolin-1-yl)piperidin-2-yl)acetonitrile

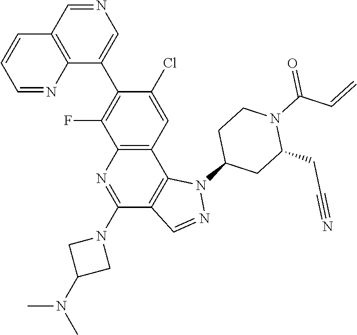 [SMILES: C=CC(=O)N1CC[C@H](n2ncc3c(N4CC(N(C)C)C4)nc4c(F)c(-c5cncc6cccnc56)c(Cl)cc4c32)C[C@H]1CC#N]

### Step 1. tert-butyl (2S,4S)-4-(7-bromo-8-chloro-4-(3-(dimethylamino)azetidin-1-yl)-6-fluoro-1H-pyrazolo[4,3-c]quinolin-1-yl)-2-(cyanomethyl)piperidine-1-carboxylate

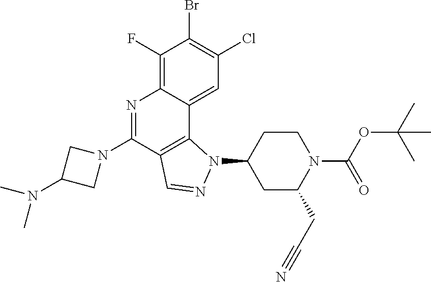 [SMILES: CN(C)C1CN(c2nc3c(F)c(Br)c(Cl)cc3c3c2cnn3[C@H]2CCN(C(=O)OC(C)(C)C)[C@H](CC#N)C2)C1]

This compound was prepared according to the procedure described in Example 3a and Example 3b, in Step 19 replacing tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate with tert-butyl (2S,4S)-4-(7-bromo-8-chloro-6-fluoro-4-(methylthio)-1H-pyrazolo[4,3-c]quinolin-1-yl)-2-(cyanomethyl)piperidine-1-carboxylate. LCMS calculated for C₂₇H₃₃BrClFN₇O₂ (M+H)⁺ m/z=620.2, 622.2; found 620.2, 622.2.

### Step 2. 2-((2S,4S)-1-acryloyl-4-(7-bromo-8-chloro-4-(3-(dimethylamino)azetidin-1-yl)-6-fluoro-1H-pyrazolo[4,3-c]quinolin-1-yl)piperidin-2-yl)acetonitrile

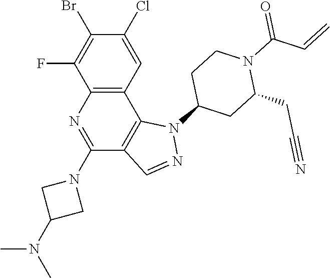 [SMILES: C=CC(=O)N1CC[C@H](n2ncc3c(N4CC(N(C)C)C4)nc4c(F)c(Br)c(Cl)cc4c32)C[C@H]1CC#N]

To a solution of tert-butyl (2S,4S)-4-(7-bromo-8-chloro-4-(3-(dimethylamino)azetidin-1-yl)-6-fluoro-1H-pyrazolo[4,3-c]quinolin-1-yl)-2-(cyanomethyl)piperidine-1-carboxylate (17 mg, 0.027 mmol) in CH₂Cl₂ (0.3 ml) was added TFA (84 μl, 1.095 mmol). The resulting mixture was stirred at rt for 1 h. The solvent was removed in vacuo. The crude was dissolved in DCM (1.0 ml). DIEA (9.4 μl, 0.054 mmol) was added to reaction vial, followed by 0.25 M acryloyl chloride (131 μl, 0.033 mmol). After stirring at 0° C. for 1 h, the solvent was removed and the residue was diluted with methanol and purified using prep-LCMS (XBridge C18 column, eluting with a gradient of acetonitrile/water containing 0.1% TFA, at flow rate of 60 mL/min) to give the desired product (10 mg, 63.5%). LCMS calculated for C₂₅H₂₇BrClFN₇O (M+H)⁺ m/z=574.1; found 574.1.

### Step 3. 2-((2S,4S)-1-acryloyl-4-(8-chloro-4-(3-(dimethylamino)azetidin-1-yl)-6-fluoro-7-(isoquinolin-4-yl)-1H-pyrazolo[4,3-c]quinolin-1-yl)piperidin-2-yl)acetonitrile

A mixture of 2-((2S,4S)-1-acryloyl-4-(7-bromo-8-chloro-4-(3-(dimethylamino)azetidin-1-yl)-6-fluoro-1H-pyrazolo[4,3-c]quinolin-1-yl)piperidin-2-yl)acetonitrile (10 mg, 0.017 mmol), isoquinolin-4-ylboronic acid (6.0 mg, 0.035 mmol), tetrakis (2.0 mg, 1.739 μmol) and sodium carbonate (4.6 mg, 0.043 mmol) in 1,4-dioxane (1.0 mL)/water (0.2 mL) was stirred at 90° C. for 2 h. The residue was dissolved in methanol and 1 N HCl and purified with prep-LCMS (XBridge C18 column, eluting with a gradient of acetonitrile/water containing 0.1% TFA, at flow rate of 60 mL/min) to give the desired product as white solid (4 mg, 37%). LCMS calculated for C₃₄H₃₃ClFN₈O (M+H)⁺: m/z=623.2; found: 623.2.

### Example 23. 2-((2S,4S)-1-acryloyl-4-(8-chloro-7-(2-chloro-3-methylphenyl)-4-(3-(dimethylamino)azetidin-1-yl)-6-fluoro-1H-pyrazolo[4,3-c]quinolin-1-yl)piperidin-2-yl)acetonitrile

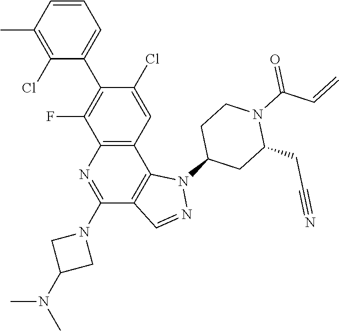 [SMILES: C=CC(=O)N1CC[C@H](n2ncc3c(N4CC(N(C)C)C4)nc4c(F)c(-c5cccc(C)c5Cl)c(Cl)cc4c32)C[C@H]1CC#N]

### Step 1. 2-((2S,4S)-4-(8-chloro-7-(2-chloro-3-methylphenyl)-4-(3-(dimethylamino)azetidin-1-yl)-6-fluoro-1H-pyrazolo[4,3-c]quinolin-1-yl)piperidin-2-yl)acetonitrile

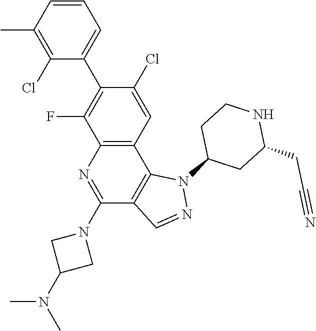 [SMILES: Cc1cccc(-c2c(Cl)cc3c(nc(N4CC(N(C)C)C4)c4cnn([C@H]5CCN[C@H](CC#N)C5)c43)c2F)c1Cl]

A mixture of tert-butyl (2S,4S)-4-(7-bromo-8-chloro-4-(3-(dimethylamino)azetidin-1-yl)-6-fluoro-1H-pyrazolo[4,3-c]quinolin-1-yl)-2-(cyanomethyl)piperidine-1-carboxylate (90 mg, 0.145 mmol), (2-chloro-3-methylphenyl)boronic acid (37.0 mg, 0.217 mmol), tetrakis (16.8 mg, 0.014 mmol) and sodium carbonate (38.4 mg, 0.362 mmol) in 1,4-dioxane (1.0 mL)/water (0.200 mL) was stirred at 90° C. for 2 h. The reaction mixture was diluted with EtOAc and water, the organic layer was separated and concentrated. The residue was dissolved in 1:1 DCM/TFA (1 mL) and stirred for 1 h. The solvent was removed and the residue was purified with prep-LCMS (XBridge C18 column, eluting with a gradient of acetonitrile/water containing 0.1% TFA, at flow rate of 60 mL/min) to give the desired product (42 mg, 43.5%). LCMS calculated for C₂₉H₃₁Cl₂FN₇ (M+H)⁺ m/z=566.2; found 566.2.

### Step 2. 2-((2S,4S)-1-acryloyl-4-(8-chloro-7-(2-chloro-3-methylphenyl)-4-(3-(dimethylamino)azetidin-1-yl)-6-fluoro-1H-pyrazolo[4,3-c]quinolin-1-yl)piperidin-2-yl)acetonitrile

This compound was prepared according to the procedure described in Example 2, step 6, replacing 1-(7-bromo-8-chloro-6-fluoro-1-(piperidin-4-yl)-1H-pyrazolo[4,3-c]quinolin-4-yl)-N,N-dimethylazetidin-3-amine with 2-((2S,4S)-4-(8-chloro-7-(2-chloro-3-methylphenyl)-4-(3-(dimethylamino)azetidin-1-yl)-6-fluoro-1H-pyrazolo[4,3-c]quinolin-1-yl)piperidin-2-yl)acetonitrile. LCMS calculated for C₃₂H₃₃Cl₂FN₇O (M+H)⁺: m/z=620.2; found: 620.2.

### Example 24. 2-((2S,4S)-4-(8-chloro-7-(2-chloro-3-methylphenyl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile

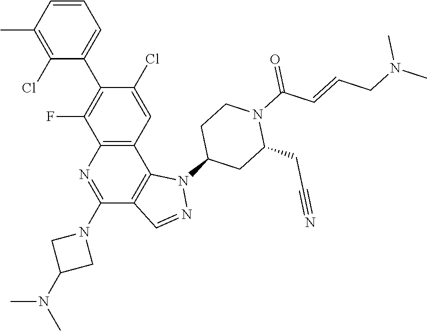

This compound was prepared according to the procedure described in Example 4a and Example 4b, step 1, replacing 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile with 2-((2S,4S)-4-(8-chloro-7-(2-chloro-3-methylphenyl)-4-(3-(dimethylamino)azetidin-1-yl)-6-fluoro-1H-pyrazolo[4,3-c]quinolin-1-yl)piperidin-2-yl)acetonitrile. LCMS calculated for C₃₅H₄₀Cl₂FN₈O (M+H)⁺: m/z=677.3; found: 677.3.

### Example 25a and Example 25b. 2-((2S,4S)-1-acryloyl-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile

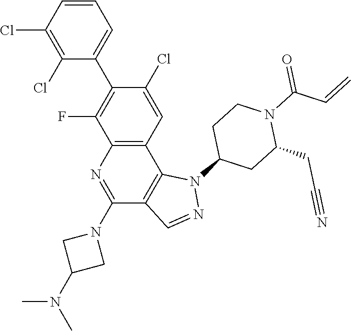 [SMILES: C=CC(=O)N1CC[C@H](n2ncc3c(N4CC(N(C)C)C4)nc4c(F)c(-c5cccc(Cl)c5Cl)c(Cl)cc4c32)C[C@H]1CC#N]

### Step 1. 2-((2S,4S)-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile

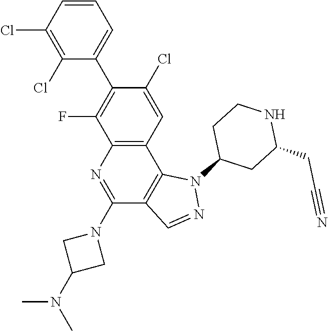 [SMILES: CN(C)C1CN(c2nc3c(F)c(-c4cccc(Cl)c4Cl)c(Cl)cc3c3c2cnn3[C@H]2CCN[C@H](CC#N)C2)C1]

This compound was prepared according to the procedure described in Example 23, step 1, replacing (2-chloro-3-methylphenyl)boronic acid with (2,3-dichlorophenyl)boronic acid. LCMS calculated for C₂₈H₂₈Cl₃FN₇ (M+H)⁺ m/z=586.1, 588.1; found 586.1, 588.1.

### Step 2. 2-((2S,4S)-1-acryloyl-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile

This compound was prepared according to the procedure described in Example 2, step 6, replacing 1-(7-bromo-8-chloro-6-fluoro-1-(piperidin-4-yl)-1H-pyrazolo[4,3-c]quinolin-4-yl)-N,N-dimethylazetidin-3-amine with 2-((2S,4S)-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile.

Example 25a. Diastereomer 1. Peak 1. LCMS calculated for C₃₁H₃₀Cl₃FN₇O (M+H)⁺: m/z=640.2, 642.2; found: 640.2, 642.2.

Example 25b. Diastereomer 2. Peak 2. LCMS calculated for C₃₁H₃₀Cl₃FN₇O (M+H)⁺: m/z=640.2, 642.2; found: 640.2, 642.2.

### Example 26a and Example 26b. 2-((2S,4S)-1-(but-2-ynoyl)-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile

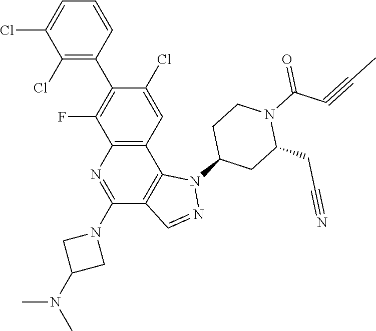 [SMILES: CC#CC(=O)N1CC[C@H](n2ncc3c(N4CC(N(C)C)C4)nc4c(F)c(-c5cccc(Cl)c5Cl)c(Cl)cc4c32)C[C@H]1CC#N]

This compound was prepare according to the procedure described in Example 9a and Example 9b, replacing 2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile with 2-((2S,4S)-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile.

Example 26a. Diastereomer 1. Peak 1. LCMS calculated for C₃₂H₃₀Cl₃FN₇O (M+H)⁺ m/z=652.2, 654.2; found 652.2, 654.2.

Example 26b. Diastereomer 2. Peak 2. LCMS calculated for C₃₂H₃₀Cl₃FN₇O (M+H)⁺ m/z=652.2, 654.2; found 652.2, 654.2.

### Example 27. 2-((2S,4S)-1-acryloyl-4-(8-chloro-4-(3-(dimethylamino)azetidin-1-yl)-6-fluoro-7-(3-methyl-2-(trifluoromethyl)phenyl)-1H-pyrazolo[4,3-c]quinolin-1-yl)piperidin-2-yl)acetonitrile

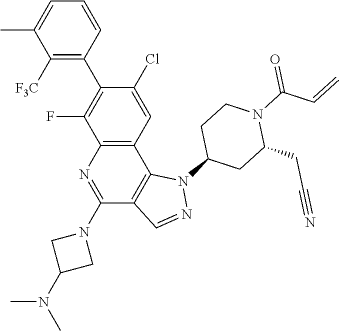 [SMILES: C=CC(=O)N1CC[C@H](n2ncc3c(N4CC(N(C)C)C4)nc4c(F)c(-c5cccc(C)c5C(F)(F)F)c(Cl)cc4c32)C[C@H]1CC#N]

### Step 1. tert-butyl (2S,4S)-4-amino-2-(2-hydroxyethyl)piperidine-1-carboxylate

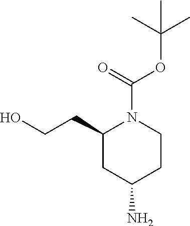 [SMILES: CC(C)(C)OC(=O)N1CC[C@H](N)C[C@H]1CCO]

To a solution of tert-butyl (2S,4S)-4-azido-2-(2-hydroxyethyl)piperidine-1-carboxylate (1.87 g, 6.92 mmol) in methanol (35 ml) was added 10% palladium on carbon (0.736 g, 0.692 mmol). The reaction mixture was evacuated under vacuum and refilled with H₂, stirred at rt for 2 h. The reaction mixture was filtered through a pad of Celite and washed with methanol. The filtrate was concentrated to give the desired product (1.6 g, 95%). LCMS calculated for (Product-Boc) C₇H₁₇N₂O (M+H)⁺: m/z=145.1; found: 145.1.

### Step 2. methyl 2-amino-3-fluoro-4-(4,4,5,5-tetramethyl-1,3,2-dioxaborolan-2-yl)benzoate

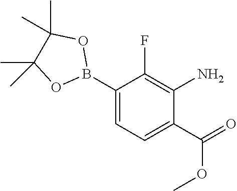 [SMILES: COC(=O)c1ccc(B2OC(C)(C)C(C)(C)O2)c(F)c1N]

A mixture of methyl 2-amino-4-bromo-3-fluorobenzoate (349 mg, 1.407 mmol), bis(pinacolato)diboron (429 mg, 1.688 mmol), dichloro[1,1′-bis(diphenylphosphino)ferrocene]palladium (II) dichloromethane adduct (115 mg, 0.141 mmol) and acetic acid, potassium salt, anhydrous (304 mg, 3.10 mmol) was charged with nitrogen and stirred at 100° C. for 4 h. The mixture was filtered through a pad of Celite and washed with DCM. The filtrate was concentrated. The residue was purified by flash chromatography to give the desired product (0.40 g, 96%). LCMS calculated for C₁₄H₂₀BFNO₄ (M+H)⁺: m/z=296.1; found: 296.1.

### Step 3. methyl 3-amino-2-fluoro-3′-methyl-2′-(trifluoromethyl)-[1,1′-biphenyl]-4-carboxylate

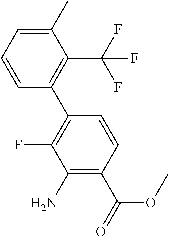 [SMILES: COC(=O)c1ccc(-c2cccc(C)c2C(F)(F)F)c(F)c1N]

A mixture of 1-bromo-3-methyl-2-(trifluoromethyl)benzene (280 mg, 1.171 mmol), methyl 2-amino-3-fluoro-4-(4,4,5,5-tetramethyl-1,3,2-dioxaborolan-2-yl)benzoate (380 mg, 1.289 mmol), tetrakis (135 mg, 0.117 mmol) and sodium bicarbonate (197 mg, 2.343 mmol) in 1,4-dioxane (8.0 mL)/water (1.6 mL) was stirred at 90° C. for 6 h. The reaction mixture was diluted with ethyl acetate and water. The organic layer was separated and dried over Na₂SO₄, filtered and concentrated and used directly in the next step without further purification. LCMS calculated for C₁₆H₁₄F₄NO₂ (M+H)⁺: m/z=328.1; found: 328.1.

### Step 4. methyl 3-amino-6-chloro-2-fluoro-3′-methyl-2′-(trifluoromethyl)-[1,1′-biphenyl]-4-carboxylate

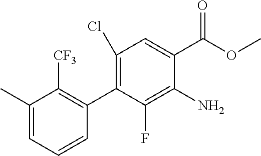

To a solution of methyl 3-amino-2-fluoro-3′-methyl-2′-(trifluoromethyl)-[1,1′-biphenyl]-4-carboxylate (380 mg, 1.161 mmol) in DMF (3.9 ml) was added NCS (171 mg, 1.277 mmol) at rt. The mixture was stirred at room temperature for 10 min. The reaction mixture was diluted with water and DCM. The organic layer was separated and dried over Na₂SO₄, filtered and concentrated and used directly in the next step without further purification. LCMS calculated for C₁₆H₁₃ClF₄NO₂ (M+H)⁺: m/z=362.1; found: 362.1.

### Step 5. methyl 6-chloro-3-(3-ethoxy-3-oxopropanamido)-2-fluoro-3′-methyl-2′-(trifluoromethyl)-[1,1′-biphenyl]-4-carboxylate

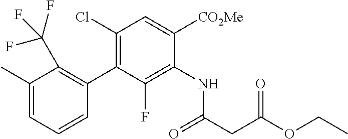 [SMILES: CCOC(=O)CC(=O)Nc1c(C(=O)OC)cc(Cl)c(-c2cccc(C)c2C(F)(F)F)c1F]

Ethyl 3-chloro-3-oxopropanoate (0.178 ml, 1.393 mmol) was added dropwise to a solution of methyl 3-amino-6-chloro-2-fluoro-3′-methyl-2′-(trifluoromethyl)-[1,1′-biphenyl]-4-carboxylate (0.420 g, 1.161 mmol) and TEA (0.194 ml, 1.393 mmol) in DCM (10 mL) at rt. The resulting mixture was stirred at rt for 4 h, The reaction was diluted with water and DCM. The organic layer was separated and dried over Na₂SO₄, filtered and concentrated. The residue was purified with flash chromatography to give the desired product (0.32 g, 58% over 3 steps). LCMS calculated for C₂₁H₁₉ClF₄NO₅ (M+H)⁺: m/z=476.1; found: 476.1.

### Step 6. ethyl 2,4,6-trichloro-8-fluoro-7-(3-methyl-2-(trifluoromethyl)phenyl)quinoline-3-carboxylate

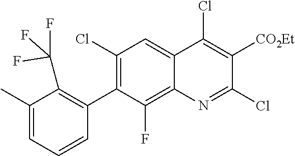 [SMILES: CCOC(=O)c1c(Cl)nc2c(F)c(-c3cccc(C)c3C(F)(F)F)c(Cl)cc2c1Cl]

21% sodium ethoxide (0.741 ml, 1.986 mmol) in EtOH was added dropwise to a solution of methyl 6-chloro-3-(3-ethoxy-3-oxopropanamido)-2-fluoro-3′-methyl-2′-(trifluoromethyl)-[1,1′-biphenyl]-4-carboxylate (0.315 g, 0.662 mmol) in EtOH (4 mL). Precipitates appeared during the addition process. The reaction was stirred at rt for 30 min. The solvent was removed under vacuum, and the crude product was used in next step without further purification.

The crude product from last step was dissolved in POCl₃ (1.24 mL, 13.3 mmol), and DIEA (0.23 ml, 1.33 mmol) was added. The resulting mixture was stirred at 100° C. for 2h. POCl₃ was removed by azeotrope with PhMe (3 times), and the residue was purified on silica gel column (EtOAc in hexanes, 0˜20% gradient) to yield the product as white solid (184 mg, 58%). LCMS calculated for C₂₀H₁₃Cl₃F₄NO₂ (M+H)⁺: m/z=480.0, 482.0; found: 480.0, 482.0.

### Step 7. ethyl 4-(((2S,4S)-1-(tert-butoxycarbonyl)-2-(2-hydroxyethyl)piperidin-4-yl)amino)-2,6-dichloro-8-fluoro-7-(3-methyl-2-(trifluoromethyl)phenyl)quinoline-3-carboxylate

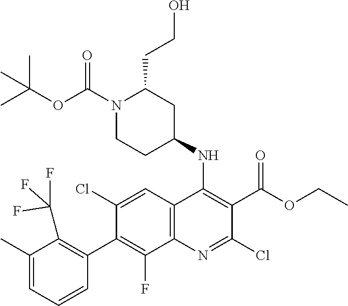 [SMILES: CCOC(=O)c1c(Cl)nc2c(F)c(-c3cccc(C)c3C(F)(F)F)c(Cl)cc2c1N[C@H]1CCN(C(=O)OC(C)(C)C)[C@H](CCO)C1]

To a solution of ethyl 2,4,6-trichloro-8-fluoro-7-(3-methyl-2-(trifluoromethyl)phenyl)-quinoline-3-carboxylate (1.04 g, 2.164 mmol) in DMF (15 ml) was added tert-butyl (2S,4S)-4-amino-2-(2-hydroxyethyl)piperidine-1-carboxylate (0.634 g, 2.60 mmol) and DIEA (0.76 ml, 4.33 mmol). The resulting mixture was stirred at 60° C. for 16 h. After cooling to room temperature, ethyl acetate and water were added. The organic layer was washed with water (2×) and brine, dried over Na₂SO₄, filtered and concentrated. The residue was purified with flash chromatography (eluting with 0%-25% ethyl acetate in hexanes) to give the desired product as foam (1.48 g, 99%). LCMS calculated for C₃₂H₃₆Cl₂F₄N₃O₅ (M+H)⁺: m/z=688.2; found: 688.2.

### Step 8. ethyl 4-(((2S,4S)-1-(tert-butoxycarbonyl)-2-(2-((tert-butyldimethylsilyl)oxy)ethyl)piperidin-4-yl)amino)-2,6-dichloro-8-fluoro-7-(3-methyl-2-(trifluoromethyl)phenyl)quinoline-3-carboxylate

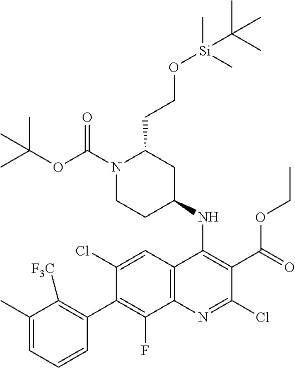 [SMILES: CCOC(=O)c1c(Cl)nc2c(F)c(-c3cccc(C)c3C(F)(F)F)c(Cl)cc2c1N[C@H]1CCN(C(=O)OC(C)(C)C)[C@H](CCO[Si](C)(C)C(C)(C)C)C1]

To a solution of ethyl 4-(((2S,4S)-1-(tert-butoxycarbonyl)-2-(2-hydroxyethyl)piperidin-4-yl)amino)-2,6-dichloro-8-fluoro-7-(3-methyl-2-(trifluoromethyl)phenyl)quinoline-3-carboxylate (101 mg, 0.147 mmol) in DMF (0.73 ml) was added imidazole (15 mg, 0.220 mmol) and TBS-CI (28.7 mg, 0.191 mmol). The resulting mixture was stirred at 60° C. for 1 h 15 min. The reaction was diluted with EtOAc and water. The organic layer was washed with water and brine, dried over Na₂SO₄, filtered and concentrated. The residue was purified with flash chromatography (eluting with 0%-25% ethyl acetate in hexanes) to give the desired product as foam (110 mg, 93%). LCMS calculated for C₃₈H₅₀Cl₂F₄N₃O₅Si (M+H)⁺: m/z=802.3; found: 802.3.

### Step 9. tert-butyl (2S,4S)-2-(2-((tert-butyldimethylsilyl)oxy)ethyl)-4-((2,6-dichloro-8-fluoro-3-(hydroxymethyl)-7-(3-methyl-2-(trifluoromethyl)phenyl)quinolin-4-yl)amino)piperidine-1-carboxylate

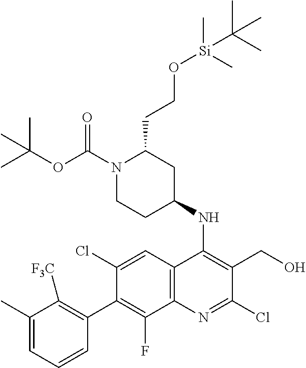 [SMILES: Cc1cccc(-c2c(Cl)cc3c(N[C@H]4CCN(C(=O)OC(C)(C)C)[C@H](CCO[Si](C)(C)C(C)(C)C)C4)c(CO)c(Cl)nc3c2F)c1C(F)(F)F]

To a solution of ethyl 4-(((2S,4S)-1-(tert-butoxycarbonyl)-2-(2-((tert-butyldimethylsilyl)oxy)ethyl)piperidin-4-yl)amino)-2,6-dichloro-8-fluoro-7-(3-methyl-2-(trifluoromethyl)phenyl)quinoline-3-carboxylate (0.95 g, 1.183 mmol) in toluene (6.0 ml) at −78° C. was added 1.0 M DIBAL-H in DCM (4.14 ml, 4.14 mmol). The resulting mixture was stirred at −78° C. for 40 min and warm to 0° C. for 1 h and 20 min, quenched with methanol (0.5 ml). Aqueous Rochelle salt solution (prepared from 4.8 g of Rochelle salt and 30 mL of water) was added to the solution at ≤10° C. The biphasic mixture was stirred vigorously for ≥1 h and separated to give organic layer. The organic layer was washed with aqueous NaCl (×2). The organic layer was dried over Na₂SO₄, filtered and concentrated. and used as is. LCMS calculated for C₃₆H₄₈Cl₂F₄N₃O₄Si (M+H)⁺: m/z=760.3; found: 760.3.

### Step 10. tert-butyl (2S,4S)-2-(2-((tert-butyldimethylsilyl)oxy)ethyl)-4-((2,6-dichloro-8-fluoro-3-formyl-7-(3-methyl-2-(trifluoromethyl)phenyl)quinolin-4-yl)amino)piperidine-1-carboxylate

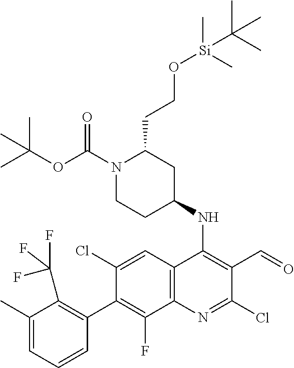 [SMILES: Cc1cccc(-c2c(Cl)cc3c(N[C@H]4CCN(C(=O)OC(C)(C)C)[C@H](CCO[Si](C)(C)C(C)(C)C)C4)c(C=O)c(Cl)nc3c2F)c1C(F)(F)F]

To a solution of tert-butyl (2S,4S)-2-(2-((tert-butyldimethylsilyl)oxy)ethyl)-4-((2,6-dichloro-8-fluoro-3-(hydroxymethyl)-7-(3-methyl-2-(trifluoromethyl)phenyl)quinolin-4-yl)amino)piperidine-1-carboxylate (0.90 g, 1.183 mmol) in DCM (11.8 ml) was added dess-martinperiodinane (0.60 g, 1.42 mmol). The resulting mixture was stirred for 1 h, to the reaction flask was added saturated NaHCO₃ and stirred for 10 min. The organic layer was separated and dried over Na₂SO₄, filtered and concentrated. The residue was purified with flash chromatography (eluting with 0%-25% ethyl acetate in hexanes) to give the desired product as foam (741 mg, 83%). LCMS calculated for C₃₆H₄₆Cl₂F₄N₃O₄Si (M+H)⁺: m/z=758.3; found: 758.3.

### Step 11. tert-butyl (2S,4S)-2-(2-((tert-butyldimethylsilyl)oxy)ethyl)-4-((2,6-dichloro-8-fluoro-3-((E)-(hydroxyimino)methyl)-7-(3-methyl-2-(trifluoromethyl)phenyl)quinolin-4-yl)amino)piperidine-1-carboxylate

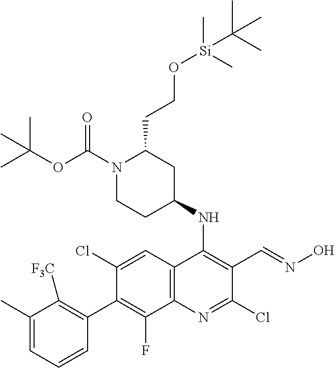 [SMILES: Cc1cccc(-c2c(Cl)cc3c(N[C@H]4CCN(C(=O)OC(C)(C)C)[C@H](CCO[Si](C)(C)C(C)(C)C)C4)c(/C=N/O)c(Cl)nc3c2F)c1C(F)(F)F]

To a mixture of tert-butyl (2S,4S)-2-(2-((tert-butyldimethylsilyl)oxy)ethyl)-4-((2,6-dichloro-8-fluoro-3-formyl-7-(3-methyl-2-(trifluoromethyl)phenyl)quinolin-4-yl)amino)piperidine-1-carboxylate (741 mg, 0.977 mmol) DCM (9.77 ml) and EtOH (9.77 ml) was added hydroxylamine hydrochloride (231 mg, 3.32 mmol) and pyridine (276 μl, 3.42 mmol). The resulting mixture was stirred at 40° C. for 16 hours. The solvent was evaporated in vacuo. The residue was dissolved in EtOAc and washed with water, brine. The organic layer was dried over MgSO₄, filtered and evaporated in vacuo. The crude mixture was purified by column chromatography on silica gel (0.46 g, 61%). LCMS calculated for C₃₆H₄₇Cl₂F₄N₄O₄Si (M+H)⁺: m/z=773.3; found: 773.3.

### Step 12. tert-butyl (2S,4S)-2-(2-((tert-butyldimethylsilyl)oxy)ethyl)-4-(4,8-dichloro-6-fluoro-7-(3-methyl-2-(trifluoromethyl)phenyl)-1H-pyrazolo[4,3-c]quinolin-1-yl)piperidine-1-carboxylate

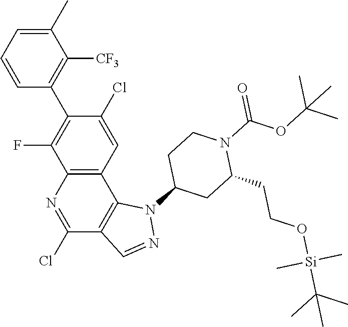 [SMILES: Cc1cccc(-c2c(Cl)cc3c(nc(Cl)c4cnn([C@H]5CCN(C(=O)OC(C)(C)C)[C@H](CCO[Si](C)(C)C(C)(C)C)C5)c43)c2F)c1C(F)(F)F]

To a mixture of (tert-butyl (2S,4S)-2-(2-((tert-butyldimethylsilyl)oxy)ethyl)-4-((2,6-dichloro-8-fluoro-3-((E)-(hydroxyimino)methyl)-7-(3-methyl-2-(trifluoromethyl)phenyl)quinolin-4-yl)amino)piperidine-1-carboxylate (462 mg, 0.597 mmol), CH₂Cl₂ (1.5 mL) and 2-aminopyridine (112 mg, 1.194 mmol)) was added Ms—Cl (93 μl, 1.194 mmol) at 0° C. After stirring at 0° C. for 2 h. The mixture was allowed to warm to room temperature overnight. The reaction mixture was diluted with water and DCM. The organic layer was washed with water, brine, dried over MgSO₄, filtered and concentrated. The crude was purified by column chromatography on silica gel (157 mg, 35%). LCMS calculated for C₃₆H₄₅Cl₂F₄N₄O₃Si (M+H)⁺: m/z=755.3; found: 755.3.

### Step 13. tert-butyl (2S,4S)-2-(2-((tert-butyldimethylsilyl)oxy)ethyl)-4-(8-chloro-6-fluoro-7-(3-methyl-2-(trifluoromethyl)phenyl)-4-(methylthio)-1H-pyrazolo[4,3-c]quinolin-1-yl)piperidine-1-carboxylate

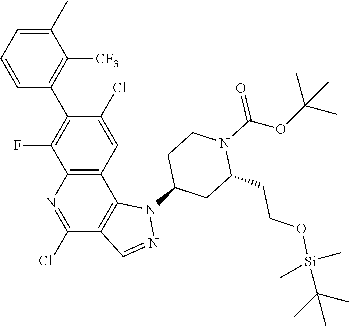 [SMILES: Cc1cccc(-c2c(Cl)cc3c(nc(Cl)c4cnn([C@H]5CCN(C(=O)OC(C)(C)C)[C@H](CCO[Si](C)(C)C(C)(C)C)C5)c43)c2F)c1C(F)(F)F]

This compound was prepared according to the procedure described in Example 3a and Example 3b, step 15, replacing tert-butyl (2S,4S)-4-(7-bromo-4,8-dichloro-6-fluoro-1H-pyrazolo[4,3-c]quinolin-1-yl)-2-(2-((tert-butyldimethylsilyl)oxy)ethyl)piperidine-1-carboxylate with tert-butyl (2S,4S)-2-(2-((tert-butyldimethylsilyl)oxy)ethyl)-4-(4,8-dichloro-6-fluoro-7-(3-methyl-2-(trifluoromethyl) phenyl)-1H-pyrazolo[4,3-c]quinolin-1-yl)piperidine-1-carboxylate. LCMS calculated for C₃₇H₄₈ClF₄N₄O₃SSi (M+H)⁺: m/z=767.3; found: 767.4.

### Step 14. tert-butyl (2S,4S)-4-(8-chloro-6-fluoro-7-(3-methyl-2-(trifluoromethyl)phenyl)-4-(methylthio)-1H-pyrazolo[4,3-c]quinolin-1-yl)-2-(2-hydroxyethyl)piperidine-1-carboxylate

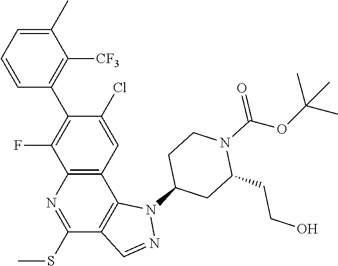 [SMILES: CSc1nc2c(F)c(-c3cccc(C)c3C(F)(F)F)c(Cl)cc2c2c1cnn2[C@H]1CCN(C(=O)OC(C)(C)C)[C@H](CCO)C1]

This compound was prepared according to the procedure described in Example 3a and Example 3b, step 16, replacing tert-butyl (2S,4S)-4-(7-bromo-8-chloro-6-fluoro-4-(methylthio)-1H-pyrazolo[4,3-c]quinolin-1-yl)-2-(2-((tert-butyldimethylsilyl)oxy)ethyl)piperidine-1-carboxylate with tert-butyl (2S,4S)-2-(2-((tert-butyldimethylsilyl)oxy)ethyl)-4-(8-chloro-6-fluoro-7-(3-methyl-2-(trifluoromethyl)phenyl)-4-(methylthio)-1H-pyrazolo[4,3-c]quinolin-1-yl)piperidine-1-carboxylate. LCMS calculated for C₃₁H₃₄ClF₄N₄O₃S (M+H)⁺: m/z=653.2; found: 653.2.

### Step 15. tert-butyl (2S,4S)-4-(8-chloro-6-fluoro-7-(3-methyl-2-(trifluoromethyl)phenyl)-4-(methylthio)-1H-pyrazolo[4,3-c]quinolin-1-yl)-2-(cyanomethyl)piperidine-1-carboxylate

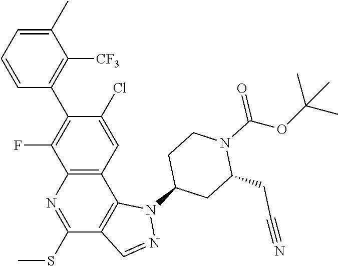 [SMILES: CSc1nc2c(F)c(-c3cccc(C)c3C(F)(F)F)c(Cl)cc2c2c1cnn2[C@H]1CCN(C(=O)OC(C)(C)C)[C@H](CC#N)C1]

This compound was prepared according to the procedure described in Example 3a and Example 3b, step 17, replacing tert-butyl (2S,4S)-4-(7-bromo-8-chloro-6-fluoro-4-(methylthio)-1H-pyrazolo[4,3-c]quinolin-1-yl)-2-(2-hydroxyethyl)piperidine-1-carboxylate with tert-butyl (2S,4S)-4-(8-chloro-6-fluoro-7-(3-methyl-2-(trifluoromethyl)phenyl)-4-(methylthio)-1H-pyrazolo[4,3-c]quinolin-1-yl)-2-(2-hydroxyethyl)piperidine-1-carboxylate. LCMS calculated for C₃₁1H₃₁ClF₄N₅O₂S (M+H)⁺: m/z=648.2; found: 648.2.

### Step 16. tert-butyl (2S,4S)-4-(8-chloro-4-(3-(dimethylamino)azetidin-1-yl)-6-fluoro-7-(3-methyl-2-(trifluoromethyl)phenyl)-1H-pyrazolo[4,3-c]quinolin-1-yl)-2-(cyanomethyl)piperidine-1-carboxylate

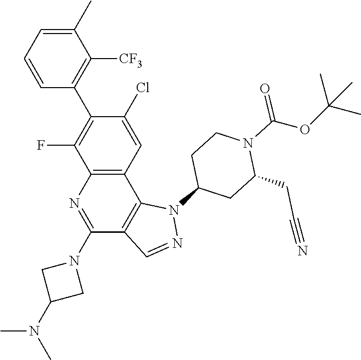 [SMILES: Cc1cccc(-c2c(Cl)cc3c(nc(N4CC(N(C)C)C4)c4cnn([C@H]5CCN(C(=O)OC(C)(C)C)[C@H](CC#N)C5)c43)c2F)c1C(F)(F)F]

This compound was prepared according to the procedure described in Example 3a and Example 3b, step 19, replacing tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate with tert-butyl (2S,4S)-4-(8-chloro-6-fluoro-7-(3-methyl-2-(trifluoromethyl)phenyl)-4-(methylthio)-1H-pyrazolo[4,3-c]quinolin-1-yl)-2-(cyanomethyl)piperidine-1-carboxylate. LCMS calculated for C₃₅H₃₉ClF₄N₇O₂ (M+H)⁺: m/z=700.3; found: 700.3.

### Step 17. 2-((2S,4S)-4-(8-chloro-4-(3-(dimethylamino)azetidin-1-yl)-6-fluoro-7-(3-methyl-2-(trifluoromethyl)phenyl)-1H-pyrazolo[4,3-c]quinolin-1-yl)piperidin-2-yl)acetonitrile

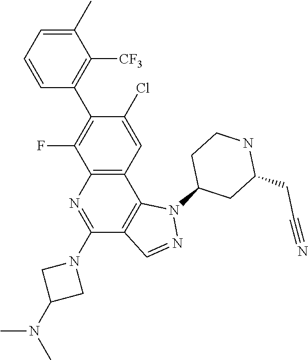 [SMILES: Cc1cccc(-c2c(Cl)cc3c(nc(N4CC(N(C)C)C4)c4cnn([C@H]5CCN[C@H](CC#N)C5)c43)c2F)c1C(F)(F)F]

This compound was prepared according to the procedure described in Example 3a and Example 3b, step 20, replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl) piperidine-1-carboxylate with. tert-butyl (2S,4S)-4-(8-chloro-4-(3-(dimethylamino)azetidin-1-yl)-6-fluoro-7-(3-methyl-2-(trifluoromethyl)phenyl)-1H-pyrazolo[4,3-c]quinolin-1-yl)-2-(cyanomethyl)piperidine-1-carboxylate. LCMS calculated for C₃₀H₃₁ClF₄N₇ (M+H)⁺: m/z=600.2; found: 600.2.

### Step 18. 2-((2S,4S)-1-acryloyl-4-(8-chloro-4-(3-(dimethylamino)azetidin-1-yl)-6-fluoro-7-(3-methyl-2-(trifluoromethyl)phenyl)-1H-pyrazolo[4,3-c]quinolin-1-yl)piperidin-2-yl)acetonitrile

This compound was prepared according to the procedure described in Example 2, step 6, replacing 1-(7-bromo-8-chloro-6-fluoro-1-(piperidin-4-yl)-1H-pyrazolo[4,3-c]quinolin-4-yl)-N,N-dimethylazetidin-3-amine with 2-((2S,4S)-4-(8-chloro-4-(3-(dimethylamino)azetidin-1-yl)-6-fluoro-7-(3-methyl-2-(trifluoromethyl)phenyl)-1H-pyrazolo[4,3-c]quinolin-1-yl)piperidin-2-yl)acetonitrile to afford the product as a mixture of diastereomers. LCMS calculated for C₃₃H₃₃ClF₄N₇O (M+H)⁺ m/z=654.2; found 654.2.

### Example 28. 2-((2S,4S)-1-acryloyl-4-(8-chloro-6-fluoro-7-(3-methyl-2-(trifluoromethyl)phenyl)-4-(((S)-1-methylpyrrolidin-2-yl)methoxy)-1H-pyrazolo[4,3-c]quinolin-1-yl)piperidin-2-yl)acetonitrile

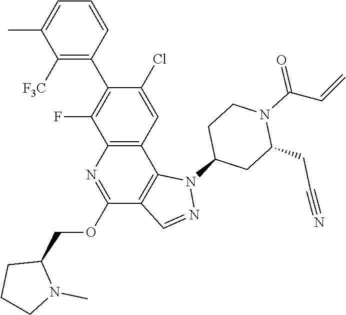 [SMILES: C=CC(=O)N1CC[C@H](n2ncc3c(OC[C@@H]4CCCN4C)nc4c(F)c(-c5cccc(C)c5C(F)(F)F)c(Cl)cc4c32)C[C@H]1CC#N]

### Step 1. tert-butyl (2S,4S)-4-(8-chloro-6-fluoro-7-(3-methyl-2-(trifluoromethyl)phenyl)-4-(((S)-1-methylpyrrolidin-2-yl)methoxy)-1H-pyrazolo[4,3-c]quinolin-1-yl)-2-(cyanomethyl)piperidine-1-carboxylate

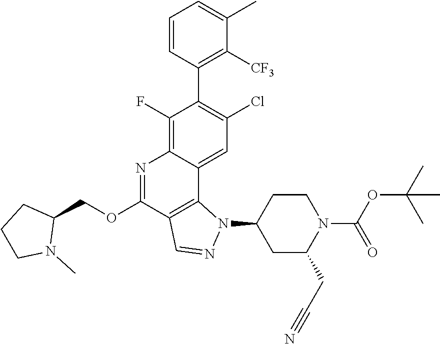 [SMILES: Cc1cccc(-c2c(Cl)cc3c(nc(OC[C@@H]4CCCN4C)c4cnn([C@H]5CCN(C(=O)OC(C)(C)C)[C@H](CC#N)C5)c43)c2F)c1C(F)(F)F]

This compound was prepared according to the procedure described in Example 17a and Example 17b, in Step 1, replacing tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate with tert-butyl (2S,4S)-4-(8-chloro-6-fluoro-7-(3-methyl-2-(trifluoromethyl)phenyl)-4-(methylthio)-1H-pyrazolo[4,3-c]quinolin-1-yl)-2-(cyanomethyl)piperidine-1-carboxylate. LCMS calculated for C₃₆H₄₀ClF₄N₆O₃ (M+H)⁺ m/z=715.3; found 715.3.

### Step 2. 2-((2S,4S)-4-(8-chloro-6-fluoro-7-(3-methyl-2-(trifluoromethyl)phenyl)-4-(((S)-1-methylpyrrolidin-2-yl)methoxy)-1H-pyrazolo[4,3-c]quinolin-1-yl)piperidin-2-yl)acetonitrile

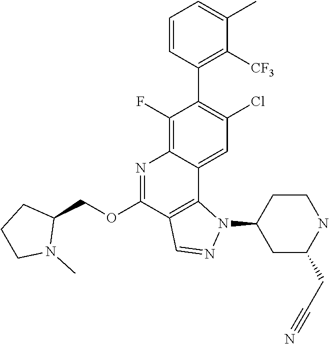 [SMILES: Cc1cccc(-c2c(Cl)cc3c(nc(OC[C@@H]4CCCN4C)c4cnn([C@H]5CCN[C@H](CC#N)C5)c43)c2F)c1C(F)(F)F]

This compound was prepared according to the procedure described in Example 3a and Example 3b, step 20, replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-4-(8-chloro-6-fluoro-7-(3-methyl-2-(trifluoromethyl)phenyl)-4-(((S)-1-methylpyrrolidin-2-yl)methoxy)-1H-pyrazolo[4,3-c]quinolin-1-yl)-2-(cyanomethyl)piperidine-1-carboxylate. LCMS calculated for C₃₁1H₃₂ClF₄N₆O (M+H)⁺: m/z=615.2; found: 615.2.

### Step 3. 2-((2S,4S)-1-acryloyl-4-(8-chloro-6-fluoro-7-(3-methyl-2-(trifluoromethyl)phenyl)-4-(((S)-1-methylpyrrolidin-2-yl)methoxy)-1H-pyrazolo[4,3-c]quinolin-1-yl)piperidin-2-yl)acetonitrile

This compound was prepared according to the procedure described in Example 2, step 6, replacing 1-(7-bromo-8-chloro-6-fluoro-1-(piperidin-4-yl)-1H-6pyrazolo[4,3c]quinolin-4-yl)-N,N-dimethylazetidin-3-amine with 2-((2S,4S)-4-(8-chloro-6-fluoro-7-(3-methyl-2-(trifluoromethyl)phenyl)-4-(((S)-1-methylpyrrolidin-2-yl)methoxy)-1H-pyrazolo[4,3-c]quinolin-1-yl)piperidin-2-yl)acetonitrile to afford the product as a mixture of diastereomers. LCMS calculated for C₃₄H₃₄ClF₄N₇O₂ (M+H)⁺ m/z=669.2; found 669.2.

### Example 29. Methyl 3-(1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinolin-2-yl)propanoate

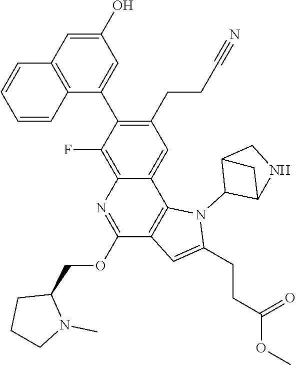 [SMILES: COC(=O)CCc1cc2c(OC[C@@H]3CCCN3C)nc3c(F)c(-c4cc(O)cc5ccccc45)c(CCC#N)cc3c2n1C1C2CNC1C2]

### Step 1. 2-amino-4-bromo-3-fluoro-5-iodobenzoic acid

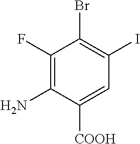 [SMILES: Nc1c(C(=O)O)cc(I)c(Br)c1F]

1-Iodopyrrolidine-2,5-dione (21.15 g, 94 mmol) was added to a solution of 2-amino-4-bromo-3-fluorobenzoic acid (20 g, 85 mmol)) in DMF (200 ml) and then the reaction was stirred at 80° C. for 3 h. The mixture was cooled with ice water and then water (500 mL) was added, the precipitate was filtered and washed with water, dried to provide the desired product as a solid.

### Step 2. 7-bromo-8-fluoro-6-iodo-2H-benzo[d][1,3]oxazine-2,4(1H)-dione

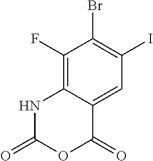 [SMILES: O=c1[nH]c2c(F)c(Br)c(I)cc2c(=O)o1]

Triphosgene (9.07 g, 30.6 mmol) was added to a solution of 2-amino-4-bromo-3-fluoro-5-iodobenzoic acid (22 g, 61.1 mmol) in dioxane (200 ml) and then the reaction was stirred at 80° C. for 2 h. The reaction mixture was cooled with ice water and then filtered. The solid was washed with ethyl acetate to provide the desired product as a solid.

### Step 3. 7-bromo-8-fluoro-6-iodo-3-nitroquinoline-2,4-diol

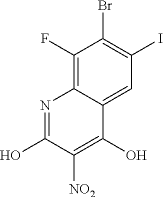 [SMILES: O=[N+]([O-])c1c(O)nc2c(F)c(Br)c(I)cc2c1O]

DIPEA (25.5 ml, 146 mmol) was added to a solution of ethyl 2-nitroacetate (16.33 ml, 146 mmol) and 7-bromo-8-fluoro-6-methyl-2H-benzo[d][1,3]oxazine-2,4(1H)-dione (20 g, 73.0 mmol) in toluene (200 ml) at r.t. and the reaction was stirred at 95° C. for 3 h. The reaction was cooled and then filtered, then washed with small amount of hexanes to provide the desired product.

### Step 4. 7-bromo-2,4-dichloro-8-fluoro-6-iodo-3-nitroquinoline

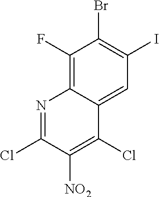 [SMILES: O=[N+]([O-])c1c(Cl)nc2c(F)c(Br)c(I)cc2c1Cl]

DIPEA (8.14 ml, 46.6 mmol) was added to a mixture of 7-bromo-8-fluoro-6-iodo-3-nitroquinoline-2,4-diol (10 g, 23.31 mmol) in POCl₃ (10.86 ml, 117 mmol) and then the reaction was stirred at 100° C. for 2 h. The solvent was removed under vacuum and then azeotroped with toluene 3 times to provide the crude material which was purified with flash column.

### Step 5. tert-butyl 5-((7-bromo-2-chloro-8-fluoro-6-iodo-3-nitroquinolin-4-yl)amino)-2-azabicyclo[2.1.1]hexane-2-carboxylate

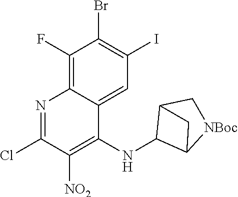 [SMILES: CC(C)(C)OC(=O)N1CC2CC1C2Nc1c([N+](=O)[O-])c(Cl)nc2c(F)c(Br)c(I)cc12]

To a solution of 7-bromo-2,4-dichloro-8-fluoro-6-iodo-3-nitroquinoline (15 g, 32.2 mmol) and tert-butyl 5-amino-2-azabicyclo[2.1.1]hexane-2-carboxylate (6.38 g, 32.2 mmol) in NMP (100 ml) was added DIPEA (8.44 ml, 48.3 mmol) and the reaction mixture was heated to 60° C. for 1 h. Water (100 mL) was added and the suspension was stirred for 15 min. The solids were filtered, rinsed with water, and air dried to afford the title compound (19.9 g, 98%). LC-MS calculated for C₁₉H₁₉BrClFIN₄O₄⁺ (M+H)⁺: m/z=626.9; found 626.9.

### Step 6. tert-butyl 5-((7-bromo-8-fluoro-6-iodo-2-(((S)-1-methylpyrrolidin-2-yl)methoxy)-3-nitroquinolin-4-yl)amino)-2-azabicyclo[2.1.1]hexane-2-carboxylate

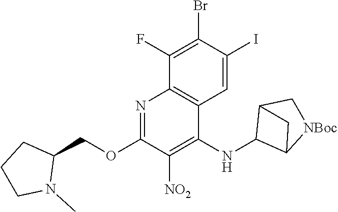 [SMILES: CN1CCC[C@H]1COc1nc2c(F)c(Br)c(I)cc2c(NC2C3CC2N(C(=O)OC(C)(C)C)C3)c1[N+](=O)[O-]]

To a suspension of sodium hydride (2.54 g, 63.4 mmol) in THF (200 ml) at 0° C. was added (S)-(1-methylpyrrolidin-2-yl)methanol (9.43 ml, 79.0 mmol), and the mixture was stirred at 0° C. for 30 min. tert-butyl 5-((7-bromo-2-chloro-8-fluoro-6-iodo-3-nitroquinolin-4-yl)amino)-2-azabicyclo[2.1.1]hexane-2-carboxylate (19.9 g, 31.7 mmol) was added in portions as a solid over 15 min., and the reaction mixture was allowed to warm to room temperature. The reaction mixture was partitioned between saturated NH₄Cl and EtOAc, and the layers were separated. The aqueous layer was extracted with EtOAc, and the combined organic layers were washed with brine, dried over MgSO₄, filtered, and concentrated. The product was used without purification. LC-MS calculated for C₂₅H₃₁BrFIN₅O₅ (M+H)⁺=706.1; found 706.2.

### Step 7. tert-butyl 5-((7-bromo-8-fluoro-6-iodo-2-(((S)-1-methylpyrrolidin-2-yl)methoxy)-3-nitroquinolin-4-yl)(tert-butoxycarbonyl)amino)-2-azabicyclo[2.1.1]hexane-2-carboxylate

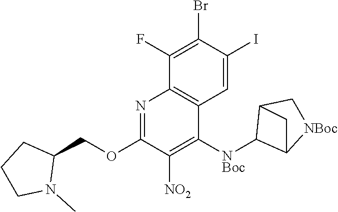 [SMILES: CN1CCC[C@H]1COc1nc2c(F)c(Br)c(I)cc2c(N(C(=O)OC(C)(C)C)C2C3CC2N(C(=O)OC(C)(C)C)C3)c1[N+](=O)[O-]]

To a solution of tert-butyl 5-((7-bromo-8-fluoro-6-iodo-2-(((S)-1-methylpyrrolidin-2-yl)methoxy)-3-nitroquinolin-4-yl)amino)-2-azabicyclo[2.1.1]hexane-2-carboxylate (22 g, 31.1 mmol) in THF (200 ml) was added triethylamine (10.9 ml, 78 mmol), DMAP (0.38 g, 3.11 mmol), and di-tert-butyl dicarbonate (13.6 g, 62.3 mmol) sequentially at room temperature. After 3 h, the reaction mixture was diluted with EtOAc, then washed with saturated NaHCO₃ and brine. The organic layer was dried over MgSO₄, filtered, and concentrated. The product was used without purification. LC-MS calculated for C₃₀H₃₉BrFIN₅O₇ (M+H)⁺=806.1; found 806.2.

### Step 8. tert-butyl 5-((3-amino-7-bromo-8-fluoro-6-iodo-2-(((S)-1-methylpyrrolidin-2-yl)methoxy)quinolin-4-yl)(tert-butoxycarbonyl)amino)-2-azabicyclo[2.1.1]hexane-2-carboxylate

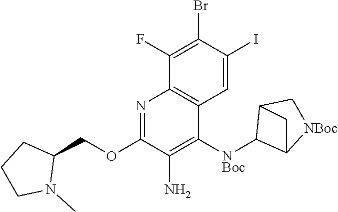 [SMILES: CN1CCC[C@H]1COc1nc2c(F)c(Br)c(I)cc2c(N(C(=O)OC(C)(C)C)C2C3CC2N(C(=O)OC(C)(C)C)C3)c1N]

A 1 L, 3-necked flask equipped with a mechanical stirrer was charged with tert-butyl 5-((7-bromo-8-fluoro-6-iodo-2-(((S)-1-methylpyrrolidin-2-yl)methoxy)-3-nitroquinolin-4-yl)(tert-butoxycarbonyl)amino)-2-azabicyclo[2.1.1]hexane-2-carboxylate (25 g, 31.0 mmol), followed by MeOH (75 ml), water (75 ml), and THF (75 ml). Iron (8.66 g, 155 mmol) and ammonium chloride (8.29 g, 155 mmol) were added, and the reaction mixture was stirred at 70° C. for 6 h. The reaction mixture was diluted with EtOAc and filtered through a pad of celite. The layers were separated and the organic layer was washed with brine, dried over MgSO₄, filtered and concentrated. The product was used without purification. LC-MS calculated for C₃₀H₄₁BrFIN₅O₅ (M+H)⁺=776.1; found 776.2.

### Step 9. tert-butyl 5-((3-amino-7-bromo-6-((E)-2-cyanovinyl)-8-fluoro-2-(((S)-1-methylpyrrolidin-2-yl)methoxy)quinolin-4-yl)(tert-butoxycarbonyl)amino)-2-azabicyclo[2.1.1]hexane-2-carboxylate

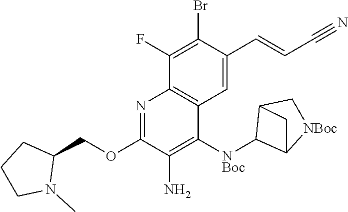 [SMILES: CN1CCC[C@H]1COc1nc2c(F)c(Br)c(/C=C/C#N)cc2c(N(C(=O)OC(C)(C)C)C2C3CC2N(C(=O)OC(C)(C)C)C3)c1N]

A mixture of tert-butyl 5-((3-amino-7-bromo-8-fluoro-6-iodo-2-(((S)-1-methylpyrrolidin-2-yl)methoxy)quinolin-4-yl)(tert-butoxycarbonyl)amino)-2-azabicyclo[2.1.1]hexane-2-carboxylate (5 g, 6.44 mmol), PdOAc₂ (0.15 g, 0.64 mmol), and tri-o-tolylphosphine (0.39 g, 1.29 mmol) was dissolved in DMF (50 ml). TEA (1.80 ml, 12.88 mmol) and acrylonitrile (0.85 ml, 12.9 mmol) were added to the reaction mixture in one portion. The headspace was purged with nitrogen and the reaction mixture was stirred at 80° C. for two hours. The reaction mixture was partitioned between water and EtOAc, and the layers were separated. The aqueous layer was extracted with EtOAc and the combined organic layers were washed with brine, dried over MgSO₄, filtered, and concentrated. The product was used without purification. LC-MS calculated for C₃₃H₄₃BrFN₆O₅ (M+H)⁺=701.2; found 701.3.

### Step 10. tert-butyl 5-((3-amino-7-bromo-6-(2-cyanoethyl)-8-fluoro-2-(((S)-1-methylpyrrolidin-2-yl)methoxy)quinolin-4-yl)(tert-butoxycarbonyl)amino)-2-azabicyclo[2.1.1]hexane-2-carboxylate

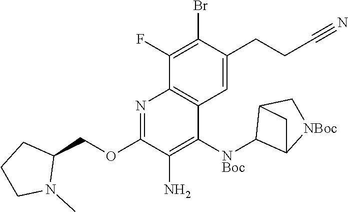 [SMILES: CN1CCC[C@H]1COc1nc2c(F)c(Br)c(CCC#N)cc2c(N(C(=O)OC(C)(C)C)C2C3CC2N(C(=O)OC(C)(C)C)C3)c1N]

tert-Butyl 5-((3-amino-7-bromo-2-cyanovinyl)-8-fluoro-2-(((S)-1-methylpyrrolidin-2-yl)methoxy)quinolin-4-yl)(tert-butoxycarbonyl)amino)-2-azabicyclo[2.1.1]hexane-2-carboxylate (4.5 g, 6.4 mmol) was taken up in THF (50 ml) and cooled to 0° C. Lithium triethylborohydride (1M/THF, 12.9 ml, 12.9 mmol) was added dropwise via additional funnel, and the reaction mixture was stirred at this temperature for 20 min. MeOH and water were added dropwise at 0° C., then the reaction mixture was warmed to room temperature and stirred for 15 min. The product was extracted with EtOAc. The combined organic layers were washed with brine, dried over MgSO₄, filtered, and concentrated. The product was used without purification. LC-MS calculated for C₃₃H₄₅BrFN₆O₅ (M+H)⁺=703.3; found 703.3.

### Step 11. tert-butyl 5-((7-bromo-6-(2-cyanoethyl)-8-fluoro-2-(((S)-1-methylpyrrolidin-2-yl)methoxy)quinolin-4-yl)(tert-butoxycarbonyl)amino)-2-azabicyclo[2.1.1]hexane-2-carboxylate

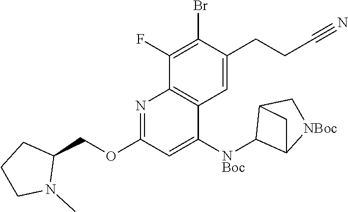 [SMILES: CN1CCC[C@H]1COc1cc(N(C(=O)OC(C)(C)C)C2C3CC2N(C(=O)OC(C)(C)C)C3)c2cc(CCC#N)c(Br)c(F)c2n1]

To a solution of tert-butyl 5-((3-amino-7-bromo-6-(2-cyanoethyl)-8-fluoro-2-(((S)-1-methylpyrrolidin-2-yl)methoxy)quinolin-4-yl)(tert-butoxycarbonyl)amino)-2-azabicyclo[2.1.1]hexane-2-carboxylate (4.8 g, 6.8 mmol) in AcOH (70 ml) and THF (20 ml) at 0° C. was added tert-butylnitrite (4.06 ml, 34.1 mmol). The reaction was allowed to warm to room temperature and stir for 1 h. The reaction mixture was partitioned between water and EtOAc, and the layers were separated. The aqueous layer was extracted with EtOAc and the combined organic layers were washed with brine, dried over MgSO₄, filtered, and concentrated. The product was used without purification. LC-MS calculated for C₃₃H₄₄BrFN₅O₅ (M+H)⁺=688.2; found 688.4.

### Step 12. 3-(4-((2-azabicyclo[2.1.1]hexan-5-yl)amino)-7-bromo-8-fluoro-2-(((S)-1-methylpyrrolidin-2-yl)methoxy)quinolin-6-yl)propanenitrile

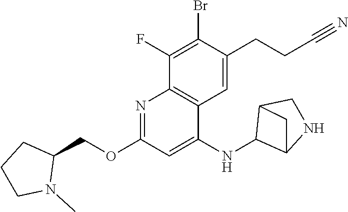 [SMILES: CN1CCC[C@H]1COc1cc(NC2C3CNC2C3)c2cc(CCC#N)c(Br)c(F)c2n1]

To a mixture of tert-butyl 5-((7-bromo-6-(2-cyanoethyl)-8-fluoro-2-(((S)-1-methylpyrrolidin-2-yl)methoxy)quinolin-4-yl)(tert-butoxycarbonyl)amino)-2-azabicyclo[2.1.1]hexane-2-carboxylate (4.7 g, 6.8 mmol) in DCM (60 ml) was added TFA (30 ml, 389 mmol) at 0° C. The reaction mixture was warmed to room temperature and stirred for 1 h. The reaction mixture was concentrated and the product was used without purification. LC-MS calculated for C₂₃H₂₈BrFN₅O (M+H)⁺=488.1; found 488.1.

### Step 13. tert-butyl 5-((7-bromo-6-(2-cyanoethyl)-8-fluoro-2-(((S)-1-methylpyrrolidin-2-yl)methoxy)quinolin-4-yl)amino)-2-azabicyclo[2.1.1]hexane-2-carboxylate

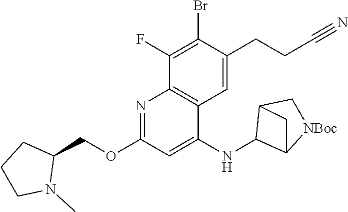 [SMILES: CN1CCC[C@H]1COc1cc(NC2C3CC2N(C(=O)OC(C)(C)C)C3)c2cc(CCC#N)c(Br)c(F)c2n1]

3-(4-((2-Azabicyclo[2.1.1]hexan-5-yl)amino)-7-bromo-8-fluoro-2-(((S)-1-methylpyrrolidin-2-yl)methoxy)quinolin-6-yl)propanenitrile (3.3 g, 6.8 mmol) was suspended in DCM (60 ml) and triethylamine (4.8 ml, 34.1 mmol) was added, resulting in a red solution.

A solution of Boc-anhydride (1.49 g, 6.83 mmol) in DCM (10 mL) was added and the reaction mixture was stirred at room temperature for 30 min. The reaction was quenched with saturated NaHCO₃ and extracted with DCM ×2. The layers were separated and the organic layer was washed with brine, dried over MgSO₄, filtered and concentrated. The residue was purified by flash chromatography (0-10-30% MeOH/DCM) to afford the title compound (1.6 g, 40% over 5 steps). LC-MS calculated for C₂₈H₃₆BrFNsO₃ (M+H)⁺=588.2; found 588.3.

### Step 14. tert-butyl 5-((6-(2-cyanoethyl)-8-fluoro-7-(3-(methoxymethoxy)naphthalen-1-yl)-2-(((S)-1-methylpyrrolidin-2-yl)methoxy)quinolin-4-yl)amino)-2-azabicyclo[2.1.1]hexane-2-carboxylate

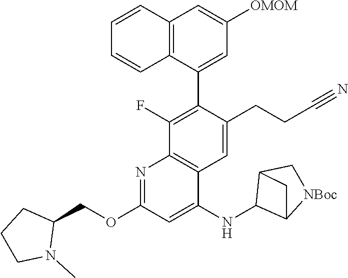

A solution of tert-butyl 5-((7-bromo-6-(2-cyanoethyl)-8-fluoro-2-(((S)-1-methylpyrrolidin-2-yl)methoxy)quinolin-4-yl)amino)-2-azabicyclo[2.1.1]hexane-2-carboxylate (523 mg, 0.89 mmol), 2-(3-(methoxymethoxy)naphthalen-1-yl)-4,4,5,5-tetramethyl-1,3,2-dioxaborolane (335 mg, 1.07 mmol), Pd(PPh₃)₄ (51.3 mg, 0.04 mmol), and sodium carbonate (283 mg, 2.67 mmol) in dioxane (6 ml) and Water (1.5 ml) was sparged with N₂ and heated to 100° C. for 2 h. The reaction mixture was partitioned between water and EtOAc, and the layers were separated. The aqueous layer was extracted with EtOAc and the combined organic layers were washed with brine, dried over MgSO₄, filtered, and concentrated. The residue was purified by flash chromatography (0-10-30% MeOH/DCM) to afford the title compound (253 mg, 41%) as a beige solid. LC-MS calculated for C₄₀H₄₇FN₅O₅ (M+H)⁺=696.4; found 696.5.

### Step 15. tert-butyl 5-((6-(2-cyanoethyl)-8-fluoro-3-iodo-7-(3-(methoxymethoxy)naphthalen-1-yl)-2-(((S)-1-methylpyrrolidin-2-yl)methoxy)quinolin-4-yl)amino)-2-azabicyclo[2.1.1]hexane-2-carboxylate

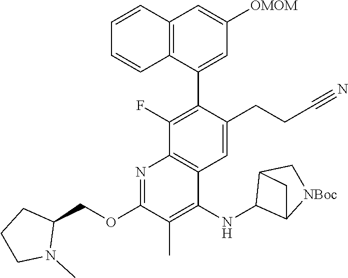

To a solution of tert-butyl 5-((6-(2-cyanoethyl)-8-fluoro-7-(3-(methoxymethoxy)-naphthalen-1-yl)-2-(((S)-1-methylpyrrolidin-2-yl)methoxy)quinolin-4-yl)amino)-2-azabicyclo[2.1.1]hexane-2-carboxylate (194 mg, 0.28 mmol) in DCM (6 mL) was added silver trifluoroacetate (92 mg, 0.42 mmol), and the reaction mixture was cooled to 0° C.

Iodine monochloride (1M/THF, 0.28 mL, 0.28 mmol) was added and stirring was continued at this temperature for 30 min. The reaction was quenched with saturated Na₂S₂O₃ and diluted with EtOAc and water. The layers were separated and the organic layer was washed with brine, dried over MgSO₄, filtered, and concentrated. The product was purified by flash chromatography (0-10-30% MeOH/DCM) to afford the title compound (176 mg, 77%) as a beige solid. LC-MS calculated for C₄₀H₄₆FIN₅O₅ (M+H)⁺=822.2; found 822.4.

### Step 16. tert-butyl 5-((6-(2-cyanoethyl)-8-fluoro-3-(5-methoxy-5-oxopent-1-yn-1-yl)-7-(3-(methoxymethoxy)naphthalen-1-yl)-2-(((S)-1-methylpyrrolidin-2-yl)methoxy)quinolin-4-yl)amino)-2-azabicyclo[2.1.1]hexane-2-carboxylate

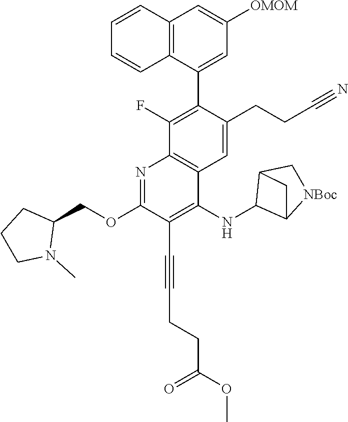

To a mixture of tert-butyl 5-((6-(2-cyanoethyl)-8-fluoro-3-iodo-7-(3-(methoxymethoxy)naphthalen-1-yl)-2-(((S)-1-methylpyrrolidin-2-yl)methoxy)quinolin-4-yl)amino)-2-azabicyclo[2.1.1]hexane-2-carboxylate (33 mg, 0.040 mmol), methyl pent-4-ynoate (15 μl, 0.12 mmol), Pd(PPh₃)₄ (2.3 mg, 2.0 μmol), and copper(I) iodide (3.8 mg, 0.02 mmol) in THF (2 ml) was added triethylamine (0.11 mL, 0.80 mmol) and the reaction mixture was stirred at 80° C. overnight. The reaction mixture was concentrated and the residue was purified by flash chromatography (0-10% MeOH/DCM) to afford the title compound (32 mg, quant.) as a yellow oil. LC-MS calculated for C₄₆H₅₃FN₅O₇ (M+H)⁺=806.4; found 806.5.

### Step 17. tert-butyl 5-(8-(2-cyanoethyl)-6-fluoro-2-(3-methoxy-3-oxopropyl)-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate

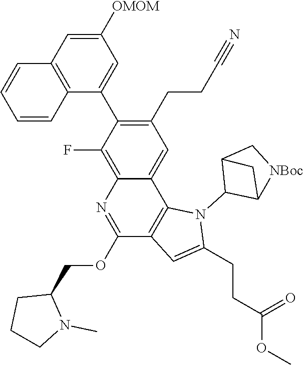

To a 40 mL reaction vial containing tert-butyl 5-((6-(2-cyanoethyl)-8-fluoro-3-(5-methoxy-5-oxopent-1-yn-1-yl)-7-(3-(methoxymethoxy)naphthalen-1-yl)-2-(((S)-1-methylpyrrolidin-2-yl)methoxy)quinolin-4-yl)amino)-2-azabicyclo[2.1.1]hexane-2-carboxylate (32 mg, 0.04 mmol) was added 1,3-bis(2,6-diisopropylphenyl-imidazol-2-ylidene)gold(I) chloride (4.9 mg, 7.9 μmol) and silver hexafluoroantimonate (2.7 mg, 7.9 μmol). The vial was evacuated and backfilled with nitrogen, and THF (3 ml) was added. The reaction mixture was heated to 70° C. for 1 h, then cooled and filtered through a thiol siliaprep cartridge. The solution was concentrated and the product was used without purification. LC-MS calculated for C₄₆H₅₃FN₅O₇ (M+H)⁺=806.4; found 806.5.

### Step 18. Methyl 3-(1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinolin-2-yl)propanoate

tert-Butyl 5-(8-(2-cyanoethyl)-6-fluoro-2-(3-methoxy-3-oxopropyl)-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate (32 mg, 0.04 mmol) was dissolved in DCM (2 mL) and treated with TFA (1.5 mL). The reaction mixture was stirred for 1 h, concentrated, and purified by prep HPLC to afford the title compound (peak 1: 8 mg, 31%). LC-MS calculated for C₃₉H₄₁FN₅O₄ (M+H)⁺=662.3; found 662.3.

The compounds in the following table were synthesized according to the procedure described for Example 29, utilizing the appropriate alkyne in Step 16.

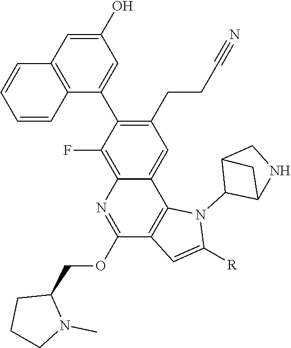

| Example | R | LC-MS |
|---|---|---|
| 30. 3-(1-(2-azabicyclo[2.1.1]hexan- 5-yl)-8-(2-cyanoethyl)-6-fluoro-7-(3- hydroxynaphthalen-1-yl)-4-(((S)-1- methylpyrrolidin-2-yl)methoxy)-1H- pyrrolo[3,2-c]quinolin-2-yl)-N,N- dimethylpropanamide | 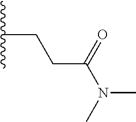 [SMILES: *CCC(=O)N(C)C] | LC-MS calculated for C₄₀H₄₄FN₆O₃ (M + H)⁺ = 675.3; found 675.5. |
| 31. 3-(1-(2-azabicyclo[2.1.1]hexan- 5-yl)-6-fluoro-7-(3- hydroxynaphthalen-1-yl)-4-(((S)-1- methylpyrrolidin-2-yl)methoxy)-2- propyl-1H-pyrrolo[3,2-c]quinolin-8- yl)propanenitrile | 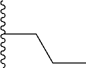 [SMILES: *CCC] | LC-MS calculated for C₃₈H₄₁FN₅O₂ (M + H)⁺ = 618.3; found 618.5. |
| 32. 3-(1-(2-azabicyclo[2.1.1]hexan- 5-y1)-6-fluoro-7-(3- hydroxynaphthalen-1-y1)-2-(1- methyl-1H-pyrazol-4-y1)-4-(((S)-1- methylpyrrolidin-2-yl)methoxy)-1H- pyrrolo[3,2-c]quinolin-8- yl)propanenitrile | 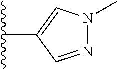 [SMILES: *c1cnn(C)c1] | LC-MS calculated for C₃₉H₃₉FN₇O₂ (M + H)⁺ = 656.3; found 656.4. |

### Example 33. 3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile

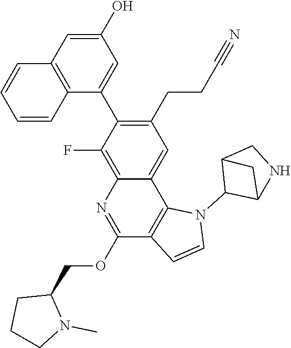 [SMILES: CN1CCC[C@H]1COc1nc2c(F)c(-c3cc(O)cc4ccccc34)c(CCC#N)cc2c2c1ccn2C1C2CNC1C2]

### Step 1. tert-butyl 5-((7-bromo-6-(2-cyanoethyl)-8-fluoro-3-iodo-2-(((S)-1-methylpyrrolidin-2-yl)methoxy)quinolin-4-yl)(tert-butoxycarbonyl)amino)-2-azabicyclo[2.1.1]hexane-2-carboxylate

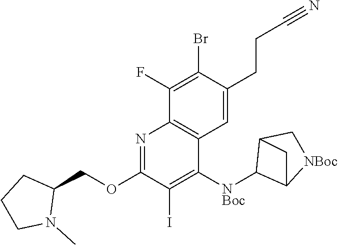 [SMILES: CN1CCC[C@H]1COc1nc2c(F)c(Br)c(CCC#N)cc2c(N(C(=O)OC(C)(C)C)C2C3CC2N(C(=O)OC(C)(C)C)C3)c1I]

To a solution of tert-butyl 5-((3-amino-7-bromo-6-(2-cyanoethyl)-8-fluoro-2-(((S)-1-methylpyrrolidin-2-yl)methoxy)quinolin-4-yl)(tert-butoxycarbonyl)amino)-2-azabicyclo[2.1.1]hexane-2-carboxylate (1.08 g, 1.54 mmol) and potassium iodide (1.27 g, 7.67 mmol) in propionic acid (10 ml) and water (2.5 ml) at −10° C. was added tert-butylnitrite, (0.91 ml, 7.67 mmol) and the reaction mixture was stirred at −10° C. for 1.5 h. The reaction was quenched with saturated Na₂S₂O₃ and extracted with EtOAc. The layers were separated and the organic layer was washed with brine, dried over MgSO₄, filtered and concentrated. The residue was purified by flash chromatography (0-5-15% MeOH/DCM) to afford the title compound (665 mg, 53%) as a brown solid. LC-MS calculated for C₃₃H₄₃BrFIN₅O₅ (M+H)⁺=814.1; found 814.2.

### Step 2. 3-(1-(2-azabicyclo[2.1.1]hexan-5-yl)-7-bromo-6-fluoro-4-(((S)-1-methylpyrrolidin-2-yl)methoxy)-1H-pyrrolo[3,2-c]quinolin-8-yl) propanenitrile

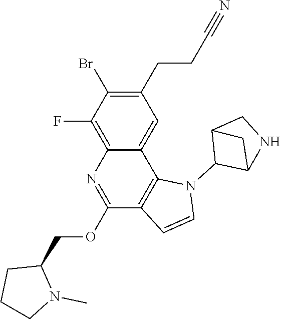 [SMILES: CN1CCC[C@H]1COc1nc2c(F)c(Br)c(CCC#N)cc2c2c1ccn2C1C2CNC1C2]

A mixture of tert-butyl 5-((7-bromo-6-(2-cyanoethyl)-8-fluoro-3-iodo-2-(((S)-1-methylpyrrolidin-2-yl)methoxy)quinolin-4-yl)(tert-butoxycarbonyl)amino)-2-azabicyclo[2.1.1]hexane-2-carboxylate (300 mg, 0.37 mmol), (E)-2-(2-ethoxyvinyl)-4,4,5,5-tetramethyl-1,3,2-dioxaborolane (109 mg, 0.55 mmol), Pd(PPh₃)₄ (42.6 mg, 0.04 mmol) and sodium carbonate (117 mg, 1.11 mmol) in dioxane (3 ml) and water (1 ml) was sparged with N₂ and heated to 80° C. for 1 h. The reaction mixture was partitioned between water and EtOAc, and the layers were separated. The aqueous layer was extracted with EtOAc and the combined organic layers were washed with brine, dried over MgSO₄, filtered, and concentrated.

The residue was dissolved in DCM (3 mL) and treated with TFA (2 mL). The reaction mixture was stirred at room temp for 1.5 h and concentrated. The product was used without purification. LC-MS calculated for C₂₅H₂₈BrFN₅O (M+H)⁺=512.1; found 512.3.

### Step 3. tert-butyl 5-(7-bromo-8-(2-cyanoethyl)-6-fluoro-4-(((S)-1-methylpyrrolidin-2-yl)methoxy)-1H-pyrrolo[3,2-c]quinolin-1-yl)-2-azabicyclo[2.1.1]hexane-2-carboxylate

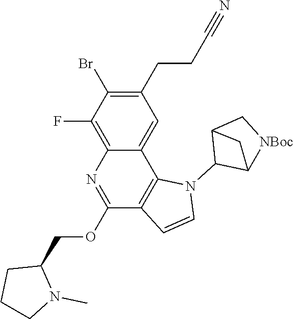 [SMILES: CN1CCC[C@H]1COc1nc2c(F)c(Br)c(CCC#N)cc2c2c1ccn2C1C2CC1N(C(=O)OC(C)(C)C)C2]

To a solution of 2-azabicyclo[2.1.1]hexan-5-yl)-7-bromo-6-fluoro-4-(((S)-1-methylpyrrolidin-2-yl)methoxy)-1H-pyrrolo[3,2-c]quinolin-8-yl)propanenitrile (189 mg, 0.37 mmol) in THF (4 ml) and water (1 mL) was added di-tert-butyl dicarbonate (121 mg, 0.55 mmol) and sodium bicarbonate (155 mg, 1.844 mmol). The reaction mixture was stirred for 1 h and quenched with saturated NaHCO₃. The product was extracted with EtOAc and the organic layer was dried over MgSO₄, filtered, and concentrated. The residue was purified by flash chromatography (0-10% MeOH/DCM) to afford the title compound (207 mg, 92% over 3 steps). LC-MS calculated for C₃₀H₃₆BrFN₅O₃ (M+H)⁺=612.2; found 612.3.

### Step 4. tert-butyl 5-(8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate

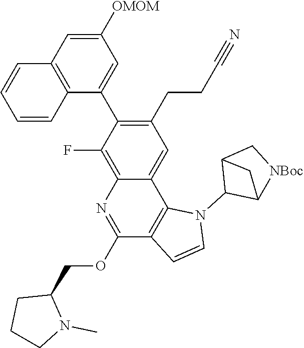

A solution of tert-butyl 5-(7-bromo-8-(2-cyanoethyl)-6-fluoro-4-(((S)-1-methylpyrrolidin-2-yl)methoxy)-1H-pyrrolo[3,2-c]quinolin-1-yl)-2-azabicyclo[2.1.1]hexane-2-carboxylate (207 mg, 0.34 mmol), 2-(3-(methoxymethoxy)naphthalen-1-yl)-4,4,5,5-tetramethyl-1,3,2-dioxaborolane (159 mg, 0.51 mmol), chloro[(tri-tert-butylphosphine)-2-(2-aminobiphenyl)] palladium(II) (17.4 mg, 0.03 mmol), and potassium phosphate, dibasic (177 mg, 1.01 mmol) in THF (3 ml) and water (1 ml) was sparged with N₂ and heated to 70° C. overnight. The reaction mixture was partitioned between water and EtOAc, and the layers were separated. The aqueous layer was extracted with EtOAc and the combined organic layers were washed with brine, dried over MgSO₄, filtered, and concentrated. The residue was purified by flash chromatography (0-25% MeOH/DCM) to afford the title compound (76 mg, 31%) as a light yellow solid. LC-MS calculated for C₄₂H₄₇FN₅O₅ (M+H)⁺=720.4; found 720.5.

### Step 5. 3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile

tert-Butyl 5-(8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate (11 mg, 16 μmol) was stirred in DCM (1.5 m) and TFA (1.5 ml) for 1 h and concentrated. The residue was purified by prep HPLC to afford the title compound. LC-MS calculated for C₃₅H₃₅FN₅O₂ (M+H)⁺=576.3; found 576.4.

### Example 34. 3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-phenyl-1H-pyrrolo[3,2-c]quinolin-8-yl)propanenitrile

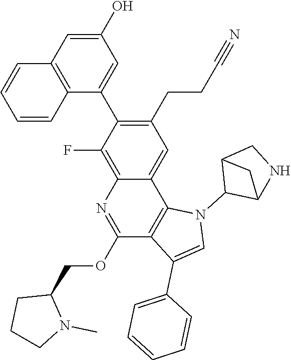 [SMILES: CN1CCC[C@H]1COc1nc2c(F)c(-c3cc(O)cc4ccccc34)c(CCC#N)cc2c2c1c(-c1ccccc1)cn2C1C2CNC1C2]

### Step 1. tert-butyl 5-(3-chloro-8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate

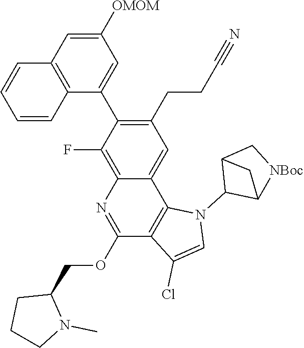

To a solution of tert-butyl 5-(8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate (76 mg, 0.11 mmol, Example 35, Step 4) in DMF (3 ml) was added NCS (14.8 mg, 0.11 mmol) and acetic acid (30 μl, 0.5 mmol). The reaction mixture was heated to 45° C. overnight. More NCS (14.8 mg, 0.11 mmol) and acetic acid (30 μl, 0.5 mmol) was added, and heating was continued for 20 min. The reaction was diluted with EtOAc, and the organic layer was washed with saturated NaHCO₃ and brine. The aqueous layer was extracted with EtOAc and the combined organic layers were dried over MgSO₄, filtered, and concentrated. The product was used without purification. LC-MS calculated for C₄₂H₄₆ClFN₅O₅ (M+H)⁺=754.3; found 754.3.

### Step 2. 3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-phenyl-1H-pyrrolo[3,2-c]quinolin-8-yl)propanenitrile

To a mixture of phenylboronic acid (4.9 mg, 0.04 mmol), XPhos Pd G2 (2.1 mg, 2.7 μmol), and sodium carbonate (4.2 mg, 0.04 mmol) was added a solution of tert-butyl 5-(3-chloro-8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate (10 mg, 0.01 mmol) in dioxane (1 ml). Water (0.3 ml) was added and the reaction mixture was sparged with N₂, then heated to 95° C. for 1 h. The reaction mixture was diluted with EtOAc, filtered through a thiol siliaprep cartridge, and concentrated. The residue was stirred in CM (1.5 ml) and TFA (1.5 ml) for 1 h and concentrated. The residue was purified by prep HPLC to afford the title compound. LC-MS calculated for C₄₁H₃₉FN₅O₂ (M+H)⁺=652.3; found 652.5.

The compounds in the following table were synthesized according to the procedure described for Example 33, utilizing the appropriate boronate or boronic acid in Step 2.

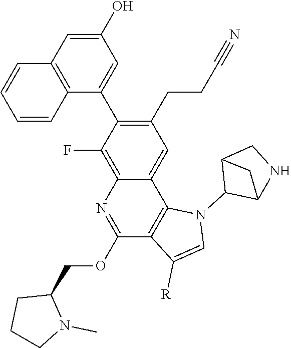

| Example | R | LC-MS |
|---|---|---|
| 35. 3-(1-(2-azabicyclo[2.1.1]hexan-5- yl)-6-fluoro-7-(3-hydroxynaphthalen- 1-yl)-4-(((S)-1-methylpyrrolidin-2- yl)methoxy)-3-(pyridin-3-yl)-1H- pyrrolo[3,2-c]quinolin-8- yl)propanenitrile | 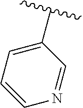 [SMILES: *c1cccnc1] | LC-MS calculated for C₄₀H₃₈FN₆O₂ (M + H)⁺ = 653.3; found 653.2. |
| 36. 3-(1-(2-azabicyclo[2.1.1]hexan-5- yl)-6-fluoro-7-(3-hydroxynaphthalen- 1-yl)-3-(2-methyloxazol-5-yl)-4-(((S)- 1-methylpyrrolidin-2-yl)methoxy)- 1H-pyrrolo[3,2-c]quinolin-8- yl)propanenitrile | 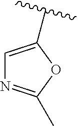 [SMILES: *c1cnc(C)o1] | LC-MS calculated for C₃₉H₃₈FN₆O₃ (M + H)⁺ = 657.3; found 657.4. |
| 37. 3-(1-(2-azabicyclo[2.1.1]hexan-5- yl)-6-fluoro-7-(3-hydroxynaphthalen- 1-yl)-4-(((S)-1-methylpyrrolidin-2- yl)methoxy)-3-(2-methylthiazol-5-yl)- 1H-pyrrolo[3,2-c]quinolin-8- yl)propanenitrile | 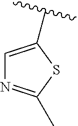 [SMILES: *c1cnc(C)s1] | LC-MS calculated for C₃₉H₃₈FN₆O₂S (M + H)⁺ = 673.3; found 673.4. |
| 38. 3-(1-(2-azabicyclo[2.1.1]hexan-5- yl)-6-fluoro-7-(3-hydroxynaphthalen- 1-yl)-3-(1-methyl-1H-pyrazol-4-yl)-4- (((S)-1-methylpyrrolidin-2- yl)methoxy)-1H-pyrrolo[3,2- c]quinolin-8-yl)propanenitrile | 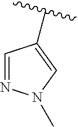 [SMILES: *c1cnn(C)c1] | LC-MS calculated for C₃₉H₃₈FN₇O₂ (M + H)⁺ = 656.3; found 656.5. |

The compounds in the following table were synthesized according to the procedure described for Example 29, utilizing the appropriate alkyne in Step 16.

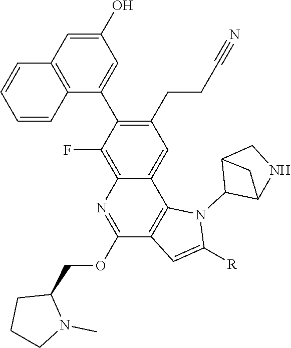

| Example | R | LC-MS |
|---|---|---|
| 39. 3-(1-(2-azabicyclo[2.1.1]hexan-5- yl)-6-fluoro-7-(3-hydroxynaphthalen- 1-yl)-2-(1-methyl-1H-pyrazol-3-yl)-4- (((S)-1-methylpyrrolidin-2- yl)methoxy)-1H-pyrrolo[3,2- c]quinolin-8-yl)propanenitrile | 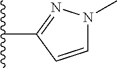 [SMILES: *c1ccn(C)n1] | LC-MS calculated for C₃₉H₃₉FN₇O₂ (M + H)⁺ = 656.3; found 656.5. |
| 40. 3-(2-benzyl-1-(2- azabicyclo[2.1.1]hexan-5-yl)-6- fluoro-7-(3-hydroxynaphthalen-1-yl)- 4-(((S)-1-methylpyrrolidin-2- yl)methoxy)-1H-pyrrolo[3,2- c]quinolin-8-yl)propanenitrile | 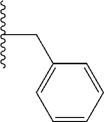 [SMILES: *Cc1ccccc1] | LC-MS calculated for C₄₂H₄₁FN₅O₂ (M + H)⁺ = 666.3; found 666.5. |

The compounds in the following table were synthesized according to the procedure described for Example 33, utilizing the appropriate boronate or boronic acid in Step 2.

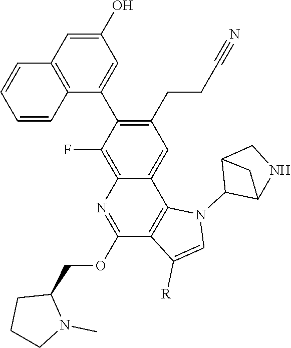

| Example | R | LC-MS |
|---|---|---|
| 41. 3-(1-(2-azabicyclo[2.1.1]hexan-5- yl)-6-fluoro-7-(3-hydroxynaphthalen- 1-yl)-4-(((S)-1-methylpyrrolidin-2- yl)methoxy)-3-(1H-pyrazol-4-yl)-1H- pyrrolo[3,2-c]quinolin-8- yl)propanenitrile | 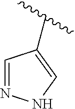 [SMILES: *c1cn[nH]c1] | LC-MS calculated for C₃₈H₃₇FN₇O₂ (M + H)⁺ = 642.3; found 642.3. |
| 42. 3-(1-(2-azabicyclo[2.1.1]hexan-5- yl)-6-fluoro-7-(3-hydroxynaphthalen- 1-yl)-4-(((S)-1-methylpyrrolidin-2- yl)methoxy)-3-(6-oxo-1,6- dihydropyridin-3-yl)-1H-pyrrolo[3,2- c]quinolin-8-yl)propanenitrile | 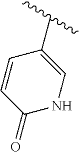 [SMILES: *c1ccc(=O)[nH]c1] | LC-MS calculated for C₄₀H₃₈FN₆O₃ (M + H)⁺ = 669.3; found 669.4. |

### Example 43. 3-(1-(2-azabicyclo[2.1.1]hexan-5-yl)-3-chloro-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile

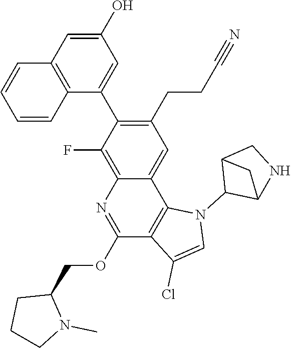 [SMILES: CN1CCC[C@H]1COc1nc2c(F)c(-c3cc(O)cc4ccccc34)c(CCC#N)cc2c2c1c(Cl)cn2C1C2CNC1C2]

tert-Butyl 5-(3-chloro-8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate (Example 34, Step 1) was stirred in DCM (2 mL) and TFA (1 mL) for 1 h and concentrated. The residue was purified by prep HPLC to afford the title compound. LC-MS calculated for C₃₅H₃₄ClFN₅O₂ (M+H)⁺=610.2; found 610.4.

### Example 44. 1-(2-azabicyclo[2.1.1]hexan-5-yl)-8-(2-cyanoethyl)-6-fluoro-N-(2-hydroxyethyl)-7-(3-hydroxynaphthalen-1-yl)-4-(((S)-1-methylpyrrolidin-2-yl)methoxy)-1H-pyrrolo[3,2-c]quinoline-3-carboxamide

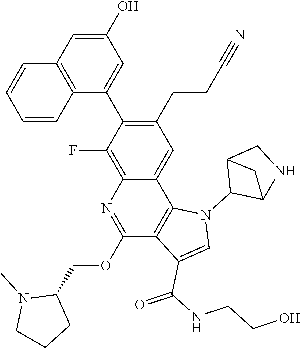 [SMILES: CN1CCC[C@H]1COc1nc2c(F)c(-c3cc(O)cc4ccccc34)c(CCC#N)cc2c2c1c(C(=O)NCCO)cn2C1C2CNC1C2]

### Step 1. tert-butyl 5-(8-(2-cyanoethyl)-6-fluoro-3-iodo-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate

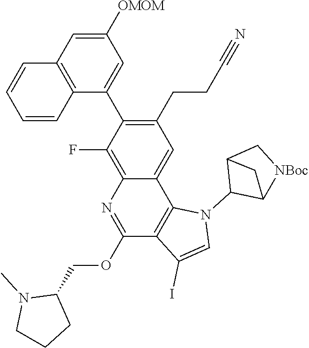

To a mixture of tert-butyl 5-(8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate (258 mg, 0.36 mmol, Example 33, Step 4) and silver trifluoroacetate (119 mg, 0.54 mmol) in THF (5 ml) at 0° C. was added iodine monochloride (0.38 ml, 0.38 mmol) and the reaction mixture was stirred at this temperature for 30 min. The reaction was quenched with saturated Na₂S₂O₃ and diluted with EtOAc. The suspension was filtered through a pad of celite. The layers were separated and the organic layer was washed with brine, dried over MgSO₄, filtered and concentrated. The residue was purified by flash chromatography (0-20% MeOH/DCM) to afford the title compound (302 mg, quant.). LC-MS calculated for C₄₂H₄₆FIN₅O₅ (M+H)⁺=846.2; found 846.1.

### Step 2. 2-(trimethylsilyl)ethyl 1-(2-(tert-butoxycarbonyl)-2-azabicyclo[2.1.1]hexan-5-yl)-8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinoline-3-carboxylate

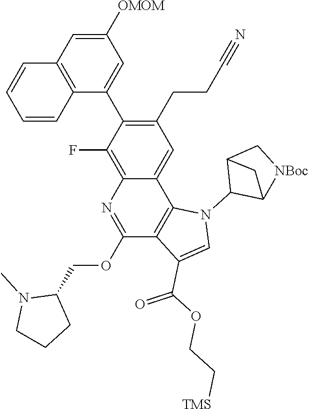

To a solution of tert-butyl 5-(8-(2-cyanoethyl)-6-fluoro-3-iodo-7-(3-(methoxymethoxy)naphthalen-1-yl)-4-(((S)-1-methylpyrrolidin-2-yl)methoxy)-1H-pyrrolo[3,2-c]quinolin-1-yl)-2-azabicyclo[2.1.1]hexane-2-carboxylate (137 mg, 0.08 mmol) and 5 PdCl₂(dppf)-CH₂Cl₂ adduct (6.6 mg, 8.1 μmol) in DMF (2.5 ml) was added 2-(trimethylsilyl)ethan-1-ol (0.5 ml, 3.5 mmol) and triethylamine (0.23 ml, 1.62 mmol). CO was bubbled through the solution for 5 minutes and the reaction mixture was heated to 90° C. under a CO balloon for 2 h. The reaction mixture was diluted with EtOAc and filtered through a thiol siliaprep cartridge. The filtrate was washed with water and brine, dried over MgSO₄, filtered, and concentrated. The product was used without purification. LC-MS calculated for C₄₃H₅₉FN₅O₇Si (M+H)⁺=864.4; found 864.4.

### Step 3. 1-(2-(tert-butoxycarbonyl)-2-azabicyclo[2.1.1]hexan-5-yl)-8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinoline-3-carboxylic acid

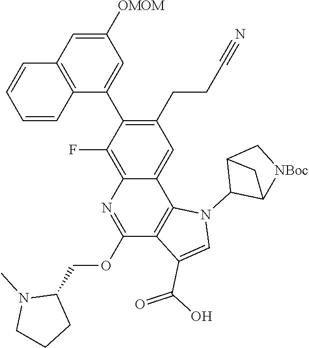

To a solution of 2-(trimethylsilyl)ethyl 1-(2-(tert-butoxycarbonyl)-2-azabicyclo[2.1.1]hexan-5-yl)-8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinoline-3-carboxylate (72 mg, 0.08 mmol) in THF (5 ml) was added TBAF (1M/THF, 0.25 mL, 0.25 mmol), and the reaction mixture was stirred at room temperature overnight. The reaction was quenched with saturated NH₄Cl and extracted with EtOAc twice. The layers were separated and the organic layer was dried over MgSO₄, filtered and concentrated. The product was used without purification. LC-MS calculated for C₄₃H₄₇FN₅O₇ (M+H)⁺=764.3; found 764.5.

### Step 4. 1-(2-azabicyclo[2.1.1]hexan-5-yl)-8-(2-cyanoethyl)-6-fluoro-N-(2-hydroxyethyl)-7-(3-hydroxynaphthalen-1-yl)-4-(((S)-1-methylpyrrolidin-2-yl)methoxy)-1H-pyrrolo[3,2-c]quinoline-3-carboxamide

To a solution of 1-(2-(tert-butoxycarbonyl)-2-azabicyclo[2.1.1]hexan-5-yl)-8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinoline-3-carboxylic acid (12 mg, 0.02 mmol) and HATU (9.0 mg, 0.02 mmol) in DMF (2 ml) was added an excess of 2-aminoethanol, followed by DIPEA (27 μl, 0.16 mmol). The reaction was stirred for 30 min, quenched with water, and extracted with EtOAc. The layers were separated and the organic layer was washed with brine, dried over MgSO₄, filtered and concentrated. LC-MS calculated for C₄₅H₅₂FN₆O₇ (M+H)⁺=807.4; found 807.3.

The residue was stirred in DCM (2 mL) and TFA (1 mL) for 30 min, concentrated, and the product was purified by prep HPLC. LC-MS calculated for C₃₈H₄₀FN₆O₄ (M+H)⁺=663.3; found 663.4.

### Example 45. N-Benzyl-1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinoline-3-carboxamide

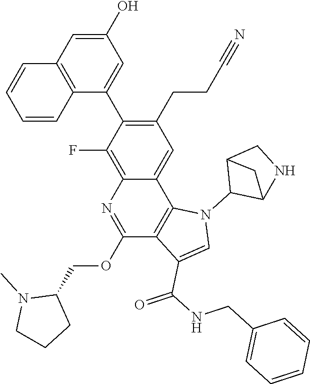 [SMILES: CN1CCC[C@H]1COc1nc2c(F)c(-c3cc(O)cc4ccccc34)c(CCC#N)cc2c2c1c(C(=O)NCc1ccccc1)cn2C1C2CNC1C2]

This compound was prepared according to the procedure described for Example 44, utilizing benzylamine instead of 2-aminoethanol in Step 4. LC-MS calculated for C₄₃H₄₂FN₆O₃ (M+H)⁺=709.3; found 709.2.

### Example 46. 3-(1-(2-Azabicyclo[2.1.1]hexan-5-yl)-6-fluoro-3-(hydroxymethyl)-7-(3-hydroxynaphthalen-1-yl)-4-(((S)-1-methylpyrrolidin-2-yl)methoxy)-1H-pyrrolo[3,2-c]quinolin-8-yl)propanenitrile

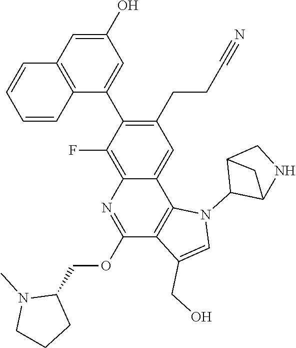 [SMILES: CN1CCC[C@H]1COc1nc2c(F)c(-c3cc(O)cc4ccccc34)c(CCC#N)cc2c2c1c(CO)cn2C1C2CNC1C2]

To a solution of 1-(2-(tert-butoxycarbonyl)-2-azabicyclo[2.1.1]hexan-5-yl)-8-(2-cyanoethyl)-6-fluoro-7-(3-(methoxymethoxy)naphthalen-1-yl)-4-(((S)-1-methylpyrrolidin-2-yl)methoxy)-1H-pyrrolo[3,2-c]quinoline-3-carboxylic acid (13 mg, 17 μmol, Example 44, Step 3) in THF (2 ml) was added oxalyl chloride (2M/DCM, 100 μl, 0.20 mmol) and 1 drop of DMF. The reaction mixture was stirred at room temperature for 15 min. The reaction mixture was cooled to 0° C. and treated with sodium borohydride (64 mg, 1.7 mmol) and a few drops of isopropanol. Upon completion, the excess NaBH₄ was carefully quenched by sequential addition of MeOH and water. Then the reaction mixture was partitioned between water and EtOAc, and the layers were separated. The aqueous layer was extracted with EtOAc and the combined organic layers were washed with brine, dried over MgSO₄, filtered, and concentrated.

The residue was stirred in DCM (2 mL) and TFA (1 mL) for 30 min and concentrated. The product was purified by prep HPLC to afford the title compound. LC-MS calculated for C₃₆H₃₇FN₅O₃ (M+H)⁺=606.3; found 606.4.

### Example 47a and Example 47b. 2-((2S,4S)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)-1-((E)-4-methoxybut-2-enoyl)piperidin-2-yl)acetonitrile

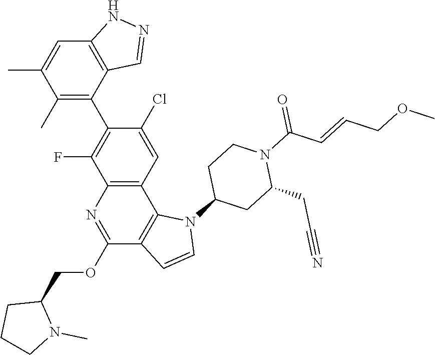 [SMILES: COC/C=C/C(=O)N1CC[C@H](n2ccc3c(OC[C@@H]4CCCN4C)nc4c(F)c(-c5c(C)c(C)cc6[nH]ncc56)c(Cl)cc4c32)C[C@H]1CC#N]

### Step 1: tert-butyl (2S,4S)-4-((7-bromo-6-chloro-8-fluoro-3-formyl-2-(methylthio)quinolin-4-yl)amino)-2-(2-((tert-butyldimethylsilyl)oxy)ethyl)piperidine-1-carboxylate

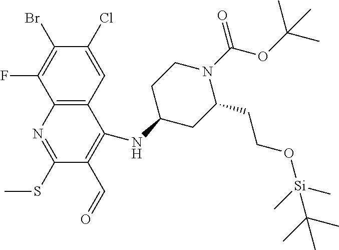 [SMILES: CSc1nc2c(F)c(Br)c(Cl)cc2c(N[C@H]2CCN(C(=O)OC(C)(C)C)[C@H](CCO[Si](C)(C)C(C)(C)C)C2)c1C=O]

To a solution of tert-butyl (2S,4S)-4-((7-bromo-2,6-dichloro-8-fluoro-3-formylquinolin-4-yl)amino)-2-(2-((tert-butyldimethylsilyl)oxy)ethyl)piperidine-1-carboxylate (1.06 g, 1.56 mmol) in MeOH (15.6 mL)/DCM (15.6 mL) was added sodium thiomethoxide (0.33 g, 4.68 mmol) and the resulting mixture was stirred at room temperature for 1 h. The reaction solution was diluted with sat'd NH₄Cl and extracted with EtOAc. The combined organic layers were dried over MgSO₄, filtered, concentrated, and purify by silica gel column (eluting with a gradient of 0-20% EtOAc in Hexanes) to give the desired product as white solid (0.85 g, 79%). LCMS calculated for C₂₉H₄₃BrClFN₃O₄SSi (M+H)⁺ m/z=690.2, 692.2; found 690.2, 692.2.

### Step 2. tert-butyl (2S,4S)-4-((7-bromo-6-chloro-8-fluoro-3-formyl-2-(((S)-1-methylpyrrolidin-2-yl)methoxy)quinolin-4-yl)amino)-2-(2-((tert-butyldimethylsilyl)oxy)ethyl)piperidine-1-carboxylate

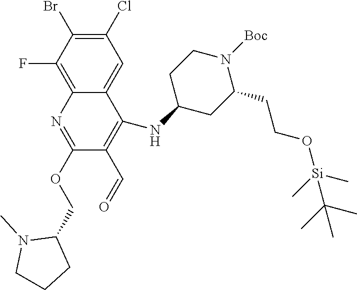 [SMILES: CN1CCC[C@H]1COc1nc2c(F)c(Br)c(Cl)cc2c(N[C@H]2CCN(C(=O)OC(C)(C)C)[C@H](CCO[Si](C)(C)C(C)(C)C)C2)c1C=O]

This compound was prepared according to the procedure described in Example 17a and Example 17b, in Step 1 replacing tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate with tert-butyl (2S,4S)-4-((7-bromo-6-chloro-8-fluoro-3-formyl-2-(methylthio)quinolin-4-yl)amino)-2-(2-((tert-butyldimethylsilyl)oxy)ethyl)piperidine-1-carboxylate. LC-MS calculated for C₃₄H₅₂BrClFN₄O₅Si (M+H)⁺: m/z=757.3, 759.3; found 757.4, 759.4.

### Step 3. tert-butyl (2S,4S)-4-((7-bromo-6-chloro-8-fluoro-3-((E)-2-methoxyvinyl)-2-(((R)-1-methylpyrrolidin-2-yl)methoxy)quinolin-4-yl)amino)-2-(2-((tert-butyldimethylsilyl)oxy)ethyl)piperidine-1-carboxylate

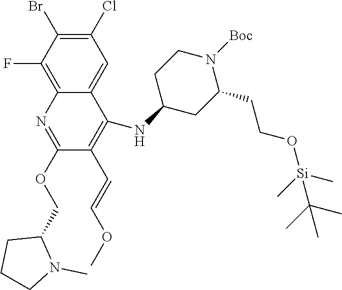 [SMILES: CO/C=C/c1c(OC[C@H]2CCCN2C)nc2c(F)c(Br)c(Cl)cc2c1N[C@H]1CCN(C(=O)OC(C)(C)C)[C@H](CCO[Si](C)(C)C(C)(C)C)C1]

To a solution of (methoxymethyl)triphenylphosphonium chloride (1.222 g, 3.57 mmol) in toluene (10 mL) was added 1.0 M potassium tert-butoxide in THF (3.57 ml, 3.57 mmol) at rt under an atmosphere of nitrogen. After stirring for 30 minutes, a solution of tert-butyl (2S,4S)-4-((7-bromo-6-chloro-8-fluoro-3-formyl-2-(((R)-1-methylpyrrolidin-2-yl)methoxy)quinolin-4-yl)amino)-2-(2-((tert-butyldimethylsilyl)oxy)ethyl)piperidine-1-carboxylate (1.04 g, 1.372 mmol) in THF (10 mL) was cannulated into reaction flask. The resulting solution was stirred at rt for 1h. The reaction was quenched with 1 N HCl and diluted with ethyl acetate. Aqueous layer was extracted with ethyl acetate once. The combined organic solutions were washed with brine, dried over Na₂SO₄, filtered and concentrated. The residue was purified with silica gel chromatography (eluting with a gradient of 0-40% ethyl acetate in hexanes) to give product as white solid (1.07 g, 99%). LC-MS calculated for C₃₆H₅₆BrClFN₄O₅Si (M+H)⁺: m/z=785.3, 787.3; found=785.4, 787.4.

### Step 4. tert-butyl (2S,4S)-4-(7-bromo-8-chloro-6-fluoro-4-(((S)-1-methylpyrrolidin-2-yl)methoxy)-1H-pyrrolo[3,2-c]quinolin-1-yl)-2-(2-hydroxyethyl)piperidine-1-carboxylate

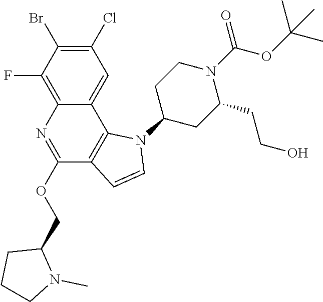 [SMILES: CN1CCC[C@H]1COc1nc2c(F)c(Br)c(Cl)cc2c2c1ccn2[C@H]1CCN(C(=O)OC(C)(C)C)[C@H](CCO)C1]

Into a flask was added tert-butyl (2S,4S)-4-((7-bromo-6-chloro-8-fluoro-3-((E)-2-methoxyvinyl)-2-(((S)-1-methylpyrrolidin-2-yl)methoxy)quinolin-4-yl)amino)-2-(2-((tert-butyldimethylsilyl)oxy)ethyl)piperidine-1-carboxylate (2.0 g, 2.54 mmol), TFA (5.88 ml, 76 mmol), and CH₂Cl₂ (15 ml). The reaction mixture was stirred at room temperature for 1 h. The solvent was removed in vacuo. The residue was dissolved in methanol and Boc-anhydride (0.886 ml, 3.82 mmol) and TEA (1.42 ml, 10.17 mmol) was added and stirred for 2 h. The solvent was removed and residue was purified with silica gel column to give the desired product (1.6 g, 98%). LC-MS calculated for C₂₉H₃₈BrClFN₄O₄ (M+H)⁺: m/z=639.2, 641.2; found 639.3, 641.3.

### Step 5. tert-butyl (2S,4S)-4-(7-bromo-8-chloro-6-fluoro-4-(((S)-1-methylpyrrolidin-2-yl)methoxy)-1H-pyrrolo[3,2-c]quinolin-1-yl)-2-(cyanomethyl)piperidine-1-carboxylate

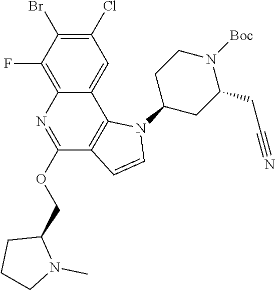 [SMILES: CN1CCC[C@H]1COc1nc2c(F)c(Br)c(Cl)cc2c2c1ccn2[C@H]1CCN(C(=O)OC(C)(C)C)[C@H](CC#N)C1]

This compound was prepared according to the procedure described in Example 3a and Example 3b, in Step 17 replacing of tert-butyl (2S,4S)-4-(7-bromo-8-chloro-6-fluoro-4-(methylthio)-1H-pyrazolo[4,3-c]quinolin-1-yl)-2-(2-hydroxyethyl)piperidine-1-carboxylate with tert-butyl (2S,4S)-4-(7-bromo-8-chloro-6-fluoro-4-(((S)-1-methylpyrrolidin-2-yl)methoxy)-1H-pyrrolo[3,2-c]quinolin-1-yl)-2-(2-hydroxyethyl)piperidine-1-carboxylate. LC-MS calculated for C₂₉H₃₅BrClFN₅O₃ (M+H)⁺: m/z=634.2, 636.2; found 634.3, 636.3.

### Step 6. 5,6-dimethyl-1-(tetrahydro-2H-pyran-2-yl)-4-(4,4,5,5-tetramethyl-1,3,2-dioxaborolan-2-yl)-1H-indazole

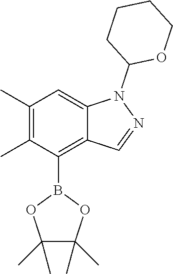 [SMILES: Cc1cc2c(cnn2C2CCCCO2)c(B2OC(C)(C)C(C)(C)O2)c1C]

A mixture of 4,4,5,5,4′,4′,5′,5′,-Octamethyl-[2,2′]bi[[1,3,2]dioxaborolanyl] (0.704 g, 2.77 mmol), potassium acetate (0.378 g, 3.85 mmol), 4-bromo-5,6-dimethyl-1-(tetrahydro-2H-pyran-2-yl)-1H-indazole (0.476 g, 1.539 mmol) and PdCl₂(dppf) (0.113 g, 0.154 mmol) in 1,4-dioxane (4.0 mL). The reaction mixture was degassed with N₂. The mixture was stirred at 105° C. for 3 h. The mixture was diluted with EtOAc and filtered. The filtrate was concentrated and the product purified by silica gel column (eluting with a gradient of 0-20% ethyl acetate in hexanes) to give the desired product as colorless oil (0.47 g, 86%). LC-MS calculated for C₂₀H₃₀BN₂O₃ (M+H)⁺: m/z=357.2; found 357.2.

### Step 7. tert-butyl (2S,4S)-4-(8-chloro-7-(5,6-dimethyl-1-(tetrahydro-2H-pyran-2-yl)-1H-indazol-4-yl)-6-fluoro-4-(((S)-1-methylpyrrolidin-2-yl)methoxy)-1H-pyrrolo[3,2-c]quinolin-1-yl)-2-(cyanomethyl)piperidine-1-carboxylate

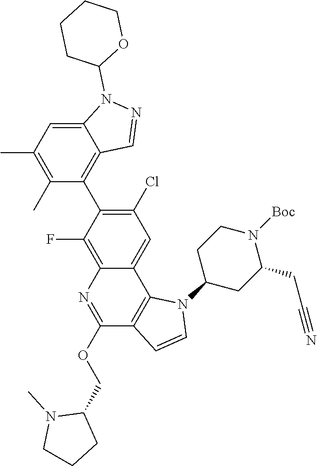 [SMILES: Cc1cc2c(cnn2C2CCCCO2)c(-c2c(Cl)cc3c(nc(OC[C@@H]4CCCN4C)c4ccn([C@H]5CCN(C(=O)OC(C)(C)C)[C@H](CC#N)C5)c43)c2F)c1C]

A microwave vial charged with tert-butyl (2S,4S)-4-(7-bromo-8-chloro-6-fluoro-4-(((S)-1-methylpyrrolidin-2-yl)methoxy)-1H-pyrrolo[3,2-c]quinolin-1-yl)-2-(cyanomethyl)piperidine-1-carboxylate (210 mg, 0.331 mmol), 5,6-dimethyl-1-(tetrahydro-2H-pyran-2-yl)-4-(4,4,5,5-tetramethyl-1,3,2-dioxaborolan-2-yl)-1H-indazole (141 mg, 0.397 mmol), tetrakis(triphenylphosphine)palladium(0) (57.3 mg, 0.050 mmol), and sodium bicarbonate (69.5 mg, 0.827 mmol) were heated in 5:1 Dioxane:water (5 ml) were heated under N2 atmosphere at 105° C. overnight. The mixture was extracted between brine/EtOAc, dried over MgSO₄, and purified by flash chromatography (eluting with a gradient of 0-30% ethyl acetate in hexanes) to give the desired product (135 mg, 68%). LC-MS calculated for C₄₃H₅₂ClFN₇O₄ (M+H)⁺: m/z=784.4; found 784.5.

### Step 8. 2-((2S,4S)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)piperidin-2-yl)acetonitrile

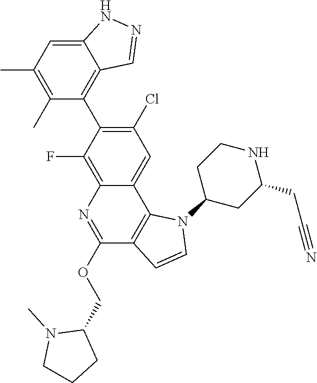 [SMILES: Cc1cc2[nH]ncc2c(-c2c(Cl)cc3c(nc(OC[C@@H]4CCCN4C)c4ccn([C@H]5CCN[C@H](CC#N)C5)c43)c2F)c1C]

This compound was prepared according to the procedure described in Example 3a and Example 3b, in Step 20 replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-4-(8-chloro-7-(5,6-dimethyl-1-(tetrahydro-2H-pyran-2-yl)-1H-indazol-4-yl)-6-fluoro-4-(((S)-1-methylpyrrolidin-2-yl)methoxy)-1H-pyrrolo[3,2-c]quinolin-1-yl)-2-(cyanomethyl) piperidine-1-carboxylate. LC-MS calculated for C₃₃H₃₆ClFN₇O (M+H)⁺: m/z=600.3; found 600.4.

### Step 9. 2-((2S,4S)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)-1-((E)-4-methoxybut-2-enoyl)piperidin-2-yl)acetonitrile

To a solution of (E)-4-methoxybut-2-enoic acid (2.4 mg, 0.020 mmol) and 2-((2S,4S)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)piperidin-2-yl)acetonitrile bis(2,2,2-trifluoroacetate) (14 mg, 0.017 mmol) in DMF (1.0 ml) was added HATU (8.4 mg, 0.022 mmol) and DIEA (14.8 μl, 0.085 mmol). The resulting mixture was stirred at rt for 1 h. The reaction was diluted with methanol and 1 N HCl (0.1 mL) and purified with prep-LCMS (XBridge C18 column, eluting with a gradient of acetonitrile/water containing 0.1% TFA, at flow rate of 60 mL/min) to give the desired products as two peaks.

Diastereomer 1. Peak 1. LC-MS calculated for C₃₈H₄₂ClFN₇O₃ (M+H)⁺: m/z=698.3; found 698.4.

Diastereomer 2. Peak 2. LC-MS calculated for C₃₈H₄₂ClFN₇O₃ (M+H)⁺: m/z=698.3; found 698.4.

### Example 48. 3-(1-((1R,4R,5S)-2-azabicyclo[2.1.1]hexan-5-yl)-8-(2-cyanoethyl)-4-ethoxy-6-fluoro-7-(3-hydroxynaphthalen-1-yl)-1H-pyrrolo[3,2-c]quinolin-2-yl)-N,N-dimethylpropanamide

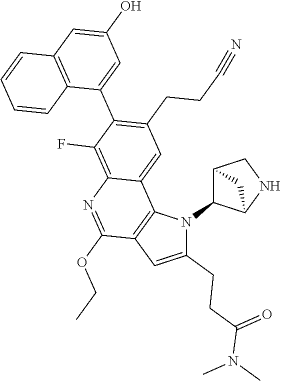 [SMILES: CCOc1nc2c(F)c(-c3cc(O)cc4ccccc34)c(CCC#N)cc2c2c1cc(CCC(=O)N(C)C)n2[C@H]1[C@H]2CN[C@@H]1C2]

### Step 1. tert-butyl (1R,4R,5S)-5-((3-amino-7-bromo-6-(2-cyanoethyl)-8-fluoro-2-methoxyquinolin-4-yl)(tert-butoxycarbonyl)amino)-2-azabicyclo[2.1.1]hexane-2-carboxylate

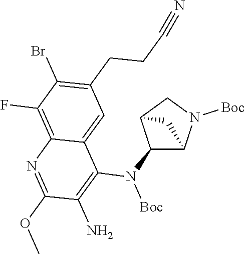 [SMILES: COc1nc2c(F)c(Br)c(CCC#N)cc2c(N(C(=O)OC(C)(C)C)[C@H]2[C@@H]3C[C@H]2N(C(=O)OC(C)(C)C)C3)c1N]

This compound was prepared according to the procedure described in Example 29, replacing (S)-(1-methylpyrrolidin-2-yl)methanol with MeOH. LCMS calculated for C₂₈H₃₆BrFN₅O₅ (M+H)⁺: m/z=620.2; found: 620.2.

### Step 2. tert-butyl (1R,4R,5S)-5-((7-bromo-6-(2-cyanoethyl)-8-fluoro-3-iodo-2-methoxyquinolin-4-yl)amino)-2-azabicyclo[2.1.1]hexane-2-carboxylate

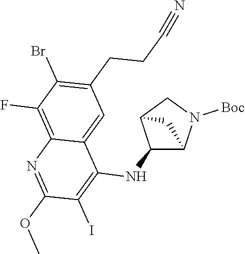 [SMILES: COc1nc2c(F)c(Br)c(CCC#N)cc2c(N[C@H]2[C@@H]3C[C@H]2N(C(=O)OC(C)(C)C)C3)c1I]

To a solution of tert-butyl (1R,4R,5S)-5-((3-amino-7-bromo-6-(2-cyanoethyl)-8-fluoro-2-methoxyquinolin-4-yl)(tert-butoxycarbonyl)amino)-2-azabicyclo[2.1.1]hexane-2-carboxylate (4 g, 6.45 mmol) in AcOH (70 ml) and THF (20 ml) at −10° C. was added potassium iodide (3.21 g, 19.34 mmol) and tert-butylnitrite (2.3 ml, 19.34 mmol). The reaction was stirred at same temperature for 1h. The reaction mixture was quenched with saturated Na₂S₂O₃, partitioned between water and EtOAc, and the layers were separated. The aqueous layer was extracted with EtOAc and the combined organic layers were washed with brine, dried over MgSO₄, filtered, and concentrated. The crude product was dissolved in TFA (10 mL) and DCM (10 mL), after stirring for 1 h, the solvent was removed. The crude material was dissolved in DCM, TEA (1.797 ml, 12.89 mmol) and Boc₂O (2.1 g, 9.67 mmol) were added. The reaction was stirred for 2 h before diluted with water, the organic layer was washed with brine, dried over MgSO₄, filtered, concentrated, and purified by flash chromatography to afford the title compound. LC-MS calculated for C₂₃H₂₆BrFIN₄O₃ (M+H)⁺=631.0; found 631.0.

### Step 3. tert-butyl (1R,4R,5S)-5-((7-bromo-6-(2-cyanoethyl)-8-fluoro-2-methoxy-3-(5-methoxy-5-oxopent-1-yn-1-yl)quinolin-4-yl)amino)-2-azabicyclo[2.1.1]hexane-2-carboxylate

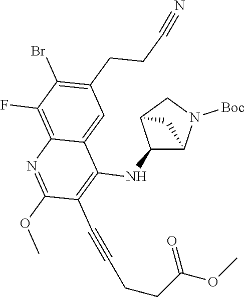 [SMILES: COC(=O)CCC#Cc1c(OC)nc2c(F)c(Br)c(CCC#N)cc2c1N[C@H]1[C@@H]2C[C@H]1N(C(=O)OC(C)(C)C)C2]

To a mixture of tert-butyl (1R,4R,5S)-5-((7-bromo-6-(2-cyanoethyl)-8-fluoro-3-iodo-2-methoxyquinolin-4-yl)amino)-2-azabicyclo[2.1.1]hexane-2-carboxylate (1.5 g, 2.376 mmol), methyl pent-4-ynoate (0.592 ml, 4.75 mmol), bis(triphenylphosphine)palladium(II) dichloride (0.166 g, 0.238 mmol), and copper(I) iodide (0.091 g, 0.475 mmol) in THF (2 ml) was added triethylamine (1.6 ml, 11.88 mmol) and the reaction mixture was stirred at 80° C. for 4h. The reaction mixture was concentrated and the residue was purified by flash chromatography to afford the title compound as a yellow oil. LC-MS calculated for C₂₉H₃₃BrFN₄O₅ (M+H)⁺=615.2; found 615.2.

### Step 4. tert-butyl (1R,4R,5S)-5-(7-bromo-8-(2-cyanoethyl)-6-fluoro-4-methoxy-2-(3-methoxy-3-oxopropyl)-1H-pyrrolo[3,2-c]quinolin-1-yl)-2-azabicyclo[2.1.1]hexane-2-carboxylate

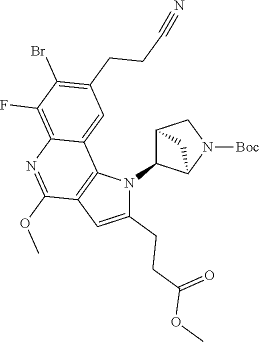 [SMILES: COC(=O)CCc1cc2c(OC)nc3c(F)c(Br)c(CCC#N)cc3c2n1[C@H]1[C@@H]2C[C@H]1N(C(=O)OC(C)(C)C)C2]

To a 40 mL reaction vial containing tert-butyl (1R,4R,5R)-5-((7-bromo-6-(2-cyanoethyl)-8-fluoro-2-methoxy-3-(5-methoxy-5-oxopent-1-yn-1-yl)quinolin-4-yl)amino)-2-azabicyclo[2.1.1]hexane-2-carboxylate (800 mg, 1.3 mmol) was added chloro(triphenylphosphine)gold (I) (32.1 mg, 0.065 mmol) and silver hexafluoroantimonate (44.7 mg, 0.130 mmol). The vial was evacuated and backfilled with nitrogen, and THF (3 ml) was added. The reaction mixture was heated to 70° C. for 1 h, then cooled and filtered through a thiol siliaprep cartridge. The solution was concentrated and the product was used without purification. LC-MS calculated for C₂₉H₃₃BrFN₄O₅ (M+H)⁺=615.1; found 615.1.

### Step 5. tert-butyl (1R,4R,5S)-5-(7-bromo-8-(2-cyanoethyl)-2-(3-(dimethylamino)-3-oxopropyl)-6-fluoro-4-methoxy-1H-pyrrolo[3,2-c]quinolin-1-yl)-2-azabicyclo[2.1.1]hexane-2-carboxylate

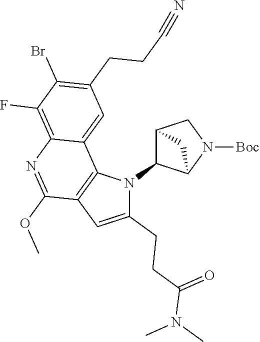 [SMILES: COc1nc2c(F)c(Br)c(CCC#N)cc2c2c1cc(CCC(=O)N(C)C)n2[C@H]1[C@@H]2C[C@H]1N(C(=O)OC(C)(C)C)C2]

To a 40 mL reaction vial containing tert-butyl (1R,4R,5S)-5-(7-bromo-8-(2-cyanoethyl)-6-fluoro-4-methoxy-2-(3-methoxy-3-oxopropyl)-1H-pyrrolo[3,2-c]quinolin-1-yl)-2-azabicyclo[2.1.1]hexane-2-carboxylate (200 mg, 0.325 mmol) in THF (1 mL), MeOH (1 mL), and water (1 mL) was added LiOH (38.9 mg, 1.625 mmol). The reaction mixture was stirred for 1h before quenched with 1N HCl. The mixture was extracted with EtOAc and the organic layer was dried over MgSO₄. The solvent was removed, and the residue was dissolved in THF. To this solution, dimethylamine (0.325 mL, 0.650 mmol), HATU (185 mg, 0.487 mmol) and DIEA (85 μl, 0.487 mmol) were added. The mixture was stirred for 2h before diluted with water. The mixture was extracted with EtOAc and the organic layer was dried over MgSO₄. The solution was concentrated and the product was used without purification. LC-MS calculated for C₃₀H₃₆BrFN₅O₄ (M+H)⁺=628.2; found 628.2.

### Step 6. tert-butyl (1R,4R,5S)-5-(7-bromo-8-(2-cyanoethyl)-2-(3-(dimethylamino)-3-oxopropyl)-6-fluoro-4-hydroxy-1H-pyrrolo[3,2-c]quinolin-1-yl)-2-azabicyclo[2.1.1]hexane-2-carboxylate

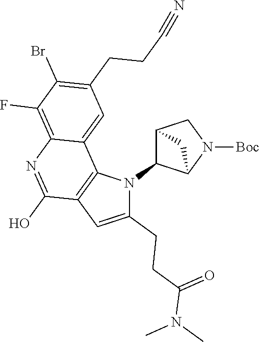 [SMILES: CN(C)C(=O)CCc1cc2c(O)nc3c(F)c(Br)c(CCC#N)cc3c2n1[C@H]1[C@@H]2C[C@H]1N(C(=O)OC(C)(C)C)C2]

To a EtOH (1 mL) solution of tert-butyl (1R,4R,5S)-5-(7-bromo-8-(2-cyanoethyl)-2-(3-(dimethylamino)-3-oxopropyl)-6-fluoro-4-methoxy-1H-pyrrolo[3,2-c]quinolin-1-yl)-2-azabicyclo[2.1.1]hexane-2-carboxylate (150 mg, 0.239 mmol) was added 40% HBr (0.5 mL). The mixture was heated to 70° C. for 30 min, then quenched with 4N NaOH. Sodium bicarbonate (200 mg, 2.386 mmol) and Boc₂O (104 mg, 0.477 mmol) were added to the mixture and stirred for 2h. The mixture was then extracted with EtOAc and the organic layer was dried over MgSO₄. The solution was concentrated and the residue was purified by flash chromatography to afford the title compound as a yellow oil. LC-MS calculated for C₂₉H₃₄BrFN₅O₄ (M+H)⁺=614.2; found 614.2.

### Step 7. tert-butyl (1R,4R,5S)-5-(7-bromo-8-(2-cyanoethyl)-2-(3-(dimethylamino)-3-oxopropyl)-4-ethoxy-6-fluoro-1H-pyrrolo[3,2-c]quinolin-1-yl)-2-azabicyclo[2.1.1]hexane-2-carboxylate

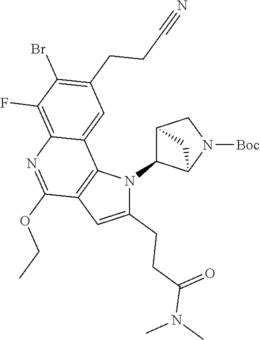 [SMILES: CCOc1nc2c(F)c(Br)c(CCC#N)cc2c2c1cc(CCC(=O)N(C)C)n2[C@H]1[C@@H]2C[C@H]1N(C(=O)OC(C)(C)C)C2]

To a DMF (1 mL) solution of tert-butyl (1R,4R,5S)-5-(7-bromo-8-(2-cyanoethyl)-2-(3-(dimethylamino)-3-oxopropyl)-6-fluoro-4-hydroxy-1H-pyrrolo[3,2-c]quinolin-1-yl)-2-azabicyclo[2.1.1]hexane-2-carboxylate (40 mg, 0.065 mmol) was added Cs₂CO₃ (42.4 mg, 0.130 mmol) and ethyl iodide (10.52 μl, 0.130 mmol). The mixture was heated to 100° C. for 12 h, then diluted with water. The mixture was then extracted with EtOAc and the organic layer was dried over MgSO₄. The solution was concentrated and the residue was purified by flash chromatography to afford the title compound. LC-MS calculated for C₃₁H₃₈BrFN₅O₄ (M+H)⁺=642.2; found 642.2.

### Step 8. 3-(1-((1R,4R,5S)-2-azabicyclo[2.1.1]hexan-5-yl)-8-(2-cyanoethyl)-4-ethoxy-6-fluoro-7-(3-hydroxynaphthalen-1-yl)-1H-pyrrolo[3,2-c]quinolin-2-yl)-N,N-dimethylpropanamide

A solution of tert-butyl (1R,4R,5S)-5-(7-bromo-8-(2-cyanoethyl)-2-(3-(dimethylamino)-3-oxopropyl)-4-ethoxy-6-fluoro-1H-pyrrolo[3,2-c]quinolin-1-yl)-2-azabicyclo[2.1.1]hexane-2-carboxylate (30.0 mg, 0.047 mmol), 4-(4,4,5,5-tetramethyl-1,3,2-dioxaborolan-2-yl)naphthalen-2-ol (12.61 mg, 0.047 mmol) Pd(PPh₃)₄ (5.40 mg, 4.67 μmol), sodium carbonate (9.90 mg, 0.093 mmol) in dioxane (2 ml) and Water (0.5 ml) was sparged with N₂ and heated to 100° C. for 2 h. The reaction mixture was partitioned between water and EtOAc, and the layers were separated. The aqueous layer was extracted with EtOAc and the combined organic layers were washed with brine, dried over MgSO₄, filtered, and concentrated. The residue was dissolved in TFA and diluted with MeOH before purified by prep-LCMS (XBridge C18 column, eluting with a gradient of acetonitrile/water containing 0.1% TFA, at flow rate of 60 mL/min) to give the desired product as a TFA salt. LC-MS calculated for C₃₆H₃₇FN₅O₃ (M+H)⁺=606.3; found 606.3.

### Example 49. methyl 3-(1-((1R,4R,5S)-2-azabicyclo[2.1.1]hexan-5-yl)-8-(2-cyanoethyl)-3-(3-(dimethylamino)azetidin-1-yl)-6-fluoro-7-(3-hydroxynaphthalen-1-yl)-4-methoxy-1H-pyrrolo[3,2-c]quinolin-2-yl)propanoate

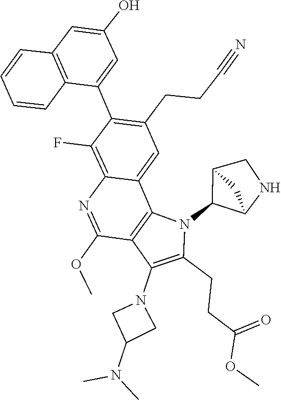 [SMILES: COC(=O)CCc1c(N2CC(N(C)C)C2)c2c(OC)nc3c(F)c(-c4cc(O)cc5ccccc45)c(CCC#N)cc3c2n1[C@H]1[C@H]2CN[C@@H]1C2]

### Step 1. tert-butyl (1R,4R,5S)-5-(8-(2-cyanoethyl)-6-fluoro-4-methoxy-2-(3-methoxy-3-oxopropyl)-7-(3-(methoxymethoxy)naphthalen-1-yl)-1H-pyrrolo[3,2-c]quinolin-1-yl)-2-azabicyclo[2.1.1]hexane-2-carboxylate

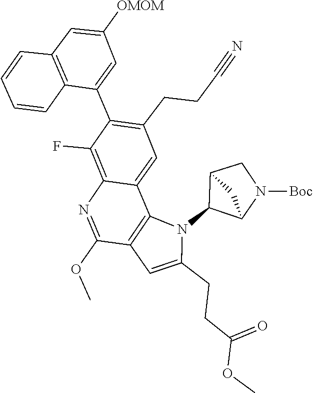

A solution of tert-butyl (1R,4R,5S)-5-(7-bromo-8-(2-cyanoethyl)-6-fluoro-4-methoxy-2-(3-methoxy-3-oxopropyl)-1H-pyrrolo[3,2-c]quinolin-1-yl)-2-azabicyclo[2.1.1]hexane-2-carboxylate (30 mg, 0.048 mmol, From example 48), 2-(3-(methoxymethoxy)naphthalen-1-yl)-4,4,5,5-tetramethyl-1,3,2-dioxaborolane (22.49 mg, 0.072 mmol), Pd(PPh₃)₄ (5.40 mg, 4.67 μmol), sodium carbonate (9.90 mg, 0.093 mmol) in dioxane (2 ml) and Water (0.5 ml) was sparged with N₂ and heated to 100° C. for 2 h. The reaction mixture was partitioned between water and EtOAc, and the layers were separated. The aqueous layer was extracted with EtOAc and the combined organic layers were washed with brine, dried over MgSO₄, filtered, and concentrated and used directly in next step. LC-MS calculated for C₄₁H₄₄FN₄O₇ (M+H)⁺=723.3; found 723.3.

### Step 2. tert-butyl (1R,4R,5S)-5-(3-bromo-8-(2-cyanoethyl)-6-fluoro-4-methoxy-2-(3-methoxy-3-oxopropyl)-7-(3-(methoxymethoxy)naphthalen-1-yl)-1H-pyrrolo[3,2-c]quinolin-1-yl)-2-azabicyclo[2.1.1]hexane-2-carboxylate

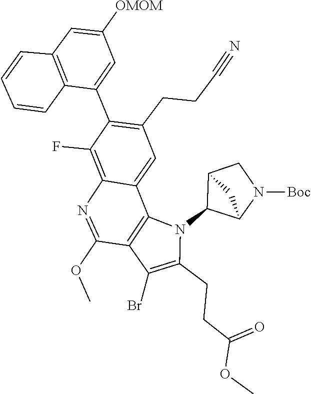

A solution of tert-butyl (1R,4R,5S)-5-(8-(2-cyanoethyl)-6-fluoro-4-methoxy-2-(3-methoxy-3-oxopropyl)-7-(3-(methoxymethoxy)naphthalen-1-yl)-1H-pyrrolo[3,2-c]quinolin-1-yl)-2-azabicyclo[2.1.1]hexane-2-carboxylate (29.4 mg, 0.040 mmol) in DMF (1 mL) was added NBS (7.12 mg, 0.040 mmol). The reaction mixture was then partitioned between water and EtOAc, and the layers were separated. The aqueous layer was extracted with EtOAc and the combined organic layers were washed with brine, dried over MgSO₄, filtered, and concentrated and used directly in next step. LC-MS calculated for C₄₁H₄₃BrFN₄O₇ (M+H)⁺=801.2; found 801.2.

### Step 3. methyl 3-(1-((1R,4R,5S)-2-azabicyclo[2.1.1]hexan-5-yl)-8-(2-cyanoethyl)-3-(3-(dimethylamino)azetidin-1-yl)-6-fluoro-7-(3-hydroxynaphthalen-1-yl)-4-methoxy-1H-pyrrolo[3,2-c]quinolin-2-yl)propanoate

A solution of tert-butyl (1R,4R,5S)-5-(3-bromo-8-(2-cyanoethyl)-6-fluoro-4-methoxy-2-(3-methoxy-3-oxopropyl)-7-(3-(methoxymethoxy)naphthalen-1-yl)-1H-pyrrolo[3,2-c]quinolin-1-yl)-2-azabicyclo[2.1.1]hexane-2-carboxylate (9.67 mg, 0.012 mmol), N,N-dimethylazetidin-3-amine (2.417 mg, 0.024 mmol), Cs₂CO₃ (7.86 mg, 0.024 mmol), and Ruphos PdG2 (2.79 mg, 3.62 μmol) in dioxane (0.5 ml) was sparged with N₂ and heated to 100° C. for 12 h. TFA (1 mL) was added to the reaction, which was then diluted with MeOH before purified by prep-LCMS (XBridge C18 column, eluting with a gradient of acetonitrile/water containing 0.1% TFA, at flow rate of 60 mL/min) to give the desired product as a TFA salt. LC-MS calculated for C₃₉H₄₂FN₆O₄ (M+H)⁺=677.3; found 677.3.

### Example 50. 3-(2-(3-(azetidin-1-yl)-3-oxopropyl)-1-(2-azabicyclo[2.1.1]hexan-5-yl)-6-fluoro-7-(7-fluoronaphthalen-1-yl)-4-(((S)-1-methylpyrrolidin-2-yl)methoxy)-1H-pyrrolo[3,2-c]quinolin-8-yl)propanenitrile

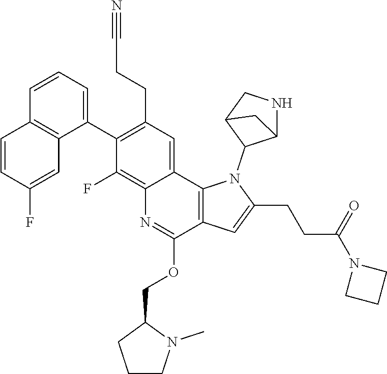 [SMILES: CN1CCC[C@H]1COc1nc2c(F)c(-c3cccc4ccc(F)cc34)c(CCC#N)cc2c2c1cc(CCC(=O)N1CCC1)n2C1C2CNC1C2]

### Step 1. tert-butyl 5-((7-bromo-6-(2-cyanoethyl)-8-fluoro-3-iodo-2-(((S)-1-methylpyrrolidin-2-yl)methoxy)quinolin-4-yl)amino)-2-azabicyclo[2.1.1]hexane-2-carboxylate

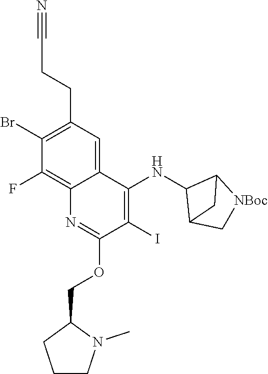 [SMILES: CN1CCC[C@H]1COc1nc2c(F)c(Br)c(CCC#N)cc2c(NC2C3CC2N(C(=O)OC(C)(C)C)C3)c1I]

To a stirred solution of tert-butyl 5-((7-bromo-6-(2-cyanoethyl)-8-fluoro-2-(((S)-1-methylpyrrolidin-2-yl)methoxy)quinolin-4-yl)amino)-2-azabicyclo[2.1.1]hexane-2-carboxylate ((Example 29, Step 13, 4.36 g, 7.41 mmol) and silver trifluoroacetate (2.455 g, 11.11 mmol) in acetic acid (4.25 mL) and DCM (10 mL) at 0° C. was added iodine monochloride (1M solution in DCM, 7.41 mL) dropwise over 3 min. The mixture was stirred for 20 min and then quenched with saturated sodium thiosulfate solution. The mixture was extracted with DCM and then purified by automated FCC (0-50% IPA in DCM) to yield the title compound as a solid (1.89 g, 2.65 mmol, 36%). LC-MS calculated for C₂₈H₃₅BrFINsO₃ (M+H)⁺: m/z=714.1; found 714.2.

### Step 2. tert-butyl 5-((7-bromo-6-(2-cyanoethyl)-8-fluoro-3-(5-methoxy-5-oxopent-1-yn-1-yl)-2-(((S)-1-methylpyrrolidin-2-yl)methoxy)quinolin-4-yl)amino)-2-azabicyclo[2.1.1]hexane-2-carboxylate

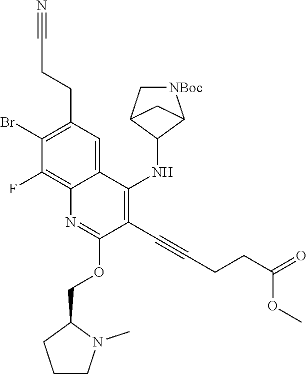 [SMILES: COC(=O)CCC#Cc1c(OC[C@@H]2CCCN2C)nc2c(F)c(Br)c(CCC#N)cc2c1NC1C2CC1N(C(=O)OC(C)(C)C)C2]

To a vial containing tert-butyl 5-((7-bromo-6-(2-cyanoethyl)-8-fluoro-3-iodo-2-(((S)-1-methylpyrrolidin-2-yl)methoxy)quinolin-4-yl)amino)-2-azabicyclo[2.1.1]hexane-2-carboxylate (1.43 g, 2.00 mmol), methyl pent-4-ynoate (0.673 g, 6.00 mmol), CuI (0.076 g, 0.40 mmol), and Pd(PPh₃)₄ (0.231 g, 0.20 mmol) was added THF (15 mL) and DIPEA (3.50 mL, 20.02 mmol). The mixture was sparged with nitrogen, sealed, and heated to 70° C. for 1 h. Volatiles were removed in vacuo and the residue was purified by automated FCC (0-40% IPA in DCM) to yield the title compound as a solid (600 mg, 43%). LC-MS calculated for C₃₄H₄₂BrFN₅O₅ (M+H)⁺: m/z=698.2; found 698.3.

### Step 3. tert-butyl 5-(7-bromo-8-(2-cyanoethyl)-6-fluoro-2-(3-methoxy-3-oxopropyl)-4-(((S)-1-methylpyrrolidin-2-yl)methoxy)-1H-pyrrolo[3,2-c]quinolin-1-yl)-2-azabicyclo[2.1.1]hexane-2-carboxylate

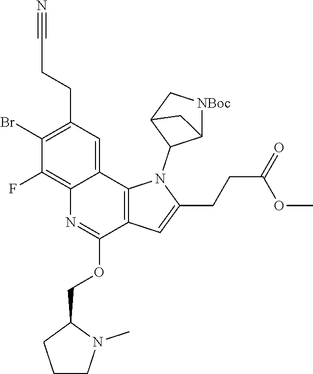 [SMILES: COC(=O)CCc1cc2c(OC[C@@H]3CCCN3C)nc3c(F)c(Br)c(CCC#N)cc3c2n1C1C2CC1N(C(=O)OC(C)(C)C)C2]

The title compound was prepared using the protocol detailed in Example 29, Step 17, replacing tert-butyl 5-((6-(2-cyanoethyl)-8-fluoro-3-(5-methoxy-5-oxopent-1-yn-1-yl)-7-(3-(methoxymethoxy)naphthalen-1-yl)-2-(((S)-1-methylpyrrolidin-2-yl)methoxy)quinolin-4-yl)amino)-2-azabicyclo[2.1.1]hexane-2-carboxylate with tert-butyl 5-((7-bromo-6-(2-cyanoethyl)-8-fluoro-3-(5-methoxy-5-oxopent-1-yn-1-yl)-2-(((S)-1-methylpyrrolidin-2-yl)methoxy)quinolin-4-yl)amino)-2-azabicyclo[2.1.1]hexane-2-carboxylate. LC-MS calculated for C₃₄H₄₂BrFN₅O₅ (M+H)⁺: m/z=698.2; found 698.2.

### Step 3. 3-(1-(2-(tert-butoxycarbonyl)-2-azabicyclo[2.1.1]hexan-5-yl)-8-(2-cyanoethyl)-6-fluoro-7-(7-fluoronaphthalen-1-yl)-4-(((S)-1-methylpyrrolidin-2-yl)methoxy)-1H-pyrrolo[3,2-c]quinolin-2-yl)propanoic acid

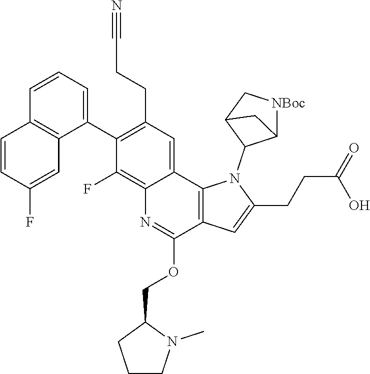 [SMILES: CN1CCC[C@H]1COc1nc2c(F)c(-c3cccc4ccc(F)cc34)c(CCC#N)cc2c2c1cc(CCC(=O)O)n2C1C2CC1N(C(=O)OC(C)(C)C)C2]

To a vial containing tert-butyl 5-(7-bromo-8-(2-cyanoethyl)-6-fluoro-2-(3-methoxy-3-oxopropyl)-4-(((S)-1-methylpyrrolidin-2-yl)methoxy)-1H-pyrrolo[3,2-c]quinolin-1-yl)-2-azabicyclo[2.1.1]hexane-2-carboxylate (200 mg, 0.286 mmol) was added K₃PO₄ (243 mg, 1.145 mmol), Pd(PPh₃)₄ (33.1 mg, 0.029 mmol), and 2-(7-fluoronaphthalen-1-yl)-4,4,5,5-tetramethyl-1,3,2-dioxaborolane (156 mg, 0.573 mmol) followed by 1,4-dioxane (0.5 mL), THF (0.5 mL), and water (0.5 mL). The vial was capped under nitrogen and stirred for 5 hours at 95° C. After this time, the mixture was cooled and filtered through a SiliaPrep Thiol Cartridge. The effluent was treated with water (0.5 mL), THF (0.5 mL), and LiOH (68 mg) and then stirred at RT for 3 h. After this time the mixture was brought to pH 5 with 10% AcOH solution and then purified by prep-HPLC (XBridge C18 column, acetonitrile/water gradient with 0.1% v/v TFA). Fractions containing the desired compound were combined and rotavapped to yield the title compound as a TFA salt (138 mg, 0.184 mmol, 64%). LC-MS calculated for C₄₃H₄₆F₂N₅O₅ (M+H)⁺: m/z=750.3; found 750.4.

### Step 4. 3-(2-(3-(azetidin-1-yl)-3-oxopropyl)-1-(2-azabicyclo[2.1.1]hexan-5-yl)-6-fluoro-7-(7-fluoronaphthalen-1-yl)-4-(((S)-1-methylpyrrolidin-2-yl)methoxy)-1H-pyrrolo[3,2-c]quinolin-8-yl)propanenitrile

To a vial containing 3-(1-(2-(tert-butoxycarbonyl)-2-azabicyclo[2.1.1]hexan-5-yl)-8-(2-cyanoethyl)-6-fluoro-7-(7-fluoronaphthalen-1-yl)-4-(((S)-1-methylpyrrolidin-2-yl)methoxy)-1H-pyrrolo[3,2-c]quinolin-2-yl)propanoic acid (20 mg, 0.027 mmol) was added PyBOP (21 mg, 0.040 mmol) followed by azetidine (4.6 mg, 0.080 mmol). DCM (1 mL) was added followed by DIPEA (0.046 mL, 0.267 mmol) and the mixture was stirred at RT for 20 min. At this time, water was added (1.5 mL) and the mixture was extracted with DCM (3×1.5 mL). Combined organic extracts were washed with sat. NaCl solution and then dried over MgSO₄. Volatiles were removed in vacuo and the residue was treated with TFA (0.5 mL). After 30 minutes the reaction mixture was diluted with acetonitrile and purified by prep-HPLC (XBridge C18 column, acetonitrile/water gradient with 0.1% v/v TFA). Fractions containing the desired compound were combined and lyophilized to yield the title compound as a TFA salt (11 mg recovered). LC-MS calculated for C₄₁H₄₃F₂N₆O₂ (M+H)⁺: m/z=689.3; found 689.3.

### Example 51a and Example 51b. 8-(1-((2S,4S)-2-(cyanomethyl)-1-((E)-4-fluorobut-2-enoyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile

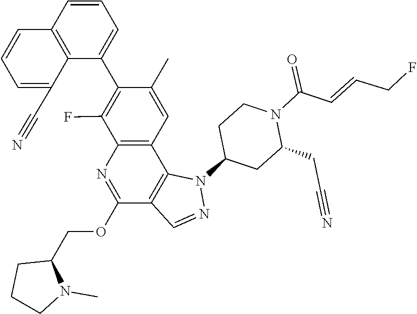 [SMILES: Cc1cc2c(nc(OC[C@@H]3CCCN3C)c3cnn([C@H]4CCN(C(=O)/C=C/CF)[C@H](CC#N)C4)c32)c(F)c1-c1cccc2cccc(C#N)c12]

### Step 1: ethyl 7-bromo-2,4-dichloro-8-fluoro-6-iodoquinoline-3-carboxylate

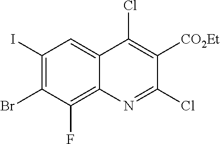 [SMILES: CCOC(=O)c1c(Cl)nc2c(F)c(Br)c(I)cc2c1Cl]

The title compound was synthesized according to the procedure described for Example 3a and 3b from step 1 to 3, utilizing 2-amino-4-bromo-3-fluoro-5-iodobenzoic acid instead of 2-amino-4-bromo-5-chloro-3-fluorobenzoic acid in Step 1. LCMS calculated for C₁₂H₇BrCl₂FINO₂ (M+H)⁺ m/z=491.80, 493.80; found 491.80, 493.80.

### Step 2. ethyl 7-bromo-4-(((2S,4S)-1-(tert-butoxycarbonyl)-2-(2-((tert-butyldimethylsilyl)oxy)ethyl)piperidin-4-yl)amino)-2-chloro-8-fluoro-6-iodoquinoline-3-carboxylate

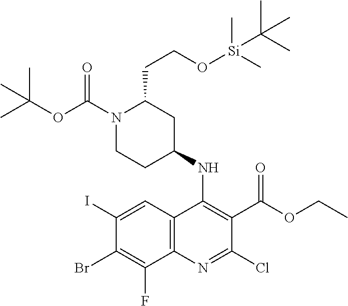 [SMILES: CCOC(=O)c1c(Cl)nc2c(F)c(Br)c(I)cc2c1N[C@H]1CCN(C(=O)OC(C)(C)C)[C@H](CCO[Si](C)(C)C(C)(C)C)C1]

This compound was prepared according to the procedure described in Example 3a and Example 3b, in Step 10 replacing ethyl 7-bromo-2,4,6-trichloro-8-fluoroquinoline-3-carboxylate with ethyl 7-bromo-2,4-dichloro-8-fluoro-6-iodoquinoline-3-carboxylate. LC-MS calculated for C₃₀H₄₄BrClFIN₃O₅Si (M+H)⁺: m/z=814.1, 816.1; found 814.1, 816.2.

### Step 3. tert-butyl (2S,4S)-4-((7-bromo-2-chloro-8-fluoro-3-(hydroxymethyl)-6-iodoquinolin-4-yl)amino)-2-(2-((tert-butyldimethylsilyl)oxy)ethyl)piperidine-1-carboxylate

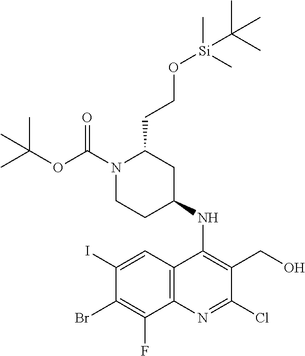 [SMILES: CC(C)(C)OC(=O)N1CC[C@H](Nc2c(CO)c(Cl)nc3c(F)c(Br)c(I)cc23)C[C@H]1CCO[Si](C)(C)C(C)(C)C]

This compound was prepared according to the procedure described in Example 3a and Example 3b, in Step 11 replacing ethyl 7-bromo-4-(((2S,4S)-1-(tert-butoxycarbonyl)-2-(2-((tert-butyldimethylsilyl)oxy)ethyl)piperidin-4-yl)amino)-2,6-dichloro-8-fluoroquinoline-3-carboxylate with ethyl 7-bromo-4-(((2S,4S)-1-(tert-butoxycarbonyl)-2-(2-((tert-butyldimethylsilyl)oxy)ethyl)piperidin-4-yl)amino)-2-chloro-8-fluoro-6-iodoquinoline-3-carboxylate. LC-MS calculated for C₂₈H₄₂BrClFIN₃O₄Si (M+H)⁺: m/z=772.1, 774.1; found 772.1, 774.1.

### Step 4. tert-butyl (2S,4S)-4-((7-bromo-2-chloro-8-fluoro-3-formyl-6-iodoquinolin-4-yl)amino)-2-(2-((tert-butyldimethylsilyl)oxy)ethyl)piperidine-1-carboxylate

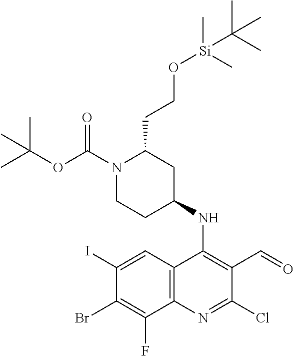 [SMILES: CC(C)(C)OC(=O)N1CC[C@H](Nc2c(C=O)c(Cl)nc3c(F)c(Br)c(I)cc23)C[C@H]1CCO[Si](C)(C)C(C)(C)C]

This compound was prepared according to the procedure described in Example 3a and Example 3b, in Step 12 replacing tert-butyl (2S,4S)-4-((7-bromo-2,6-dichloro-8-fluoro-3-(hydroxymethyl)quinolin-4-yl)amino)-2-(2-((tert-butyldimethylsilyl)oxy)ethyl)piperidine-1-carboxylate with tert-butyl (2S,4S)-4-((7-bromo-2-chloro-8-fluoro-3-(hydroxymethyl)-6-iodoquinolin-4-yl)amino)-2-(2-((tert-butyldimethylsilyl)oxy)ethyl)piperidine-1-carboxylate. LC-MS calculated for C₂₈H₄₀BrClFIN₃O₄Si (M+H)⁺: m/z=770.1, 772.1; found 770.1, 772.1.

### Step 5. tert-butyl (2S,4S)-4-((7-bromo-2-chloro-8-fluoro-3-((E)-(hydroxyimino)methyl)-6-iodoquinolin-4-yl)amino)-2-(2-((tert-butyldimethylsilyl)oxy)ethyl)piperidine-1-carboxylate

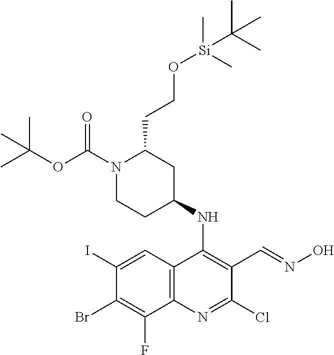 [SMILES: CC(C)(C)OC(=O)N1CC[C@H](Nc2c(/C=N/O)c(Cl)nc3c(F)c(Br)c(I)cc23)C[C@H]1CCO[Si](C)(C)C(C)(C)C]

This compound was prepared according to the procedure described in Example 3a and Example 3b, in Step 13 replacing tert-butyl (2S,4S)-4-((7-bromo-2,6-dichloro-8-fluoro-3-formylquinolin-4-yl)amino)-2-(2-((tert-butyldimethylsilyl)oxy)ethyl)piperidine-1-carboxylate with tert-butyl (2S,4S)-4-((7-bromo-2-chloro-8-fluoro-3-formyl-6-iodoquinolin-4-yl)amino)-2-(2-((tert-butyldimethylsilyl)oxy)ethyl)piperidine-1-carboxylate. LC-MS calculated for C₂₈H₄₁BrClFIN₄O₄Si (M+H)⁺: m/z=785.1, 787.1; found 785.2, 787.2.

### Step 6. tert-butyl (2S,4S)-4-(7-bromo-4-chloro-6-fluoro-8-iodo-1H-pyrazolo[4,3-c]quinolin-1-yl)-2-(2-((tert-butyldimethylsilyl)oxy)ethyl)piperidine-1-carboxylate

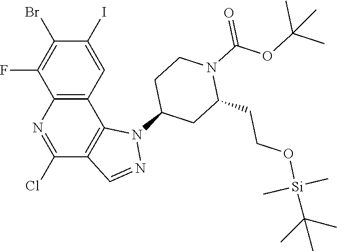 [SMILES: CC(C)(C)OC(=O)N1CC[C@H](n2ncc3c(Cl)nc4c(F)c(Br)c(I)cc4c32)C[C@H]1CCO[Si](C)(C)C(C)(C)C]

This compound was prepared according to the procedure described in Example 3a and Example 3b, in Step 14 replacing (tert-butyl (2S,4S)-4-((7-bromo-2,6-dichloro-8-fluoro-3-((E)-(hydroxyimino)methyl)quinolin-4-yl)amino)-2-(2-((tert-butyldimethylsilyl)oxy)ethyl)piperidine-1-carboxylate with tert-butyl (2S,4S)-4-((7-bromo-2-chloro-8-fluoro-3-((E)-(hydroxyimino)methyl)-6-iodoquinolin-4-yl)amino)-2-(2-((tert-butyldimethylsilyl)oxy)ethyl)piperidine-1-carboxylate. LC-MS calculated for C₂₈H₃₉BrClFIN₄O₃Si (M+H)⁺: m/z=767.1, 769.1; found 767.1, 769.1.

### Step 7. tert-butyl (2S,4S)-4-(7-bromo-6-fluoro-8-iodo-4-(methylthio)-1H-pyrazolo[4,3-c]quinolin-1-yl)-2-(2-((tert-butyldimethylsilyl)oxy)ethyl)piperidine-1-carboxylate

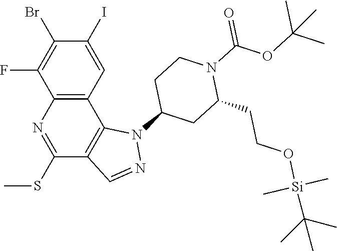 [SMILES: CSc1nc2c(F)c(Br)c(I)cc2c2c1cnn2[C@H]1CCN(C(=O)OC(C)(C)C)[C@H](CCO[Si](C)(C)C(C)(C)C)C1]

This compound was prepared according to the procedure described in Example 3a and Example 3b, in Step 15 replacing of tert-butyl (2S,4S)-4-(7-bromo-4,8-dichloro-6-fluoro-1H-pyrazolo[4,3-c]quinolin-1-yl)-2-(2-((tert-butyldimethylsilyl)oxy)ethyl)piperidine-1-carboxylate with tert-butyl (2S,4S)-4-(7-bromo-4-chloro-6-fluoro-8-iodo-1H-pyrazolo[4,3-c]quinolin-1-yl)-2-(2-((tert-butyldimethylsilyl)oxy)ethyl)piperidine-1-carboxylate. LC-MS calculated for C₂₉H₄₂BrFIN₄O₃SSi (M+H)⁺: m/z=779.1, 781.1; found 779.1, 781.1.

### Step 8. tert-butyl (2S,4S)-4-(7-bromo-6-fluoro-8-iodo-4-(methylthio)-1H-pyrazolo[4,3-c]quinolin-1-yl)-2-(2-hydroxyethyl)piperidine-1-carboxylate

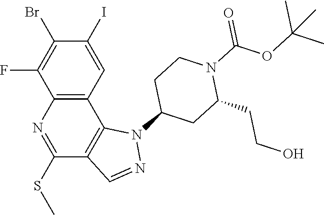 [SMILES: CSc1nc2c(F)c(Br)c(I)cc2c2c1cnn2[C@H]1CCN(C(=O)OC(C)(C)C)[C@H](CCO)C1]

This compound was prepared according to the procedure described in Example 3a and Example 3b, in Step 16 replacing of tert-butyl (2S,4S)-4-(7-bromo-8-chloro-6-fluoro-4-(methylthio)-1H-pyrazolo[4,3-c]quinolin-1-yl)-2-(2-((tert-butyldimethylsilyl)oxy)ethyl)piperidine-1-carboxylate with tert-butyl (2S,4S)-4-(7-bromo-6-fluoro-8-iodo-4-(methylthio)-1H-pyrazolo[4,3-c]quinolin-1-yl)-2-(2-((tert-butyldimethylsilyl)oxy)ethyl)piperidine-1-carboxylate. LC-MS calculated for C₂₃H₂₈BrFIN₄O₃S (M+H)⁺: m/z=665.0, 667.0; found 665.1, 667.1.

### Step 9. tert-butyl (2S,4S)-4-(7-bromo-6-fluoro-8-iodo-4-(methylthio)-1H-pyrazolo[4,3-c]quinolin-1-yl)-2-(cyanomethyl)piperidine-1-carboxylate

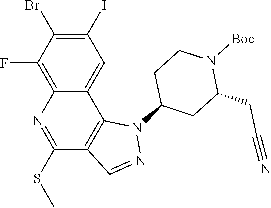 [SMILES: CSc1nc2c(F)c(Br)c(I)cc2c2c1cnn2[C@H]1CCN(C(=O)OC(C)(C)C)[C@H](CC#N)C1]

This compound was prepared according to the procedure described in Example 3a and Example 3b, in Step 17 replacing of tert-butyl (2S,4S)-4-(7-bromo-8-chloro-6-fluoro-4-(methylthio)-1H-pyrazolo[4,3-c]quinolin-1-yl)-2-(2-hydroxyethyl)piperidine-1-carboxylate with tert-butyl (2S,4S)-4-(7-bromo-6-fluoro-8-iodo-4-(methylthio)-1H-pyrazolo[4,3-c]quinolin-1-yl)-2-(2-hydroxyethyl)piperidine-1-carboxylate. LC-MS calculated for C₂₃H₂₅BrFIN₅O₂S (M+H)⁺: m/z=660.0, 662.0; found 660.0, 662.0.

### Step 10. tert-butyl (2S,4S)-4-(7-bromo-6-fluoro-8-methyl-4-(methylthio)-1H-pyrazolo[4,3-c]quinolin-1-yl)-2-(cyanomethyl)piperidine-1-carboxylate

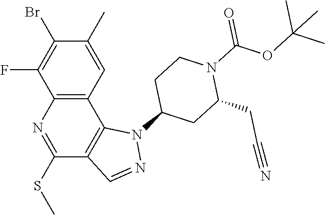 [SMILES: CSc1nc2c(F)c(Br)c(C)cc2c2c1cnn2[C@H]1CCN(C(=O)OC(C)(C)C)[C@H](CC#N)C1]

To a solution of tert-butyl (2S,4S)-4-(7-bromo-6-fluoro-8-iodo-4-(methylthio)-1H-pyrazolo[4,3-c]quinolin-1-yl)-2-(cyanomethyl)piperidine-1-carboxylate (2.75 g, 4.16 mmol) in 1,4-dioxane (36 ml) was added water (6.0 ml), methylboronic acid (1.496 g, 24.99 mmol), K₂CO₃ (1.151 g, 8.33 mmol) and Pd(PPh₃)₂Cl₂ (0.292 g, 0.416 mmol) at rt. The reaction mixture was stirred at 90° C. for 10 h under N₂ atmosphere. After the reaction was complete, the reaction mixture was quenched with water and extracted with EtOAc. The organic phase was dried over anhydrous Na₂SO₄ and concentrated and then purified by column chromatography on silica gel (Eluents:Hexanes:Ethyl acetate=5:1) to get compound (1.9 g, 83%) as a white solid. LC-MS calculated for C₂₄H₂₈BrFN₅O₂S (M+H)⁺: m/z=548.1, 550.1; found 548.2, 550.2.

### Step 11. tert-butyl (2S,4S)-4-(7-bromo-6-fluoro-8-methyl-4-(((S)-1-methylpyrrolidin-2-yl)methoxy)-1H-pyrazolo[4,3-c]quinolin-1-yl)-2-(cyanomethyl)piperidine-1-carboxylate

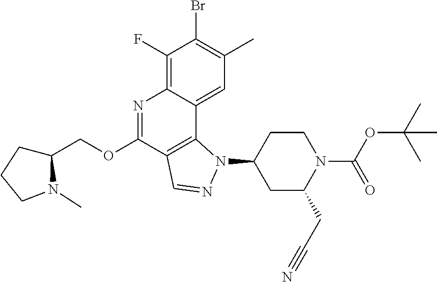 [SMILES: Cc1cc2c(nc(OC[C@@H]3CCCN3C)c3cnn([C@H]4CCN(C(=O)OC(C)(C)C)[C@H](CC#N)C4)c32)c(F)c1Br]

m-CPBA (57.9 mg, 0.335 mmol) was added to a solution of tert-butyl (2S,4S)-4-(7-bromo-6-fluoro-8-methyl-4-(methylthio)-1H-pyrazolo[4,3-c]quinolin-1-yl)-2-(cyanomethyl)piperidine-1-carboxylate (160 mg, 0.292 mmol) in CH2Cl2 (2.92 ml) at 0° C. and then the reaction was stirred at this temperature for 20 min. The reaction was quenched by adding sat'd Na₂S₂O₃, diluted with ethyl acetate and washed with sat'd NaHCO₃, brine, filtered, dried and concentrated. 1.0 M LiHMDS in THF (753 μl, 0.753 mmol) was added to a solution of (S)-(1-methylpyrrolidin-2-yl)methanol (87 mg, 0.753 mmol) in THF (1 mL). The resulting mixture was stirred at rt for 30 min. A solution of tert-butyl (2S,4S)-4-(7-bromo-6-fluoro-8-methyl-4-(methylsulfinyl)-1H-pyrazolo[4,3-c]quinolin-1-yl)-2-(cyanomethyl)piperidine-1-carboxylate (170 mg, 0.301 mmol) in THF (2.0 ml) was added to reaction vial and then the reaction was stirred at room temperature for 2 h. The reaction mixture was diluted with ethyl acetate and water. The organic layer was dried over Na₂SO₄, filtered and concentrated. The residue was purified with silica gel column (eluting with a gradient of 0-20% methanol in DCM) to give the desired product as yellow foam (185 mg, 100%). LC-MS calculated for C₂₉H₃₇BrFN₆O₃ (M+H)⁺: m/z=615.2, 617.2; found 615.3, 617.3.

### Step 12. tert-butyl (2S,4S)-2-(cyanomethyl)-4-(7-(8-cyanonaphthalen-1-yl)-6-fluoro-8-methyl-4-(((S)-1-methylpyrrolidin-2-yl)methoxy)-1H-pyrazolo[4,3-c]quinolin-1-yl)piperidine-1-carboxylate

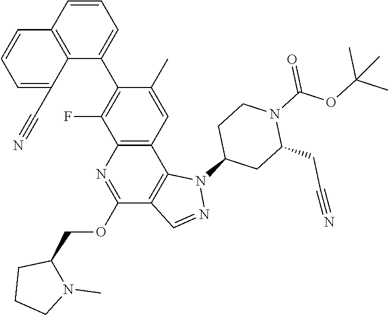 [SMILES: Cc1cc2c(nc(OC[C@@H]3CCCN3C)c3cnn([C@H]4CCN(C(=O)OC(C)(C)C)[C@H](CC#N)C4)c32)c(F)c1-c1cccc2cccc(C#N)c12]

A mixture of tert-butyl (2S,4S)-4-(7-bromo-6-fluoro-8-methyl-4-(((S)-1-methylpyrrolidin-2-yl)methoxy)-1H-pyrazolo[4,3-c]quinolin-1-yl)-2-(cyanomethyl)piperidine-1-carboxylate (185 mg, 0.301 mmol), 8-(4,4,5,5-tetramethyl-1,3,2-dioxaborolan-2-yl)-1-naphthonitrile (92 mg, 0.331 mmol), SPhos Pd G4 (23.87 mg, 0.030 mmol) and tripotassium phosphate hydrate (152 mg, 0.661 mmol) in 1,4-dioxane (2.0 mL)/water (0.400 mL) was stirred at 80° C. under N₂ atmosphere for 2 h. The solution was diluted with ethyl acetate and water. The organic layer was concentrated and the residue was purified with prep-LCMS (XBridge C18 column, eluting with a gradient of acetonitrile/water containing 0.1% NH4OH, at flow rate of 60 mL/min) to give the desired product as two peaks (120 mg, 58%).

Diastereomer 1. Peak 1. LC-MS calculated for C₄₀H₄₃FN₇O₃ (M+H)⁺: m/z=688.3; found 688.3.

Diastereomer 2. Peak 2. LC-MS calculated for C₄₀H₄₃FN₇O₃ (M+H)⁺: m/z=688.3; found 688.3.

### Step 13. 8-(1-((2S,4S)-2-(cyanomethyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile

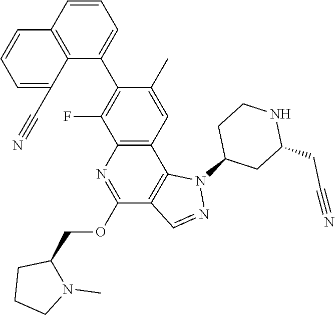 [SMILES: Cc1cc2c(nc(OC[C@@H]3CCCN3C)c3cnn([C@H]4CCN[C@H](CC#N)C4)c32)c(F)c1-c1cccc2cccc(C#N)c12]

Two Diastereomers from last step were treated with 1:1 DCM/TFA (2 mL) for 40 min, The volatiles were removed in vacuo and residue was used in the next step as is.

Diastereomer 1. Peak 1. LC-MS calculated for C₃₅H₃₅FN₇O (M+H)⁺: m/z=588.3; found 588.3.

Diastereomer 2. Peak 2. LC-MS calculated for C₃₅H₃₅FN₇O (M+H)⁺: m/z=588.3; found 588.3.

### Step 14. 8-(1-((2S,4S)-2-(cyanomethyl)-1-((E)-4-fluorobut-2-enoyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile

To a solution of (E)-4-fluorobut-2-enoic acid (0.92 mg, 8.83 μmol) and 8-(1-((2S,4S)-2-(cyanomethyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile bis(2,2,2-trifluoroacetate) (6.0 mg, 7.36 μmol) (Diastereomer 1 peak 1 from last step) in DMF (1.0 ml) was added HATU (3.5 mg, 9.19 μmol) and DIEA (6.4 μl, 0.037 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was prepared in similar way using 8-(1-((2S,4S)-2-(cyanomethyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile bis(2,2,2-trifluoroacetate) (diastereomer 2 peak 2 from last step).

Example 51a. Diastereomer 1. Peak 1. LCMS calculated for C₃₉H₃₈F₂N₇O₂ (M+H)⁺ m/z=674.3; found 674.3.

Example 51b. Diastereomer 2. Peak 2. LCMS calculated for C₃₉H₃₈F₂N₇O₂ (M+H)⁺ m/z=674.3; found 674.3.

### Example 52a and Example 52b. 8-(1-((2S,4S)-2-(cyanomethyl)-1-(2-fluoroacryloyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile

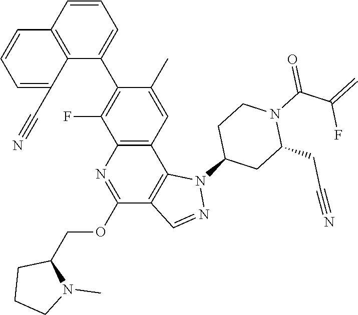 [SMILES: C=C(F)C(=O)N1CC[C@H](n2ncc3c(OC[C@@H]4CCCN4C)nc4c(F)c(-c5cccc6cccc(C#N)c56)c(C)cc4c32)C[C@H]1CC#N]

This compound was prepared according to the procedure described in Example 51a and Example 51b, step 14, replacing (E)-4-fluorobut-2-enoic acid with 2-fluoroacrylic acid.

Example 52a. Diastereomer 1. Peak 1. LCMS calculated for C₃₈H₃₆F₂N₇O₂ (M+H)⁺ m/z=660.3; found 660.4.

Example 52b. Diastereomer 2. Peak 2. LCMS calculated for C₃₈H₃₆F₂N₇O₂ (M+H)⁺ m/z=660.3; found 660.4.

### Example 53a and Example 53b. 8-(1-((2S,4S)-1-(but-2-ynoyl)-2-(cyanomethyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile

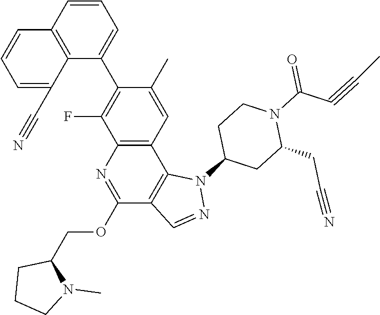 [SMILES: CC#CC(=O)N1CC[C@H](n2ncc3c(OC[C@@H]4CCCN4C)nc4c(F)c(-c5cccc6cccc(C#N)c56)c(C)cc4c32)C[C@H]1CC#N]

This compound was prepared according to the procedure described in Example 51a and Example 51b, step 14, replacing (E)-4-fluorobut-2-enoic acid with but-2-ynoic acid.

Example 53a. Diastereomer 1. Peak 1. LCMS calculated for C₃₉H₃₇FN₇O₂ (M+H)⁺ m/z=654.3; found 654.3.

Example 53b. Diastereomer 2. Peak 2. LCMS calculated for C₃₉H₃₇FN₇O₂ (M+H)⁺ m/z=654.3; found 654.3.

### Example 54a and Example 54b. 8-(1-((2S,4S)-2-(cyanomethyl)-1-((E)-4-methoxybut-2-enoyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile

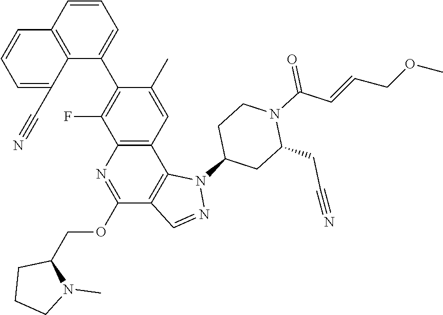 [SMILES: COC/C=C/C(=O)N1CC[C@H](n2ncc3c(OC[C@@H]4CCCN4C)nc4c(F)c(-c5cccc6cccc(C#N)c56)c(C)cc4c32)C[C@H]1CC#N]

This compound was prepared according to the procedure described in Example 51a and Example 51b, step 14, replacing (E)-4-fluorobut-2-enoic acid with (E)-4-methoxybut-2-enoic acid.

Example 54a. Diastereomer 1. Peak 1. LCMS calculated for C₄₀H₄₁FN₇O₃ (M+H)⁺ m/z=686.3; found 686.4.

Example 54b. Diastereomer 2. Peak 2. LCMS calculated for C₄₀H₄₁FN₇O₃ (M+H)⁺ m/z=686.3; found 686.4.

### Example 55a and Example 55b. 8-(1-((2S,4S)-2-(cyanomethyl)-1-((E)-4-fluorobut-2-enoyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile

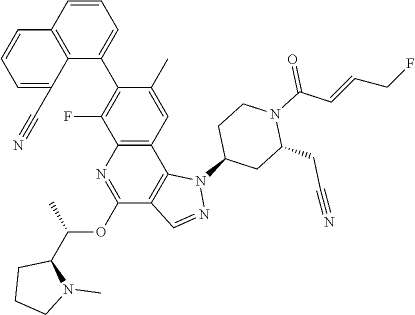 [SMILES: Cc1cc2c(nc(O[C@@H](C)[C@@H]3CCCN3C)c3cnn([C@H]4CCN(C(=O)/C=C/CF)[C@H](CC#N)C4)c32)c(F)c1-c1cccc2cccc(C#N)c12]

### Step 1. tert-butyl (2S,4S)-4-(7-bromo-6-fluoro-8-methyl-4-((S)-1-((S)-1-methylpyrrolidin-2-yl)ethoxy)-1H-pyrazolo[4,3-c]quinolin-1-yl)-2-(cyanomethyl)piperidine-1-carboxylate

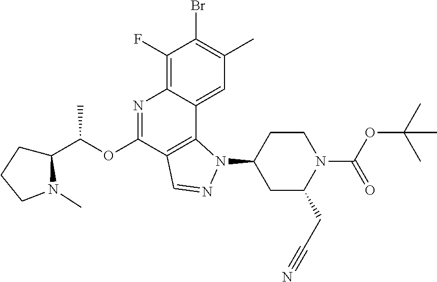 [SMILES: Cc1cc2c(nc(O[C@@H](C)[C@@H]3CCCN3C)c3cnn([C@H]4CCN(C(=O)OC(C)(C)C)[C@H](CC#N)C4)c32)c(F)c1Br]

This compound was prepared according to the procedure described in Example 51a and Example 51b, step 11, replacing (S)-(1-methylpyrrolidin-2-yl)methanol with (S)-1-((S)-1-methylpyrrolidin-2-yl)ethan-1-ol. LC-MS calculated for C₃₀H₃₉BrFN₆O₃ (M+H)⁺: m/z=629.2, 631.2; found 629.3, 631.3.

### Step 2. tert-butyl (2S,4S)-2-(cyanomethyl)-4-(7-(8-cyanonaphthalen-1-yl)-6-fluoro-8-methyl-4-((S)-1-((S)-1-methylpyrrolidin-2-yl)ethoxy)-1H-pyrazolo[4,3-c]quinolin-1-yl)piperidine-1-carboxylate

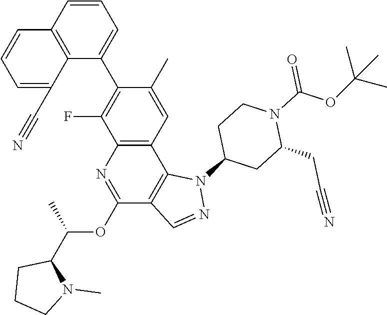 [SMILES: Cc1cc2c(nc(O[C@@H](C)[C@@H]3CCCN3C)c3cnn([C@H]4CCN(C(=O)OC(C)(C)C)[C@H](CC#N)C4)c32)c(F)c1-c1cccc2cccc(C#N)c12]

The mixture of tert-butyl (2S,4S)-4-(7-bromo-6-fluoro-8-methyl-4-((S)-1-((S)-1-methylpyrrolidin-2-yl)ethoxy)-1H-pyrazolo[4,3-c]quinolin-1-yl)-2-(cyanomethyl)piperidine-1-carboxylate (150 mg, 0.238 mmol), 8-(4,4,5,5-tetramethyl-1,3,2-dioxaborolan-2-yl)-1-naphthonitrile (86 mg, 0.310 mmol), SPhos Pd G4 (18.9 mg, 0.024 mmol) and tripotassium phosphate hydrate (121 mg, 0.524 mmol) in 1,4-dioxane (2.0 mL)/water (0.400 mL) was stirred at 80° C. for 2 h The solution was diluted with ethyl acetate and water. The organic layer was concentrated and the residue was purified with prep-LCMS (XBridge C18 column, eluting with a gradient of acetonitrile/water containing 0.1% TFA, at flow rate of 60 mL/min) to give the desired product as two peaks (105 mg, 63%).

Diastereomer 1. Peak 1. LC-MS calculated for C₄₁H₄₅FN₇O₃ (M+H)⁺: m/z=702.4; found 702.4.

Diastereomer 2. Peak 2. LC-MS calculated for C₄₁H₄₅FN₇O₃ (M+H)⁺: m/z=702.4; found 702.4.

### Step 3. 8-(1-((2S,4S)-2-(cyanomethyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile

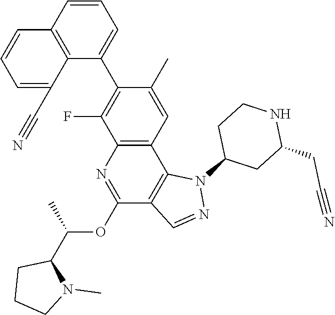 [SMILES: Cc1cc2c(nc(O[C@@H](C)[C@@H]3CCCN3C)c3cnn([C@H]4CCN[C@H](CC#N)C4)c32)c(F)c1-c1cccc2cccc(C#N)c12]

Two Diastereomers from last step were treated with 1:1 DCM/TFA (2 mL) for 40 min, The volatiles were removed in vacuo and residue was used in the next step as is.

Diastereomer 1. Peak 1. LC-MS calculated for C₃₆H₃₇FN₇O (M+H)⁺: m/z=602.3; found 602.3.

Diastereomer 2. Peak 2. LC-MS calculated for C₃₆H₃₇FN₇O (M+H)⁺: m/z=602.3; found 602.3.

### Step 4. 8-(1-((2S,4S)-2-(cyanomethyl)-1-((E)-4-fluorobut-2-enoyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile

To a solution of (E)-4-fluorobut-2-enoic acid (0.90 mg, 8.68 μmol) and 8-(1-((2S,4S)-2-(cyanomethyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile bis(2,2,2-trifluoroacetate) (6.0 mg, 7.23 μmol) (Diastereomer 1, peak1 from last step) in DMF (1.0 ml) was added HATU (3.4 mg, 9.04 μmol) and DIEA (6.3 μl, 0.036 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 8-(1-((2S,4S)-2-(cyanomethyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile bis(2,2,2-trifluoroacetate) (diastereomer 2 peak 2 from last step).

Example 55a. Diastereomer 1. Peak 1. LCMS calculated for C₄₀H₄₀F₂N₇O₂ (M+H)⁺ m/z=688.3; found 688.3.

Example 55b. Diastereomer 2. Peak 2. LCMS calculated for C₄₀H₄₀F₂N₇O₂ (M+H)⁺ m/z=688.3; found 688.3.

### Example 56a and Example 56b. 8-(1-((2S,4S)-2-(cyanomethyl)-1-(2-fluoroacryloyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile

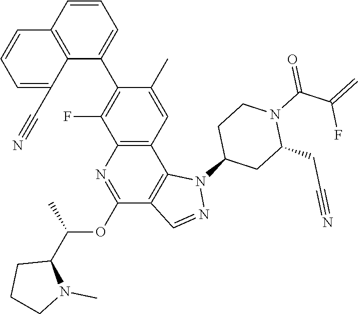 [SMILES: C=C(F)C(=O)N1CC[C@H](n2ncc3c(O[C@@H](C)[C@@H]4CCCN4C)nc4c(F)c(-c5cccc6cccc(C#N)c56)c(C)cc4c32)C[C@H]1CC#N]

This compound was prepared according to the procedure described in Example 55a and Example 55b, step 4, replacing (E)-4-fluorobut-2-enoic acid with 2-fluoroacrylic acid.

Example 56a. Diastereomer 1. Peak 1. LCMS calculated for C₃₉H₃₈F₂N₇O₂ (M+H)⁺ m/z=674.3; found 674.3.

Example 56b. Diastereomer 2. Peak 2. LCMS calculated for C₃₉H₃₈F₂N₇O₂ (M+H)⁺ m/z=674.3; found 674.3.

### Example 57a and Example 57b. 8-(1-((2S,4S)-2-(cyanomethyl)-1-((E)-4-methoxybut-2-enoyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile

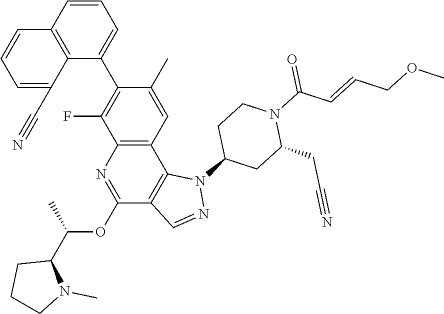 [SMILES: COC/C=C/C(=O)N1CC[C@H](n2ncc3c(O[C@@H](C)[C@@H]4CCCN4C)nc4c(F)c(-c5cccc6cccc(C#N)c56)c(C)cc4c32)C[C@H]1CC#N]

This compound was prepared according to the procedure described in Example 55a and Example 55b, step 4, replacing (E)-4-fluorobut-2-enoic acid with (E)-4-methoxybut-2-enoic acid.

Example 57a. Diastereomer 1. Peak 1. LCMS calculated for C₄₁H₄₃FN₇O₃ (M+H)⁺ m/z=700.3; found 700.3.

Example 57b. Diastereomer 2. Peak 2. LCMS calculated for C₄₁H₄₃FN₇O₃ (M+H)⁺ m/z=700.3; found 700.3.

### Example 58a and Example 58b. 8-(1-((2S,4S)-2-(cyanomethyl)-1-((E)-4-fluorobut-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile

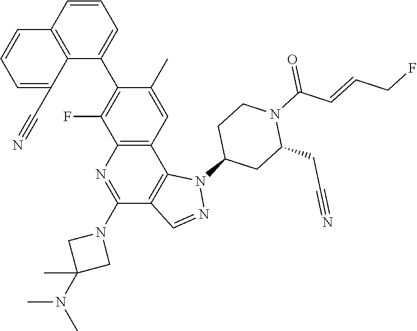 [SMILES: Cc1cc2c(nc(N3CC(C)(N(C)C)C3)c3cnn([C@H]4CCN(C(=O)/C=C/CF)[C@H](CC#N)C4)c32)c(F)c1-c1cccc2cccc(C#N)c12]

### Step 1. tert-butyl (2S,4S)-4-(7-bromo-4-(3-(dimethylamino)-3-methylazetidin-1-yl)-6-fluoro-8-methyl-1H-pyrazolo[4,3-c]quinolin-1-yl)-2-(cyanomethyl)piperidine-1-carboxylate

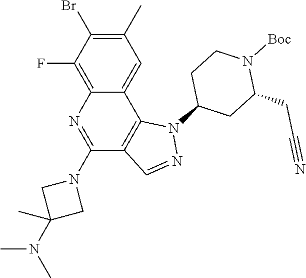 [SMILES: Cc1cc2c(nc(N3CC(C)(N(C)C)C3)c3cnn([C@H]4CCN(C(=O)OC(C)(C)C)[C@H](CC#N)C4)c32)c(F)c1Br]

m-CPBA (100 mg, 0.577 mmol) was added to a solution of tert-butyl (2S,4S)-4-(7-bromo-6-fluoro-8-methyl-4-(methylthio)-1H-pyrazolo[4,3-c]quinolin-1-yl)-2-(cyanomethyl)piperidine-1-carboxylate (275 mg, 0.501 mmol) in DCM (5.0 mL) at 0° C. and then the reaction was stirred at this temperature for 20 min. The reaction was quenched by adding saturated Na₂S₂O₃, diluted with ethyl acetate and washed with saturated NaHCO₃, brine, dried over Na₂SO₄, filtered, and concentrated. The crude was dissolved in acetonitrile (2 mL), triethylamine (287 μl, 2.062 mmol) and N,N,3-trimethylazetidin-3-amine hydrochloride (116 mg, 0.773 mmol) was added and then stirred at 80° C. for 2 h. The volatiles were evaporated under reduced pressure, the residue was purified by silica gel column (eluting with a gradient of 0-15% CH₂Cl₂ in MeOH to give the desired product as yellow foam (300 mg, 95%). LC-MS calculated for C₂₉H₃₈BrFN₇O₂ (M+H)⁺: m/z=614.2, 616.2; found 614.3, 616.3.

### Step 2. tert-butyl (2S,4S)-2-(cyanomethyl)-4-(7-(8-cyanonaphthalen-1-yl)-4-(3-(dimethylamino)-3-methylazetidin-1-yl)-6-fluoro-8-methyl-1H-pyrazolo[4,3-c]quinolin-1-yl)piperidine-1-carboxylate

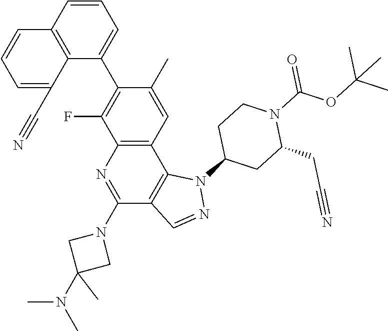 [SMILES: Cc1cc2c(nc(N3CC(C)(N(C)C)C3)c3cnn([C@H]4CCN(C(=O)OC(C)(C)C)[C@H](CC#N)C4)c32)c(F)c1-c1cccc2cccc(C#N)c12]

A mixture of tert-butyl (2S,4S)-4-(7-bromo-4-(3-(dimethylamino)-3-methylazetidin-1-yl)-6-fluoro-8-methyl-1H-pyrazolo[4,3-c]quinolin-1-yl)-2-(cyanomethyl) piperidine-1-carboxylate (165 mg, 0.268 mmol), 8-(4,4,5,5-tetramethyl-1,3,2-dioxaborolan-2-yl)-1-naphthonitrile (112 mg, 0.403 mmol), SPhos Pd G4 (21.3 mg, 0.027 mmol) and tripotassium phosphate hydrate (136 mg, 0.591 mmol) in 1,4-dioxane (2.0 mL)/water (0.400 mL) was stirred at 80° C. under nitrogen atmosphere for 2 h. The reaction solution was diluted with ethyl acetate and water. The organic layer was concentrated and purified with silica gel column to give the desired product (185 mg, 100%). LC-MS calculated for C₄₀H₄₄FN₈O₂ (M+H)⁺: m/z=687.4; found 687.5.

### Step 3. 8-(1-((2S,4S)-2-(cyanomethyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile

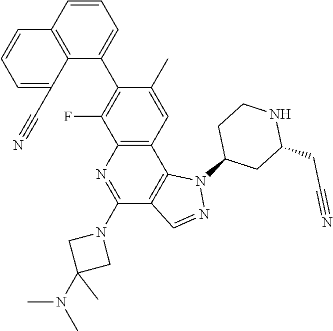 [SMILES: Cc1cc2c(nc(N3CC(C)(N(C)C)C3)c3cnn([C@H]4CCN[C@H](CC#N)C4)c32)c(F)c1-c1cccc2cccc(C#N)c12]

tert-butyl (2S,4S)-2-(cyanomethyl)-4-(7-(8-cyanonaphthalen-1-yl)-4-(3-(dimethylamino)-3-methylazetidin-1-yl)-6-fluoro-8-methyl-1H-pyrazolo[4,3-c]quinolin-1-yl)piperidine-1-carboxylate (184 mg, 0.268 mmol) in DCM (1 ml) was treated with TFA (826 μl, 10.72 mmol) for 40 min. The volatiles were removed in vacuo. The residue was dissolved in acetonitrile and purified with prep-LCMS (XBridge C18 column, eluting with a gradient of acetonitrile/water containing 0.1% TFA, at flow rate of 60 mL/min) to give two peaks (80 mg, 51%)

Diastereomer 1. Peak 1. LC-MS calculated for C₃₅H₃₆FN₈ (M+H)⁺: m/z=587.3; found 587.4.

Diastereomer 2. Peak 2. LC-MS calculated for C₃₅H₃₆FN₈(M+H)⁺: m/z=587.3; found 587.4.

### Step 4. 8-(1-((2S,4S)-2-(cyanomethyl)-1-((E)-4-fluorobut-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile

To a solution of (E)-4-fluorobut-2-enoic acid (0.95 mg, 9.13 μmol) and 8-(1-((2S,4S)-2-(cyanomethyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile bis(2,2,2-trifluoroacetate) (6.2 mg, 7.61 μmol) (Diastereomer 2, peak2 from last step) in DMF (1.0 ml) was added HATU (3.76 mg, 9.89 μmol) and DIEA (6.7 μl, 0.038 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 8-(1-((2S,4S)-2-(cyanomethyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile bis(2,2,2-trifluoroacetate) (diastereomer 1 peak 1 from last step).

Example 58a. Diastereomer 1. Peak 1. LCMS calculated for C₃₉H₃₉F₂N₈O (M+H)⁺ m/z=673.3; found 673.3. ¹H NMR (600 MHz, DMSO-d₆) δ 8.50-8.46 (m, 1H), 8.32-8.25 (m, 2H), 8.14-8.08 (m, 2H), 7.77-7.72 (m, 2H), 7.61 (t, J=7.2 Hz, 1H), 6.83 (m, 2H), 5.75 (m, 1H), 5.24 (m, 1H), 5.20 (s, 1H), 5.12 (s, 1H), 4.72 (m, 2H), 4.28 (m, 2H), 3.64 (m, 2H), 3.34 (m, 2H), 2.81 (s, 6H), 2.32-2.21 (m, 3H), 2.16 (s, 3H), 2.03 (m, 1H), 1.69 (s, 3H).

Example 58b. Diastereomer 2. Peak 2. LCMS calculated for C₃₉H₃₉F₂N₈O (M+H)⁺ m/z=673.3; found 673.3.

### Example 59a and Example 59b. 8-(1-((2S,4S)-2-(cyanomethyl)-1-(2-fluoroacryloyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile

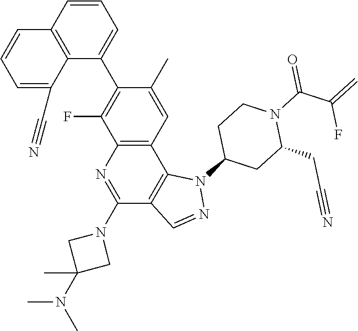 [SMILES: C=C(F)C(=O)N1CC[C@H](n2ncc3c(N4CC(C)(N(C)C)C4)nc4c(F)c(-c5cccc6cccc(C#N)c56)c(C)cc4c32)C[C@H]1CC#N]

This compound was prepared according to the procedure described in Example 58a and Example 58b, step 4, replacing (E)-4-fluorobut-2-enoic acid with 2-fluoroacrylic acid.

Example 59a. Diastereomer 1. Peak 1. LCMS calculated for C₃₈H₃₇F₂N₈O (M+H)⁺ m/z=659.3; found 659.4. ¹H NMR (500 MHz, DMSO-d₆) δ 8.45 (m, 1H), 8.29-8.22 (m, 2H), 8.10-8.03 (m, 2H), 7.87-7.80 (m, 1H), 7.73 (m, 1H), 7.58 (m, 1H), 5.81-5.73 (m, 1H), 5.38-5.30 (m, 2H), 4.61 (m, 2H), 4.38 (d, J=9.7 Hz, 1H), 4.32 (d, J=9.8 Hz, 2H), 3.51-3.44 (m, 5H), 2.82 (s, 6H), 2.34 (s, 1H), 2.26 (m, 1H), 2.19 (s, 3H), 1.72 (s, 3H).

Example 59b. Diastereomer 2. Peak 2. LCMS calculated for C₃₈H₃₇F₂N₈O (M+H)⁺ m/z=659.3; found 659.4.

### Example 60a and Example 60b. 8-(1-((2S,4S)-1-(but-2-ynoyl)-2-(cyanomethyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile

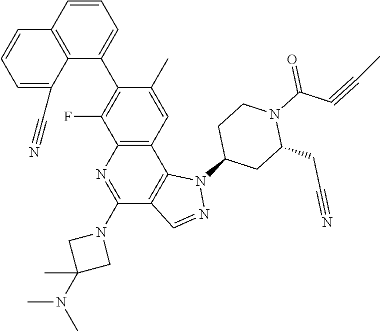 [SMILES: CC#CC(=O)N1CC[C@H](n2ncc3c(N4CC(C)(N(C)C)C4)nc4c(F)c(-c5cccc6cccc(C#N)c56)c(C)cc4c32)C[C@H]1CC#N]

This compound was prepared according to the procedure described in Example 58a and Example 58b, step 4, replacing (E)-4-fluorobut-2-enoic acid with but-2-ynoic acid.

Example 60a. Diastereomer 1. Peak 1. LCMS calculated for C₃₉H₃₈FN₈O (M+H)⁺ m/z=653.3; found 653.3.

Example 60b. Diastereomer 2. Peak 2. LCMS calculated for C₃₉H₃₈FN₈O (M+H)⁺ m/z=653.3; found 653.3.

### Example 61a and Example 61b. 8-(1-((2S,4S)-2-(cyanomethyl)-1-((E)-4-methoxybut-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile

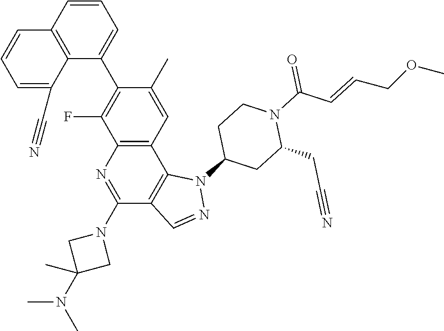 [SMILES: COC/C=C/C(=O)N1CC[C@H](n2ncc3c(N4CC(C)(N(C)C)C4)nc4c(F)c(-c5cccc6cccc(C#N)c56)c(C)cc4c32)C[C@H]1CC#N]

This compound was prepared according to the procedure described in Example 58a and Example 58b, step 4, replacing (E)-4-fluorobut-2-enoic acid with (E)-4-methoxybut-2-enoic acid.

Example 61a. Diastereomer 1. Peak 1. LCMS calculated for C₄₀H₄₂FN₈O₂ (M+H)⁺ m/z=685.3; found 685.4.

Example 61b. Diastereomer 2. Peak 2. LCMS calculated for C₄₀H₄₂FN₈O₂ (M+H)⁺ m/z=685.3; found 685.4.

### Example 62a and Example 62b. 8-(1-((2S,4S)-2-(cyanomethyl)-1-((E)-4-(dimethylamino)but-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile

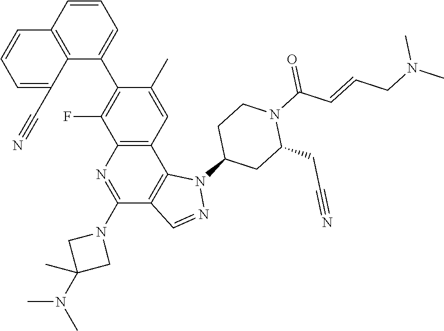 [SMILES: Cc1cc2c(nc(N3CC(C)(N(C)C)C3)c3cnn([C@H]4CCN(C(=O)/C=C/CN(C)C)[C@H](CC#N)C4)c32)c(F)c1-c1cccc2cccc(C#N)c12]

This compound was prepared according to the procedure described in Example 58a and Example 58b, step 4, replacing (E)-4-fluorobut-2-enoic acid with (E)-4-(dimethylamino)but-2-enoic acid hydrochloride.

Example 62a. Diastereomer 1. Peak 1. LCMS calculated for C₄₁H₄₅FN₉O (M+H)⁺ m/z=698.4; found 698.5.

Example 62b. Diastereomer 2. Peak 2. LCMS calculated for C41H₄₅FN₉O (M+H)⁺ m/z=698.4; found 698.5.

### Example 63. 2-((2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile

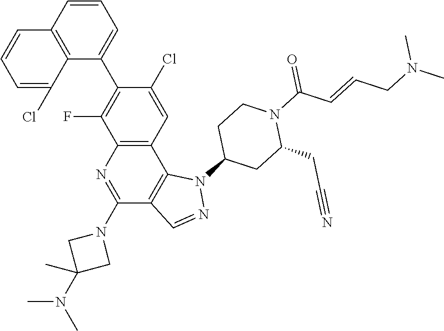 [SMILES: CN(C)C/C=C/C(=O)N1CC[C@H](n2ncc3c(N4CC(C)(N(C)C)C4)nc4c(F)c(-c5cccc6cccc(Cl)c56)c(Cl)cc4c32)C[C@H]1CC#N]

### Step 1. methyl 2-amino-4-(8-chloronaphthalen-1-yl)-3-fluorobenzoate

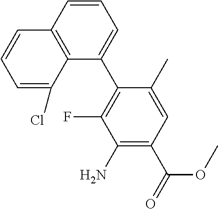 [SMILES: COC(=O)c1cc(C)c(-c2cccc3cccc(Cl)c23)c(F)c1N]

The title compound was synthesized according to the procedure described for Example 27 in step 3, utilizing 1-bromo-8-chloronaphthalene instead of 1-bromo-3-methyl-2-(trifluoromethyl)benzene. LCMS calculated for C₁₈H₁₄ClFNO₂ (M+H)⁺ m/z=330.1; found 330.1.

### Step 2. methyl 2-amino-5-chloro-4-(8-chloronaphthalen-1-yl)-3-fluorobenzoate

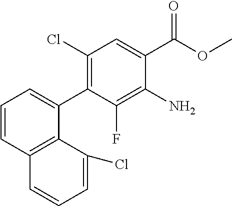 [SMILES: COC(=O)c1cc(Cl)c(-c2cccc3cccc(Cl)c23)c(F)c1N]

The title compound was synthesized according to the procedure described for Example 27 in step 4, utilizing methyl 2-amino-4-(8-chloronaphthalen-1-yl)-3-fluorobenzoate instead of methyl 3-amino-2-fluoro-3′-methyl-2′-(trifluoromethyl)-[1,1′-biphenyl]-4-carboxylate. LCMS calculated for C₁₈H₁₃Cl₂FNO₂ (M+H)⁺ m/z=364.0; found 364.0.

### Step 3. methyl 5-chloro-4-(8-chloronaphthalen-1-yl)-2-(3-ethoxy-3-oxopropanamido)-3-fluorobenzoate

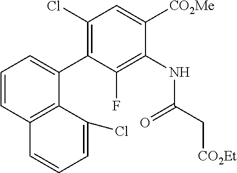 [SMILES: CCOC(=O)CC(=O)Nc1c(C(=O)OC)cc(Cl)c(-c2cccc3cccc(Cl)c23)c1F]

This compound was prepared according to the procedure described in Example 27, in Step 5 replacing methyl 3-amino-6-chloro-2-fluoro-3′-methyl-2′-(trifluoromethyl)-[1,1′-biphenyl]-4-carboxylate with methyl 2-amino-5-chloro-4-(8-chloronaphthalen-1-yl)-3-fluorobenzoate. LC-MS calculated for C₂₃H₁₉Cl₂FNO₅ (M+H)⁺: m/z=478.1; found 478.1.

### Step 4. ethyl 2,4,6-trichloro-7-(8-chloronaphthalen-1-yl)-8-fluoroquinoline-3-carboxylate

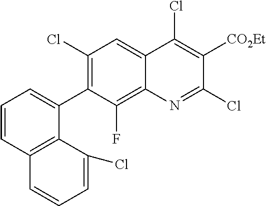 [SMILES: CCOC(=O)c1c(Cl)nc2c(F)c(-c3cccc4cccc(Cl)c34)c(Cl)cc2c1Cl]

This compound was prepared according to the procedure described in Example 27, in Step 6 replacing methyl 6-chloro-3-(3-ethoxy-3-oxopropanamido)-2-fluoro-3′-methyl-2′-(trifluoromethyl)-[1,1′-biphenyl]-4-carboxylate with methyl 5-chloro-4-(8-chloronaphthalen-1-yl)-2-(3-ethoxy-3-oxopropanamido)-3-fluorobenzoate. LC-MS calculated for C₂₂H₁₃Cl4FNO₂ (M+H)⁺: m/z=482.0, 484.0; found 482.0, 484.0.

### Step 5. ethyl 4-(((2S,4S)-1-(tert-butoxycarbonyl)-2-(2-((tert-butyldimethylsilyl)oxy)ethyl)piperidin-4-yl)amino)-2,6-dichloro-7-(8-chloronaphthalen-1-yl)-8-fluoroquinoline-3-carboxylate

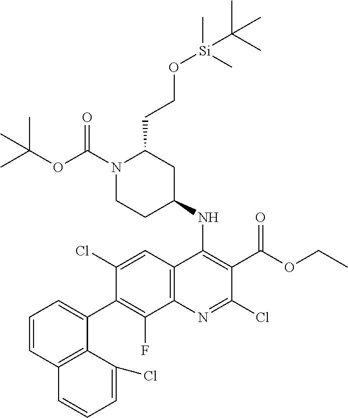 [SMILES: CCOC(=O)c1c(Cl)nc2c(F)c(-c3cccc4cccc(Cl)c34)c(Cl)cc2c1N[C@H]1CCN(C(=O)OC(C)(C)C)[C@H](CCO[Si](C)(C)C(C)(C)C)C1]

This compound was prepared according to the procedure described in Example 3a and Example 3b, in Step 10 replacing ethyl 7-bromo-2,4,6-trichloro-8-fluoroquinoline-3-carboxylate with ethyl 2,4,6-trichloro-7-(8-chloronaphthalen-1-yl)-8-fluoroquinoline-3-carboxylate. LC-MS calculated for C₄₀H₅₀Cl₃FN₃O₅Si (M+H)⁺: m/z=804.3, 806.3; found 804.3, 806.3.

### Step 6. tert-butyl (2S,4S)-2-(2-((tert-butyldimethylsilyl)oxy)ethyl)-4-((2,6-dichloro-7-(8-chloronaphthalen-1-yl)-8-fluoro-3-(hydroxymethyl)quinolin-4-yl)amino)piperidine-1-carboxylate

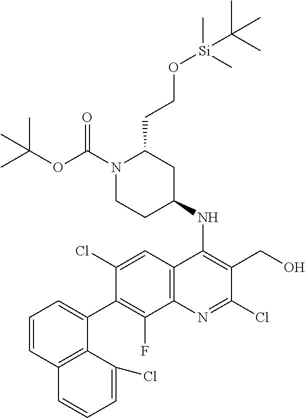 [SMILES: CC(C)(C)OC(=O)N1CC[C@H](Nc2c(CO)c(Cl)nc3c(F)c(-c4cccc5cccc(Cl)c45)c(Cl)cc23)C[C@H]1CCO[Si](C)(C)C(C)(C)C]

This compound was prepared according to the procedure described in Example 27, in Step 9 replacing ethyl 4-(((2S,4S)-1-(tert-butoxycarbonyl)-2-(2-((tert-butyldimethylsilyl)oxy)ethyl)piperidin-4-yl)amino)-2,6-dichloro-8-fluoro-7-(3-methyl-2-(trifluoromethyl)phenyl)quinoline-3-carboxylate with ethyl 4-(((2S,4S)-1-(tert-butoxycarbonyl)-2-(2-((tert-butyldimethylsilyl)oxy)ethyl)piperidin-4-yl)amino)-2,6-dichloro-7-(8-chloronaphthalen-1-yl)-8-fluoroquinoline-3-carboxylate. LC-MS calculated for C₃₈H₄₈Cl₃FN₃O₄Si (M+H)⁺: m/z=762.2, 764.2; found 762.2, 764.2.

### Step 7. tert-butyl (2S,4S)-2-(2-((tert-butyldimethylsilyl)oxy)ethyl)-4-((2,6-dichloro-7-(8-chloronaphthalen-1-yl)-8-fluoro-3-formylquinolin-4-yl)amino)piperidine-1-carboxylate

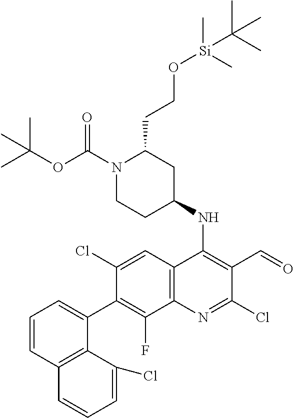 [SMILES: CC(C)(C)OC(=O)N1CC[C@H](Nc2c(C=O)c(Cl)nc3c(F)c(-c4cccc5cccc(Cl)c45)c(Cl)cc23)C[C@H]1CCO[Si](C)(C)C(C)(C)C]

This compound was prepared according to the procedure described in Example 3a and Example 3b, in Step 12 replacing tert-butyl (2S,4S)-4-((7-bromo-2,6-dichloro-8-fluoro-3-(hydroxymethyl)quinolin-4-yl)amino)-2-(2-((tert-butyldimethylsilyl)oxy)ethyl)piperidine-1-carboxylate with tert-butyl (2S,4S)-2-(2-((tert-butyldimethylsilyl)oxy)ethyl)-4-((2,6-dichloro-7-(8-chloronaphthalen-1-yl)-8-fluoro-3-(hydroxymethyl)quinolin-4-yl)amino)piperidine-1-carboxylate. LC-MS calculated for C₃₈H₄₆Cl₃FN₃O₄Si (M+H)⁺: m/z=760.2, 762.2; found 760.3, 762.3.

### Step 8. tert-butyl (2S,4S)-2-(2-((tert-butyldimethylsilyl)oxy)ethyl)-4-((2,6-dichloro-7-(8-chloronaphthalen-1-yl)-8-fluoro-3-((E)-(hydroxyimino)methyl)quinolin-4-yl)amino)piperidine-1-carboxylate

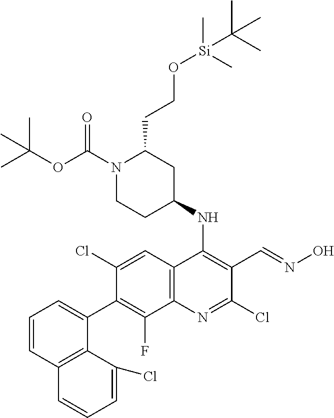 [SMILES: CC(C)(C)OC(=O)N1CC[C@H](Nc2c(/C=N/O)c(Cl)nc3c(F)c(-c4cccc5cccc(Cl)c45)c(Cl)cc23)C[C@H]1CCO[Si](C)(C)C(C)(C)C]

This compound was prepared according to the procedure described in Example 3a and Example 3b, in Step 13 replacing tert-butyl (2S,4S)-4-((7-bromo-2,6-dichloro-8-fluoro-3-formylquinolin-4-yl)amino)-2-(2-((tert-butyldimethylsilyl)oxy)ethyl)piperidine-1-carboxylate with tert-butyl (2S,4S)-2-(2-((tert-butyldimethylsilyl)oxy)ethyl)-4-((2,6-dichloro-7-(8-chloronaphthalen-1-yl)-8-fluoro-3-formylquinolin-4-yl)amino)piperidine-1-carboxylate. LC-MS calculated for C₃₈H₄₇Cl₃FN₄O₄Si (M+H)⁺: m/z=775.2, 777.2; found 775.3, 777.3.

### Step 9. tert-butyl (2S,4S)-2-(2-((tert-butyldimethylsilyl)oxy)ethyl)-4-(4,8-dichloro-7-(8-chloronaphthalen-1-yl)-6-fluoro-1H-pyrazolo[4,3-c]quinolin-1-yl)piperidine-1-carboxylate

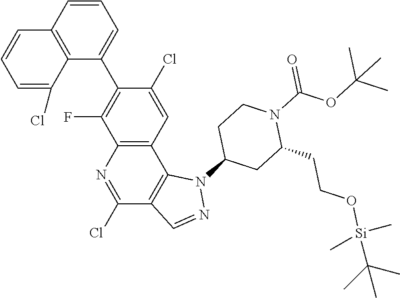 [SMILES: CC(C)(C)OC(=O)N1CC[C@H](n2ncc3c(Cl)nc4c(F)c(-c5cccc6cccc(Cl)c56)c(Cl)cc4c32)C[C@H]1CCO[Si](C)(C)C(C)(C)C]

This compound was prepared according to the procedure described in Example 3a and Example 3b, in Step 14 replacing (tert-butyl (2S,4S)-4-((7-bromo-2,6-dichloro-8-fluoro-3-((E)-(hydroxyimino)methyl)quinolin-4-yl)amino)-2-(2-((tert-butyldimethylsilyl)oxy)ethyl)piperidine-1-carboxylate with tert-butyl (2S,4S)-2-(2-((tert-butyldimethylsilyl)oxy)ethyl)-4-((2,6-dichloro-7-(8-chloronaphthalen-1-yl)-8-fluoro-3-((E)-(hydroxyimino)methyl)quinolin-4-yl)amino)piperidine-1-carboxylate. LC-MS calculated for C₃₈H₄₅Cl₃FN₄O₃Si (M+H)⁺: m/z=757.2, 759.2; found 757.3, 759.3.

### Step 10. tert-butyl (2S,4S)-2-(2-((tert-butyldimethylsilyl)oxy)ethyl)-4-(8-chloro-7-(8-chloronaphthalen-1-yl)-6-fluoro-4-(methylthio)-1H-pyrazolo[4,3-c]quinolin-1-yl)piperidine-1-carboxylate

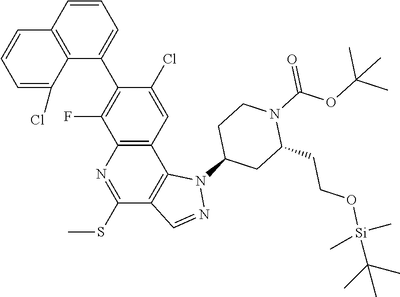 [SMILES: CSc1nc2c(F)c(-c3cccc4cccc(Cl)c34)c(Cl)cc2c2c1cnn2[C@H]1CCN(C(=O)OC(C)(C)C)[C@H](CCO[Si](C)(C)C(C)(C)C)C1]

This compound was prepared according to the procedure described in Example 3a and Example 3b, in Step 15 replacing tert-butyl (2S,4S)-4-(7-bromo-4,8-dichloro-6-fluoro-1H-pyrazolo[4,3-c]quinolin-1-yl)-2-(2-((tert-butyldimethylsilyl)oxy)ethyl)piperidine-1-carboxylate with tert-butyl (2S,4S)-2-(2-((tert-butyldimethylsilyl)oxy)ethyl)-4-(4,8-dichloro-7-(8-chloronaphthalen-1-yl)-6-fluoro-1H-pyrazolo[4,3-c]quinolin-1-yl)piperidine-1-carboxylate. LC-MS calculated for C₃₉H₄₈Cl₂FN₄O₃SSi (M+H)⁺: m/z=769.3, 771.3; found 769.3, 771.3.

### Step 11. tert-butyl (2S,4S)-4-(8-chloro-7-(8-chloronaphthalen-1-yl)-6-fluoro-4-(methylthio)-1H-pyrazolo[4,3-c]quinolin-1-yl)-2-(2-hydroxyethyl)piperidine-1-carboxylate

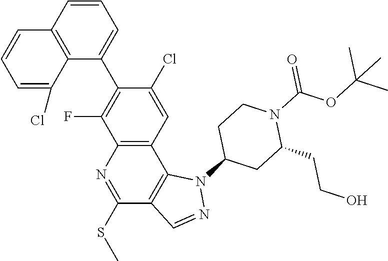 [SMILES: CSc1nc2c(F)c(-c3cccc4cccc(Cl)c34)c(Cl)cc2c2c1cnn2[C@H]1CCN(C(=O)OC(C)(C)C)[C@H](CCO)C1]

This compound was prepared according to the procedure described in Example 3a and Example 3b, in Step 16 replacing tert-butyl (2S,4S)-4-(7-bromo-8-chloro-6-fluoro-4-(methylthio)-1H-pyrazolo[4,3-c]quinolin-1-yl)-2-(2-((tert-butyldimethylsilyl)oxy)ethyl)piperidine-1-carboxylate with tert-butyl (2S,4S)-2-(2-((tert-butyldimethylsilyl)oxy)ethyl)-4-(8-chloro-7-(8-chloronaphthalen-1-yl)-6-fluoro-4-(methylthio)-1H-pyrazolo[4,3-c]quinolin-1-yl)piperidine-1-carboxylate. LC-MS calculated for C₃₃H₃₄Cl₂FN₄O₃S (M+H)⁺: m/z=655.2, 657.2; found 655.3, 657.2.

### Step 12. tert-butyl (2S,4S)-4-(8-chloro-7-(8-chloronaphthalen-1-yl)-6-fluoro-4-(methylthio)-1H-pyrazolo[4,3-c]quinolin-1-yl)-2-(cyanomethyl)piperidine-1-carboxylate

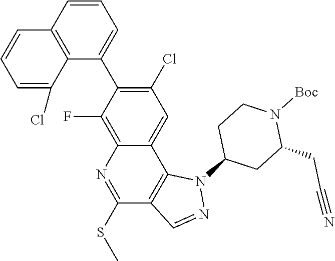 [SMILES: CSc1nc2c(F)c(-c3cccc4cccc(Cl)c34)c(Cl)cc2c2c1cnn2[C@H]1CCN(C(=O)OC(C)(C)C)[C@H](CC#N)C1]

This compound was prepared according to the procedure described in Example 3a and Example 3b, in Step 17 replacing tert-butyl (2S,4S)-4-(7-bromo-8-chloro-6-fluoro-4-(methylthio)-1H-pyrazolo[4,3-c]quinolin-1-yl)-2-(2-hydroxyethyl)piperidine-1-carboxylate with tert-butyl (2S,4S)-4-(8-chloro-7-(8-chloronaphthalen-1-yl)-6-fluoro-4-(methylthio)-1H-pyrazolo[4,3-c]quinolin-1-yl)-2-(2-hydroxyethyl)piperidine-1-carboxylate. LC-MS calculated for C₃₃H₃₁Cl₂FN₅O₂S (M+H)⁺: m/z=650.2, 652.2; found 650.2, 652.3.

### Step 13. tert-butyl (2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)-2-(cyanomethyl)piperidine-1-carboxylate

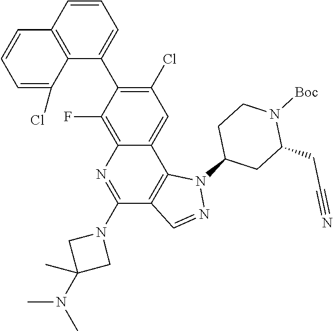 [SMILES: CN(C)C1(C)CN(c2nc3c(F)c(-c4cccc5cccc(Cl)c45)c(Cl)cc3c3c2cnn3[C@H]2CCN(C(=O)OC(C)(C)C)[C@H](CC#N)C2)C1]

This compound was prepared according to the procedure described in Example 58a and Example 58b, in Step 1 replacing tert-butyl (2S,4S)-4-(7-bromo-6-fluoro-8-methyl-4-(methylthio)-1H-pyrazolo[4,3-c]quinolin-1-yl)-2-(cyanomethyl)piperidine-1-carboxylate with tert-butyl (2S,4S)-4-(8-chloro-7-(8-chloronaphthalen-1-yl)-6-fluoro-4-(methylthio)-1H-pyrazolo[4,3-c]quinolin-1-yl)-2-(cyanomethyl)piperidine-1-carboxylate. LC-MS calculated for C₃₈H₄₁Cl₂FN₇O₂ (M+H)⁺: m/z=716.3, 718.3; found 716.3, 718.3.

### Step 14. 2-((2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)piperidin-2-yl)acetonitrile

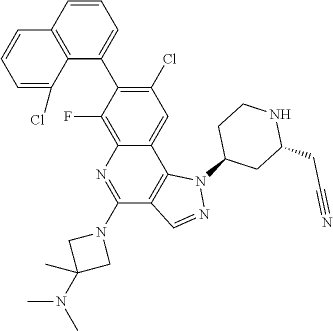 [SMILES: CN(C)C1(C)CN(c2nc3c(F)c(-c4cccc5cccc(Cl)c45)c(Cl)cc3c3c2cnn3[C@H]2CCN[C@H](CC#N)C2)C1]

This compound was prepared according to the procedure described in Example 21a and Example 21b, in Step 4 replacing tert-butyl (2S,4S)-4-(8-chloro-4-(3-(dimethylamino)azetidin-1-yl)-6-fluoro-7-(5-fluoroquinolin-8-yl)-1H-pyrazolo[4,3-c]quinolin-1-yl)-2-(cyanomethyl)piperidine-1-carboxylate with tert-butyl (2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)-2-(cyanomethyl)piperidine-1-carboxylate. LC-MS calculated for C₃₃H₃₃Cl₂FN₇ (M+H)⁺: m/z=616.2, 618.2; found 616.3, 618.3.

### Step 15. 2-((2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile

To a solution of (E)-4-(dimethylamino)but-2-enoic acid hydrochloride (3.3 mg, 0.020 mmol) and 2-((2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)piperidin-2-yl)acetonitrile bis(2,2,2-trifluoroacetate) (14 mg, 0.017 mmol) in DMF (1.0 ml) was added HATU (8.2 mg, 0.022 mmol) and DIEA (14.5 μl, 0.083 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired product (7.0 mg, 58%). LC-MS calculated for C₃₉H₄₂Cl₂FN₈O (M+H)⁺: m/z=727.3, 729.3; found 727.4, 729.3.

### Example 64a and Example 64b. 2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-fluorobut-2-enoyl)piperidin-2-yl)acetonitrile

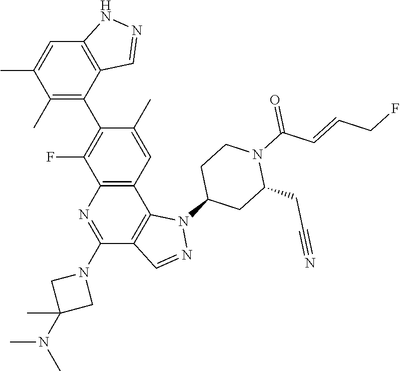 [SMILES: Cc1cc2[nH]ncc2c(-c2c(C)cc3c(nc(N4CC(C)(N(C)C)C4)c4cnn([C@H]5CCN(C(=O)/C=C/CF)[C@H](CC#N)C5)c43)c2F)c1C]

### Step 1. tert-butyl (2S,4S)-2-(cyanomethyl)-4-(7-(5,6-dimethyl-1-(tetrahydro-2H-pyran-2-yl)-1H-indazol-4-yl)-6-fluoro-8-methyl-4-(methylthio)-1H-pyrazolo[4,3-c]quinolin-1-yl)piperidine-1-carboxylate

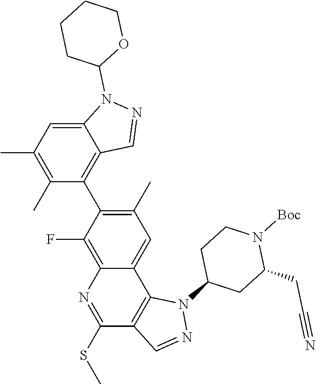 [SMILES: CSc1nc2c(F)c(-c3c(C)c(C)cc4c3cnn4C3CCCCO3)c(C)cc2c2c1cnn2[C@H]1CCN(C(=O)OC(C)(C)C)[C@H](CC#N)C1]

tert-butyl (2S,4S)-4-(7-bromo-6-fluoro-8-methyl-4-(methylthio)-1H-pyrazolo[4,3-c]quinolin-1-yl)-2-(cyanomethyl)piperidine-1-carboxylate (465 mg, 0.848 mmol), 5,6-dimethyl-1-(tetrahydro-2H-pyran-2-yl)-4-(4,4,5,5-tetramethyl-1,3,2-dioxaborolan-2-yl)-1H-indazole (362 mg, 1.017 mmol), tetrakis(triphenylphosphine)palladium(0) (147 mg, 0.127 mmol), and sodium bicarbonate (178 mg, 2.12 mmol) were heated in 5:1 dioxane:water (6 ml) at 105° C. overnight. The mixture was extracted between brine/EtOAc, dried over MgSO₄, and purified by flash chromatography (480 mg, 81%). LC-MS calculated for C₃₈H₄₅FN₇O₃S (M+H)⁺: m/z=698.3; found 698.4.

### Step 2. tert-butyl (2S,4S)-2-(cyanomethyl)-4-(7-(5,6-dimethyl-1-(tetrahydro-2H-pyran-2-yl)-1H-indazol-4-yl)-4-(3-(dimethylamino)-3-methylazetidin-1-yl)-6-fluoro-8-methyl-1H-pyrazolo[4,3-c]quinolin-1-yl)piperidine-1-carboxylate

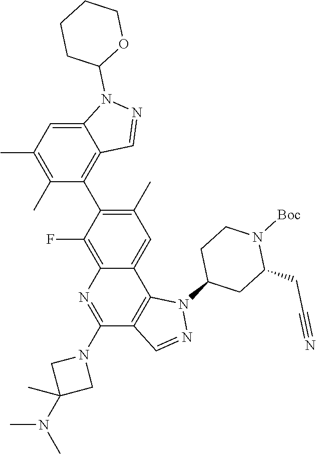 [SMILES: Cc1cc2c(cnn2C2CCCCO2)c(-c2c(C)cc3c(nc(N4CC(C)(N(C)C)C4)c4cnn([C@H]5CCN(C(=O)OC(C)(C)C)[C@H](CC#N)C5)c43)c2F)c1C]

This compound was prepared according to the procedure described in Example 58a and Example 58b, in Step 1 replacing tert-butyl (2S,4S)-4-(7-bromo-6-fluoro-8-methyl-4-(methylthio)-1H-pyrazolo[4,3-c]quinolin-1-yl)-2-(cyanomethyl)piperidine-1-carboxylate with tert-butyl (2S,4S)-2-(cyanomethyl)-4-(7-(5,6-dimethyl-1-(tetrahydro-2H-pyran-2-yl)-1H-indazol-4-yl)-6-fluoro-8-methyl-4-(methylthio)-1H-pyrazolo[4,3-c]quinolin-1-yl)piperidine-1-carboxylate. LC-MS calculated for C₄₃H₅₅FN₉O₃ (M+H)⁺: m/z=764.4; found 764.5.

### Step 3. 2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)piperidin-2-yl)acetonitrile

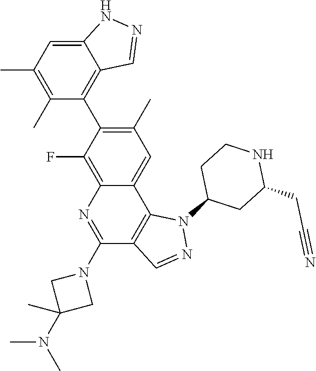 [SMILES: Cc1cc2[nH]ncc2c(-c2c(C)cc3c(nc(N4CC(C)(N(C)C)C4)c4cnn([C@H]5CCN[C@H](CC#N)C5)c43)c2F)c1C]

This compound was prepared according to the procedure described in Example 3a and Example 3b, in Step 20 replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-2-(cyanomethyl)-4-(7-(5,6-dimethyl-1-(tetrahydro-2H-pyran-2-yl)-1H-indazol-4-yl)-4-(3-(dimethylamino)-3-methylazetidin-1-yl)-6-fluoro-8-methyl-1H-pyrazolo[4,3-c]quinolin-1-yl)piperidine-1-carboxylate.

Diastereomer 1. Peak 1. LC-MS calculated for C₃₃H₃₉FN₉ (M+H)⁺: m/z=580.3; found 580.4.

Diastereomer 2. Peak 2. LC-MS calculated for C₃₃H₃₉FN₉ (M+H)⁺: m/z=580.3; found 580.4.

### Step 4. 2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-fluorobut-2-enoyl)piperidin-2-yl)acetonitrile

To a solution of (E)-4-fluorobut-2-enoic acid (0.96 mg, 9.21 μmol) and 2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)piperidin-2-yl)acetonitrile bis(2,2,2-trifluoroacetate) (diastereomer 2, peak 2 from last step) (6.2 mg, 7.68 μmol) in DMF (1.0 ml) was added HATU (3.8 mg, 9.98 μmol) and DIEA (6.70 μl, 0.038 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)piperidin-2-yl)acetonitrile bis(2,2,2-trifluoroacetate) (diastereomer 1, peak 1 from last step) from last step).

Example 64a. Diastereomer 1. Peak 1. LCMS calculated for C₃₇H₄₂F₂N₉O (M+H)⁺ m/z=666.3; found 666.4.

Example 64b. Diastereomer 2. Peak 2. LCMS calculated for C₃₇H₄₂F₂N₉O (M+H)⁺ m/z=666.3; found 666.4.

### Example 65a and Example 65b. 2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-methoxybut-2-enoyl)piperidin-2-yl)acetonitrile

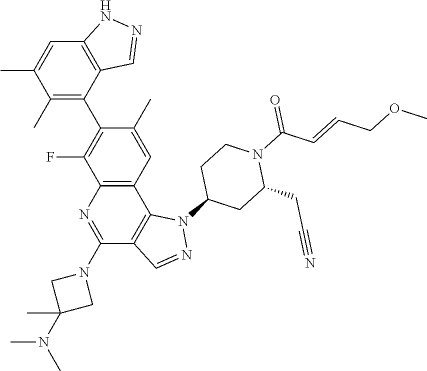

This compound was prepared according to the procedure described in Example 64a and Example 64b, step 4, replacing (E)-4-fluorobut-2-enoic acid with (E)-4-methoxybut-2-enoic acid.

Example 65a. Diastereomer 1. Peak 1. LCMS calculated for C₃₉H₄₅FN₉O₂ (M+H)⁺ m/z=678.4; found 678.4.

Example 65b. Diastereomer 2. Peak 2. LCMS calculated for C₃₉H₄₅FN₉O₂ (M+H)⁺ m/z=678.4; found 678.4.

### Example 66a and Example 66b. 2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-(dimethylamino)but-2-enoyl)piperidin-2-yl)acetonitrile

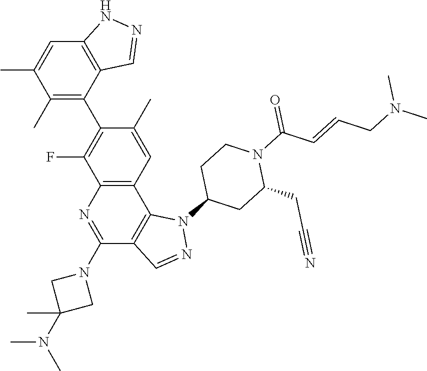

This compound was prepared according to the procedure described in Example 64a and Example 64b, step 4, replacing (E)-4-fluorobut-2-enoic acid with (E)-4-(dimethylamino)but-2-enoic acid hydrochloride.

Example 66a. Diastereomer 1. Peak 1. LCMS calculated for C₃₉H₄₈FN₁₀O (M+H)⁺ m/z=691.4; found 691.5.

Example 66b. Diastereomer 2. Peak 2. LCMS calculated for C₃₉H₄₈FN₁₀O (M+H)⁺ m/z=691.4; found 691.5.

### Example 67a and Example 67b. 2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-fluorobut-2-enoyl)piperidin-2-yl)acetonitrile

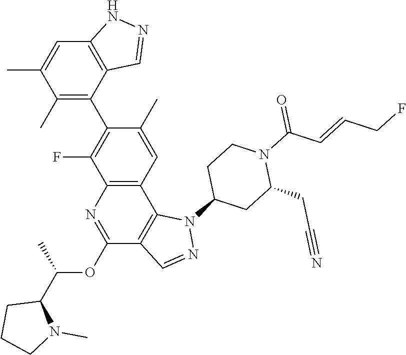 [SMILES: Cc1cc2[nH]ncc2c(-c2c(C)cc3c(nc(O[C@@H](C)[C@@H]4CCCN4C)c4cnn([C@H]5CCN(C(=O)/C=C/CF)[C@H](CC#N)C5)c43)c2F)c1C]

### Step 1. tert-butyl (2S,4S)-2-(cyanomethyl)-4-(7-(5,6-dimethyl-1-(tetrahydro-2H-pyran-2-yl)-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidine-1-carboxylate

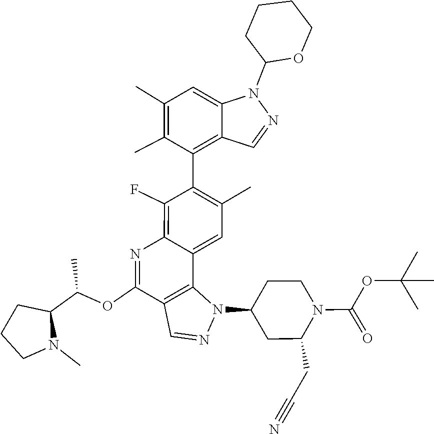 [SMILES: Cc1cc2c(cnn2C2CCCCO2)c(-c2c(C)cc3c(nc(O[C@@H](C)[C@@H]4CCCN4C)c4cnn([C@H]5CCN(C(=O)OC(C)(C)C)[C@H](CC#N)C5)c43)c2F)c1C]

m-CPBA (131 mg, 0.757 mmol) was added to a solution of tert-butyl (2S,4S)-2-(cyanomethyl)-4-(7-(5,6-dimethyl-1-(tetrahydro-2H-pyran-2-yl)-1H-indazol-4-yl)-6-fluoro-8-methyl-4-(methylthio)-1H-pyrazolo[4,3-c]quinolin-1-yl)piperidine-1-carboxylate (240 mg, 0.34 mmol) in DCM (3.5 mL) at 0° C. and then the reaction was stirred at this temperature for 20 min. The reaction was quenched by adding saturated Na₂S₂O₃, diluted with ethyl acetate and washed with saturated NaHCO₃, brine, filtered, dried and concentrated and the crude was used in the next step directly.

1.0 M LiHMDS in THF (770 μl, 0.770 mmol) was added to a solution of (S)-1-((S)-1-methylpyrrolidin-2-yl)ethan-1-ol (100 mg, 0.770 mmol) in THF (1 mL). The resulting mixture was stirred at rt for 30 min. A solution of tert-butyl (2S,4S)-2-(cyanomethyl)-4-(7-(5,6-dimethyl-1-(tetrahydro-2H-pyran-2-yl)-1H-indazol-4-yl)-6-fluoro-8-methyl-4-(methylsulfinyl)-1H-pyrazolo[4,3-c]quinolin-1-yl)piperidine-1-carboxylate (250 mg, 0.350 mmol) in THF (2.0 ml) was added to reaction vial and then stirred at 60° C. for 2 h. The reaction mixture was diluted with ethyl acetate and water. The organic layer was dried over Na₂SO₄, filtered and concentrated. The residue was purified with silica gel column (eluting with a gradient of 0-20% methanol in DCM) to give the desired product as yellow foam (105 mg, 39%). LC-MS calculated for C₄₄H₅₅FN₈O₄ (M+H)⁺: m/z=779.4; found 779.5.

### Step 2. 2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidin-2-yl)acetonitrile

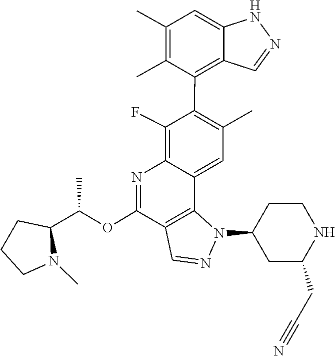 [SMILES: Cc1cc2[nH]ncc2c(-c2c(C)cc3c(nc(O[C@@H](C)[C@@H]4CCCN4C)c4cnn([C@H]5CCN[C@H](CC#N)C5)c43)c2F)c1C]

This compound was prepared according to the procedure described in Example 3a and Example 3b, in Step 20 replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-2-(cyanomethyl)-4-(7-(5,6-dimethyl-1-(tetrahydro-2H-pyran-2-yl)-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidine-1-carboxylate.

Diastereomer 1. Peak 1. LC-MS calculated for C₃₄H₄₀FN₈O (M+H)⁺: m/z=595.3; found 595.4.

Diastereomer 2. Peak 2. LC-MS calculated for C₃₄H₄₀FN₈O (M+H)⁺: m/z=595.3; found 595.4.

### Step 3. 2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-fluorobut-2-enoyl)piperidin-2-yl)acetonitrile

To a solution of (E)-4-fluorobut-2-enoic acid (0.91 mg, 8.75 μmol) and 2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidin-2-yl)acetonitrile bis(2,2,2-trifluoroacetate) (6.0 mg, 7.3 μmol) (Diastereomer 1, peak 1 from last step) in DMF (1.0 ml) was added HATU (3.6 mg, 9.5 μmol) and DIEA (6.4 μl, 0.036 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidin-2-yl)acetonitrile bis(2,2,2-trifluoroacetate) (diastereomer 2 peak 2 from last step).

Example 67a. Diastereomer 1. Peak 1. LCMS calculated for C₃₈H₄₃F₂N₈O₂ (M+H)⁺ m/z=681.3; found 681.4.

Example 67b. Diastereomer 2. Peak 2. LCMS calculated for C₃₈H₄₃F₂N₈O₂ (M+H)⁺ m/z=681.3; found 681.4.

### Example 68a and Example 68b. 2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-(2-fluoroacryloyl)piperidin-2-yl)acetonitrile

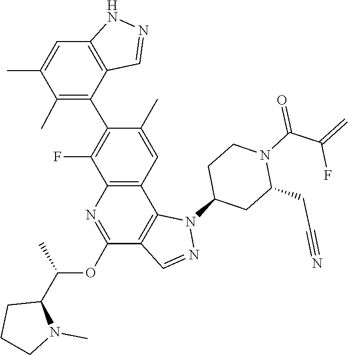 [SMILES: C=C(F)C(=O)N1CC[C@H](n2ncc3c(O[C@@H](C)[C@@H]4CCCN4C)nc4c(F)c(-c5c(C)c(C)cc6[nH]ncc56)c(C)cc4c32)C[C@H]1CC#N]

This compound was prepared according to the procedure described in Example 67a and Example 67b, step 3, replacing (E)-4-fluorobut-2-enoic acid with 2-fluoroacrylic acid.

Example 68a. Diastereomer 1. Peak 1. LCMS calculated for C₃₇H₄₁F₂N₈O₂ (M+H)⁺ m/z=667.3; found 667.4.

Example 68b. Diastereomer 2. Peak 2. LCMS calculated for C₃₇H₄₁F₂N₈O₂ (M+H)⁺ m/z=667.3; found 667.4.

### Example 69a and Example 69b. 2-((2S,4S)-1-(but-2-ynoyl)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidin-2-yl)acetonitrile

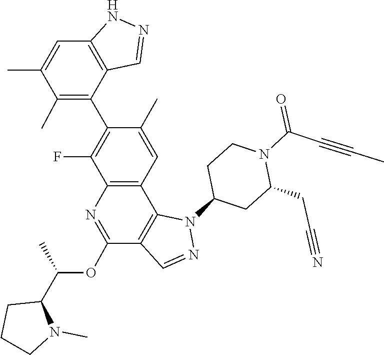 [SMILES: CC#CC(=O)N1CC[C@H](n2ncc3c(O[C@@H](C)[C@@H]4CCCN4C)nc4c(F)c(-c5c(C)c(C)cc6[nH]ncc56)c(C)cc4c32)C[C@H]1CC#N]

This compound was prepared according to the procedure described in Example 67a and Example 67b, step 3, replacing (E)-4-fluorobut-2-enoic acid with but-2-ynoic acid.

Example 69a. Diastereomer 1. Peak 1. LCMS calculated for C₃₈H₄₂FN₈O₂ (M+H)⁺ m/z=661.3; found 661.4.

Example 69b. Diastereomer 2. Peak 2. LCMS calculated for C₃₈H₄₂FN₈O₂ (M+H)⁺ m/z=661.3; found 661.4.

### Example 70a and Example 70b. 2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-methoxybut-2-enoyl)piperidin-2-yl)acetonitrile

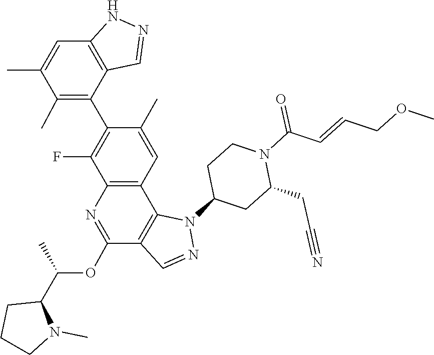 [SMILES: COC/C=C/C(=O)N1CC[C@H](n2ncc3c(O[C@@H](C)[C@@H]4CCCN4C)nc4c(F)c(-c5c(C)c(C)cc6[nH]ncc56)c(C)cc4c32)C[C@H]1CC#N]

This compound was prepared according to the procedure described in Example 67a and Example 67b, step 3, replacing (E)-4-fluorobut-2-enoic acid with (E)-4-methoxybut-2-enoic acid.

Example 70a. Diastereomer 1. Peak 1. LCMS calculated for C₃₉H₄₆FN₈O₃ (M+H)⁺ m/z=693.4; found 693.5.

Example 70b. Diastereomer 2. Peak 2. LCMS calculated for C₃₉H₄₆FN₈O₃ (M+H)⁺ m/z=693.4; found 693.5.

### Example 71a and Example 71b. 2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-(dimethylamino)but-2-enoyl)piperidin-2-yl)acetonitrile

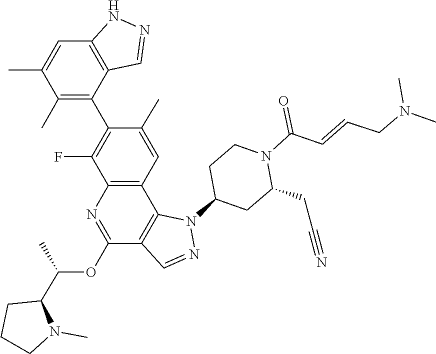 [SMILES: Cc1cc2[nH]ncc2c(-c2c(C)cc3c(nc(O[C@@H](C)[C@@H]4CCCN4C)c4cnn([C@H]5CCN(C(=O)/C=C/CN(C)C)[C@H](CC#N)C5)c43)c2F)c1C]

This compound was prepared according to the procedure described in Example 67a and Example 67b, step 3, replacing (E)-4-fluorobut-2-enoic acid with (E)-4-(dimethylamino)but-2-enoic acid hydrochloride.

Example 71a. Diastereomer 1. Peak 1. LCMS calculated for C₄₀H₄₉FN₉O₂ (M+H)⁺ m/z=706.4; found 706.4.

Example 71b. Diastereomer 2. Peak 2. LCMS calculated for C₄₀H₄₉FN₉O₂ (M+H)⁺ m/z=706.4; found 706.4.

### Example 72a and Example 72b. 2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile

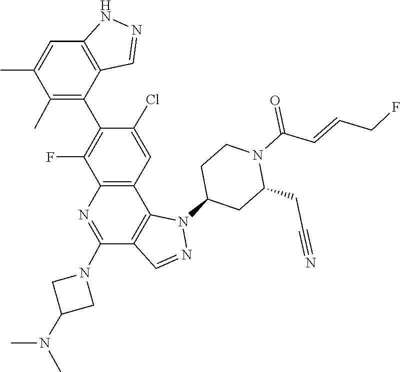 [SMILES: Cc1cc2[nH]ncc2c(-c2c(Cl)cc3c(nc(N4CC(N(C)C)C4)c4cnn([C@H]5CCN(C(=O)/C=C/CF)[C@H](CC#N)C5)c43)c2F)c1C]

### Step 1. tert-butyl (2S,4S)-4-(8-chloro-7-(5,6-dimethyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate

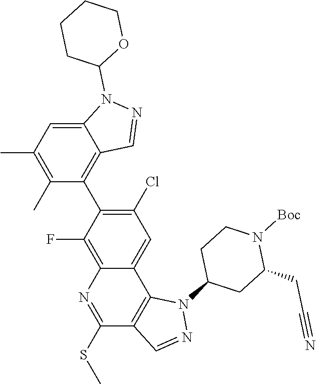 [SMILES: CSc1nc2c(F)c(-c3c(C)c(C)cc4c3cnn4C3CCCCO3)c(Cl)cc2c2c1cnn2[C@H]1CCN(C(=O)OC(C)(C)C)[C@H](CC#N)C1]

A microwave vial charged with tert-butyl (2S,4S)-4-(7-bromo-8-chloro-6-fluoro-4-(methylthio)-1H-pyrazolo[4,3-c]quinolin-1-yl)-2-(cyanomethyl)piperidine-1-carboxylate (1.05 g, 1.846 mmol), 5,6-dimethyl-1-(tetrahydro-2H-pyran-2-yl)-4-(4,4,5,5-tetramethyl-1,3,2-dioxaborolan-2-yl)-1H-indazole (0.921 g, 2.58 mmol), tetrakis(triphenylphosphine)palladium(0) (0.320 g, 0.277 mmol), sodium carbonate (0.782 g, 7.38 mmol) and 5:1 dioxane: water (12 ml) were heated under N2 atmosphere at 105° C. overnight. The mixture was extracted between brine/EtOAc, dried over MgSO₄, and purified by flash chromatography (eluting with a gradient of 0-30% ethyl acetate in hexanes) to give the desired product (1.3 g, 98%). LC-MS calculated for C₃₇H₄₂ClFN₇O₃S (M+H)⁺: m/z=718.3; found 718.4.

### Step 2. tert-butyl (2S,4S)-4-(8-chloro-7-(5,6-dimethyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate

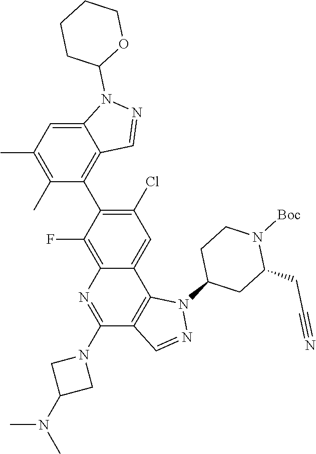 [SMILES: Cc1cc2c(cnn2C2CCCCO2)c(-c2c(Cl)cc3c(nc(N4CC(N(C)C)C4)c4cnn([C@H]5CCN(C(=O)OC(C)(C)C)[C@H](CC#N)C5)c43)c2F)c1C]

This compound was prepared according to the procedure described in Example 21a and Example 21b, step 19, replacing tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate with tert-butyl (2S,4S)-4-(8-chloro-7-(5,6-dimethyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate. LC-MS calculated for C₄₁H₅₀ClFN₉O₃ (M+H)⁺: m/z=770.4; found 770.5.

### Step 3. 2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)piperidin-2-yl)acetonitrile

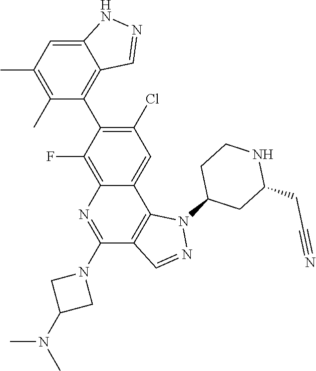 [SMILES: Cc1cc2[nH]ncc2c(-c2c(Cl)cc3c(nc(N4CC(N(C)C)C4)c4cnn([C@H]5CCN[C@H](CC#N)C5)c43)c2F)c1C]

This compound was prepared according to the procedure described in Example 3a and Example 3b, in Step 20 replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-4-(8-chloro-7-(5,6-dimethyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate.

Diastereomer 1. Peak 1. LC-MS calculated for C₃₁H₃₄ClFN₉ (M+H)⁺: m/z=586.3; found 586.4.

Diastereomer 2. Peak 2. LC-MS calculated for C₃₁H₃₄ClFN₉ (M+H)⁺: m/z=586.3; found 586.4.

### Step 4. 2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile

To a solution of (E)-4-fluorobut-2-enoic acid (0.951 mg, 9.14 μmol) and 2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)piperidin-2-yl)acetonitrile bis(2,2,2-trifluoroacetate) (diastereomer 2 peak 2 from last step) (6.2 mg, 7.62 μmol) in DMF (1.0 ml) was added HATU (3.8 mg, 9.90 μmol) and DIEA (6.7 μl, 0.038 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)piperidin-2-yl)acetonitrile bis(2,2,2-trifluoroacetate) (diastereomer 1 peak 1 from last step).

Example 72a. Diastereomer 1. Peak 1. LCMS calculated for C₃₅H₃₇ClF₂N₉O (M+H)⁺ m/z=672.3; found 672.3.

Example 72b. Diastereomer 2. Peak 2. LCMS calculated for C₃₅H₃₇ClF₂N₉O (M+H)⁺ m/z=672.3; found 672.3.

### Example 73a and Example 73b. 2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-(2-fluoroacryloyl)piperidin-2-yl)acetonitrile

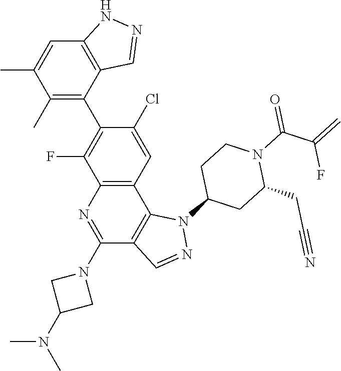 [SMILES: C=C(F)C(=O)N1CC[C@H](n2ncc3c(N4CC(N(C)C)C4)nc4c(F)c(-c5c(C)c(C)cc6[nH]ncc56)c(Cl)cc4c32)C[C@H]1CC#N]

This compound was prepared according to the procedure described in Example 72a and Example 72b, step 4, replacing (E)-4-fluorobut-2-enoic acid with 2-fluoroacrylic acid.

Example 73a. Diastereomer 1. Peak 1. LCMS calculated for C₃₄H₃₅ClF₂N₉O (M+H)⁺ m/z=658.3; found 658.4. ¹H NMR (500 MHz, DMSO-d₆) δ 8.32 (s, 2H), 7.49 (s, 1H), 7.37 (s, 1H), 5.83-5.62 (m, 1H), 5.52-5.26 (m, 2H), 4.99 (s, 1H), 4.78-4.65 (m, 1H), 4.57 (m, 1H), 4.29 (s, 1H), 4.14-3.34 (m, 5H), 3.26 (m, 1H), 2.85 (s, 6H), 2.46 (s, 3H), 2.40-2.21 (m, 4H), 2.10 (s, 3H).

Example 73b. Diastereomer 2. Peak 2. LCMS calculated for C₃₄H₃₅ClF₂N90 (M+H)⁺ m/z=658.3; found 658.4.

### Example 74a and Example 74b. 2-((2S,4S)-1-(but-2-ynoyl)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)piperidin-2-yl)acetonitrile

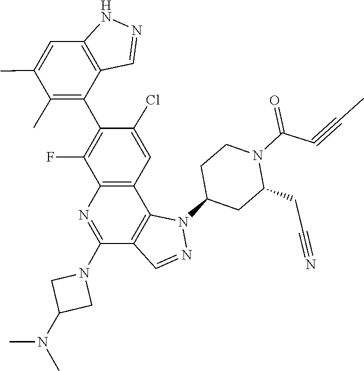 [SMILES: CC#CC(=O)N1CC[C@H](n2ncc3c(N4CC(N(C)C)C4)nc4c(F)c(-c5c(C)c(C)cc6[nH]ncc56)c(Cl)cc4c32)C[C@H]1CC#N]

This compound was prepared according to the procedure described in Example 72a and Example 72b, step 4, replacing (E)-4-fluorobut-2-enoic acid with but-2-ynoic acid.

Example 74a. Diastereomer 1. Peak 1. LCMS calculated for C₃₅H₃₆ClFN₉O (M+H)⁺ m/z=652.3; found 652.3.

Example 74b. Diastereomer 2. Peak 2. LCMS calculated for C₃₅H₃₆ClFN₉O (M+H)⁺ m/z=652.3; found 652.3.

### Example 75a and Example 75b. 2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-methoxybut-2-enoyl)piperidin-2-yl)acetonitrile

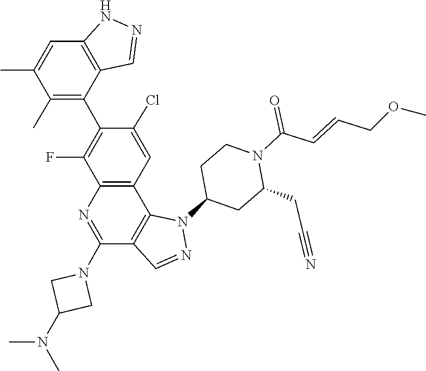

This compound was prepared according to the procedure described in Example 72a and Example 72b, step 4, replacing (E)-4-fluorobut-2-enoic acid with (E)-4-methoxybut-2-enoic acid.

Example 75a. Diastereomer 1. Peak 1. LCMS calculated for C₃₆H₄₀ClFN₉O₂ (M+H)⁺ m/z=684.3; found 684.3.

Example 75b. Diastereomer 2. Peak 2. LCMS calculated for C₃₆H₄₀ClFN₉O₂ (M+H)⁺ m/z=684.3; found 684.3.

### Example 76a and Example 76b. 2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile

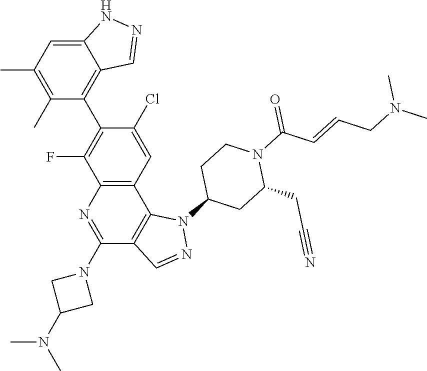

This compound was prepared according to the procedure described in Example 72a and Example 72b, step 4, replacing (E)-4-fluorobut-2-enoic acid with (E)-4-(dimethylamino)but-2-enoic acid hydrochloride.

Example 76a. Diastereomer 1. Peak 1. LCMS calculated for C₃₇H₄₃ClFN₁₀O (M+H)⁺ m/z=697.3; found 697.4.

Example 76b. Diastereomer 2. Peak 2. LCMS calculated for C₃₇H₄₃ClFN₁₀O (M+H)⁺ m/z=697.3; found 697.4.

### Example 77a and Example 77b. 2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-(2-fluoroacryloyl)piperidin-2-yl)acetonitrile

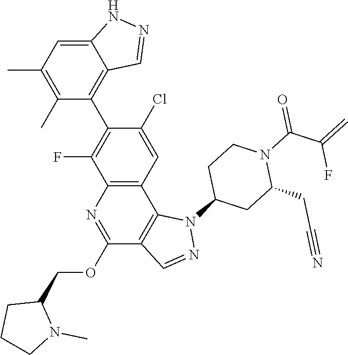

### Step 1. tert-butyl (2S,4S)-4-(8-chloro-7-(5,6-dimethyl-1-(tetrahydro-2H-pyran-2-yl)-1H-indazol-4-yl)-6-fluoro-4-(((S)-1-methylpyrrolidin-2-yl)methoxy)-1H-pyrazolo[4,3-c]quinolin-1-yl)-2-(cyanomethyl)piperidine-1-carboxylate

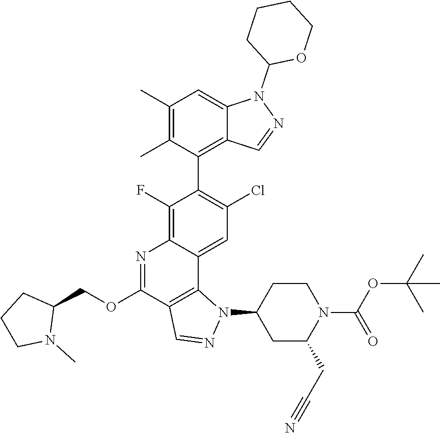 [SMILES: Cc1cc2c(cnn2C2CCCCO2)c(-c2c(Cl)cc3c(nc(OC[C@@H]4CCCN4C)c4cnn([C@H]5CCN(C(=O)OC(C)(C)C)[C@H](CC#N)C5)c43)c2F)c1C]

This compound was prepared according to the procedure described in Example 17a and Example 17b, in Step 1 replacing tert-butyl (2S,4S)-4-(8-chloro-7-(6-chloro-5-methyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate with tert-butyl (2S,4S)-4-(8-chloro-7-(5,6-dimethyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate. LC-MS calculated for C₄₂H₅₁ClFN₈O₄ (M+H)⁺: m/z=785.4; found 785.4.

### Step 2. 2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile

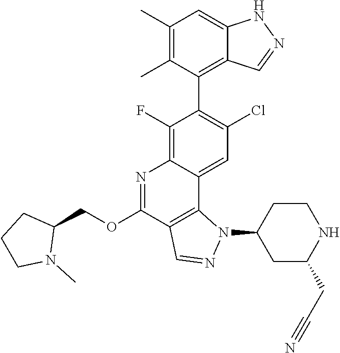 [SMILES: Cc1cc2[nH]ncc2c(-c2c(Cl)cc3c(nc(OC[C@@H]4CCCN4C)c4cnn([C@H]5CCN[C@H](CC#N)C5)c43)c2F)c1C]

This compound was prepared according to the procedure described in Example 3a and Example 3b, in Step 20 replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-4-(8-chloro-7-(5,6-dimethyl-1-(tetrahydro-2H-pyran-2-yl)-1H-indazol-4-yl)-6-fluoro-4-(((S)-1-methylpyrrolidin-2-yl)methoxy)-1H-pyrazolo[4,3-c]quinolin-1-yl)-2-(cyanomethyl)piperidine-1-carboxylate.

Diastereomer 1. Peak 1. LC-MS calculated for C₃₂H₃₅ClFN₈O (M+H)⁺: m/z=601.3; found 601.4.

Diastereomer 2. Peak 2. LC-MS calculated for C₃₂H₃₅ClFN₈O (M+H)⁺: m/z=601.3; found 601.4.

### Step 3. 2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-(2-fluoroacryloyl)piperidin-2-yl)acetonitrile

To a solution of 2-fluoroacrylic acid (0.81 mg, 8.97 μmol) and 2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile bis(2,2,2-trifluoroacetate) (diastereomer 2 peak 2 from last step) (6.2 mg, 7.48 μmol) in DMF (1.0 ml) was added HATU (3.7 mg, 9.7 μmol) and DIEA (6.5 μl, 0.037 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidin-2-yl)acetonitrile bis(2,2,2-trifluoroacetate) (diastereomer 1 peak 1 from last step).

Example 77a. Diastereomer 1. Peak 1. LCMS calculated for C₃₅H₃₆ClF₂N₈O₂ (M+H)⁺ m/z=673.3; found 673.3. ¹H NMR (500 MHz, DMSO-d₆) δ 8.50 (s, 1H), 8.43 (s, 1H), 7.51 (s, 1H), 7.39 (s, 1H), 5.76 (m, 1H), 5.37-5.28 (m, 2H), 5.01 (m, 1H), 4.85 (m, 2H), 4.28 (m, 1H), 3.92 (m, 1H), 3.70-3.52 (2H), 3.48 (m, 1H), 3.33-3.21 (m, 2H), 3.02 (s, 3H), 2.49 (s, 3H), 2.39-2.27 (m, 5H), 2.11 (s, 3H), 2.05 (m, 3H).

Example 77b. Diastereomer 2. Peak 2. LCMS calculated for C₃₅H₃₆ClF₂N₈O₂ (M+H)⁺ m/z=673.3; found 673.3.

### Example 78a and Example 78b. 2-((2S,4S)-1-(but-2-ynoyl)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile

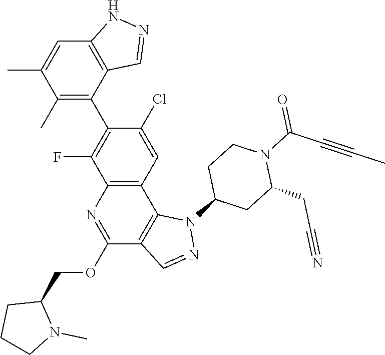 [SMILES: CC#CC(=O)N1CC[C@H](n2ncc3c(OC[C@@H]4CCCN4C)nc4c(F)c(-c5c(C)c(C)cc6[nH]ncc56)c(Cl)cc4c32)C[C@H]1CC#N]

This compound was prepared according to the procedure described in Example 77a and Example 77b, step 3, replacing 2-fluoroacrylic acid with but-2-ynoic acid.

Example 78a. Diastereomer 1. Peak 1. LCMS calculated for C₃₆H₃₇ClFN₈O₂ (M+H)⁺ m/z=667.3; found 667.3.

Example 78b. Diastereomer 2. Peak 2. LCMS calculated for C₃₆H₃₇ClFN₈O₂ (M+H)⁺ m/z=667.3; found 667.3.

### Example 79a and Example 79b. 2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-((E)-4-methoxybut-2-enoyl)piperidin-2-yl)acetonitrile

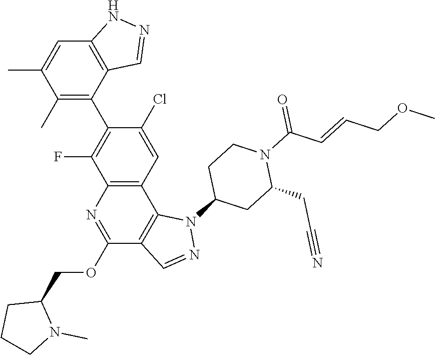 [SMILES: COC/C=C/C(=O)N1CC[C@H](n2ncc3c(OC[C@@H]4CCCN4C)nc4c(F)c(-c5c(C)c(C)cc6[nH]ncc56)c(Cl)cc4c32)C[C@H]1CC#N]

This compound was prepared according to the procedure described in Example 77a and Example 77b, step 3, replacing 2-fluoroacrylic acid with (E)-4-methoxybut-2-enoic acid.

Example 79a. Diastereomer 1. Peak 1. LCMS calculated for C₃₇H₄₁ClFN₈O₃ (M+H)⁺ m/z=699.3; found 699.3.

Example 79b. Diastereomer 2. Peak 2. LCMS calculated for C₃₇H₄₁ClFN₈O₃ (M+H)⁺ m/z=699.3; found 699.3.

### Example 80a and Example 80b. 2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-((E)-4-(dimethylamino)but-2-enoyl)piperidin-2-yl)acetonitrile

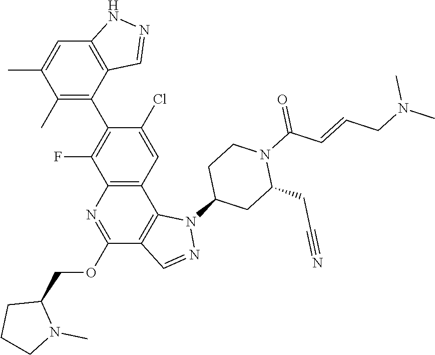 [SMILES: Cc1cc2[nH]ncc2c(-c2c(Cl)cc3c(nc(OC[C@@H]4CCCN4C)c4cnn([C@H]5CCN(C(=O)/C=C/CN(C)C)[C@H](CC#N)C5)c43)c2F)c1C]

This compound was prepared according to the procedure described in Example 77a and Example 77b, step 3, replacing 2-fluoroacrylic acid with (E)-4-(dimethylamino)but-2-enoic acid hydrochloride.

Example 80a. Diastereomer 1. Peak 1. LCMS calculated for C₃₈H₄₄ClFN₉O₂ (M+H)⁺ m/z=712.3; found 712.4.

Example 80b. Diastereomer 2. Peak 2. LCMS calculated for C₃₈H₄₄ClFN₉O₂ (M+H)⁺ m/z=712.3; found 712.4.

### Example 81a and Example 81b. 2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-(2-fluoroacryloyl)piperidin-2-yl)acetonitrile

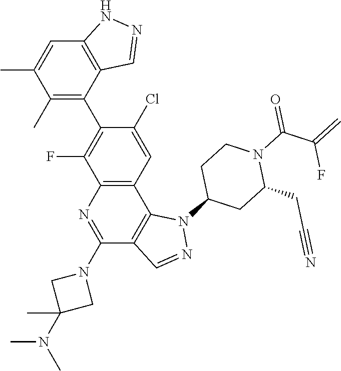 [SMILES: C=C(F)C(=O)N1CC[C@H](n2ncc3c(N4CC(C)(N(C)C)C4)nc4c(F)c(-c5c(C)c(C)cc6[nH]ncc56)c(Cl)cc4c32)C[C@H]1CC#N]

### Step 1. tert-butyl (2S,4S)-4-(8-chloro-7-(5,6-dimethyl-1-(tetrahydro-2H-pyran-2-yl)-1H-indazol-4-yl)-4-(3-(dimethylamino)-3-methylazetidin-1-yl)-6-fluoro-1H-pyrazolo[4,3-c]quinolin-1-yl)-2-(cyanomethyl)piperidine-1-carboxylate

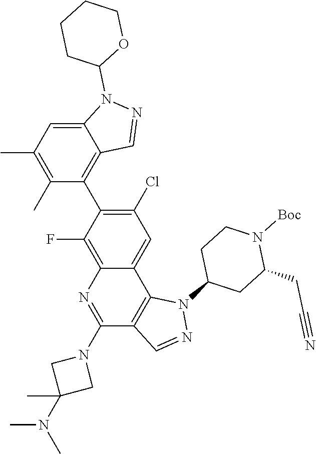 [SMILES: Cc1cc2c(cnn2C2CCCCO2)c(-c2c(Cl)cc3c(nc(N4CC(C)(N(C)C)C4)c4cnn([C@H]5CCN(C(=O)OC(C)(C)C)[C@H](CC#N)C5)c43)c2F)c1C]

This compound was prepared according to the procedure described in Example 58a and Example 58b, in Step 1 replacing tert-butyl (2S,4S)-4-(7-bromo-6-fluoro-8-methyl-4-(methylthio)-1H-pyrazolo[4,3-c]quinolin-1-yl)-2-(cyanomethyl)piperidine-1-carboxylate with tert-butyl (2S,4S)-4-(8-chloro-7-(5,6-dimethyl-1-(tetrahydro-2H-pyran-2-yl)-1H-indazol-4-yl)-6-fluoro-4-(methylthio)-1H-pyrazolo[4,3-c]quinolin-1-yl)-2-(cyanomethyl)piperidine-1-carboxylate. LC-MS calculated for C₄₂H₅₂ClFN₉O₃ (M+H)⁺: m/z=784.4; found 784.5.

### Step 2. 2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile

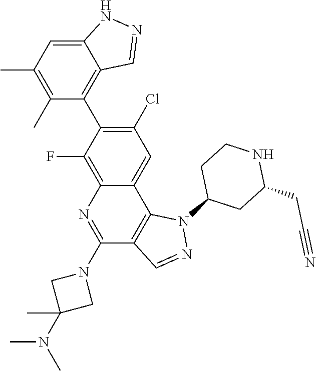 [SMILES: Cc1cc2[nH]ncc2c(-c2c(Cl)cc3c(nc(N4CC(C)(N(C)C)C4)c4cnn([C@H]5CCN[C@H](CC#N)C5)c43)c2F)c1C]

This compound was prepared according to the procedure described in Example 3a and Example 3b, in Step 20 replacing tert-butyl (2S,4S)-4-(8-chloro-7-(6-chloro-5-methyl-1-(tetrahydro-2H-pyran-2-yl)-1H-indazol-4-yl)-4-(3-(dimethylamino)azetidin-1-yl)-6-fluoro-1H-pyrazolo[4,3-c]quinolin-1-yl)-2-(cyanomethyl)piperidine-1-carboxylate with tert-butyl (2S,4S)-4-(8-chloro-7-(5,6-dimethyl-1-(tetrahydro-2H-pyran-2-yl)-1H-indazol-4-yl)-4-(3-(dimethylamino)-3-methylazetidin-1-yl)-6-fluoro-1H-pyrazolo[4,3-c]quinolin-1-yl)-2-(cyanomethyl)piperidine-1-carboxylate.

Diastereomer 1. Peak 1. LC-MS calculated for C₃₂H₃₆ClFN₉ (M+H)⁺: m/z=600.3; found 600.4.

Diastereomer 2. Peak 2. LC-MS calculated for C₃₂H₃₆ClFN₉ (M+H)⁺: m/z=600.3; found 600.4.

### Step 3. 2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-(2-fluoroacryloyl)piperidin-2-yl)acetonitrile

To a solution of 2-fluoroacrylic acid (0.91 mg, 10.1 μmol) and 2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile bis(2,2,2-trifluoroacetate) (Diastereomer 2 peak 2 from last step) (7.0 mg, 8.5 μmol) in DMF (1.0 ml) was added HATU (4.0 mg, 10.6 μmol) and DIEA (5.9 μl, 0.034 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired diastereomer 1.

Diastereomer 2 was synthesized in similar way using 2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile bis(2,2,2-trifluoroacetate) (diastereomer 1, peak 1 from last step).

Example 81a. Diastereomer 1. Peak 1. LCMS calculated for C₃₅H₃₇ClF₂N₉O (M+H)⁺ m/z=672.3; found 672.4. ¹H NMR (600 MHz, DMSO-d₆) δ 8.34 (s, 2H), 7.49 (s, 1H), 7.38 (s, 1H), 5.71 (m, 1H), 5.39 (m, 2H), 5.35-3.50 (m, 8H), 3.28 (m, 1H), 2.82 (s, 6H), 2.47 (s, 3H), 2.31 (m, 4H), 2.06 (s, 3H), 1.68 (s, 3H).

Example 81b. Diastereomer 2. Peak 2. LCMS calculated for C₃₅H₃₇ClF₂N₉O (M+H)⁺ m/z=672.3; found 672.4.

### Example 82a and Example 82b. 2-((2S,4S)-1-(but-2-ynoyl)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile

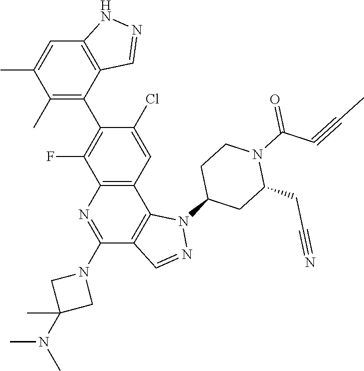 [SMILES: CC#CC(=O)N1CC[C@H](n2ncc3c(N4CC(C)(N(C)C)C4)nc4c(F)c(-c5c(C)c(C)cc6[nH]ncc56)c(Cl)cc4c32)C[C@H]1CC#N]

This compound was prepared according to the procedure described in Example 81a and Example 81b, step 3, replacing 2-fluoroacrylic acid with but-2-ynoic acid.

Example 82a. Diastereomer 1. Peak 1. LCMS calculated for C₃₆H₃₈ClFN₉O (M+H)⁺ m/z=666.3; found 666.4.

Example 82b. Diastereomer 2. Peak 2. LCMS calculated for C₃₆H₃₈ClFN₉O (M+H)⁺ m/z=666.3; found 666.4.

### Example 83a and Example 83b. 2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-((E)-4-methoxybut-2-enoyl)piperidin-2-yl)acetonitrile

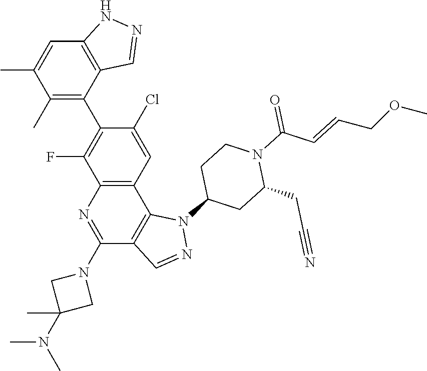

This compound was prepared according to the procedure described in Example 81a and Example 81b, step 3, replacing 2-fluoroacrylic acid with (E)-4-methoxybut-2-enoic acid.

Example 83a. Diastereomer 1. Peak 1. LCMS calculated for C₃₇H₄₂ClFN₉O₂ (M+H)⁺ m/z=698.3; found 698.4. ¹H NMR (600 MHz, DMSO-d₆) δ 8.35 (m, 2H), 7.49 (s, 1H), 7.39 (s, 1H), 6.78-6.71 (m, 2H), 5.68 (m, 1H), 5.27 (s, 0.5H), 4.89 (s, 0.5H), 4.68-4.20 (m, 5H), 4.10 (m, 2H), 3.71-3.44 (m, 1H), 3.33 (s, 3H), 3.29-3.18 (m, 2H), 2.82 (s, 6H), 2.47 (s, 3H), 2.27 (m, 3H), 2.18 (s, 3H), 2.18-2.13 (m, 1H), 1.68 (s, 3H).

Example 83b. Diastereomer 2. Peak 2. LCMS calculated for C₃₇H₄₂ClFN₉O₂ (M+H)⁺ m/z=698.3; found 698.4.

### Example 84a and Example 84b. 2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile

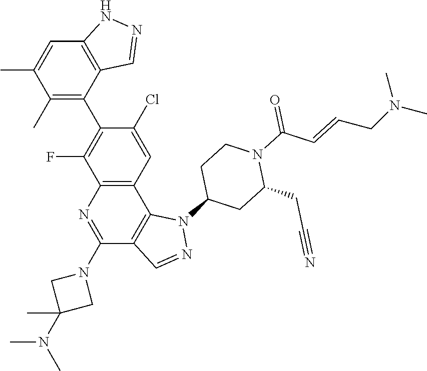

This compound was prepared according to the procedure described in Example 81a and Example 81b, step 3, replacing 2-fluoroacrylic acid with (E)-4-(dimethylamino)but-2-enoic acid hydrochloride.

Example 84a. Diastereomer 1. Peak 1. LCMS calculated for C₃₈H₄₅ClFN₁₀O (M+H)⁺ m/z=711.3; found 711.4.

Example 84b. Diastereomer 2. Peak 2. LCMS calculated for C₃₈H₄₅ClFN₁₀O (M+H)⁺ m/z=711.3; found 711.4.

### Example 85. 2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-fluorobut-2-enoyl)piperidin-2-yl)acetonitrile

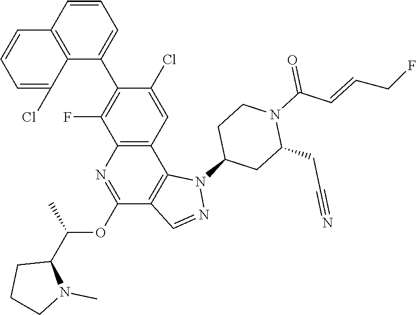 [SMILES: C[C@H](Oc1nc2c(F)c(-c3cccc4cccc(Cl)c34)c(Cl)cc2c2c1cnn2[C@H]1CCN(C(=O)/C=C/CF)[C@H](CC#N)C1)[C@@H]1CCCN1C]

### Step 1. tert-butyl (2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-2-(cyanomethyl)piperidine-1-carboxylate

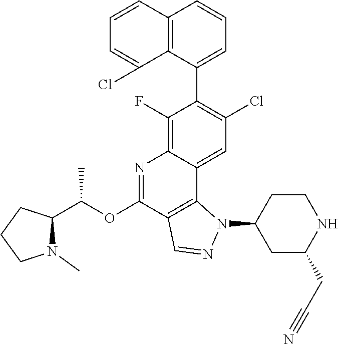 [SMILES: C[C@H](Oc1nc2c(F)c(-c3cccc4cccc(Cl)c34)c(Cl)cc2c2c1cnn2[C@H]1CCN[C@H](CC#N)C1)[C@@H]1CCCN1C]

This compound was prepared according to the procedure described in Example 67a and Example 67b, in Step 1 replacing tert-butyl (2S,4S)-2-(cyanomethyl)-4-(7-(5,6-dimethyl-1-(tetrahydro-2H-pyran-2-yl)-1H-indazol-4-yl)-6-fluoro-8-methyl-4-(methylthio)-1H-pyrazolo[4,3-c]quinolin-1-yl)piperidine-1-carboxylate with tert-butyl (2S,4S)-4-(8-chloro-7-(8-chloronaphthalen-1-yl)-6-fluoro-4-(methylthio)-1H-pyrazolo[4,3-c]quinolin-1-yl)-2-(cyanomethyl)piperidine-1-carboxylate. LC-MS calculated for C₃₉H₄₂Cl₂FN₆O₃ (M+H)⁺: m/z=731.3, 733.3; found 731.4, 733.4.

### Step 2. 2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)piperidin-2-yl)acetonitrile

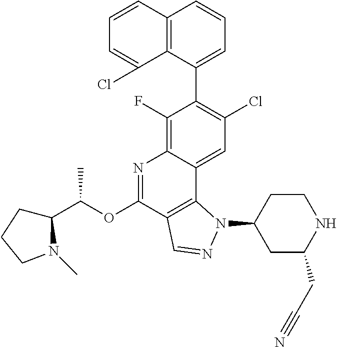 [SMILES: C[C@H](Oc1nc2c(F)c(-c3cccc4cccc(Cl)c34)c(Cl)cc2c2c1cnn2[C@H]1CCN[C@H](CC#N)C1)[C@@H]1CCCN1C]

This compound was prepared according to the procedure described in Example 21a and Example 21b, in Step 4 replacing tert-butyl (2S,4S)-4-(8-chloro-4-(3-(dimethylamino)azetidin-1-yl)-6-fluoro-7-(5-fluoroquinolin-8-yl)-1H-pyrazolo[4,3-c]quinolin-1-yl)-2-(cyanomethyl)piperidine-1-carboxylate with tert-butyl (2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-2-(cyanomethyl)piperidine-1-carboxylate. LC-MS calculated for C₃₄H₃₄Cl₂FN₆O (M+H)⁺: m/z=631.2, 633.2; found 631.3, 633.3.

### Step 3. 2-((2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile

To a solution of (E)-4-fluorobut-2-enoic acid (1.2 mg, 0.011 mmol) and 2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)piperidin-2-yl)acetonitrile bis(2,2,2-trifluoroacetate) (8.0 mg, 9.31 μmol) in DMF (1.0 ml) was added HATU (4.4 mg, 0.012 mmol) and DIEA (8.2 μl, 0.047 mmol). The resulting mixture was stirred at rt for 1 h. The reaction was diluted with methanol and 1 N HCl (0.1 mL) and purified using prep-LCMS (XBridge C18 column, eluting with a gradient of acetonitrile/water containing 0.1% TFA, at flow rate of 60 mL/min) to afford the desired product (2.0 mg, 30%). LC-MS calculated for C₃₈H₃₇Cl₂F₂N₆O₂ (M+H)⁺: m/z=717.2, 719.2; found 717.2, 719.2.

### Example 86. 2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-(2-fluoroacryloyl)piperidin-2-yl)acetonitrile

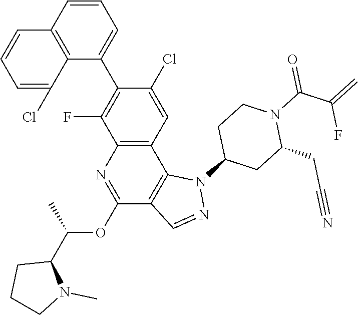 [SMILES: C=C(F)C(=O)N1CC[C@H](n2ncc3c(O[C@@H](C)[C@@H]4CCCN4C)nc4c(F)c(-c5cccc6cccc(Cl)c56)c(Cl)cc4c32)C[C@H]1CC#N]

This compound was prepared according to the procedure described in Example 85, step 3, replacing (E)-4-fluorobut-2-enoic acid with 2-fluoroacrylic acid. LC-MS calculated for C₃₇H₃₅Cl₂F₂N₆O₂ (M+H)⁺: m/z=703.2, 705.2; found 703.2, 705.2.

### Example 87. 2-((2S,4S)-1-(but-2-ynoyl)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)piperidin-2-yl)acetonitrile

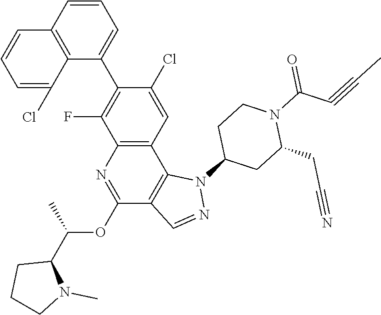 [SMILES: CC#CC(=O)N1CC[C@H](n2ncc3c(O[C@@H](C)[C@@H]4CCCN4C)nc4c(F)c(-c5cccc6cccc(Cl)c56)c(Cl)cc4c32)C[C@H]1CC#N]

This compound was prepared according to the procedure described Example 85, step 3, replacing (E)-4-fluorobut-2-enoic acid with but-2-ynoic acid. LC-MS calculated for C₃₈H₃₆Cl₂FN₆O₂ (M+H)⁺: m/z=697.2, 699.2; found 697.2, 699.2.

### Example 88. 2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-methoxybut-2-enoyl)piperidin-2-yl)acetonitrile

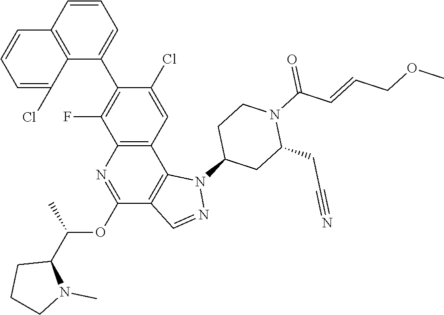 [SMILES: COC/C=C/C(=O)N1CC[C@H](n2ncc3c(O[C@@H](C)[C@@H]4CCCN4C)nc4c(F)c(-c5cccc6cccc(Cl)c56)c(Cl)cc4c32)C[C@H]1CC#N]

This compound was prepared according to the procedure described in Example 85, step 3, replacing (E)-4-fluorobut-2-enoic acid with (E)-4-methoxybut-2-enoic acid. LC-MS calculated for C₃₉H₄₀Cl₂FN₆O₃ (M+H)⁺: m/z=729.2, 731.2; found 729.2, 731.2.

### Example 89. 2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-(dimethylamino)but-2-enoyl)piperidin-2-yl)acetonitrile

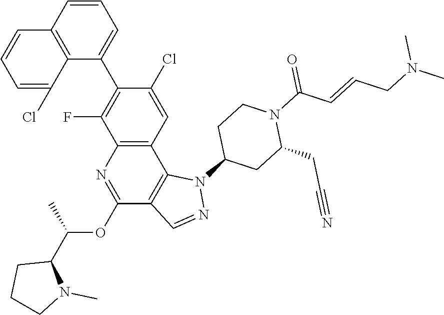 [SMILES: C[C@H](Oc1nc2c(F)c(-c3cccc4cccc(Cl)c34)c(Cl)cc2c2c1cnn2[C@H]1CCN(C(=O)/C=C/CN(C)C)[C@H](CC#N)C1)[C@@H]1CCCN1C]

This compound was prepared according to the procedure described in Example 85, step 3, replacing (E)-4-fluorobut-2-enoic acid with (E)-4-(dimethylamino)but-2-enoic acid hydrochloride. LC-MS calculated for C₄₀H₄₃Cl₂FN₇O₂ (M+H)⁺: m/z=742.3, 744.3; found 742.3, 744.3.

### Example 90a and Example 90b. 2-((2S,4S)-1-(but-2-ynoyl)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)piperidin-2-yl)acetonitrile

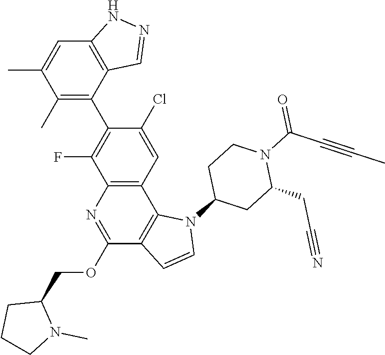 [SMILES: CC#CC(=O)N1CC[C@H](n2ccc3c(OC[C@@H]4CCCN4C)nc4c(F)c(-c5c(C)c(C)cc6[nH]ncc56)c(Cl)cc4c32)C[C@H]1CC#N]

This compound was prepared according to the procedure described Example 47a and Example 47b, step 9, replacing (E)-4-methoxybut-2-enoic acid with but-2-ynoic acid.

Diastereomer 1. Peak 1. LC-MS calculated for C₃₇H₃₈ClFN₇O₂ (M+H)⁺: m/z=666.3; found 666.4.

Diastereomer 2. Peak 2. LC-MS calculated for C₃₇H₃₈ClFN₇O₂ (M+H)⁺: m/z=666.3; found 666.4.

### Example A. GDP-GTP Exchange Assay

The inhibitor potency of the exemplified compounds was determined in a fluorescence based guanine nucleotide exchange assay, which measures the exchange of bodipy-GDP (fluorescently labeled GDP) for GppNHp (Non-hydrolyzable GTP analog) to generate the active state of KRAS in the presence of SOS1 (guanine nucleotide exchange factor). Inhibitors were serially diluted in DMSO and a volume of 0.1 μL was transferred to the wells of a black low volume 384-well plate. 5 μL/well volume of bodipy-loaded KRAS G12C diluted to 5 nM in assay buffer (25 mM Hepes pH 7.5, 50 mM NaCl, 10 mM MgCl2 and 0.01% Brij-35) was added to the plate and pre-incubated with inhibitor for 2 hours at ambient temperature. Appropriate controls (enzyme with no inhibitor or with a G12C inhibitor (AMG-510)) were included on the plate. The exchange was initiated by the addition of a 5 μL/well volume containing 1 mM GppNHp and 300 nM SOS1 in assay buffer. The 10 μL/well reaction concentration of the bodipy-loaded KRAS G12C, GppNHp, and SOS1 were 2.5 nM, 500 uM, and 150 nM, respectively. The reaction plates were incubated at ambient temperature for 2 hours, a time estimated for complete GDP-GTP exchange in the absence of inhibitor. For the KRAS G12D and G12V mutants, similar guanine nucleotide exchange assays were used with 2.5 nM as final concentration for the bodipy loaded KRAS proteins and with 4 hours and 3 hours incubation after adding GppNHp-SOS1 mixture for G12D and G12V respectively. A cyclic peptide described to selectively bind G12D mutant (Sakamoto et al., BBRC 484.3 (2017), 605-611) or internal compounds with confirmed binding were used as positive controls in the assay plates. Fluorescence intensities were measured on a PheraStar plate reader instrument (BMG Labtech) with excitation at 485 nm and emission at 520 nm.

Either GraphPad prism or XLfit was used to analyze the data. The IC₅₀ values were derived by fitting the data to a four parameter logistic equation producing a sigmoidal dose-response curve with a variable Hill coefficient. Prism equation: Y=Bottom+(Top−Bottom)/(1+10\hat{ }((Log IC₅₀−X)*Hill slope)); XLfit equation: Y=(A+((B−A)/(1+((X/C)\hat{ }D)))) where X is the logarithm of inhibitor concentration and Y is the response.

The KRAS_G12C exchange assay IC₅₀ data and KRAS_G12C pERK assay IC₅₀ data are provided in Table 1 below. The symbol “†” indicates IC₅₀≤100 nM, “†\” indicates IC₅₀>100 nM but ≤1 μM; and “†††” indicates IC₅₀ is >1 μM but ≤5 μM. “NA” indicates IC₅₀ not available.

TABLE 1

| Ex. No. | G12C_exchange | G12C_pERK |
|---|---|---|
| 1 | † | †† |
| 2 | † | † |
| 3a | † | † |
| 3b | † | † |
| 4a | † | † |
| 4b | †† | NA |
| 5a | † | † |
| 5b | †† | NA |
| 6a | † | † |
| 6b | †† | NA |
| 7a | † | † |
| 7b | †† | NA |
| 8a | † | † |
| 8b | †† | NA |
| 9a | † | † |
| 9b | †† | NA |
| 10a | † | † |
| 10b | †† | NA |
| 11a | † | † |
| 11b | †† | NA |
| 12a | † | † |
| 12b | †† | NA |
| 13a | † | † |
| 13b | †† | NA |
| 14a | † | † |
| 14b | †† | NA |
| 15a | † | † |
| 15b | †† | NA |
| 16a | † | † |
| 16b | †† | NA |
| 17a | † | † |
| 18a | † | † |
| 18b | †† | NA |
| 19a | † | † |
| 19b | †† | NA |
| 20a | † | † |
| 20b | † | NA |
| 21a | † | † |
| 21b | † | † |
| 22 | † | † |
| 23 | † | † |
| 24 | † | † |
| 25a | † | † |
| 25b | † | † |
| 26a | † | † |
| 26b | † | † |
| 27 | † | † |
| 28 | † | † |
| 47a | † | † |
| 51a | † | † |
| 52a | † | † |
| 53a | † | † |
| 54a | † | † |
| 55b | † | † |
| 56b | † | † |
| 57b | † | † |
| 58a | † | † |
| 59a | † | † |
| 60a | † | † |
| 61a | † | † |
| 62a | † | † |
| 63 | † | † |
| 64a | † | † |
| 65a | † | † |
| 66a | † | † |
| 67b | † | † |
| 68b | † | † |
| 69b | † | † |
| 70b | † | † |
| 71b | † | † |
| 72a | † | † |
| 73a | † | † |
| 74a | † | † |
| 75a | † | † |
| 76a | † | † |
| 77a | † | † |
| 78a | † | † |
| 79a | † | † |
| 80a | † | † |
| 81a | † | † |
| 82a | † | † |
| 83a | † | † |
| 84a | † | † |
| 85 | † | † |
| 86 | † | † |
| 87 | † | † |
| 88 | † | † |
| 89 | † | † |
| 90a | † | † |

The KRAS_G12D and G12V exchange assay 1050 data are provided in Table 2 below. The symbol “†” indicates IC₅₀≤100 nM, “††” indicates IC₅₀>100 nM but ≤1 μM; and “†††” indicates IC₅₀ is >1 μM but ≤5 μM, “††\\” indicates IC₅₀ is ≥5 μM but ≤10 μM. “NA” indicates IC₅₀ not available.

TABLE 2

| Ex. No. | G12D_exchange | G12V_exchange |
|---|---|---|
| 29 | † | † |
| 30 | † | † |
| 31 | † | † |
| 32 | † | † |
| 33 | † | †† |
| 34 | †† | ††† |
| 35 | † | †† |
| 36 | † | †† |
| 37 | † | †† |
| 38 | † | †† |
| 39 | † | † |
| 40 | †† | ††† |
| 41 | † | † |
| 42 | † | †† |
| 43 | † | †† |
| 44 | † | †† |
| 45 | † | †† |
| 46 | † | †† |
| 48 | † | † |
| 49 | †† | †† |
| 50 | † | † |

### Example B: Luminescent Viability Assay

MIA PaCa-2 (KRAS G12C; ATCC® CRL-1420), A427 (KRAS G12D; ATCC® HTB53) and NCI-H838 (KRAS WT; ATCC® CRL-5844) cells are cultured in RPMI 1640 media supplemented with 10% FBS (Gibco/Life Technologies). The cells are seeded (5×10³ cells/well/in 50 uL) into black, clear bottomed 96-well Greiner tissue culture plates and cultured overnight at 37° C., 5% CO₂. After overnight culture, 50 uL per well of serially diluted test compounds (2× final concentration) are added to the plates and incubated for 3 days. At the end of the assay, 100 ul/well of CellTiter-Glo reagent (Promega) is added. Luminescence is read after 15 minutes with a TopCount (PerkinElmer). IC₅₀ determination is performed by fitting the curve of percent inhibition versus the log of the inhibitor concentration using the GraphPad Prism 7 software.

### Example C: Cellular pERK HTRF Assay

MIA PaCa-2 (KRAS G12C; ATCC® CRL-1420), A427 (KRAS G12D; ATCC® HTB53), HPAF-II (KRAS G12D; ATCC® CRL-1997) and NCI-H838 (KRAS WT; ATCC® CRL-5844) cells are purchased from ATCC and maintained in RPMI 1640 media supplemented with 10% FBS (Gibco/Life Technologies). The cells are plated at 5000 cells per well (8 uL) into Greiner 384-well low volume, flat-bottom, tissue culture treated white plates and incubated overnight at 37° C., 5% CO₂. The next morning, test compound stock solutions are diluted in media at 3× the final concentration, and 4 uL are added to the cells. The plate is mixed by gentle rotation for 30 seconds (250 rpm) at room temperature. The cells are incubated with the KRAS G12C and G12D compounds for 4 hours or 2 hours respectively at 37° C., 5% CO₂.

4 uL of 4× lysis buffer with blocking reagent (1:25) (Cisbio) are added to each well and plates are rotated gently (300 rpm) for 30 minutes at room temperature. 4 uL per well of Cisbio anti Phospho-ERK 1/2 d2 is mixed with anti Phospho-ERK 1/2 Cryptate (1:1) are added to each well, mixed by rotation and incubated overnight in the dark at room temperature. Plates are read on the Pherastar plate reader at 665 nm and 620 nm wavelengths. IC₅₀ determination is performed by fitting the curve of inhibitor percent inhibition versus the log of the inhibitor concentration using the GraphPad Prism 7 software.

### Example D: Whole Blood pERK1/2 HTRF Assay

MIA PaCa-2 cells (KRAS G12C; ATCC® CRL-1420) and HPAF-II (KRAS G12D; ATCC® CRL-1997) are maintained in RPMI 1640 with 10% FBS (Gibco/Life Technologies). The cells are seeded into 96 well tissue culture plates (Corning #3596) at 25000 cells per well in 100 uL media and cultured for 2 days at 37° C., 5% CO₂ so that they are approximately 80% confluent at the start of the assay. Whole Blood are added to the 1 uL dots of compounds (prepared in DMSO) in 96 well plates and mixed gently by pipetting up and down so that the concentration of the compound in blood is 1× of desired concentration. The media is aspirated from the cells and 50 uL per well of whole blood with G12C or G12D compound is added and incubated for 4 or 2 hours respectively at 37° C., 5% CO₂. After dumping the blood, the plates are gently washed twice by adding PBS to the side of the wells and dumping the PBS from the plate onto a paper towel, tapping the plate to drain well. 50 ul/well of 1× lysis buffer #1 (Cisbio) with blocking reagent (1:25) (Cisbio) is then added and incubated at room temperature for 30 minutes with shaking (250 rpm). Following lysis, 16 uL of lysate is transferred into 384-well Greiner small volume white plate using an Assist Plus (Integra Biosciences, NH). 4 uL of 1:1 mixture of anti Phospho-ERK 1/2 d2 and anti Phospho-ERK 1/2 Cryptate (Cisbio) is added to the wells using the Assist Plus and incubated at room temperature overnight in the dark. Plates are read on the Pherastar plate reader at 665 nm and 620 nm wavelengths. IC₅₀ determination is performed by fitting the curve of inhibitor percent inhibition versus the log of the inhibitor concentration using the GraphPad Prism 7 software.

### Example E: Ras Activation Elisa

The 96-Well Ras Activation ELISA Kit (Cell Biolabs Inc; #STA441) uses the Raf1 RBD (Rho binding domain) bound to a 96-well plate to selectively pull down the active form of Ras from cell lysates. The captured GTP-Ras is then detected by a pan-Ras antibody and HRP-conjugated secondary antibody.

MIA PaCa-2 cells (KRAS G12C; ATCC® CRL-1420) and HPAF-II (KRAS G12D; ATCC® CRL-1997) are maintained in RPMI 1640 with 10% FBS (Gibco/Life Technologies). The cells are seeded into 96 well tissue culture plates (Corning #3596) at 25000 cells per well in 100 uL media and cultured for 2 days at 37° C., 5% CO₂ so that they are approximately 80% confluent at the start of the assay. The cells are treated with compounds for either 2 hours or overnight at 37° C., 5% CO₂. At the time of harvesting, the cells are washed with PBS, drained well and then lysed with 50 uL of the 1× Lysis buffer (provided by the kit) plus added Halt Protease and Phosphatase inhibitors (1:100) for 1 hour on ice.

The Raf-1 RBD is diluted 1:500 in Assay Diluent (provided in kit) and 100 μL of the diluted Raf-1 RBD is added to each well of the Raf-1 RBD Capture Plate. The plate is covered with a plate sealing film and incubated at room temperature for 1 hour on an orbital shaker. The plate is washed 3 times with 250 μL 1× Wash Buffer per well with thorough aspiration between each wash. 50 μL of Ras lysate sample (10-100 μg) is added per well in duplicate. A “no cell lysate” control is added in a couple of wells for background determination. 50 μL of Assay Diluent is added to all wells immediately to each well and the plate is incubated at room temperature for 1 hour on an orbital shaker. The plate is washed 5 times with 250 μL 1× Wash Buffer per well with thorough aspiration between each wash. 100 μL of the diluted Anti-pan-Ras Antibody is added to each well and the plate is incubated at room temperature for 1 hour on an orbital shaker. The plate is washed 5 times as previously. 100 μL of the diluted Secondary Antibody, HRP Conjugate is added to each well and the plate is incubated at room temperature for 1 hour on an orbital shaker. The plate is washed 5 times as previously and drained well. 100 μL of Chemiluminescent Reagent (provided in the kit) is added to each well, including the blank wells. The plate is incubated at room temperature for 5 minutes on an orbital shaker before the luminescence of each microwell is read on a plate luminometer. The % inhibition is calculated relative to the DMSO control wells after a background level of the “no lysate control” is subtracted from all the values. IC₅₀ determination is performed by fitting the curve of inhibitor percent inhibition versus the log of the inhibitor concentration using the GraphPad Prism 7 software.

### Example F: Inhibition of RAS-RAF and PI3K-AKT Pathways

The cellular potency of compounds was determined by measuring phosphorylation of KRAS downstream effectors extracellular-signal-regulated kinase (ERK), ribosomal S6 kinase (RSK), AKT (also known as protein kinase B, PKB) and downstream substrate S6 ribosomal protein.

To measure phosphorylated extracellular-signal-regulated kinase (ERK), ribosomal S6 kinase (RSK), AKT and S6 ribosomal protein, cells (details regarding the cell lines and types of data produced are further detailed in Table 4 were seeded overnight in Corning 96-well tissue culture treated plates in RPMI medium with 10% FBS at 4×10⁴ cells/well. The following day, cells were incubated in the presence or absence of a concentration range of test compounds for 4 hours at 37° C., 5% CO₂. Cells were washed with PBS and lysed with 1× lysis buffer (Cisbio) with protease and phosphatase inhibitors. 10 μg of total protein lysates was subjected to SDS-PAGE and immunoblot analysis using following antibodies: phospho-ERK1/2-Thr202/Tyr204 (#9101L), total-ERK1/2 (#9102L), phosphor-AKT-Ser473 (#4060L), phospho-p90RSK-Ser380 (#11989S) and phospho-S6 ribosomal protein-Ser235/Ser236 (#2211S) are from Cell Signaling Technologies (Danvers, Mass.).

TABLE 4

| Cell Line | Histology | KRAS alteration | Readout |
|---|---|---|---|
| H358 | Lung | G12C | pERK, pAKT |
| MIA PaCa-2 | Pancreas | G12C | pERK, pAKT |
| HPAF II | Pancreas | G12D | pERK, pAKT |
| SU.86.86 | Pancreas | G12D | pERK, pAKT |
| PaTu 8988s | Pancreas | G12V | pERK, pAKT |
| H441 | Lung | G12V | pERK, pAKT |

### Example G: In Vivo Efficacy Studies

Mia-Paca-2 human pancreatic cancer cells were obtained from the American Type Culture Collection and maintained in RPMI media supplemented with 10% FBS. For efficacy studies experiments, 5×10⁶ Mia-Paca-2 cells were inoculated subcutaneously into the right hind flank of 6- to 8-week-old BALB/c nude mice (Charles River Laboratories, Wilmington, Mass., USA). When tumor volumes were approximately 150-250 mm3, mice were randomized by tumor volume and compounds were orally administered. Tumor volume was calculated using the formula (L×W²)/2, where L and W refer to the length and width dimensions, respectively. Tumor growth inhibition was calculated using the formula (1−(V_{T}/V_{C}))×100, where V_{T} is the tumor volume of the treatment group on the last day of treatment, and V_{C} is the tumor volume of the control group on the last day of treatment. Two-way analysis of variance with Dunnett's multiple comparisons test was used to determine statistical differences between treatment groups (GraphPad Prism). Mice were housed at 10-12 animals per cage, and were provided enrichment and exposed to 12-hour light/dark cycles. Mice whose tumor volumes exceeded limits (10% of body weight) were humanely euthanized by CO₂ inhalation. Animals were maintained in a barrier facility fully accredited by the Association for Assessment and Accreditation of Laboratory Animal Care, International. All of the procedures were conducted in accordance with the US Public Service Policy on Human Care and Use of Laboratory Animals and with Incyte Animal Care and Use Committee Guidelines.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound of Formula I: 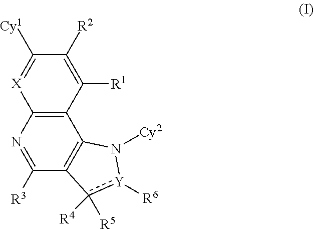
or a pharmaceutically acceptable salt thereof,
wherein:
each [symbol] independently represents a single bond or a double bond;
X is N or CR⁷;
Y is N or C;
R¹ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, and BR^{h1}Rⁱ¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R^{g};
R² is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, and BR^{h2}Rⁱ²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R²²;
Cy¹ is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R¹⁰;
R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{f3}, SR^{a3}, C(O)R^{b3}, C(O)NR^{c3}R^{d3}, C(O)OR^{a3}OC(O)R^{b3}, OC(O)NR^{c3}R^{d3}, NR^{c3}R^{j3}, NR^{c3}C(O)R^{b3}, NR C(O)OR^{a3}, NR^{c3}C(O)NR^{c3}R^{d3}, C(═NR^{e3})R^{b3}, C(═NOR^{a3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, and BR^{h3}Rⁱ³; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R³⁰;
when R⁴R⁵C [symbol] is a single bond and Y is C, then YR⁶ is selected from C═O and C═S; and
R⁴ is selected from H, D, C₁₋₆ alkyl, C₂₋₆ alkenyl, C₂₋₆ alkynyl, C₁₋₆ haloalkyl, C₃₋₆ cycloalkyl, 4-6 membered heterocycloalkyl, phenyl, 5-6 membered heteroaryl, halo, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, and BR^{h4}Rⁱ⁴; wherein said C₁₋₆ alkyl, C₂₋₆ alkenyl, C₂₋₆ alkynyl, C₃₋₆ cycloalkyl, 4-6 membered heterocycloalkyl, phenyl, and 5-6 membered heteroaryl, are each optionally substituted with 1, 2, 3, or 4 substituents independently selected from R^{g};
R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a5}, SR^{a5}, C(O)R^{b5}, C(O)NR^{c5}R^{d5}, C(O)OR^{a5}OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NOR^{a5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, and BR^{h5}Rⁱ⁵; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R⁵⁰;
when R⁴R⁵C [symbol] YR⁶ is a double bond and Y is N, then R⁴ and R⁶ are absent;
when R⁴R⁵C [symbol] YR⁶ is a double bond and Y is C, then R⁴ is absent; and
R⁶ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NOR^{a6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, and BR^{h6}Rⁱ⁶; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R⁶⁰;
R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NOR^{a7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, and BR^{h7}Rⁱ⁷; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R⁷⁰;
Cy² is selected from C₃₋₁₀ cycloalkyl, 4-14 membered heterocycloalkyl, C₆₋₁₀ aryl and 5-10 membered heteroaryl; wherein the 4-14 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-14 membered heterocycloalkyl is optionally substituted by oxo to form a carbonyl group; and wherein the C₃₋₁₀ cycloalkyl, 4-14 membered heterocycloalkyl, C₆₋₁₀ aryl and 5-10 membered heteroaryl are each optionally substituted with 1, 2, 3 or 4 substituents independently selected from R²⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, C(═NR^{e10})R^{b10}, C(═NOR^{a10}R^{b10}, C(═NR^{e10})NR^{c10}R^{d10}, NR^{c10}C(═NR^{e10})NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, S(O)₂NR^{c10}R^{d10}, and BR^{h10}Rⁱ¹⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, and BR^{h11}Rⁱ¹¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₆ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²;
each R¹² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a12}, SR^{a12}, C(O)R^{b12}, C(O)NR^{c12}R^{d12}, C(O)OR^{a1}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)R^{b12}, NR^{c12}S(O)₂R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, and BR^{h12}Rⁱ¹²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};
each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a20}, SR^{a20}, C(O)R^{b20}, C(O)NR^{c20}R^{d20}, C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, C(═NR^{e20})R^{b20}, C(═NOR^{a20})R^{b20}, C(═NR^{e20})NR^{c20}R^{d20}, NR^{c20}C(═NR^{e20})NR^{c20}R^{d20}, NR^{c20}S(O)R^{b20}, NR^{c20}S(O)₂R^{b20}, NR^{c20}S(O)₂NR^{c20}R^{d20}, S(O)R^{b20}, S(O)NR^{c20}R^{d20}, S(O)₂R^{b20}, S(O)₂NR^{c20}R^{d20}, and BR^{h20}Rⁱ²⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a21}, SR^{a21}, C(O)R^{b21}, C(O)NR^{c21}R^{d21} C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)R^{b21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, and BR^{h21}Rⁱ²¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R^{g};
each R²² is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)R^{b22}, NR^{c22}S(O)₂R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, and BR^{h22}Rⁱ²²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²³;
each R²³ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a23}, SR^{a23}, C(O)R^{b23}, C(O)NR^{c23}R^{d23}, C(O)OR^{a23}, OC(O)R^{b23}, OC(O)NR^{c23}R^{d23}, NR^{c23}R^{d23}, NR^{c23}C(O)R^{b23}, NR^{c23}C(O)OR^{a23}, NR^{c23}C(O)NR^{c23}R^{d23}, NR^{c23}S(O)R^{b23}, NR^{c23}S(O)₂R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, and BR^{h23}Rⁱ²³; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²⁴;
each R²⁴ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a24} SR^{a24}, C(O)R^{b24}, C(O)NR^{c24}R^{d24}, C(O)OR^{a24}, OC(O)R^{b24}, OC(O)NR^{c24}R^{d24}, NR^{c24}R^{d24}, NR^{c24}C(O)R^{b24}, NR^{c24}C(O)OR^{a24}, NR^{c24}C(O)NR^{c24}R^{d24}, NR^{c24}S(O)R^{b24}, NR^{c24}S(O)₂R^{b24}, NR^{c24}S(O)₂NR^{c24}R^{d24}, S(O)R^{b24}, S(O)NR^{c24}R^{d24}, S(O)₂R^{b24}, S(O)₂NR^{c24}R^{d24}, and BR^{h24}Rⁱ²⁴ wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a30}, SR^{a30}, C(O)R^{b30}, C(O)NR^{c30}R^{d30}, C(O)OR^{a30}, OC(O)R^{b30}, OC(O)NR^{c3}R^{d30}, NR^{c30}R^{d30}, NR^{c30}C(O)R^{b30}, NR^{d30}C(O)OR^{a30}, NR^{c30}C(O)NR^{c30}R^{d30}, NR^{c30}S(O)R^{b30}, NR^{c30}S(O)₂R^{b30}, NR^{c30}S(O)₂NR^{c30}R^{d30}, S(O)R^{b30}, S(O)NR^{c30}R^{d30}, S(O)₂R^{b30}, S(O)₂NR^{c30}R^{d30}, and BR^{h30}Rⁱ³⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³¹;
each R³¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a31}, SR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR 1C(O)R^{b31}, NR^{c31}C(O)OR^{a31}, NR^{c31}C(O)NR^{c31}R^{d31}, NR^{c31}S(O)R^{b31}, NR^{c31}S(O)₂R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, and BR^{h31}Rⁱ³¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³²;
each R³² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a32}, SR^{a32}, C(O)R^{b32}, C(O)NR^{c32}R^{d32}, C(O)OR^{a32}, OC(O)R^{b32}, OC(O)NR^{c32}R^{d32}, NR^{c32}R^{d32}, NR^{c32}C(O)R^{b32}, NR^{c32}C(O)OR^{a32}, NR^{c32}C(O)NR^{c32}R^{d32}, NR^{c32}S(O)R^{b32}, NR³²S(O)₂R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32}, and BR^{h32}Rⁱ³²; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁵⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a50}, SR^{a50}, C(O)R^{b50}, C(O)NR^{c50}R^{d50}, C(O)OR^{a50}, OC(O)R^{b50}, OC(O)NR^{c50}R^{d50}, NR^{c50}R^{d50}, NR^{c50}C(O)R^{b50}, NR^{c50}C(O)OR^{a50}, NR^{c50}C(O)NR^{c50}R^{d50}, NR^{c50}S(O)R^{b50}, NR^{c50}S(O)₂R^{b50}, NR^{c50}S(O)₂NR^{c50}, R^{d50} S(O)R^{b50}, S(O)NR^{c50}R^{d50}, S(O)₂R^{b50}, S(O)₂NR^{c50}R^{d50}, and BR^{h50}Rⁱ⁵⁰ wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵¹;
each R⁵¹ is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a51}, SR^{a51}, C(O)R^{b51}, C(O)NR^{c51}R^{d51}, C(O)OR^{a51}, C(O)R^{b51}, OC(O)NR^{c51}R^{d51}, NR^{c51}R^{d51}, NR^{c51}C(O)R^{b51}, NR^{c51}C(O)OR^{a51}, NR^{c51}C(O)NR^{c51}R^{d51}, NR^{c51}S(O)R^{b51}, NR^{c51}S(O)₂R^{b51}, NR^{c51}, S(O)₂NR^{c51}, R^{d51}, S(O)R^{b51}, S(O)NR^{c51}R^{d51}, S(O)₂R^{b51}, S(O)₂NR^{c51}R^{d51}, and BR^{h51}, Rⁱ⁵¹; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R⁵²;
each R⁵² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a52}, SR^{a52}, C(O)R^{b52}, C(O)NR^{c52}R^{d52}, (O)OR^{a52}, OC(O)R^{b52}, OC(O)NR^{c52}R^{d52}, NR^{c52}R^{d52}, NR^{c52}C(O)R^{b52}, NR^{c52}C(O)OR^{a52}, NR^{c52}C(O)NR^{c52}R^{d52}, NR^{c52}S(O)R^{b52}, NR^{c52}S(O)₂R^{b52}, NR^{c52}S(O)₂NR^{c52}R^{d52}, S(O)R^{b52}, S(O)NR^{c52}R^{d52}, S(O)₂R^{b52}, S(O)₂NR^{c52}R^{d52}, and BR^{h52}Rⁱ⁵² wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};
each R⁶⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a60}, SR^{a60}, C(O)R^{b60}, C(O)NR^{c60}R^{d60}, C(O)OR^{a60}, OC(O)R^{b60}, OC(O)NR^{c60}R^{d60}, NR^{c60}R^{d60}, NR^{c60}C(O)R^{b60}, NR^{c60}C(O)OR^{a60}, NR^{c60}C(O)NR^{c60}R^{d60}, NR^{c60}S(O)R^{b60}, NR^{c60}S(O)₂R^{b60}, NR^{c60}S(O)₂NR^{c60}R^{d60}, S(O)R^{b60}, S(O)NR^{c60}R^{d60}, S(O)₂R^{b60}, S(O)₂NR^{c60}R^{d60}, and BR^{h60}Rⁱ⁶⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶¹;
each R⁶¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}, C(O)NR^{c61}R^{d61}, NR^{c61}S(O)R^{b61}, NR^{c61}S(O)₂R^{b61}, NR^{c61}S(O)₂NR^{c61}R^{d61}, S(O)R^{b61}, S(O)NR^{c61}R^{d61}, S(O)₂R^{b61}, S(O)₂NR^{c1}R^{d61}, and BR^{h61}Rⁱ⁶¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶²;
each R⁶² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a62}, SR^{a62}, C(O)R^{b62}, C(O)NR^{c52}R^{d62}, C(O)OR^{a62}, OC(O)R^{b62}, OC(O)NR^{c2}R^{d62}, NR^{c2}R^{d62}, NR^{c62}C(O)R^{b62}, NR^{c62}C(O)OR^{a62}, NR^{c62}C(O)NR^{c62}R^{d62}, NR^{c62}S(O)R^{b62}, NR^{c62}S(O)₂R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, and BR^{h62}Rⁱ⁶²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};
each R⁷⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a70}, SR^{a70}, C(O)R^{b70}, C(O)NR^{c70}R^{d70}, C(O)OR^{a70}, OC(O)R^{b70}, OC(O)NR^{c70}R^{d70}, NR^{c70}R^{d70}, NR^{c70}C(O)R^{b70}, NR^{c70}C(O)OR^{a70}, NR^{c70}C(O)NR^{c70}R^{d70}, NR^{c70}S(O)R^{b70}, NR^{c70}S(O)₂R^{b70}, NR^{c70}S(O)₂NR^{c70}R^{d70}S(O)R^{b70}, S(O)NR^{c70}R^{d70}, S(O)₂R^{b70}, S(O)₂NR^{c70}R^{d70}, and BR^{h70}Rⁱ⁷⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷¹;
each R⁷¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}, R^{d71}, NR^{c71}C(O) R^{b71}, NR^{c71}C(O)OR^{a71}, NR^{c71}C(O)NR^{c71}R^{d71}, NR^{c71}S(O)R^{b71}, NR^{c71}S(O)₂R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, and BR^{h71}Rⁱ⁷¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁷²;
each R⁷² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a72}, SR^{a72}, C(O)R^{b72}, C(O)NR^{c72}R^{d72}, C(O)OR^{a72}, OC(O)R^{b72}, OC(O)NR^{c72}R^{d72}, NR^{c72}R^{d72}, NR^{c72}C(O)R^{b72}, NR^{c72}C(O)OR^{a72}, NR^{c72}C(O)NR^{c72}R^{d72}, NR^{c72}S(O)R^{b72}, NR^{c72}S(O)₂R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72}, and BR^{h72}Rⁱ⁷²; wherein said C₁₋₆ alkyl, C₂₋₆ alkenyl, C₂₋₆ alkynyl, C₃₋₆ cycloalkyl, phenyl, 5-6 membered heteroaryl, and 4-7 membered heterocycloalkyl, are each optionally substituted with 1, 2, 3, or 4 substituents independently selected from R^{g};
each R^{a1}, R^{b1}, R^{c1}, and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};
or any R^{c1} and R^{d1} attached to the same N atom, together with the N atom to which they are attached, form a 4-, 5-, 6- or 7-membered heterocycloalkyl group optionally substituted with 1, 2 or 3 substituents independently selected from R^{g};
each R^{h1} and Rⁱ¹ is independently selected from OH, C₁₋₆ alkoxy, and C₁₋₆ haloalkoxy; or
any R^{h1} and Rⁱ¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a2}, R^{b2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²²;
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R²²;
each R^{e2} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h2} and Rⁱ² is independently selected from OH, C₁₋₆ alkoxy, and C₁₋₆ haloalkoxy; or
any R^{h2} and Rⁱ² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a3}, R^{b3}, R^{c3} and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R³⁰;
each R^{e3} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{f3} and Rⁱ³ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰;
each R^{h3} and Rⁱ³ is independently selected from OH, C₁₋₆ alkoxy, and C₁₋₆ haloalkoxy; or
any R^{h3} and Rⁱ³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a4}, R^{b4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};
or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R^{g};
each R^{h4} and Rⁱ⁴ is independently selected from OH, C₁₋₆ alkoxy, and C₁₋₆ haloalkoxy; or
any R^{h4} and Rⁱ⁴ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a5}, R^{b5}, R^{c5} and R^{d5} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁵⁰;
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R⁵⁰;
each R^{e5} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h5} and Rⁱ⁵ is independently selected from OH, C₁₋₆ alkoxy, and C₁₋₆ haloalkoxy; or any R^{h5} and Rⁱ⁵ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a6}, R^{b6}, R6 and R^{d6} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶⁰;
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R⁶⁰;
each R^{e6} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h6} and Rⁱ⁶ is independently selected from OH, C₁₋₆ alkoxy, and C₁₋₆ haloalkoxy; or
any R^{h6} and Rⁱ⁶ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a7}, R^{b7}, R^{c7} and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁷⁰;
or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R⁷⁰;
each R^{e7} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h7} and Rⁱ⁷ is independently selected from OH, C₁₋₆ alkoxy, and C₁₋₆ haloalkoxy; or any R^{h7} and Rⁱ⁷ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a10}, R^{b10}, R^{c10} and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R¹¹;
each R^{e10} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{b10} and R^{c10} is independently selected from OH, C₁₋₆ alkoxy, and C₁₋₆ haloalkoxy; or
any R^{b10} and R^{c10} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a11}, R^{b11}, R^{c11} and R^{d11}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;
or any R^{c11} and R^{d11} attached to the same N atom, together with the N atom to which they are attached, form a 4-, 5-, 6- or 7-membered heterocycloalkyl group optionally substituted with 1, 2 or 3 substituents independently selected from R¹²;
each R^{h11} and Rⁱ¹¹ is independently selected from OH, C₁₋₆ alkoxy, and C₁₋₆ haloalkoxy; or
any R^{h11} and Rⁱ¹¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a12}, R^{b12}, R^{c12} and R^{d12}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{h12} and Rⁱ¹² is independently selected from OH, C₁₋₆ alkoxy, and C₁₋₆ haloalkoxy; or any R^{h12} and Rⁱ¹² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a20}, R^{b20}, R^{c20} and R^{d20} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²¹;
or any R^{c20} and R^{d20} attached to the same N atom, together with the N atom to which they are attached, form a 4-, 5-, 6- or 7-membered heterocycloalkyl group optionally substituted with 1, 2, 3, or 4 substituents independently selected from R²¹;
each R^{e20} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h20} and Rⁱ²⁰ is independently selected from OH, C₁₋₆ alkoxy, and C₁₋₆ haloalkoxy; or any R^{h20} and Rⁱ²⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a21}, R^{b21}, R^{c21} and R^{d21}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R^{g};
or any R^{c21} and R^{d21} attached to the same N atom, together with the N atom to which they are attached, form a 4-, 5-, 6- or 7-membered heterocycloalkyl group optionally substituted with 1, 2 or 3 substituents independently selected from R^{g};
each R^{h21} and Rⁱ²¹ is independently selected from OH, C₁₋₆ alkoxy, and C₁₋₆ haloalkoxy; or any R^{h21} and Rⁱ²¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a22}, R^{b22}, R^{c22} and R^{d22} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²³;
or any R^{c22} and R^{d22} attached to the same N atom, together with the N atom to which they are attached, form a 4-, 5-, 6- or 7-membered heterocycloalkyl group optionally substituted with 1, 2, 3, or 4 substituents independently selected from R²³;
each R^{h22} and Rⁱ²² is independently selected from OH, C₁₋₆ alkoxy, and C₁₋₆ haloalkoxy; or any R^{h22} and Rⁱ²² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a23}, R^{b23}, R^{c23} and R^{d23}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²⁴;
or any R^{c23} and R^{d23} attached to the same N atom, together with the N atom to which they are attached, form a 4-, 5-, 6- or 7-membered heterocycloalkyl group optionally substituted with 1, 2 or 3 substituents independently selected from R²⁴;
each R^{h23} and Rⁱ²³ is independently selected from OH, C₁₋₆ alkoxy, and C₁₋₆ haloalkoxy; or
any R^{h23} and Rⁱ²³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a24}R^{b24}, R^{c24} and R^{d24}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{h24} and Rⁱ²⁴ is independently selected from OH, C₁₋₆ alkoxy, and C₁₋₆ haloalkoxy; or any R^{h24} and Rⁱ²⁴ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a30}, R^{b30}, R^{c30} and R^{d30}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³¹;
or any R^{c30} and R^{d30}, attached to the same N atom, together with the N atom to which they are attached, form a 4-, 5-, 6- or 7-membered heterocycloalkyl group optionally substituted with 1, 2, 3, or 4 substituents independently selected from R³¹;
each R^{h30} and Rⁱ³⁰ is independently selected from OH, C₁₋₆ alkoxy, and C₁₋₆ haloalkoxy; or
any R^{h30} and Rⁱ³⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a31}, R^{b31}, R^{c31} and R^{d31}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R³²;
or any R^{c31} and R^{d31} attached to the same N atom, together with the N atom to which they are attached, form a 4-, 5-, 6- or 7-membered heterocycloalkyl group optionally substituted with 1, 2 or 3 substituents independently selected from R³²;
each R^{h31} and Rⁱ³¹ is independently selected from OH, C₁₋₆ alkoxy, and C₁₋₆ haloalkoxy; or
any R^{h31} and Rⁱ³¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a32}, R^{b32}, R^{c32} and R^{d32}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{h32} and Rⁱ³² is independently selected from OH, C₁₋₆ alkoxy, and C₁₋₆ haloalkoxy; or any R^{h32} and Rⁱ³² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a50}, R^{b50}, R^{c50} and R^{d50}, is independently selected from H, C₁₋₆ alkyl, C₂₋₆ alkenyl, C₂₋₆ alkynyl, C₁₋₆haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R⁵¹;
or any R^{c50} and R^{d50}, attached to the same N atom, together with the N atom to which they are attached, form a 4-, 5-, 6- or 7-membered heterocycloalkyl group optionally substituted with 1, 2 or 3 substituents independently selected from R⁵¹;
each R^{h50} and Rⁱ⁵⁰ is independently selected from OH, C₁₋₆ alkoxy, and C₁₋₆ haloalkoxy; or any R^{h50} and Rⁱ⁵⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a51}, R^{b51}, R^{c51} and R^{d51}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R⁵²;
or any R^{c51} and R^{d51} attached to the same N atom, together with the N atom to which they are attached, form a 4-, 5-, 6- or 7-membered heterocycloalkyl group optionally substituted with 1, 2, 3, or 4 substituents independently selected from R⁵²;
each R^{h51} and Rⁱ⁵¹ is independently selected from OH, C₁₋₆ alkoxy, and C₁₋₆ haloalkoxy; or
any R^{h51} and Rⁱ⁵¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a52}, R^{b52}, R^{c52} and R^{d52}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{h52} and Rⁱ⁵² is independently selected from OH, C₁₋₆ alkoxy, and C₁₋₆ haloalkoxy; or any R^{h52} and Rⁱ⁵² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a60}, R^{b60}, R^{c60} and R^{d60} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶¹;
or any R^{c60} and R^{d60} attached to the same N atom, together with the N atom to which they are attached, form a 4-, 5-, 6- or 7-membered heterocycloalkyl group optionally substituted with 1, 2, 3, or 4 substituents independently selected from R⁶¹;
each R^{h60} and Rⁱ⁶⁰ is independently selected from OH, C₁₋₆ alkoxy, and C₁₋₆ haloalkoxy; or
any R^{h60} and Rⁱ⁶⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a61}, R^{b61}, R^{c61} and R^{d61}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R⁶²;
or any R^{c61} and R^{d61} attached to the same N atom, together with the N atom to which they are attached, form a 4-, 5-, 6- or 7-membered heterocycloalkyl group optionally substituted with 1, 2 or 3 substituents independently selected from R⁶²;
each R^{h61} and Rⁱ⁶¹ is independently selected from OH, C₁₋₆ alkoxy, and C₁₋₆ haloalkoxy; or any R^{h61} and Rⁱ⁶¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a62}, R^{b62}, R^{c62} and R^{d62}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
or any R^{c62} and R^{d62} attached to the same N atom, together with the N atom to which they are attached, form a 4-, 5-, 6- or 7-membered heterocycloalkyl group optionally substituted with 1, 2, 3, or 4 substituents independently selected from R^{g};
each R^{h62} and Rⁱ⁶² is independently selected from OH, C₁₋₆ alkoxy, and C₁₋₆ haloalkoxy; or any R^{h62} and Rⁱ⁶² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a70}, R^{b70}, R^{c70} and R^{d70} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁷¹;
or any R^{c70} and R^{d70} attached to the same N atom, together with the N atom to which they are attached, form a 4-, 5-, 6- or 7-membered heterocycloalkyl group optionally substituted with 1, 2, 3, or 4 substituents independently selected from R⁷¹;
each R^{h70} and Rⁱ⁷⁰ is independently selected from OH, C₁₋₆ alkoxy, and C₁₋₆ haloalkoxy; or
any R^{h70} and Rⁱ⁷⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a71}, R^{b71}, R^{c71} and R^{d71}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R⁷²;
or any R^{c71} and R^{d71} attached to the same N atom, together with the N atom to which they are attached, form a 4-, 5-, 6- or 7-membered heterocycloalkyl group optionally substituted with 1, 2 or 3 substituents independently selected from R⁷²;
each R^{h71} and Rⁱ⁷¹ is independently selected from OH, C₁₋₆ alkoxy, and C₁₋₆ haloalkoxy; or
any R^{h71} and Rⁱ⁷¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a72}, R^{b72}, R^{c72} and R^{d72}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{h72} and Rⁱ⁷² is independently selected from OH, C₁₋₆ alkoxy, and C₁₋₆ haloalkoxy; or
any R^{h72} and Rⁱ⁷² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; and
each R⁹ is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
provided that, when R⁴R⁵C [symbol] ⁶ is a double bond and Y is N, then Cy¹ is other than 3,5-dimethylisoxazol-4-yl.

2. The compound of claim 1, or a pharmaceutically acceptable salt thereof, wherein
each [symbol] independently represents a single bond or a double bond;
X is N or CR⁷;
Y is N or C;
R¹ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, and BR^{h1}Rⁱ¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R^{g};
R² is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃ alkylene, 5-10 membered heteroaryl-C₁₋₃alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, and BR^{h2}Rⁱ²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R²²;
Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3 or 4 substituents independently selected from R¹⁰;
R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{f3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{j3}, NR^{c3}C(O)R^{b3}, NR^{c3}C(O)OR^{a3}, NR^{c3}C(O)NR^{c3}R^{d3}, C(═NR^{e3})R^{b3}, C(═NOR^{a3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, and BR^{h3}Rⁱ³; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R³⁰;
when R⁴R⁵C [symbol] YR⁶ is a single bond and Y is C, then YR⁶ is selected from C═O and C═S; and
R⁴ is selected from H, D, C₁₋₆ alkyl, C₂₋₆ alkenyl, C₂₋₆ alkynyl, C₁₋₆ haloalkyl, C₃₋₆ cycloalkyl, 4-6 membered heterocycloalkyl, phenyl, 5-6 membered heteroaryl, halo, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, and BR^{h4}Rⁱ⁴;
R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}SR^{d5}, C(═NR^{e5})R^{b5}, C(═NOR^{a5})R^{b5}, C(═NR^{e5})NR^{c5}R^{d5}, NR^{c5}C(═NR^{e5})NR^{b5}R^{d5}, NR^{c5}C(═NR^{e5})R^{b5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, and BR^{h5}Rⁱ⁵; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R⁵⁰;
when R⁴R⁵C [symbol] YR⁶ is a double bond and Y is N, then R⁴ and R⁶ are absent;
when R⁴R⁵C [symbol] YR⁶ is a double bond and Y is C, then R⁴ is absent; and
R⁶ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NOR^{a6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, and BR^{h6}Rⁱ⁶; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R⁶⁰;
R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a6}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NOR^{a7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}; and BR^{h7}Rⁱ⁷; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R⁷⁰;
Cy² is selected from C₃₋₁₀ cycloalkyl, 4-14 membered heterocycloalkyl, C₆₋₁₀ aryl and 5-10 membered heteroaryl; wherein the 4-14 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-14 membered heterocycloalkyl is optionally substituted by oxo to form a carbonyl group; and wherein the C₃₋₁₀ cycloalkyl, 4-14 membered heterocycloalkyl, C₆₋₁₀ aryl and 5-10 membered heteroaryl are each optionally substituted with 1, 2, 3 or 4 substituents independently selected from R²⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, C(═NR^{e10})R^{b10}, C(═NOR^{a10})R^{b10}, C(═NR^{e10})NR^{c10}R^{d10}, NR^{c10}C(═NR^{e10})NR^{c10}R^{d10}, NR^{c10}S(O)R^{b10}, NR^{c10}S(O)₂R^{b10}, NR^{c10}S(O)₂NR^{c10}R^{d10}, S(O)R^{b10}; S(O)NR^{c10}R^{d10}, S(O)₂R^{b10}, S(O)₂NR^{c10}R^{d10}, and BR^{h10}Rⁱ¹⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₆ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, and BR^{h11}Rⁱ¹¹;
each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a20}, SR^{a20}, C(O)R^{b20}, C(O)NR^{c20}R^{d20}, C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, C(═NR^{e20})R^{b20}, C(═NOR^{a20})R^{b20}, C(═NR^{e20})NR^{c20}R^{d20}, NR^{c20}C(═NR^{e20})NR^{c20}R^{d20}, NR^{c20}S(O)R^{b20}, NR^{c20}S(O)₂R^{b20}, NR^{c20}S(O)₂NR^{c20}R^{d20}, S(O)R^{b20}, S(O)NR^{c20}R^{d20}, S(O)₂R^{b20}, S(O)₂NR^{c20}R^{d20}, and BR^{h20}Rⁱ²⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)R^{b21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, and BR^{h21}Rⁱ²¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R^{g};
each R²² is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)R^{b22}, NR^{c22}S(O)₂R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, and BR^{h22}Rⁱ²²; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²³;
each R²³ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a23}, SR^{a23}, C(O)R^{b23}, C(O)NR^{c23}R^{d23}, C(O)OR^{a23}, OC(O)R^{b23}, OC(O)NR^{c23}R^{d23}, NR^{c23}R^{d23}, NR^{c23}C(O)R^{b23}, NR^{c23}C(O)OR^{a23}, NR^{c23}C(O)NR^{c23}R^{d23}, NR^{c23}S(O)R^{b23}, NR^{c23}S(O)₂R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, and BR^{h23}Rⁱ²³;
each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a30}, SR^{a30}, C(O)R^{b30}, C(O)NR^{c30}R^{d30}, C(O)OR^{a30}, OC(O)R^{b30}, OC(O)NR^{c3}R^{d30}, NR^{c30}R^{d30}, NR^{c30}C(O)R^{b30}, NR^{d30}C(O)OR^{a30}, NR^{c30}C(O)NR^{c30}R^{d30}, NR^{c30}S(O)R^{b30}, NR^{c30}S(O)₂R^{b30}, NR^{c30}S(O)₂NR^{c30}R^{d30}, S(O)R^{b30}, S(O)NR^{c30}R^{d30}, S(O)₂R^{b30}, S(O)₂NR^{c30}R^{d30}, and BR^{h30}R³⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³¹;
each R³¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a31}, SR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR^{d31}C(O)R^{b31}, NR^{d31}C(O)OR^{a31}, NR^{c31}C(O)NR^{c31}R^{d31}, NR^{c31}S(O)R^{b31}, NR^{c31}S(O)₂R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, and BR^{h31}Rⁱ³¹; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³²;
each R³² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a32}, SR^{a32}, C(O)R^{b32}, C(O)NR^{c32}R^{d32}, C(O)OR^{a32}C(O)R^{b32}, OC(O)NR^{c2}R^{d32}, NR^{c32}R^{d32}, NR^{c32}C(O)R^{b32}, NR^{c32}C(O)OR^{a32}, NR^{c32}C(O)NR^{c32}R^{d32}, NR^{c32}S(O)R^{b32}, NR^{c32}S(O)₂R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32}, and BR^{b32}Rⁱ³²;
each R⁵⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a50}, SR^{a50}, C(O)R^{b50}, C(O)NR^{c50}R^{d50}, C(O)OR^{a50}, OC(O)R^{b50}, OC(O)NR^{c50}R^{d50}, NR^{c50}R^{d50}, NR^{c50}C(O)R^{b50}, NR^{c50}C(O)OR^{a50}, NR^{c50}C(O)NR^{c50}R^{d50}, NR^{c50}S(O)R^{b50}, NR^{c50}S(O)₂R^{b50}, NR^{c50}S(O)₂NR^{c50}, R^{d50}, S(O)R^{b50}, S(O)NR^{c50}R^{d50}, S(O)₂R^{b50}, S(O)₂NR^{c50}R^{d50}, and BR^{h50}Rⁱ⁵⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵¹;
each R⁵¹ is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c51}C(O)NR^{c51}R^{d51}, NR^{c51}S(O)R^{b51}, NR^{c51}S(O)₂R^{b51}, NR^{c51}, S(O)₂NR^{c51}R^{d51}, S(O)R^{b51}, S(O)NR^{c51}R^{d51}, S(O)₂R^{b51}, S(O)₂NR^{c51}R^{d51}, and BR^{h51}Rⁱ⁵¹;
each R⁶⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{a60}, SR^{a60}, C(O)R^{b60}, C(O)NR^{c60}R^{d60}, C(O)OR^{a60}, OC(O)R^{b60}, OC(O)NR^{c60}R^{d60}, NR^{c60}R^{d60}, NR^{c60}C(O)R^{b60}, NR OC(O)OR^{a60}, NR^{c60}C(O)NR^{c60}R^{d60}, NR^{c60}S(O)R^{b60}, NR^{c60}S(O)₂R^{b60}, NR^{c60}S(O)₂NR^{c60}R^{d60}, S(O)R^{b60}, S(O)NR^{c60}R^{d60}, S(O)₂R^{b60}, S(O)₂NR^{c60}R^{d60}, and BR^{h60}Rⁱ⁶⁰; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶¹;
each R⁶¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O) R^{b61}, NR^{c61}C(O)OR^{a61}, NR^{c61}C(O)NR^{c61}R^{d61}, NR^{c61}S(O)R^{b61}, NR^{c61}S(O)₂R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, and BR^{h61}Rⁱ⁶¹;
each R⁷⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, NO₂, OR^{c70}, SR^{a70}, C(O)R^{b70}, C(O)NR^{c70}R^{d70}, C(O)OR^{a70}, OC(O)R^{b70}, OC(O)NR^{c70}R^{d70}, NR^{c70}R^{d70}, NR^{c70}C(O)R^{b70}, NR^{c70}C(O)OR^{a70}, NR^{c70}C(O)NR^{c70}R^{d70}, NR^{c70}S(O)R^{b70}, NR^{c70}S(O)₂R^{b70}, NR^{c70}S(O)₂NR^{c70}R^{d70}, S(O)R^{b70}, S(O)NR^{c70}R^{d70}, S(O)₂R^{b70}, S(O)₂NR^{c70}R^{d70}, and BR^{h70}Rⁱ⁷⁰;
each R^{a1}, R^{b1}, R^{c1}, and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};
or any R^{c1} and R^{d1} attached to the same N atom, together with the N atom to which they are attached, form a 4-, 5-, 6- or 7-membered heterocycloalkyl group optionally substituted with 1, 2 or 3 substituents independently selected from R^{g};
each R^{h1} and Rⁱ¹ is independently selected from OH, C₁₋₆ alkoxy, and C₁₋₆ haloalkoxy; or any R^{h1} and Rⁱ¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a2}, R^{b2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²²;
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R²²;
each R^{e2} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h2} and Rⁱ² is independently selected from OH, C₁₋₆ alkoxy, and C₁₋₆ haloalkoxy; or any R^{h2} and Rⁱ² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a3}, R^{b3}, R3 and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R³⁰;
each R^{e3} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{f3} and Rⁱ³ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰;
each R^{h3} and Rⁱ³ is independently selected from OH, C₁₋₆ alkoxy, and C₁₋₆ haloalkoxy; or any R^{h3} and Rⁱ³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a4}, R^{b4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R^{g};
or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R^{g};
each R^{h4} and Rⁱ⁴ is independently selected from OH, C₁₋₆ alkoxy, and C₁₋₆ haloalkoxy; or any R^{h4} and Rⁱ⁴ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a5}, R^{b5}, R^{c5} and R^{d5} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁵⁰;
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R⁵⁰;
each R^{e5} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h5} and Rⁱ⁵ is independently selected from OH, C₁₋₆ alkoxy, and C₁₋₆ haloalkoxy; or any R^{h5} and Rⁱ⁵ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a6}, R^{b6}, R6 and R^{d6} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶⁰;
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R⁶⁰;
each R^{e6} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h6} and Rⁱ⁶ is independently selected from OH, C₁₋₆ alkoxy, and C₁₋₆ haloalkoxy; or any R^{h6} and Rⁱ⁶ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a7}, R^{b7}, R^{c7} and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁷⁰;
or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R⁷⁰;
each R^{e7} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h7} and Rⁱ⁷ is independently selected from OH, C₁₋₆ alkoxy, and C₁₋₆ haloalkoxy; or any R^{h7} and Rⁱ⁷ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a10}, R^{b10}, R^{c10} and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R¹¹;
each R^{e10} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{b10} and R^{c10} is independently selected from OH, C₁₋₆ alkoxy, and C₁₋₆ haloalkoxy; or any R^{b10} and R^{c10} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a11}, R^{b11}, R^{c11} and R^{d11}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;
or any R^{c11} and R^{d11} attached to the same N atom, together with the N atom to which they are attached, form a 4-, 5-, 6- or 7-membered heterocycloalkyl group;
each R^{h11} and Rⁱ¹¹ is independently selected from OH, C₁₋₆ alkoxy, and C₁₋₆ haloalkoxy; or any R^{h11} and Rⁱ¹¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a20}, R^{b20}, R^{c20} and R^{d20} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²¹;
or any R^{c20} and R^{d20} attached to the same N atom, together with the N atom to which they are attached, form a 4-, 5-, 6- or 7-membered heterocycloalkyl group optionally substituted with 1, 2, 3, or 4 substituents independently selected from R²¹;
each R^{e20} is independently selected from H, CN, C₁₋₆ alkyl, C₂₋₆ alkenyl, C₂₋₆ alkynyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{h20} and Rⁱ²⁰ is independently selected from OH, C₁₋₆ alkoxy, and C₁₋₆ haloalkoxy; or
any R^{h20} and Rⁱ²⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a21}, R^{b21}, R^{c21} and R^{d21}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R^{g};
or any R^{c21} and R^{d21} attached to the same N atom, together with the N atom to which they are attached, form a 4-, 5-, 6- or 7-membered heterocycloalkyl group optionally substituted with 1, 2 or 3 substituents independently selected from R^{g};
each R^{h21} and Rⁱ²¹ is independently selected from OH, C₁₋₆ alkoxy, and C₁₋₆ haloalkoxy; or any R^{h21} and Rⁱ²¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a22}, R^{b22}, R^{c22} and R^{d22} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²³;
or any R^{c22} and R^{d22} attached to the same N atom, together with the N atom to which they are attached, form a 4-, 5-, 6- or 7-membered heterocycloalkyl group optionally substituted with 1, 2, 3, or 4 substituents independently selected from R²³;
each R^{h22} and Rⁱ²² is independently selected from OH, C₁₋₆ alkoxy, and C₁₋₆ haloalkoxy; or any R^{h22} and Rⁱ²² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a23}, R^{b23}, R^{c23} and R^{d23}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;
or any R^{c23} and R^{d23} attached to the same N atom, together with the N atom to which they are attached, form a 4-, 5-, 6- or 7-membered heterocycloalkyl group;
each R^{h23} and Rⁱ²³ is independently selected from OH, C₁₋₆ alkoxy, and C₁₋₆ haloalkoxy; or any R^{h23} and Rⁱ²³ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a30}, R^{b30}, R^{c30} and R^{d30}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³¹;
or any R^{c30} and R^{d30} attached to the same N atom, together with the N atom to which they are attached, form a 4-, 5-, 6- or 7-membered heterocycloalkyl group optionally substituted with 1, 2, 3, or 4 substituents independently selected from R³¹;
each R^{h30} and Rⁱ³⁰ is independently selected from OH, C₁₋₆ alkoxy, and C₁₋₆ haloalkoxy; or
any R^{h30} and Rⁱ³⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a31}, R^{b31}, R^{c31} and R^{d31}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R³²;
or any R^{c31} and R^{d31} attached to the same N atom, together with the N atom to which they are attached, form a 4-, 5-, 6- or 7-membered heterocycloalkyl group optionally substituted with 1, 2 or 3 substituents independently selected from R³²;
each R^{h31} and Rⁱ³¹ is independently selected from OH, C₁₋₆ alkoxy, and C₁₋₆ haloalkoxy; or any R^{h31} and Rⁱ³¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a32}, R^{b32}, R^{e32} and R^{d32}, is independently selected from H, C₁₋₆ alkyl, C₂₋₆ alkenyl, C₂₋₆ alkynyl and C₁₋₆ haloalkyl;
each R^{h32} and Rⁱ³² is independently selected from OH, C₁₋₆ alkoxy, and C₁₋₆ haloalkoxy; or any R^{h32} and Rⁱ³² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a50}, R^{b50}, R^{c50} and R^{d50}, is independently selected from H, C₁₋₆ alkyl, C₂₋₆ alkenyl, C₂₋₆ alkynyl, C₁₋₆haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R⁵¹;
or any R^{c50} and R^{d50} attached to the same N atom, together with the N atom to which they are attached, form a 4-, 5-, 6- or 7-membered heterocycloalkyl group optionally substituted with 1, 2 or 3 substituents independently selected from R⁵¹;
each R^{h50} and Rⁱ⁵⁰ is independently selected from OH, C₁₋₆ alkoxy, and C₁₋₆ haloalkoxy; or any R^{h50} and Rⁱ⁵⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a51}, R^{b51}, R^{c51} and R^{d51}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;
or any R^{c51} and R^{d51} attached to the same N atom, together with the N atom to which they are attached, form a 4-, 5-, 6- or 7-membered heterocycloalkyl group;
each R^{h51} and Rⁱ⁵¹ is independently selected from OH, C₁₋₆ alkoxy, and C₁₋₆ haloalkoxy; or any R^{h51} and Rⁱ⁵¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a60}, R^{b60}, R^{c60} and R^{d60} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶¹;
or any R^{c60} and R^{d60} attached to the same N atom, together with the N atom to which they are attached, form a 4-, 5-, 6- or 7-membered heterocycloalkyl group optionally substituted with 1, 2, 3, or 4 substituents independently selected from R⁶¹;
each R^{h60} and Rⁱ⁶⁰ is independently selected from OH, C₁₋₆ alkoxy, and C₁₋₆ haloalkoxy; or any R^{h60} and Rⁱ⁶⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a61}, R^{b61}, R^{c61} and R^{d61}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;
or any R^{c61} and R^{d61} attached to the same N atom, together with the N atom to which they are attached, form a 4-, 5-, 6- or 7-membered heterocycloalkyl group;
each R^{h61} and Rⁱ⁶¹ is independently selected from OH, C₁₋₆ alkoxy, and C₁₋₆ haloalkoxy; or any R^{h61} and Rⁱ⁶¹ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{a70}, R^{b70}, R^{c70} and R^{d70} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c70} and R^{d70} attached to the same N atom, together with the N atom to which they are attached, form a 4-, 5-, 6- or 7-membered heterocycloalkyl group;
each R^{h70} and Rⁱ⁷⁰ is independently selected from OH, C₁₋₆ alkoxy, and C₁₋₆ haloalkoxy; or
any R^{h70} and Rⁱ⁷⁰ attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; and
each R^{g} is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

3. The compound of claim 1, wherein the compound of Formula I is a compound of Formula Ia: 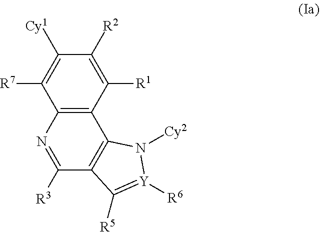
or a pharmaceutically acceptable salt thereof,
wherein:
Y is N or C;
R¹ is selected from H, D, C₁₋₆ alkyl, C₁₋₆ haloalkyl, halo, and CN;
R² is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;
Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3 or 4 substituents independently selected from R¹⁰;
R³ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{f3}, SR^{a3}, C(O)R^{b3}, C(O)NR^{c3}R^{d3}, C(O)OR^{a3}, OC(O)R^{b3}OC(O)NR^{c3}R^{d3}, NR^{c3}R^{j3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R³⁰;
R⁵ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁵⁰;
when R⁴R⁵C [symbol] YR⁶ is a double bond and Y is N, then R⁴ and R⁶ are absent;
when R⁴R⁵C [symbol] YR⁶ is a double bond and Y is C, then R⁴ is absent; and
R⁶ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a6}, SR^{a6}, C(O)R^{b6}, C(O)NR^{c6}R^{d6}, C(O)OR^{a6}, OC(O)R^{b6}OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂R^{b6}, NR^{c6}S(O)₂NR^{c6}R^{d6}, S(O)₂R^{b6}, and S(O)₂NR^{c6}R^{d6}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R⁶⁰;
R⁷ is selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7};
Cy² is selected from C₃₋₁₀ cycloalkyl, 4-14 membered heterocycloalkyl, C₆₋₁₀ aryl and 5-10 membered heteroaryl; wherein the 4-14 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-14 membered heterocycloalkyl is optionally substituted by oxo to form a carbonyl group; and wherein the C₃₋₁₀ cycloalkyl, 4-14 membered heterocycloalkyl, C₆₋₁₀ aryl and 5-10 membered heteroaryl are each optionally substituted with 1, 2, 3 or 4 substituents independently selected from R²⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a20}, SR^{a20}, C(O)R^{b20}, C(O)NR^{c20}R^{d20}, C(O)OR^{a20}, OC(O)R^{b20}, OC(O)NR^{c20}R^{d20}, NR^{c20}R^{d20}, NR^{c20}C(O)R^{b20}, NR^{c20}C(O)OR^{a20}, NR^{c20}C(O)NR^{c20}R^{d20}, NR^{c20}S(O)₂R^{b20}, NR^{c20}S(O)₂NR^{c20}R^{d20}, S(O)₂R^{b20}, and S(O)₂NR^{c20}R^{d20}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{a21}S(O)₂R^{b21}, NR^{c21}, S(O)₂NR^{c21}R^{d21}, S(O)₂R^{b21}, and S(O)₂NR^{c21}R^{d21},
each R²² is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a22}, SR^{a22}, C(O)R^{b22}, C(O)NR^{c22}R^{d22}, C(O)OR^{a22}, OC(O)R^{b22}, OC(O)NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, NR^{c22}S(O)₂R^{b22}, NR^{c22}S(O)₂NR^{c22}R^{d22}, S(O)₂R^{b22}, and S(O)₂NR^{c22}R^{d22};
each R³⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a30}, SR^{a30}, C(O)R^{b30}, C(O)NR^{c30}R^{d30}, C(O)OR^{a30}, OC(O)R^{b30}, OC(O)NR^{c30}R^{d30}, NR^{c30}R^{d30}, NR^{c30}C(O)R^{b30}, NR^{c30}C(O)OR^{a30}, NR^{c30}C(O)NR^{c30}R^{d30}, NR^{c30}S(O)₂R^{b30}, NR^{c30}S(O)₂NR^{c30}R^{d30}, S(O)₂R^{b30}, and S(O)₂NR^{c30}R^{d30}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃alkylene, C₆₋₁₀ aryl-C₁₋₃alkylene and 5-10 membered heteroaryl-C₁₋₃ alkylene are each optionally substituted with 1, 2, 3, or 4 substituents independently selected from R³¹;
each R³¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a31}, SR^{a31}, C(O)R^{b31}, C(O)NR^{c31}R^{d31}, C(O)OR^{a31}, OC(O)R^{b31}, OC(O)NR^{c31}R^{d31}, NR^{c31}R^{d31}, NR^{c31}C(O)R^{b31}, NR^{d31}C(O)OR^{a31}, NR^{c31}C(O)NR^{c31}, R^{c31}NR^{d31}S(O)₂R^{b31}, NR^{c31}S(O)₂NR^{c31}R^{d31}, S(O)₂R^{b31}, and S(O)₂NR^{c31}R^{d31};
each R⁵⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₅₀ aryl-C₁₋₃ alkylene, 5-10 membered heteroaryl-C₁₋₃alkylene, halo, D, CN, OR^{a50}, SR^{a50}, C(O)R^{b50}, C(O)NR^{c50}R^{d50}, C(O)OR^{a50}, OC(O)R^{b50}, OC(O)NR^{c50}R^{d50}, NR^{c50}R^{d50}, NR^{c50}C(O)R^{b50}, NR^{c50}C(O)OR^{a50}, NR^{c50}C(O)NR^{c50}R^{d50}, NR^{c50}S(O)₂R^{b50}, NR^{c50}S(O)₂NR^{c50}R^{d50}, S(O)₂R^{b50}, and S(O)₂NR^{c50}R^{d50};
each R⁶⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃alkylene, halo, D, CN, OR^{a60}, SR^{a60}, C(O)R^{b60}, C(O)NR^{c60}R^{d60}, C(O)OR^{a60}, OC(O)R^{b60}, OC(O)NR^{c60}R^{d60}, NR^{c60}R^{d60}, NR^{c60}C(O)R^{b60}, NR^{c60}C(O)OR^{a60}, NR^{c60}C(O)NR^{c60}R^{d60}, NR^{c60}S(O)₂R^{b60}, NR^{c60}S(O)₂NR^{c60}R^{d60}, S(O)₂R^{b60}, and S(O)₂NR^{c60}, R^{d60};
each R^{a2}, R^{b2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²²;
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R²²;
each R^{a3}, R^{b3}, R^{c3} and R^{d3} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R³⁰;
each R^{f3} and R^{j3} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c3} and R^{j3} attached to the same N atom, together with the N atom to which they are attached, form a 4-, 5-, 6- or 7-membered heterocycloalkyl group optionally substituted with 1, 2, 3, or 4 substituents independently selected from R³⁰;
each R^{a5}, R^{b5}, R^{c5} and R^{d}s is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁵⁰;
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R⁵⁰;
each R^{a6}, R^{b6}, R^{c6} and R^{d6} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁶⁰;
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R⁶⁰;
each R^{a7}, R^{b7}, R^{c7} and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c7} and R^{d7} attached to the same N atom, together with the N atom to which they are attached, form a 4-, 5-, 6- or 7-membered heterocycloalkyl group;
each R^{a10}, R^{b10}, R^{c10} and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c10} and R^{d10} attached to the same N atom, together with the N atom to which they are attached, form a 4-, 5-, 6- or 7-membered heterocycloalkyl group;
each R^{a20}, R^{b20}, R^{c20} and R^{d20} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R²¹;
or any R^{c20} and R^{d20} attached to the same N atom, together with the N atom to which they are attached, form a 4-, 5-, 6- or 7-membered heterocycloalkyl group optionally substituted with 1, 2, 3, or 4 substituents independently selected from R²¹;
each R^{a21}, R^{b21}, R^{c21} and R^{d21}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;
or any R^{c21} and R^{d21} attached to the same N atom, together with the N atom to which they are attached, form a 4-, 5-, 6- or 7-membered heterocycloalkyl group;
each R^{a22}, R^{b22}, R^{c22} and R^{d22} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c22} and R^{d22} attached to the same N atom, together with the N atom to which they are attached, form a 4-, 5-, 6- or 7-membered heterocycloalkyl group;
each R^{a30}, R^{b30}, R^{c30} and R^{d30}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³¹;
or any R^{c30} and R^{d30}, attached to the same N atom, together with the N atom to which they are attached, form a 4-, 5-, 6- or 7-membered heterocycloalkyl group optionally substituted with 1, 2, 3, or 4 substituents independently selected from R³¹;
each R^{a31}, R^{b31}, R^{c31} and R^{d31}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;
or any R^{c31} and R^{d31} attached to the same N atom, together with the N atom to which they are attached, form a 4-, 5-, 6- or 7-membered heterocycloalkyl group;
each R^{a50}, R^{b50}, R^{c50} and R^{d50}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c50} and R^{d50}, attached to the same N atom, together with the N atom to which they are attached, form a 4-, 5-, 6- or 7-membered heterocycloalkyl group; and
each R^{a60}, R^{b60}, R^{c60} and R^{d60} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c60} and R^{d60} attached to the same N atom, together with the N atom to which they are attached, form a 4-, 5-, 6- or 7-membered heterocycloalkyl group.

4. The compound of claim 3, of a pharmaceutically acceptable salt thereof, wherein
Y is N or C;
R¹ is selected from H, D, and C₁₋₆ alkyl;
R² is selected from H, C₁₋₆ alkyl, C₁₋₆ haloalkyl, halo, D, CN, OR^{a2}, and NR^{c2}R^{d2}; wherein said C₁₋₆ alkyl, is optionally substituted with 1 or 2 substituents independently selected from R²²;
Cy¹ is selected from C₆₋₁₀ aryl and 6-10 membered heteroaryl; wherein the 6-10 membered heteroaryl has at least one ring-forming carbon atom and 1, 2, 3, or 4 ring-forming heteroatoms independently selected from N, O, and S; wherein a ring-forming carbon atom of 6-10 membered heteroaryl is optionally substituted by oxo to form a carbonyl group; and wherein the C₆₋₁₀ aryl and 6-10 membered heteroaryl are each optionally substituted with 1, 2, or 3 substituents independently selected from R¹⁰;
R³ is selected from H, C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{f3}, and NR^{c3}R^{j3}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R³⁰;
R⁵ is selected from H, C₁₋₆ alkyl, C₁₋₆ haloalkyl, C₃₋₆ cycloalkyl, 4-10 membered heterocycloalkyl, C₆₋₁₀ aryl, 5-10 membered heteroaryl, halo, D, CN, OR^{a5}, C(O)NR^{c5}R^{d5}, and NR^{c5}R^{d5}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R⁵⁰;
when R⁴R⁵C [symbol] YR⁶ is a double bond and Y is N, then R⁴ and R⁶ are absent;
when R⁴R5 [symbol] YR⁶ is a double bond and Y is C, then R⁴ is absent; and
R⁶ is selected from H, C₁₋₆ alkyl, C₁₋₆ haloalkyl, C₃₋₆ cycloalkyl, 4-10 membered heterocycloalkyl, C₆₋₁₀ aryl, 5-10 membered heteroaryl, halo, D, CN, OR^{a6}, and NR^{c6}R^{d6}; wherein said C₁₋₆ alkyl, C₃₋₆ cycloalkyl, 4-10 membered heterocycloalkyl, C₆₋₁₀ aryl, and 5-10 membered heteroaryl, are each optionally substituted with 1, 2, or 3 substituents independently selected from R⁶⁰;
R⁷ is selected from H, C₁₋₆ alkyl, C₁₋₆ haloalkyl, halo, D, and CN;
Cy² is selected from 4-10 membered heterocycloalkyl; wherein the 4-10 membered heterocycloalkyl has at least one ring-forming carbon atom and 1, 2, 3, or 4 ring-forming heteroatoms independently selected from N, O, and S; wherein a ring-forming carbon atom of the 4-10 membered heterocycloalkyl is optionally substituted by oxo to form a carbonyl group;
and wherein the 4-10 membered heterocycloalkyl, is optionally substituted with 1, 2 or 3 substituents independently selected from R²⁰;
each R¹⁰ is independently selected from C₁₋₆ alkyl, C₁₋₆ haloalkyl, halo, D, CN, OR^{a10}, and NR^{c10}R^{d10};
each R²⁰ is independently selected from C₁₋₆ alkyl, C₂₋₆ alkenyl, C₂₋₆ alkynyl, C₁₋₆ haloalkyl, halo, D, CN, OR^{a20}, C(O)R^{b20}, C(O)NR^{c20}R^{d20}, and NR^{c20}R^{d20}; wherein said C₁₋₆ alkyl, C₂₋₆ alkenyl, and C₂₋₆ alkynyl, are each optionally substituted with 1, 2, or 3 substituents independently selected from R²¹;
each R²¹ is independently selected from C₁₋₆ alkyl, C₁₋₆ haloalkyl, halo, D, CN, OR^{a21}, and NR^{c21}R^{d21};
each R²² is independently selected from C₁₋₆ alkyl, C₁₋₆ haloalkyl, halo, D, CN, OR^{a22}, and NR^{c22}R^{d22};
each R³⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a30}, C(O)NR^{c30}R^{d30}, and NR^{c30}R^{d30}, wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R³¹;
each R³¹ is independently selected from C₁₋₆ alkyl, C₁₋₆ haloalkyl, halo, D, CN, OR^{a31}, and NR^{c31}R^{d31};
each R⁵⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a50}, and NR^{c50}R^{d50};
each R⁶⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a60}, C(O)NR^{c60}R^{d60} (O)OR^{a60}, and NR^{c60}R^{d60};
each R^{a2}, R^{c2} and R^{d2} is independently selected from H, C₁₋₆ alkyl, and C₁₋₆ haloalkyl;
wherein said C₁₋₆ alkyl is optionally substituted with 1 or 2 substituents independently selected from R²²;
each R^{c3} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³⁰;
each R^{f3} and R^{j3} is independently selected from C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³⁰;
or any R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;
each R^{a5}, R^{c5} and R^{d5} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R⁵⁰;
or any R^{c5} and R^{d5} attached to the same N atom, together with the N atom to which they are attached, form a 4-, 5-, or 6-membered heterocycloalkyl group optionally substituted with 1, 2, or 3 substituents independently selected from R⁵⁰;
each R^{a6}, R^{c6} and R^{d6} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R⁶⁰;
or any R^{c6} and R^{d6} attached to the same N atom, together with the N atom to which they are attached, form a 4-, 5-, or 6-membered heterocycloalkyl group optionally substituted with 1, 2, or 3 substituents independently selected from R⁶⁰;
each R^{a10}, R^{c10} and R^{d10} is independently selected from H, C₁₋₆ alkyl, and C₁₋₆ haloalkyl;
each R^{a20}, R^{b20}, R^{c20} and R^{d20} is independently selected from H, C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1, 2, or 3 substituents independently selected from R²¹;
each R^{a21}, R^{c21} and R^{d21}, is independently selected from H, C₁₋₆ alkyl, and C₁₋₆ haloalkyl;
each R^{a22}, R^{c22} and R^{d22} is independently selected from H, C₁₋₆ alkyl, and C₁₋₆ haloalkyl;
each R^{a30}, R^{c30} and R^{d30} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³¹;
or any R^{c30} and R^{d30} attached to the same N atom, together with the N atom to which they are attached, form a 4-, 5-, or 6-membered heterocycloalkyl group optionally substituted with 1, 2, or 3 substituents independently selected from R³¹;
each R^{a31}, R^{c31} and R^{d31}, is independently selected from H, C₁₋₆ alkyl, and C₁₋₆ haloalkyl;
each R^{a50}, R^{c50} and R^{d50}, is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl;
or any R^{c50} and R^{d50} attached to the same N atom, together with the N atom to which they are attached, form a 4-, 5-, or 6-membered heterocycloalkyl group; and
each R^{a60}, R^{c60} and R^{d60} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl;
or any R^{c60} and R^{d60} attached to the same N atom, together with the N atom to which they are attached, form a 4-, 5-, or 6-membered heterocycloalkyl group.

5. The compound of claim 1, or pharmaceutically acceptable salt thereof, wherein
Y is N or C;
R¹ is H;
R² is selected from H, C₁₋₆ alkyl, C₁₋₆ haloalkyl, halo, D, and CN;
Cy¹ is selected from C₆₋₁₀ aryl and 6-10 membered heteroaryl; wherein the 6-10 membered heteroaryl has at least one ring-forming carbon atom and 1 or 2 ring-forming heteroatoms independently selected from N and O; and wherein the C₆₋₁₀ aryl and 6-10 membered heteroaryl are each optionally substituted with 1, 2, or 3 substituents independently selected from R¹⁰;
R³ is selected from H, C₁₋₆ alkyl, C₁₋₆ haloalkyl, C₃₋₁₀ cycloalkyl, 4-10 membered heterocycloalkyl, halo, D, CN, OR^{f3}, and NR^{c3}R^{j3}; wherein said C₁₋₆ alkyl, C₃₋₁₀cycloalkyl, and 4-10 membered heterocycloalkyl, are each optionally substituted with 1, 2, or 3 substituents independently selected from R³⁰;
R⁵ is selected from H, C₁₋₆ alkyl, C₁₋₆ haloalkyl, halo, D, and CN;
when R⁴R⁵C [symbol] YR⁶ is a double bond and Y is N, then R⁶ is absent;
R⁶ is selected from H, C₁₋₆ alkyl, C₁₋₆ haloalkyl, halo, D, and CN;
R⁷ is selected from H, C₁₋₆ alkyl, C₁₋₆ haloalkyl, halo, D, and CN;
Cy² is selected from 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted with 1, 2 or 3 substituents independently selected from R²⁰;
each R¹⁰ is independently selected from C₁₋₆ alkyl, C₁₋₆ haloalkyl, halo, D, CN, OR^{a10}, and NR^{c10}R^{d10};
each R²⁰ is independently selected from C₁₋₆ alkyl, C₁₋₆ haloalkyl, halo, D, CN, OR^{a20}, C(O)R^{b20}, C(O)NR^{c20}R^{d20}, and NR^{c20}R^{d20}; wherein said C₁₋₆ alkyl, is optionally substituted with 1 or 2 substituents independently selected from R²¹;
each R²¹ is independently selected from C₁₋₆ alkyl, C₁₋₆ haloalkyl, halo, D, CN, OR^{a21}, and NR^{c21}R^{d21};
each R³⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a30}, C(O)NR^{c30}R^{d30}, and NR^{c30}R^{d30} wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R³¹;
each R³¹ is independently selected from C₁₋₆ alkyl, C₁₋₆ haloalkyl, halo, D, CN, OR^{a31}, and NR^{c31}R^{d31};
each R^{c3} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³⁰;
each R^{f3} and R^{j3} is independently selected from C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³⁰;
or any R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;
each R^{a10}, R^{c10} and R^{d10} is independently selected from H, C₁₋₆ alkyl, and C₁₋₆ haloalkyl;
each R^{a20}, R^{b20}, R^{c20} and R^{d20} is independently selected from H, C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1, 2, or 3 substituents independently selected from R²¹;
each R^{a21}, R^{c21} and R^{d21}, is independently selected from H, C₁₋₆ alkyl, and C₁₋₆ haloalkyl;
each R^{a30}, R^{c30} and R^{d30} is independently selected from H, C₁₋₆ alkyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, or 3 substituents independently selected from R³¹;
or any R^{c30} and R^{d30} attached to the same N atom, together with the N atom to which they are attached, form a 4-, 5-, or 6-membered heterocycloalkyl group optionally substituted with 1, 2, or 3 substituents independently selected from R³¹; and
each R^{a31}, R^{b31} and R^{d31}, is independently selected from H, C₁₋₆ alkyl, and C₁₋₆ haloalkyl.

6. The compound of claim 1, wherein the compound of Formula I is a compound of Formula II: 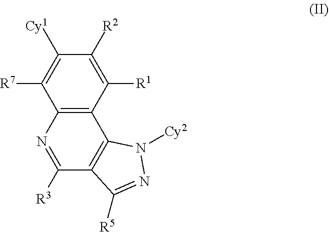
or a pharmaceutically acceptable salt thereof.

7. The compound of claim 1, wherein
X is CR⁷;
R¹ is selected from H;
R² is selected from H, C₁₋₃ haloalkyl, and halo;
Cy¹ is C₁₀ aryl; and wherein the C₁₀ aryl is optionally substituted with 1 or 2 substituents independently selected from R¹⁰;
R³ is selected from H and 4-6 membered heterocycloalkyl; wherein said 4-6 membered heterocycloalkyl, is optionally substituted with 1 or 2 substituents independently selected from R³⁰;
R⁵ is H;
R⁴R⁵C [symbol] YR⁶ is a double bond, Y is N, and R⁴ and R⁶ are absent;
R⁷ is selected from H or halo;
Cy² is 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted with 1 or 2 substituents independently selected from R²⁰;
each R¹⁰ is independently selected from OR^{a10};
each R²⁰ is independently selected from C(O)R^{b20};
each R³⁰ is independently selected from NR^{c30}R^{d30};
each R^{a10} is independently selected from H and C₁₋₃ alkyl;
each R^{b20} is C₁₋₃ alkyl or C₂₋₄ alkenyl; and
each R^{c30} and R^{d30} is independently selected from C₁₋₃ alkyl.

8. The compound of claim 1, wherein the compound of Formula I is a compound of Formula III: 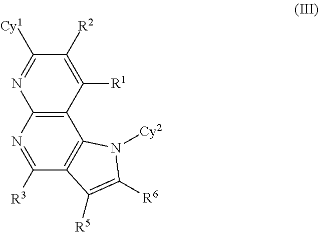
or a pharmaceutically acceptable salt thereof.

9. The compound of claim 1, wherein the compound of Formula I is a compound of Formula IV: 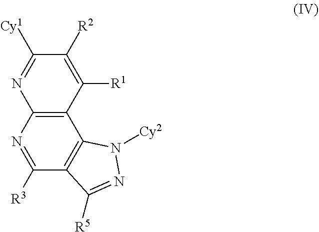
or a pharmaceutically acceptable salt thereof.

10. The compound of claim 1, wherein the compound of Formula I is a compound of Formula V: 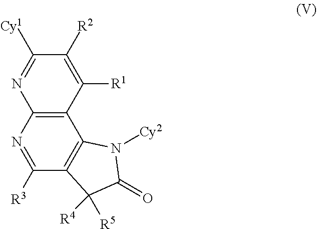
or a pharmaceutically acceptable salt thereof.

11. The compound of claim 1, wherein the compound of Formula I is a compound of Formula VI: 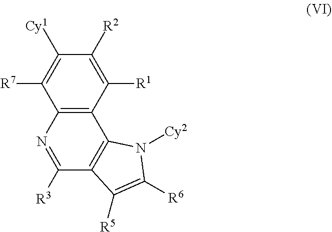
or a pharmaceutically acceptable salt thereof.

12. The compound of claim 1, wherein the compound of Formula I is a compound of Formula VII: 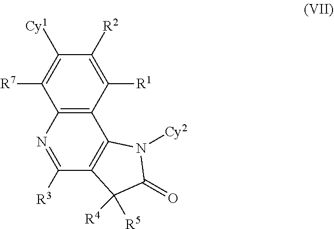
or a pharmaceutically acceptable salt thereof.

13. The compound of claim 1, wherein X is CR⁷.

14. The compound of claim 1, wherein X is N.

15. The compound of claim 1, wherein R⁴R⁵C [symbol] YR⁶ is a double bond, Y is N, and R⁴ and R⁶ are absent.

16. The compound of claim 1, wherein R⁴R⁵C [symbol] YR⁶ is a double bond, Y is C, and R⁴ is absent.

17. The compound of claim 1, wherein R¹ is selected from H, D, C₁₋₆ alkyl, C₁₋₆ haloalkyl, halo, OR^{a1}, and NR^{c1}R^{d1}.

18. The compound of claim 1, wherein R¹ is H.

19. The compound of claim 1, wherein R² is selected from H, C₁₋₆ alkyl, C₁₋₆ haloalkyl, halo, D, and CN; wherein said C₁₋₆ alkyl is optionally substituted with 1 or 2 substituents independently selected from R²².

20. The compound of claim 1, wherein each R²² is independently selected from C₁₋₆ alkyl, C₁₋₆ haloalkyl, halo, and CN.

21. The compound of claim 1, wherein R² is halo.

22. The compound of claim 1, wherein Cy¹ is selected from C₆₋₁₀ aryl and 6-10 membered heteroaryl; wherein the 6-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 6-10 membered heteroaryl is optionally substituted by oxo to form a carbonyl group; and wherein the C₆₋₁₀ aryl and 6-10 membered heteroaryl are each optionally substituted with 1 or 2 substituents independently selected from R¹⁰.

23. The compound of claim 1, wherein Cy¹ is C₆₋₁₀ aryl optionally substituted with 1 or 2 substituents independently selected from R¹⁰.

24. The compound of claim 1, wherein Cy¹ is 6-10 membered heteroaryl optionally substituted with 1 or 2 substituents independently selected from R¹⁰.

25. The compound of claim 1, wherein R¹⁰ is selected from C₁₋₃ alkyl, C₁₋₃ haloalkyl, halo, D, CN, and OR^{a10}.

26. The compound of claim 1, wherein R³ is selected from H, 4-10 membered heterocycloalkyl, C₆₋₁₀ aryl, and OR^{f3}; wherein said 4-10 membered heterocycloalkyl, and C₆₋₁₀ aryl, are each optionally substituted with 1 or 2 substituents independently selected from R³⁰.

27. The compound of claim 1, wherein R³ is selected from H, 4-6 membered heterocycloalkyl, and OR^{f3}; wherein said 4-6 membered heterocycloalkyl is optionally substituted with 1 or 2 substituents independently selected from R³⁰.

28. The compound of claim 1, wherein each R³⁰ is independently selected from C₁₋₆ alkyl, C₁₋₆ haloalkyl, 4-6 membered heterocycloalkyl, 5-6 membered heteroaryl, halo, D, CN, OR^{a30}, and NR^{c30}R^{d30}; wherein said C₁₋₆ alkyl, 4-6 membered heterocycloalkyl, and 5-6 membered heteroaryl, are each optionally substituted with 1 or 2 substituents independently selected from R³¹.

29. The compound of claim 1, wherein each R³¹ is independently selected from C₁₋₆ alkyl, C₁₋₆ haloalkyl, halo, D, and CN.

30. The compound of claim 1, wherein R⁵ is selected from H, C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a5}, C(O)NR^{c5}R^{d5}, and NR^{c5}R^{d5}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R⁵⁰.

31. The compound of claim 1, wherein R⁵ is H.

32. The compound of claim 1, wherein each R⁵⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a50}, and NR^{c50}R^{d50}.

33. The compound of claim 1, wherein R⁶ is selected from H, C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a6}, and NR^{c6}R^{d6}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2, or 3 substituents independently selected from R⁶⁰.

34. The compound of claim 1, wherein each R⁶⁰ is independently selected from C₁₋₆ alkyl, C₁₋₆ haloalkyl, C₃₋₁₀ cycloalkyl, 4-10 membered heterocycloalkyl, C₆₋₁₀ aryl, 5-10 membered heteroaryl, halo, D, CN, OR^{a60}, C(O)NR^{c60}R^{d60}, C(O)OR^{a60}, and NR^{c60}R^{d60}.

35. The compound of claim 1, wherein R⁷ is selected from H, C₁₋₃ alkyl, C₁₋₃ haloalkyl, and halo.

36. The compound of claim 1, wherein R⁷ is halo.

37. The compound of claim 1, wherein Cy² is 4-6 membered heterocycloalkyl; wherein the 4-6 membered heterocycloalkyl has at least one ring-forming carbon atom and 1 or 2 ring-forming heteroatoms independently selected from N and O; and wherein the 4-6 membered heterocycloalkyl, is optionally substituted with 1 or 2 substituents independently selected from R²⁰.

38. The compound of claim 1, wherein Cy² is selected from 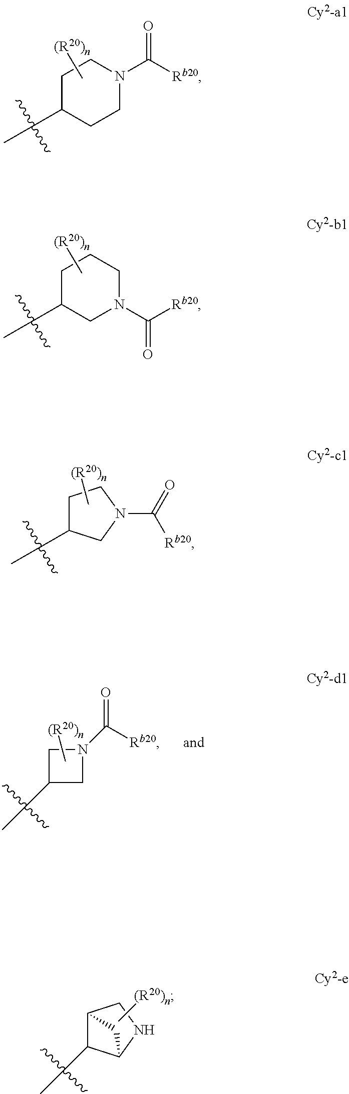
wherein n is 0, 1 or 2.

39. The compound of claim 38, wherein Cy² is Cy²-a1.

40. The compound of claim 38, wherein Cy² is Cy².

41. The compound of claim 1, wherein each R²⁰ is independently selected from C₁₋₆ alkyl, C₁₋₆ haloalkyl, halo, D, CN, and C(O)R^{b20}; wherein said C₁₋₆ alkyl, is optionally substituted with 1 or 2 substituents independently selected from R²¹.

42. The compound of claim 1, wherein each R²¹ is independently selected from C₁₋₆ alkyl, C₁₋₆ haloalkyl, halo, D, CN, OR^{a21}, and NR^{c21}R^{d21}.

43. The compound of claim 1, wherein the compound of Formula I is
1-(4-(8-chloro-6-fluoro-7-(3-hydroxynaphthalen-1-yl)-1H-pyrazolo[4,3-c]-quinolin-1-yl)-piperidin-1-yl)prop-2-en-1-one; or
1-(4-(8-chloro-4-(3-(dimethylamino)azetidin-1-yl)-6-fluoro-7-(3-hydroxynaphthalen-1-yl)-1H-pyrazolo[4,3-c]quinolin-1-yl)piperidin-1-yl)prop-2-en-1-one;
or a pharmaceutically acceptable salt thereof.

44. The compound of claim 1, wherein the compound of Formula I is selected from
2-((2S,4S)-1-acryloyl-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-methoxybut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4,4-difluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-((E)-4,4-difluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(6-chloro-5-methyl-1H-indazol-4-yl)-4-(3-(dimethylamino)azetidin-1-yl)-6-fluoro-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-1-(but-2-ynoyl)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-methoxybut-2-enoyl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4-fluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-((E)-4,4-difluorobut-2-enoyl)piperidin-2-yl)acetonitrile;
2-((2S,4S)-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)-1-(2-fluoroacryloyl)piperidin-2-yl)acetonitrile;
2-((2S,4S)-1-acryloyl-4-(8-chloro-7-(6-chloro-5-methyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)-1-((E)-4-(dimethylamino)but-2-enoyl)piperidin-2-yl)acetonitrile;
2-((2S,4S)-1-(but-2-ynoyl)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile;
2-((2S,4S)-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)-1-((E)-4-methoxybut-2-enoyl)piperidin-2-yl)acetonitrile;
2-((2S,4S)-1-acryloyl-4-(8-chloro-7-(6-chloro-5-methyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile;
2-((2S,4S)-1-acryloyl-4-(8-chloro-4-(3-(dimethylamino)azetidin-1-yl)-6-fluoro-7-(5-fluoroquinolin-8-yl)-1H-pyrazolo[4,3-c]quinolin-1-yl)piperidin-2-yl)acetonitrile;
2-((2S,4S)-1-acryloyl-4-(8-chloro-4-(3-(dimethylamino)azetidin-1-yl)-6-fluoro-7-(isoquinolin-4-yl)-1H-pyrazolo[4,3-c]quinolin-1-yl)piperidin-2-yl)acetonitrile;
2-((2S,4S)-1-acryloyl-4-(8-chloro-7-(2-chloro-3-methylphenyl)-4-(3-(dimethylamino)azetidin-1-yl)-6-fluoro-1H-pyrazolo[4,3-c]quinolin-1-yl)piperidin-2-yl)acetonitrile;
2-((2S,4S)-4-(8-chloro-7-(2-chloro-3-methylphenyl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-1-acryloyl-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile;
2-((2S,4S)-1-(but-2-ynoyl)-4-(8-chloro-7-(2,3-dichlorophenyl)-4-(3-(dimethylamino)azetidin-1-yl)-6-fluoro-1H-pyrazolo[4,3-c]quinolin-1-yl)piperidin-2-yl)acetonitrile;
2-((2S,4S)-1-acryloyl-4-(8-chloro-4-(3-(dimethylamino)azetidin-1-yl)-6-fluoro-7-(3-methyl-2-(trifluoromethyl)phenyl)-1H-pyrazolo[4,3-c]quinolin-1-yl)piperidin-2-yl)acetonitrile;
2-((2S,4S)-1-acryloyl-4-(8-chloro-6-fluoro-7-(3-methyl-2-(trifluoromethyl)phenyl)-4-(((S)-1-methylpyrrolidin-2-yl)methoxy)-1H-pyrazolo[4,3-c]quinolin-1-yl)piperidin-2-yl)acetonitrile;
methyl 3-(1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinolin-2-yl)propanoate;
3-(1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinolin-2-yl)-N,N-dimethylpropanamide;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-2-propyl-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-2-(1-methyl-1H-pyrazol-4-yl)-4-(((S)-1-methylpyrrolidin-2-yl)methoxy)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-phenyl-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-(pyridin-3-yl)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-3-(2-methyloxazol-5-yl)-4-(((S)-1-methylpyrrolidin-2-yl)methoxy)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-(2-methylthiazol-5-yl)-1H-pyrrolo[3,2-c]quinolin-8-yl)propanenitrile; and
3-(1-(2-azabicyclo[2.1.1]hexan-5-yl)-6-fluoro-7-(3-hydroxynaphthalen-1-yl)-3-(1-methyl-1H-pyrazol-4-yl)-4-(((S)-1-methylpyrrolidin-2-yl)methoxy)-1H-pyrrolo[3,2-c]quinolin-8-yl)propanenitrile;
or a pharmaceutically acceptable salt thereof.

45. The compound of claim 1, wherein the compound of Formula I is selected from
3-(1-(2-azabicyclo[2.1.1]hexan-5-yl)-6-fluoro-7-(3-hydroxynaphthalen-1-yl)-2-(1-methyl-1H-pyrazol-3-yl)-4-(((S)-1-methylpyrrolidin-2-yl)methoxy)-1H-pyrrolo[3,2-c]quinolin-8-yl)propanenitrile;
3-(2-benzyl-1-(2-azabicyclo[2.1.1]hexan-5-yl)-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-(1H-pyrazol-4-yl)-1H-pyrrolo[3,2-c]quinolin-8-yl)propanenitrile;
3-(1-(2-azabicyclo[2.1.1]hexan-5-yl)-6-fluoro-7-(3-hydroxynaphthalen-1-yl)-4-(((S)-1-methylpyrrolidin-2-yl)methoxy)-3-(6-oxo-1,6-dihydropyridin-3-yl)-1H-pyrrolo[3,2-c]quinolin-8-yl)propanenitrile;
3-(1-(2-azabicyclo[2.1.1]hexan-5-yl)-3-chloro-6-fluoro-7-(3-hydroxynaphthalen-1-yl)-4-(((S)-1-methylpyrrolidin-2-yl)methoxy)-1H-pyrrolo[3,2-c]quinolin-8-yl)propanenitrile;
1-(2-azabicyclo[2.1.1]hexan-5-yl)-8-(2-cyanoethyl)-6-fluoro-N-(2-hydroxyethyl)-7-(3-hydroxynaphthalen-1-yl)-4-(((S)-1-methylpyrrolidin-2-yl)methoxy)-1H-pyrrolo[3,2-c]quinoline-3-carboxamide;
N-Benzyl-1-(2-azabicyclo[2.1.1]hexan-5-yl)-8-(2-cyanoethyl)-6-fluoro-7-(3-hydroxynaphthalen-1-yl)-4-(((S)-1-methylpyrrolidin-2-yl)methoxy)-1H-pyrrolo[3,2-c]quinoline-3-carboxamide;
3-(1-(2-Azabicyclo[2.1.1]hexan-5-yl)-6-fluoro-3-(hydroxymethyl)-7-(3-hydroxynaphthalen-1-yl)-4-(((S)-1-methylpyrrolidin-2-yl)methoxy)-1H-pyrrolo[3,2-c]quinolin-8-yl)propanenitrile;
2-((2S,4S)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)-1-((E)-4-methoxybut-2-enoyl)piperidin-2-yl)acetonitrile;
3-(1-((1R,4R,5S)-2-azabicyclo[2.1.1]hexan-5-yl)-8-(2-cyanoethyl)-4-ethoxy-6-fluoro-7-(3-hydroxynaphthalen-1-yl)-1H-pyrrolo[3,2-c]quinolin-2-yl)-N,N-dimethylpropanamide;
methyl 3-(1-((1R,4R,5S)-2-azabicyclo[2.1.1]hexan-5-yl)-8-(2-cyanoethyl)-3-(3-(dimethylamino)azetidin-1-yl)-6-fluoro-7-(3-hydroxynaphthalen-1-yl)-4-methoxy-1H-pyrrolo[3,2-c]quinolin-2-yl)propanoate;
3-(2-(3-(azetidin-1-yl)-3-oxopropyl)-1-(2-azabicyclo[2.1.1]hexan-5-yl)-6-fluoro-7-(7-fluoronaphthalen-1-yl)-4-(((S)-1-methylpyrrolidin-2-yl)methoxy)-1H-pyrrolo[3,2-c]quinolin-8-yl)propanenitrile;
8-(1-((2S,4S)-2-(cyanomethyl)-1-((E)-4-fluorobut-2-enoyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile;
8-(1-((2S,4S)-2-(cyanomethyl)-1-(2-fluoroacryloyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile;
8-(1-((2S,4S)-1-(but-2-ynoyl)-2-(cyanomethyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile;
8-(1-((2S,4S)-2-(cyanomethyl)-1-((E)-4-methoxybut-2-enoyl)piperidin-4-yl)-6-fluoro-8-methyl-4-(((S)-1-methylpyrrolidin-2-yl)methoxy)-1H-pyrazolo[4,3-c]quinolin-7-yl)-1-naphthonitrile;
8-(1-((2S,4S)-2-(cyanomethyl)-1-((E)-4-fluorobut-2-enoyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile;
8-(1-((2S,4S)-2-(cyanomethyl)-1-(2-fluoroacryloyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile;
8-(1-((2S,4S)-2-(cyanomethyl)-1-((E)-4-methoxybut-2-enoyl)piperidin-4-yl)-6-fluoro-8-methyl-4-((S)-1-((S)-1-methylpyrrolidin-2-yl)ethoxy)-1H-pyrazolo[4,3-c]quinolin-7-yl)-1-naphthonitrile;
8-(1-((2S,4S)-2-(cyanomethyl)-1-((E)-4-fluorobut-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
8-(1-((2S,4S)-2-(cyanomethyl)-1-(2-fluoroacryloyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
8-(1-((2S,4S)-1-(but-2-ynoyl)-2-(cyanomethyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
8-(1-((2S,4S)-2-(cyanomethyl)-1-((E)-4-methoxybut-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
8-(1-((2S,4S)-2-(cyanomethyl)-1-((E)-4-(dimethylamino)but-2-enoyl)piperidin-4-yl)-4-(3-(dimethylamino)-3-methylazetidin-1-yl)-6-fluoro-8-methyl-1H-pyrazolo[4,3-c]quinolin-7-yl)-1-naphthonitrile;
2-((2S,4S)-4-(8-chloro-7-(8-chloronaphthalen-1-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-fluorobut-2-enoyl)piperidin-2-yl)acetonitrile;
2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-methoxybut-2-enoyl)piperidin-2-yl)acetonitrile;
2-((2S,4S)-4-(7-(5,6-dimethyl-1H-indazol-4-yl)-4-(3-(dimethylamino)-3-methylazetidin-1-yl)-6-fluoro-8-methyl-1H-pyrazolo[4,3-c]quinolin-1-yl)-1-((E)-4-(dimethylamino)but-2-enoyl)piperidin-2-yl)acetonitrile;
2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-fluorobut-2-enoyl)piperidin-2-yl)acetonitrile;
2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-(2-fluoroacryloyl)piperidin-2-yl)acetonitrile;
2-((2S,4S)-1-(but-2-ynoyl)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)piperidin-2-yl)acetonitrile;
2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-methoxybut-2-enoyl)piperidin-2-yl)acetonitrile;
2-((2S,4S)-4-(7-(5,6-dimethyl-1H-indazol-4-yl)-6-fluoro-8-methyl-4-((S)-1-((S)-1-methylpyrrolidin-2-yl)ethoxy)-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-fluorobut-2-enoyl)piperidin-2-yl)acetonitrile;
2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)piperidin-2-yl)acetonitrile;
2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-methoxybut-2-enoyl)piperidin-2-yl)acetonitrile;
2-((2S,4S)-4-(8-chloro-7-(5,6-dimethyl-1H-indazol-4-yl)-4-(3-(dimethylamino)azetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)piperidin-2-yl)acetonitrile;
2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-((E)-4-methoxybut-2-enoyl)piperidin-2-yl)acetonitrile;
2-((2S,4S)-4-(8-chloro-7-(5,6-dimethyl-1H-indazol-4-yl)-6-fluoro-4-(((S)-1-methylpyrrolidin-2-yl)methoxy)-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)piperidin-2-yl)acetonitrile;
2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-((E)-4-methoxybut-2-enoyl)piperidin-2-yl)acetonitrile;
2-((2S,4S)-4-(8-chloro-7-(5,6-dimethyl-1H-indazol-4-yl)-4-(3-(dimethylamino)-3-methylazetidin-1-yl)-6-fluoro-1H-pyrazolo[4,3-c]quinolin-1-yl)-1-((E)-4-(dimethylamino)but-2-enoyl)piperidin-2-yl)acetonitrile;
2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-fluorobut-2-enoyl)piperidin-2-yl)acetonitrile;
2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-(2-fluoroacryloyl)piperidin-2-yl)acetonitrile;
2-((2S,4S)-1-(but-2-ynoyl)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)piperidin-2-yl)acetonitrile;
2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-methoxybut-2-enoyl)piperidin-2-yl)acetonitrile;
2-((2S,4S)-4-(8-chloro-7-(8-chloronaphthalen-1-yl)-6-fluoro-4-((S)-1-((S)-1-methylpyrrolidin-2-yl)ethoxy)-1H-pyrazolo[4,3-c]quinolin-1-yl)-1-((E)-4-(dimethylamino)but-2-enoyl)piperidin-2-yl)acetonitrile; and
2-((2S,4S)-1-(but-2-ynoyl)-4-(8-chloro-7-(5,6-dimethyl-1H-indazol-4-yl)-6-fluoro-4-(((S)-1-methylpyrrolidin-2-yl)methoxy)-1H-pyrrolo[3,2-c]quinolin-1-yl)piperidin-2-yl)acetonitrile;
or a pharmaceutically acceptable salt thereof.

46. A pharmaceutical composition comprising a compound of claim 1, or a pharmaceutically acceptable salt thereof, and at least one pharmaceutically acceptable carrier or excipient.

47. A method of inhibiting KRAS activity, said method comprising contacting a compound of claim 1, with KRAS.

48. The method of claim 47, wherein the contacting comprises administering the compound to a patient.

49. A method of treating a disease or disorder associated with inhibition of KRAS interaction, said method comprising administering to a patient in need thereof a therapeutically effective amount of a compound of claim 1.

50. The method of claim 49, wherein the disease or disorder is an immunological or inflammatory disorder.

51. The method of claim 50, wherein the immunological or inflammatory disorder is Ras-associated lymphoproliferative disorder and juvenile myelomonocytic leukemia caused by somatic mutations of KRAS.

52. A method for treating a cancer in a patient, said method comprising administering to the patient a therapeutically effective amount of the compound of claim 1.

53. The method of claim 52, wherein the cancer is selected from carcinomas, hematological cancers, sarcomas, and glioblastoma.

54. The method of claim 53, wherein the hematological cancer is selected from myeloproliferative neoplasms, myelodysplastic syndrome, chronic and juvenile myelomonocytic leukemia, acute myeloid leukemia, acute lymphocytic leukemia, and multiple myeloma.

55. The method of claim 53, wherein the carcinoma is selected from pancreatic, colorectal, lung, bladder, gastric, esophageal, breast, head and neck, cervical, skin, and thyroid.

56. A method of treating a disease or disorder associated with inhibiting a KRAS protein harboring a G12C mutation, said method comprising administering to a patient in need thereof a therapeutically effective amount of the compound of claim 1.
